SEQUENCE LISTING (504 sequences)

SEQ ID NO: 1 - protein, 12 aa, synthetic construct
  REGION 1..12: note = Description of Artificial Sequence: Syntheticpeptide
LLADTTHHRPWT

SEQ ID NO: 2 - protein, 12 aa, synthetic construct
  REGION 1..12: note = Description of Artificial Sequence: Syntheticpeptide
VIGESTHHRPWS

SEQ ID NO: 3 - protein, 12 aa, synthetic construct
  REGION 1..12: note = Description of Artificial Sequence: Syntheticpeptide
LIADSTHHSPWT

SEQ ID NO: 4 - protein, 12 aa, synthetic construct
  REGION 1..12: note = Description of Artificial Sequence: Syntheticpeptide
ILAESTHHKPWT

SEQ ID NO: 5 - protein, 12 aa, synthetic construct
  REGION 1..12: note = Description of Artificial Sequence: Syntheticpeptide
ILAETTHHRPWS

SEQ ID NO: 6 - protein, 12 aa, synthetic construct
  REGION 1..12: note = Description of Artificial Sequence: Syntheticpeptide
IIGESSHHKPFT

SEQ ID NO: 7 - protein, 12 aa, synthetic construct
  REGION 1..12: note = Description of Artificial Sequence: Syntheticpeptide
GLGDTTHHRPWG

SEQ ID NO: 8 - protein, 12 aa, synthetic construct
  REGION 1..12: note = Description of Artificial Sequence: Syntheticpeptide
VLGDTTHHKPWT

SEQ ID NO: 9 - protein, 12 aa, synthetic construct
  REGION 1..12: note = Description of Artificial Sequence: Syntheticpeptide
IVADSTHHRPWT

SEQ ID NO: 10 - protein, 11 aa, synthetic construct
  REGION 1..11: note = Description of Artificial Sequence: Syntheticpeptide
STADTSHHRPS

SEQ ID NO: 11 - protein, 12 aa, synthetic construct
  REGION 1..12: note = Description of Artificial Sequence: Syntheticpeptide
TSGGESTHHRPS

SEQ ID NO: 12 - protein, 12 aa, synthetic construct
  REGION 1..12: note = Description of Artificial Sequence: Syntheticpeptide
TSGGESSHHKPS

SEQ ID NO: 13 - protein, 12 aa, synthetic construct
  REGION 1..12: note = Description of Artificial Sequence: Syntheticpeptide
TGSGDSSHHRPS

SEQ ID NO: 14 - protein, 13 aa, synthetic construct
  REGION 1..13: note = Description of Artificial Sequence: Syntheticpeptide
GSSGESTHHKPST

SEQ ID NO: 15 - protein, 12 aa, synthetic construct
  REGION 1..12: note = Description of Artificial Sequence: Syntheticpeptide
VGADSTHHRPVT

SEQ ID NO: 16 - protein, 12 aa, synthetic construct
  REGION 1..12: note = Description of Artificial Sequence: Syntheticpeptide
GAADTTHHRPVT

SEQ ID NO: 17 - protein, 12 aa, synthetic construct
  REGION 1..12: note = Description of Artificial Sequence: Syntheticpeptide
AGADTTHHRPVT

SEQ ID NO: 18 - protein, 12 aa, synthetic construct
  REGION 1..12: note = Description of Artificial Sequence: Syntheticpeptide
GGADTTHHRPAT

SEQ ID NO: 19 - protein, 12 aa, synthetic construct
  REGION 1..12: note = Description of Artificial Sequence: Syntheticpeptide
GGADTTHHRPGT

SEQ ID NO: 20 - protein, 24 aa, synthetic construct
  REGION 1..24: note = Description of Artificial Sequence: Syntheticpeptide
LLADTTHHRPWTVIGESTHHRPWS

SEQ ID NO: 21 - protein, 36 aa, synthetic construct
  REGION 1..36: note = Description of Artificial Sequence: Syntheticpolypeptide
LLADTTHHRPWTVIGESTHHRPWSIIGESSHHKPFT

SEQ ID NO: 22 - protein, 60 aa, synthetic construct
  REGION 1..60: note = Description of Artificial Sequence: Syntheticpolypeptide
LLADTTHHRPWTVIGESTHHRPWSIIGESSHHKPFTGLGDTTHHRPWGILAESTHHKPWT

SEQ ID NO: 23 - protein, 24 aa, synthetic construct
  REGION 1..24: note = Description of Artificial Sequence: Syntheticpeptide
LLADTTHHRPWTILAESTHHKPWT

SEQ ID NO: 24 - protein, 60 aa, synthetic construct
  REGION 1..60: note = Description of Artificial Sequence: Syntheticpolypeptide
LLADTTHHRPWTILAESTHHKPWTLLADTTHHRPWTILAESTHHKPWTLLADTTHHRPWT

SEQ ID NO: 25 - protein, 24 aa, synthetic construct
  REGION 1..24: note = Description of Artificial Sequence: Syntheticpeptide
LLADTTHHRPWTGLGDTTHHRPWG

SEQ ID NO: 26 - protein, 36 aa, synthetic construct
  REGION 1..36: note = Description of Artificial Sequence: Syntheticpolypeptide
LLADTTHHRPWTGLGDTTHHRPWGLLADTTHHRPWT

SEQ ID NO: 27 - protein, 60 aa, synthetic construct
  REGION 1..60: note = Description of Artificial Sequence: Syntheticpolypeptide
LLADTTHHRPWTGLGDTTHHRPWGLLADTTHHRPWTGLGDTTHHRPWGLLADTTHHRPWT

SEQ ID NO: 28 - protein, 84 aa, synthetic construct
  REGION 1..84: note = Description of Artificial Sequence: Syntheticpolypeptide
LLADTTHHRPWTGLGDTTHHRPWGLLADTTHHRPWTGLGDTTHHRPWGLLADTTHHRPWTGLGDTTHHRPWGLLADTTHHRPWT

SEQ ID NO: 29 - protein, 60 aa, synthetic construct
  REGION 1..60: note = Description of Artificial Sequence: Syntheticpolypeptide
STADTSHHRPSTSGGESTHHRPSTSGGESSHHKPSTGSGDSSHHRPSGSSGESTHHKPST

SEQ ID NO: 30 - protein, 60 aa, synthetic construct
  REGION 1..60: note = Description of Artificial Sequence: Syntheticpolypeptide
VGADSTHHRPVTGAADTTHHRPVTAGADTTHHRPVTGGADTTHHRPATGGADTTHHRPGT

SEQ ID NO: 31 - protein, 108 aa, synthetic construct
  REGION 1..108: note = Description of Artificial Sequence: Syntheticpolypeptide
STADTSHHRPSLLADTTHHRPWTTSGGESTHHRPSVGADSTHHRPVTTSGGESSHHKPSGAADTTHHRPVTTGSGDSSHHRPSGSSGESTHHKPSTGGADTTHHRPAT

SEQ ID NO: 32 - protein, 12 aa, synthetic construct
  REGION 1..12: note = Description of Artificial Sequence: Syntheticpeptide
AAADTTHHRPWT

SEQ ID NO: 33 - protein, 60 aa, synthetic construct
  REGION 1..60: note = Description of Artificial Sequence: Syntheticpolypeptide
AAADTTHHRPWTAAADTTHHRPWTAAADTTHHRPWTAAADTTHHRPWTAAADTTHHRPWT

SEQ ID NO: 34 - protein, 60 aa, synthetic construct
  REGION 1..60: note = Description of Artificial Sequence: Syntheticpolypeptide
LLADAAHHRPWTLLADAAHHRPWTLLADAAHHRPWTLLADAAHHRPWTLLADAAHHRPWT

SEQ ID NO: 35 - protein, 60 aa, synthetic construct
  REGION 1..60: note = Description of Artificial Sequence: Syntheticpolypeptide
LLADTTAARPWTLLADTTAARPWTLLADTTAARPWTLLADTTAARPWTLLADTTAARPWT

SEQ ID NO: 36 - protein, 24 aa, synthetic construct
  REGION 1..24: note = Description of Artificial Sequence: Syntheticpeptide
LLADTTHHRPWTLLADTTHHRPWT

SEQ ID NO: 37 - protein, 36 aa, synthetic construct
  REGION 1..36: note = Description of Artificial Sequence: Syntheticpolypeptide
LLADTTHHRPWTLLADTTHHRPWTLLADTTHHRPWT

SEQ ID NO: 38 - protein, 60 aa, synthetic construct
  REGION 1..60: note = Description of Artificial Sequence: Syntheticpolypeptide
LLADTTHHRPWTLLADTTHHRPWTLLADTTHHRPWTLLADTTHHRPWTLLADTTHHRPWT

SEQ ID NO: 39 - protein, 120 aa, synthetic construct
  REGION 1..120: note = Description of Artificial Sequence: Syntheticpolypeptide
STSGSTVIGESTHHRPWSLIADSTHHSPWTILAESTHHKPWTILAETTHHRPWSIIGESSHHKPFTGLGDTTHHRPWGVLGDTTHHKPWTIVADSTHHRPWTGQVLPTTTPSSPSTTSGS

SEQ ID NO: 40 - protein, 48 aa, synthetic construct
  REGION 1..48: note = Description of Artificial Sequence: Syntheticpolypeptide
LLADTTHHRPWTVIGESTHHRPWSIIGESSHHKPFTGLGDTTHHRPWG

SEQ ID NO: 41 - protein, 48 aa, synthetic construct
  REGION 1..48: note = Description of Artificial Sequence: Syntheticpolypeptide
VIGESTHHRPWSIIGESSHHKPFTGLGDTTHHRPWGILAESTHHKPWT

SEQ ID NO: 42 - protein, 132 aa, synthetic construct
  VARIANT 13..132: note = May be "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS" or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLA DTTHHRPWTILAESTHHKPWTLLADTTHHRPWT" or "LLADTTHHRPWTGLGDTTHHRPWG" or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or
  REGION 1..132: note = Description of Artificial Sequence: Syntheticpolypeptide
  VARIANT 13..132: note = (continued) "LLADTTHHRPWTVIGESTHHRPWSIIGESSHHKPFTGLGDTTHHRPWG" or
  VARIANT 13..132: note = (continued) "LLADTTHHRPWTGLGDTTHHRPWGLLADTTHHRPWTGL GDTTHHRPWGLLADTTHHRPWTGLGDTTHHRPWGLLADTTHHRPWT" or "STADTSHHRPSTSGGESTHHRPSTSGGESSHHKPSTGSGDSSHHRPSGSSGESTHHK PST" or "VGADSTHHRPVTGAADTTHHRPVTAGADTTHHRPVTGGADTTHHRPATG GADTTHHRPGT" or "STADTSHHRPSLLADTTHHRPWTTSGGESTHHRPSVGADST HHRPVTTSGGESSHHKPSGAADTTHHRPVTTGSGDSSHHRPSGSSGESTHHKPSTGGA DTTHHRPAT" or "AAADTTHHRPWT" or "AAADTTHHRPWTAAADTTHHRPWTA AADTTHHRPWTAAADTTHHRPWTAAADTTHHRPWT" or "LLADAAHHRPWTLLADA AHHRPWTLLADAAHHRPWTLLADAAHHRPWTLLADAAHHRPWT" or "LLADTTAAR PWTLLADTTAARPWTLLADTTAARPWTLLADTTAARPWTLLADTTAARPWT" or "LLADTTHHRPWTLLADTTHHRPWT" or "LLADTTHHRPWTLLADTTHHRPWTLLADTTHHRPWT" or "LLADTTHHRPWTLLA DTTHHRPWTLLADTTHHRPWTLLADTTHHRPWTLLADTTHHRPWT" or "STSGSTV IGESTHHRPWSLIADSTHHSPWTILAESTHHKPWTILAETTHHRPWSIIGESSHHKPF TGLGDTTHHRPWGVLGDTTHHKPWTIVADSTHHRPWTGQVLPTTTPSSPSTTSGS" or
  VARIANT 13..132: note = (continued) "VIGESTHHRPWSIIGESSHHKPFTGLGDTTHHRPWGILAESTHHKPWT"
LLADTTHHRPWTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 43 - protein, 132 aa, synthetic construct
  VARIANT 13..132: note = May be "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS" or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLA DTTHHRPWTILAESTHHKPWTLLADTTHHRPWT" or "LLADTTHHRPWTGLGDTTHHRPWG" or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or
  VARIANT 13..132: note = (continued) "VIGESTHHRPWSIIGESSHHKPFTGLGDTTHHRPWGILAESTHHKPWT"
  REGION 1..132: note = Description of Artificial Sequence: Syntheticpolypeptide
  VARIANT 13..132: note = (continued) "LLADTTHHRPWTGLGDTTHHRPWGLLADTTHHRPWTGL GDTTHHRPWGLLADTTHHRPWTGLGDTTHHRPWGLLADTTHHRPWT" or "STADTSHHRPSTSGGESTHHRPSTSGGESSHHKPSTGSGDSSHHRPSGSSGESTHHK PST" or "VGADSTHHRPVTGAADTTHHRPVTAGADTTHHRPVTGGADTTHHRPATG GADTTHHRPGT" or "STADTSHHRPSLLADTTHHRPWTTSGGESTHHRPSVGADST HHRPVTTSGGESSHHKPSGAADTTHHRPVTTGSGDSSHHRPSGSSGESTHHKPSTGGA DTTHHRPAT" or "AAADTTHHRPWT" or "AAADTTHHRPWTAAADTTHHRPWTA AADTTHHRPWTAAADTTHHRPWTAAADTTHHRPWT" or "LLADAAHHRPWTLLADA AHHRPWTLLADAAHHRPWTLLADAAHHRPWTLLADAAHHRPWT" or "LLADTTAAR PWTLLADTTAARPWTLLADTTAARPWTLLADTTAARPWTLLADTTAARPWT" or "LLADTTHHRPWTLLADTTHHRPWT" or "LLADTTHHRPWTLLADTTHHRPWTLLADTTHHRPWT" or "LLADTTHHRPWTLLA DTTHHRPWTLLADTTHHRPWTLLADTTHHRPWTLLADTTHHRPWT" or "STSGSTV IGESTHHRPWSLIADSTHHSPWTILAESTHHKPWTILAETTHHRPWSIIGESSHHKPF TGLGDTTHHRPWGVLGDTTHHKPWTIVADSTHHRPWTGQVLPTTTPSSPSTTSGS" or
  VARIANT 13..132: note = (continued) "LLADTTHHRPWTVIGESTHHRPWSIIGESSHHKPFTGLGDTTHHRPWG" or
VIGESTHHRPWSXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 44 - protein, 132 aa, synthetic construct
  VARIANT 13..132: note = May be "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS" or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLA DTTHHRPWTILAESTHHKPWTLLADTTHHRPWT" or "LLADTTHHRPWTGLGDTTHHRPWG" or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or
  REGION 1..132: note = Description of Artificial Sequence: Syntheticpolypeptide
  VARIANT 13..132: note = (continued) "LLADTTHHRPWTVIGESTHHRPWSIIGESSHHKPFTGLGDTTHHRPWG" or
  VARIANT 13..132: note = (continued) "LLADTTHHRPWTGLGDTTHHRPWGLLADTTHHRPWTGL GDTTHHRPWGLLADTTHHRPWTGLGDTTHHRPWGLLADTTHHRPWT" or "STADTSHHRPSTSGGESTHHRPSTSGGESSHHKPSTGSGDSSHHRPSGSSGESTHHK PST" or "VGADSTHHRPVTGAADTTHHRPVTAGADTTHHRPVTGGADTTHHRPATG GADTTHHRPGT" or "STADTSHHRPSLLADTTHHRPWTTSGGESTHHRPSVGADST HHRPVTTSGGESSHHKPSGAADTTHHRPVTTGSGDSSHHRPSGSSGESTHHKPSTGGA DTTHHRPAT" or "AAADTTHHRPWT" or "AAADTTHHRPWTAAADTTHHRPWTA AADTTHHRPWTAAADTTHHRPWTAAADTTHHRPWT" or "LLADAAHHRPWTLLADA AHHRPWTLLADAAHHRPWTLLADAAHHRPWTLLADAAHHRPWT" or "LLADTTAAR PWTLLADTTAARPWTLLADTTAARPWTLLADTTAARPWTLLADTTAARPWT" or "LLADTTHHRPWTLLADTTHHRPWT" or "LLADTTHHRPWTLLADTTHHRPWTLLADTTHHRPWT" or "LLADTTHHRPWTLLA DTTHHRPWTLLADTTHHRPWTLLADTTHHRPWTLLADTTHHRPWT" or "STSGSTV IGESTHHRPWSLIADSTHHSPWTILAESTHHKPWTILAETTHHRPWSIIGESSHHKPF TGLGDTTHHRPWGVLGDTTHHKPWTIVADSTHHRPWTGQVLPTTTPSSPSTTSGS" or
  VARIANT 13..132: note = (continued) "VIGESTHHRPWSIIGESSHHKPFTGLGDTTHHRPWGILAESTHHKPWT"
ILAESTHHKPWTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 45 - protein, 132 aa, synthetic construct
  VARIANT 13..132: note = May be "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS" or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLA DTTHHRPWTILAESTHHKPWTLLADTTHHRPWT" or "LLADTTHHRPWTGLGDTTHHRPWG" or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or
  VARIANT 13..132: note = (continued) "LLADTTHHRPWTVIGESTHHRPWSIIGESSHHKPFTGLGDTTHHRPWG" or
  VARIANT 13..132: note = (continued) "VIGESTHHRPWSIIGESSHHKPFTGLGDTTHHRPWGILAESTHHKPWT"
  REGION 1..132: note = Description of Artificial Sequence: Syntheticpolypeptide
  VARIANT 13..132: note = (continued) "LLADTTHHRPWTGLGDTTHHRPWGLLADTTHHRPWTGL GDTTHHRPWGLLADTTHHRPWTGLGDTTHHRPWGLLADTTHHRPWT" or "STADTSHHRPSTSGGESTHHRPSTSGGESSHHKPSTGSGDSSHHRPSGSSGESTHHK PST" or "VGADSTHHRPVTGAADTTHHRPVTAGADTTHHRPVTGGADTTHHRPATG GADTTHHRPGT" or "STADTSHHRPSLLADTTHHRPWTTSGGESTHHRPSVGADST HHRPVTTSGGESSHHKPSGAADTTHHRPVTTGSGDSSHHRPSGSSGESTHHKPSTGGA DTTHHRPAT" or "AAADTTHHRPWT" or "AAADTTHHRPWTAAADTTHHRPWTA AADTTHHRPWTAAADTTHHRPWTAAADTTHHRPWT" or "LLADAAHHRPWTLLADA AHHRPWTLLADAAHHRPWTLLADAAHHRPWTLLADAAHHRPWT" or "LLADTTAAR PWTLLADTTAARPWTLLADTTAARPWTLLADTTAARPWTLLADTTAARPWT" or "LLADTTHHRPWTLLADTTHHRPWT" or "LLADTTHHRPWTLLADTTHHRPWTLLADTTHHRPWT" or "LLADTTHHRPWTLLA DTTHHRPWTLLADTTHHRPWTLLADTTHHRPWTLLADTTHHRPWT" or "STSGSTV IGESTHHRPWSLIADSTHHSPWTILAESTHHKPWTILAETTHHRPWSIIGESSHHKPF TGLGDTTHHRPWGVLGDTTHHKPWTIVADSTHHRPWTGQVLPTTTPSSPSTTSGS" or
IIGESSHHKPFTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 46 - protein, 132 aa, synthetic construct
  VARIANT 13..132: note = (continued) "LLADTTHHRPWTGLGDTTHHRPWGLLADTTHHRPWTGL GDTTHHRPWGLLADTTHHRPWTGLGDTTHHRPWGLLADTTHHRPWT" or "STADTSHHRPSTSGGESTHHRPSTSGGESSHHKPSTGSGDSSHHRPSGSSGESTHHK PST" or "VGADSTHHRPVTGAADTTHHRPVTAGADTTHHRPVTGGADTTHHRPATG GADTTHHRPGT" or "STADTSHHRPSLLADTTHHRPWTTSGGESTHHRPSVGADST HHRPVTTSGGESSHHKPSGAADTTHHRPVTTGSGDSSHHRPSGSSGESTHHKPSTGGA DTTHHRPAT" or "AAADTTHHRPWT" or "AAADTTHHRPWTAAADTTHHRPWTA AADTTHHRPWTAAADTTHHRPWTAAADTTHHRPWT" or "LLADAAHHRPWTLLADA AHHRPWTLLADAAHHRPWTLLADAAHHRPWTLLADAAHHRPWT" or "LLADTTAAR PWTLLADTTAARPWTLLADTTAARPWTLLADTTAARPWTLLADTTAARPWT" or "LLADTTHHRPWTLLADTTHHRPWT" or "LLADTTHHRPWTLLADTTHHRPWTLLADTTHHRPWT" or "LLADTTHHRPWTLLA DTTHHRPWTLLADTTHHRPWTLLADTTHHRPWTLLADTTHHRPWT" or "STSGSTV IGESTHHRPWSLIADSTHHSPWTILAESTHHKPWTILAETTHHRPWSIIGESSHHKPF TGLGDTTHHRPWGVLGDTTHHKPWTIVADSTHHRPWTGQVLPTTTPSSPSTTSGS" or
  VARIANT 13..132: note = May be "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS" or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLA DTTHHRPWTILAESTHHKPWTLLADTTHHRPWT" or "LLADTTHHRPWTGLGDTTHHRPWG" or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or
  REGION 1..132: note = Description of Artificial Sequence: Syntheticpolypeptide
  VARIANT 13..132: note = (continued) "LLADTTHHRPWTVIGESTHHRPWSIIGESSHHKPFTGLGDTTHHRPWG" or
  VARIANT 13..132: note = (continued) "VIGESTHHRPWSIIGESSHHKPFTGLGDTTHHRPWGILAESTHHKPWT"
GLGDTTHHRPWGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 47 - protein, 24 aa, synthetic construct
  REGION 1..24: note = Description of Artificial Sequence: Syntheticpeptide
  VARIANT 13..24: note = This region may encompass one of the followingsequences: "VIGESTHHRPWS" or "ILAESTHHKPWT" or "IIGESSHHKPFT"or "GLGDTTHHRPWG"
LLADTTHHRPWTXXXXXXXXXXXX

SEQ ID NO: 48 - protein, 24 aa, synthetic construct
  VARIANT 13..24: note = This region may encompass one of the followingsequences: "LLADTTHHRPWT" or "ILAESTHHKPWT" or "IIGESSHHKPFT"or "GLGDTTHHRPWG"
  REGION 1..24: note = Description of Artificial Sequence: Syntheticpeptide
VIGESTHHRPWSXXXXXXXXXXXX

SEQ ID NO: 49 - protein, 24 aa, synthetic construct
  REGION 1..24: note = Description of Artificial Sequence: Syntheticpeptide
  VARIANT 13..24: note = This region may encompass one of the followingsequences: "LLADTTHHRPWT" or "VIGESTHHRPWS" or "IIGESSHHKPFT"or "GLGDTTHHRPWG"
ILAESTHHKPWTXXXXXXXXXXXX

SEQ ID NO: 50 - protein, 24 aa, synthetic construct
  REGION 1..24: note = Description of Artificial Sequence: Syntheticpeptide
  VARIANT 13..24: note = This region may encompass one of the followingsequences: "LLADTTHHRPWT" or "ILAESTHHKPWT" or "VIGESTHHRPWS"or "GLGDTTHHRPWG"
IIGESSHHKPFTXXXXXXXXXXXX

SEQ ID NO: 51 - protein, 24 aa, synthetic construct
  REGION 1..24: note = Description of Artificial Sequence: Syntheticpeptide
  VARIANT 13..24: note = This region may encompass one of the followingsequences: "LLADTTHHRPWT" or "ILAESTHHKPWT" or "IIGESSHHKPFT"or "VIGESTHHRPWS"
GLGDTTHHRPWGXXXXXXXXXXXX

SEQ ID NO: 52 - protein, 36 aa, synthetic construct
  VARIANT 13..36: note = This region may encompass two of the followingsequences: "VIGESTHHRPWS" or "ILAESTHHKPWT" or "IIGESSHHKPFT"or "GLGDTTHHRPWG"
  REGION 1..36: note = Description of Artificial Sequence: Syntheticpolypeptide
LLADTTHHRPWTXXXXXXXXXXXXXXXXXXXXXXXX

SEQ ID NO: 53 - protein, 36 aa, synthetic construct
  REGION 1..36: note = Description of Artificial Sequence: Syntheticpolypeptide
  VARIANT 13..36: note = This region may encompass two of the followingsequences: "LLADTTHHRPWT" or "ILAESTHHKPWT" or "IIGESSHHKPFT"or "GLGDTTHHRPWG"
VIGESTHHRPWSXXXXXXXXXXXXXXXXXXXXXXXX

SEQ ID NO: 54 - protein, 36 aa, synthetic construct
  REGION 1..36: note = Description of Artificial Sequence: Syntheticpolypeptide
  VARIANT 13..36: note = This region may encompass two of the followingsequences: "LLADTTHHRPWT" or "VIGESTHHRPWS" or "IIGESSHHKPFT"or "GLGDTTHHRPWG"
ILAESTHHKPWTXXXXXXXXXXXXXXXXXXXXXXXX

SEQ ID NO: 55 - protein, 36 aa, synthetic construct
  REGION 1..36: note = Description of Artificial Sequence: Syntheticpolypeptide
  VARIANT 13..36: note = This region may encompass two of the followingsequences: "LLADTTHHRPWT" or "ILAESTHHKPWT" or "VIGESTHHRPWS"or "GLGDTTHHRPWG"
IIGESSHHKPFTXXXXXXXXXXXXXXXXXXXXXXXX

SEQ ID NO: 56 - protein, 36 aa, synthetic construct
  REGION 1..36: note = Description of Artificial Sequence: Syntheticpolypeptide
  VARIANT 13..36: note = This region may encompass two of the followingsequences: "LLADTTHHRPWT" or "ILAESTHHKPWT" or "IIGESSHHKPFT"or "VIGESTHHRPWS"
GLGDTTHHRPWGXXXXXXXXXXXXXXXXXXXXXXXX

SEQ ID NO: 57 - protein, 48 aa, synthetic construct
  REGION 1..48: note = Description of Artificial Sequence: Syntheticpolypeptide
  VARIANT 13..48: note = This region may encompass three of the followingsequences: "VIGESTHHRPWS" or "ILAESTHHKPWT" or "IIGESSHHKPFT"or "GLGDTTHHRPWG"
LLADTTHHRPWTXXXXXXXXXXXXXXXXXXXXXXXXXXXXXXXXXXXX

SEQ ID NO: 58 - protein, 48 aa, synthetic construct
  VARIANT 13..48: note = This region may encompass three of the followingsequences: "LLADTTHHRPWT" or "ILAESTHHKPWT" or "IIGESSHHKPFT"or "GLGDTTHHRPWG"
  REGION 1..48: note = Description of Artificial Sequence: Syntheticpolypeptide
VIGESTHHRPWSXXXXXXXXXXXXXXXXXXXXXXXXXXXXXXXXXXXX

SEQ ID NO: 59 - protein, 48 aa, synthetic construct
  REGION 1..48: note = Description of Artificial Sequence: Syntheticpolypeptide
  VARIANT 13..48: note = This region may encompass three of the followingsequences: "LLADTTHHRPWT" or "VIGESTHHRPWS" or "IIGESSHHKPFT"or "GLGDTTHHRPWG"
ILAESTHHKPWTXXXXXXXXXXXXXXXXXXXXXXXXXXXXXXXXXXXX

SEQ ID NO: 60 - protein, 48 aa, synthetic construct
  REGION 1..48: note = Description of Artificial Sequence: Syntheticpolypeptide
  VARIANT 13..48: note = This region may encompass three of the followingsequences: "LLADTTHHRPWT" or "ILAESTHHKPWT" or "VIGESTHHRPWS"or "GLGDTTHHRPWG"
IIGESSHHKPFTXXXXXXXXXXXXXXXXXXXXXXXXXXXXXXXXXXXX

SEQ ID NO: 61 - protein, 48 aa, synthetic construct
  REGION 1..48: note = Description of Artificial Sequence: Syntheticpolypeptide
  VARIANT 13..48: note = This region may encompass three of the followingsequences: "LLADTTHHRPWT" or "ILAESTHHKPWT" or "IIGESSHHKPFT"or "VIGESTHHRPWS"
GLGDTTHHRPWGXXXXXXXXXXXXXXXXXXXXXXXXXXXXXXXXXXXX

SEQ ID NO: 62 - protein, 10 aa, synthetic construct
  REGION 1..10: note = Description of Artificial Sequence: Syntheticpeptide
ACAPLMFSQC

SEQ ID NO: 63 - protein, 10 aa, synthetic construct
  REGION 1..10: note = Description of Artificial Sequence: Syntheticpeptide
ACHASLKHRC

SEQ ID NO: 64 - protein, 10 aa, synthetic construct
  REGION 1..10: note = Description of Artificial Sequence: Syntheticpeptide
ACLSTKTNIC

SEQ ID NO: 65 - protein, 10 aa, synthetic construct
  REGION 1..10: note = Description of Artificial Sequence: Syntheticpeptide
ACTTPSKHQC

SEQ ID NO: 66 - protein, 11 aa, synthetic construct
  REGION 1..11: note = Description of Artificial Sequence: Syntheticpeptide
AHFSPNLLLGG

SEQ ID NO: 67 - protein, 12 aa, synthetic construct
  REGION 1..12: note = Description of Artificial Sequence: Syntheticpeptide
AHSLKSITNHGL

SEQ ID NO: 68 - protein, 7 aa, synthetic construct
  REGION 1..7: note = Description of Artificial Sequence: Syntheticpeptide
AKQTVPV

SEQ ID NO: 69 - protein, 12 aa, synthetic construct
  REGION 1..12: note = Description of Artificial Sequence: Syntheticpeptide
AKTLMPSPFPRT

SEQ ID NO: 70 - protein, 12 aa, synthetic construct
  REGION 1..12: note = Description of Artificial Sequence: Syntheticpeptide
AMSQTMTAAIEK

SEQ ID NO: 71 - protein, 7 aa, synthetic construct
  REGION 1..7: note = Description of Artificial Sequence: Syntheticpeptide
ANPPLSL

SEQ ID NO: 72 - protein, 7 aa, synthetic construct
  REGION 1..7: note = Description of Artificial Sequence: Syntheticpeptide
ANPYHRH

SEQ ID NO: 73 - protein, 7 aa, synthetic construct
  REGION 1..7: note = Description of Artificial Sequence: Syntheticpeptide
APLSLSL

SEQ ID NO: 74 - protein, 16 aa, synthetic construct
  REGION 1..16: note = Description of Artificial Sequence: Syntheticpeptide
APYHPTIPASVHGGGK

SEQ ID NO: 75 - protein, 12 aa, synthetic construct
  VARIANT 12: note = Any amino acid
  REGION 1..12: note = Description of Artificial Sequence: Syntheticpeptide
ASAVGSLSIRWX

SEQ ID NO: 76 - protein, 7 aa, synthetic construct
  REGION 1..7: note = Description of Artificial Sequence: Syntheticpeptide
ASGPTNV

SEQ ID NO: 77 - protein, 7 aa, synthetic construct
  REGION 1..7: note = Description of Artificial Sequence: Syntheticpeptide
ASHNPKL

SEQ ID NO: 78 - protein, 12 aa, synthetic construct
  REGION 1..12: note = Description of Artificial Sequence: Syntheticpeptide
ASWVDSRQPSAA

SEQ ID NO: 79 - protein, 7 aa, synthetic construct
  REGION 1..7: note = Description of Artificial Sequence: Syntheticpeptide
ATFSPPL

SEQ ID NO: 80 - protein, 12 aa, synthetic construct
  REGION 1..12: note = Description of Artificial Sequence: Syntheticpeptide
ATWSHHLSSAGL

SEQ ID NO: 81 - protein, 16 aa, synthetic construct
  REGION 1..16: note = Description of Artificial Sequence: Syntheticpeptide
ATWSHHLSSAGLGGGS

SEQ ID NO: 82 - protein, 9 aa, synthetic construct
  REGION 1..9: note = Description of Artificial Sequence: Syntheticpeptide
CAHLSPHKC

SEQ ID NO: 83 - protein, 9 aa, synthetic construct
  REGION 1..9: note = Description of Artificial Sequence: Syntheticpeptide
CDIPWRNEC

SEQ ID NO: 84 - protein, 9 aa, synthetic construct
  REGION 1..9: note = Description of Artificial Sequence: Syntheticpeptide
CDPLRQHSC

SEQ ID NO: 85 - protein, 9 aa, synthetic construct
  REGION 1..9: note = Description of Artificial Sequence: Syntheticpeptide
CDSLGHWLC

SEQ ID NO: 86 - protein, 9 aa, synthetic construct
  REGION 1..9: note = Description of Artificial Sequence: Syntheticpeptide
CDYTTRHSC

SEQ ID NO: 87 - protein, 9 aa, synthetic construct
  REGION 1..9: note = Description of Artificial Sequence: Syntheticpeptide
CHGTLNPEC

SEQ ID NO: 88 - protein, 9 aa, synthetic construct
  REGION 1..9: note = Description of Artificial Sequence: Syntheticpeptide
CHHNLSWEC

SEQ ID NO: 89 - protein, 9 aa, synthetic construct
  REGION 1..9: note = Description of Artificial Sequence: Syntheticpeptide
CHIWTLASC

SEQ ID NO: 90 - protein, 9 aa, synthetic construct
  REGION 1..9: note = Description of Artificial Sequence: Syntheticpeptide
CHNTFSPRC

SEQ ID NO: 91 - protein, 9 aa, synthetic construct
  REGION 1..9: note = Description of Artificial Sequence: Syntheticpeptide
CIPLHASLC

SEQ ID NO: 92 - protein, 9 aa, synthetic construct
  REGION 1..9: note = Description of Artificial Sequence: Syntheticpeptide
CITTTSLSC

SEQ ID NO: 93 - protein, 9 aa, synthetic construct
  REGION 1..9: note = Description of Artificial Sequence: Syntheticpeptide
CKLTTCKDC

SEQ ID NO: 94 - protein, 9 aa, synthetic construct
  REGION 1..9: note = Description of Artificial Sequence: Syntheticpeptide
CKNHTTFWC

SEQ ID NO: 95 - protein, 9 aa, synthetic construct
  REGION 1..9: note = Description of Artificial Sequence: Syntheticpeptide
CLKLLSRSC

SEQ ID NO: 96 - protein, 9 aa, synthetic construct
  REGION 1..9: note = Description of Artificial Sequence: Syntheticpeptide
CLLKAHPSC

SEQ ID NO: 97 - protein, 9 aa, synthetic construct
  REGION 1..9: note = Description of Artificial Sequence: Syntheticpeptide
CLNQLKQAC

SEQ ID NO: 98 - protein, 9 aa, synthetic construct
  REGION 1..9: note = Description of Artificial Sequence: Syntheticpeptide
CLSTKTNIC

SEQ ID NO: 99 - protein, 9 aa, synthetic construct
  REGION 1..9: note = Description of Artificial Sequence: Syntheticpeptide
CMNFPSPHC

SEQ ID NO: 100 - protein, 9 aa, synthetic construct
  REGION 1..9: note = Description of Artificial Sequence: Syntheticpeptide
CNYPTLKSC

SEQ ID NO: 101 - protein, 11 aa, synthetic construct
  REGION 1..11: note = Description of Artificial Sequence: Syntheticpeptide
CPQLTVGQHRT

SEQ ID NO: 102 - protein, 9 aa, synthetic construct
  REGION 1..9: note = Description of Artificial Sequence: Syntheticpeptide
CPQSPTYTC

SEQ ID NO: 103 - protein, 9 aa, synthetic construct
  REGION 1..9: note = Description of Artificial Sequence: Syntheticpeptide
CPSSAIHTC

SEQ ID NO: 104 - protein, 9 aa, synthetic construct
  REGION 1..9: note = Description of Artificial Sequence: Syntheticpeptide
CPTSTARIC

SEQ ID NO: 105 - protein, 9 aa, synthetic construct
  REGION 1..9: note = Description of Artificial Sequence: Syntheticpeptide
CQASSFPSC

SEQ ID NO: 106 - protein, 9 aa, synthetic construct
  REGION 1..9: note = Description of Artificial Sequence: Syntheticpeptide
CQPYFWYRC

SEQ ID NO: 107 - protein, 9 aa, synthetic construct
  REGION 1..9: note = Description of Artificial Sequence: Syntheticpeptide
CQTLTPSIC

SEQ ID NO: 108 - protein, 9 aa, synthetic construct
  REGION 1..9: note = Description of Artificial Sequence: Syntheticpeptide
CSKLGHLWC

SEQ ID NO: 109 - protein, 9 aa, synthetic construct
  REGION 1..9: note = Description of Artificial Sequence: Syntheticpeptide
  VARIANT 9: note = Any amino acid
CSKTPERIX

SEQ ID NO: 110 - protein, 9 aa, synthetic construct
  REGION 1..9: note = Description of Artificial Sequence: Syntheticpeptide
CSNNNRMTC

SEQ ID NO: 111 - protein, 9 aa, synthetic construct
  REGION 1..9: note = Description of Artificial Sequence: Syntheticpeptide
CSPILSLSC

SEQ ID NO: 112 - protein, 9 aa, synthetic construct
  REGION 1..9: note = Description of Artificial Sequence: Syntheticpeptide
CSPTNFTRC

SEQ ID NO: 113 - protein, 9 aa, synthetic construct
  REGION 1..9: note = Description of Artificial Sequence: Syntheticpeptide
CSRPAMNVC

SEQ ID NO: 114 - protein, 9 aa, synthetic construct
  REGION 1..9: note = Description of Artificial Sequence: Syntheticpeptide
CSTKAYPNC

SEQ ID NO: 115 - protein, 9 aa, synthetic construct
  REGION 1..9: note = Description of Artificial Sequence: Syntheticpeptide
CSTSSCGSC

SEQ ID NO: 116 - protein, 9 aa, synthetic construct
  REGION 1..9: note = Description of Artificial Sequence: Syntheticpeptide
CSYWGHRDC

SEQ ID NO: 117 - protein, 9 aa, synthetic construct
  REGION 1..9: note = Description of Artificial Sequence: Syntheticpeptide
CTAHDANAC

SEQ ID NO: 118 - protein, 9 aa, synthetic construct
  REGION 1..9: note = Description of Artificial Sequence: Syntheticpeptide
CTANSEKTC

SEQ ID NO: 119 - protein, 9 aa, synthetic construct
  REGION 1..9: note = Description of Artificial Sequence: Syntheticpeptide
CTHPKASMC

SEQ ID NO: 120 - protein, 9 aa, synthetic construct
  REGION 1..9: note = Description of Artificial Sequence: Syntheticpeptide
CTKTINGKC

SEQ ID NO: 121 - protein, 9 aa, synthetic construct
  REGION 1..9: note = Description of Artificial Sequence: Syntheticpeptide
CTNMQSPLC

SEQ ID NO: 122 - protein, 9 aa, synthetic construct
  REGION 1..9: note = Description of Artificial Sequence: Syntheticpeptide
CTPFTKLPC

SEQ ID NO: 123 - protein, 9 aa, synthetic construct
  REGION 1..9: note = Description of Artificial Sequence: Syntheticpeptide
CTPTTDSIC

SEQ ID NO: 124 - protein, 9 aa, synthetic construct
  REGION 1..9: note = Description of Artificial Sequence: Syntheticpeptide
CTQQNGHPC

SEQ ID NO: 125 - protein, 9 aa, synthetic construct
  REGION 1..9: note = Description of Artificial Sequence: Syntheticpeptide
CTTPSKHQC

SEQ ID NO: 126 - protein, 9 aa, synthetic construct
  REGION 1..9: note = Description of Artificial Sequence: Syntheticpeptide
CTYNVAKPC

SEQ ID NO: 127 - protein, 7 aa, synthetic construct
  REGION 1..7: note = Description of Artificial Sequence: Syntheticpeptide
DKLHRLA

SEQ ID NO: 128 - protein, 12 aa, synthetic construct
  REGION 1..12: note = Description of Artificial Sequence: Syntheticpeptide
DLNYFTLSSKRE

SEQ ID NO: 129 - protein, 12 aa, synthetic construct
  REGION 1..12: note = Description of Artificial Sequence: Syntheticpeptide
DLPPTLHTTGSP

SEQ ID NO: 130 - protein, 7 aa, synthetic construct
  REGION 1..7: note = Description of Artificial Sequence: Syntheticpeptide
DMRQQRS

SEQ ID NO: 131 - protein, 7 aa, synthetic construct
  REGION 1..7: note = Description of Artificial Sequence: Syntheticpeptide
DQYWGLR

SEQ ID NO: 132 - protein, 12 aa, synthetic construct
  REGION 1..12: note = Description of Artificial Sequence: Syntheticpeptide
DSSNPIFWRPSS

SEQ ID NO: 133 - protein, 16 aa, synthetic construct
  REGION 1..16: note = Description of Artificial Sequence: Syntheticpeptide
DSSNPIFWRPSSGGGS

SEQ ID NO: 134 - protein, 12 aa, synthetic construct
  REGION 1..12: note = Description of Artificial Sequence: Syntheticpeptide
EFLGVPASLVNP

SEQ ID NO: 135 - protein, 7 aa, synthetic construct
  REGION 1..7: note = Description of Artificial Sequence: Syntheticpeptide
EPNHTRF

SEQ ID NO: 136 - protein, 9 aa, synthetic construct
  REGION 1..9: note = Description of Artificial Sequence: Syntheticpeptide
EPRRAVAAL

SEQ ID NO: 137 - protein, 9 aa, synthetic construct
  REGION 1..9: note = Description of Artificial Sequence: Syntheticpeptide
EPRRAVAEL

SEQ ID NO: 138 - protein, 9 aa, synthetic construct
  REGION 1..9: note = Description of Artificial Sequence: Syntheticpeptide
EPRREVAEL

SEQ ID NO: 139 - protein, 9 aa, synthetic construct
  REGION 1..9: note = Description of Artificial Sequence: Syntheticpeptide
EPRREVCEL

SEQ ID NO: 140 - protein, 12 aa, synthetic construct
  REGION 1..12: note = Description of Artificial Sequence: Syntheticpeptide
ESDLTHALHWLG

SEQ ID NO: 141 - protein, 7 aa, synthetic construct
  REGION 1..7: note = Description of Artificial Sequence: Syntheticpeptide
ESLKSIS

SEQ ID NO: 142 - protein, 7 aa, synthetic construct
  REGION 1..7: note = Description of Artificial Sequence: Syntheticpeptide
ETRTQLL

SEQ ID NO: 143 - protein, 7 aa, synthetic construct
  REGION 1..7: note = Description of Artificial Sequence: Syntheticpeptide
ETVCASS

SEQ ID NO: 144 - protein, 7 aa, synthetic construct
  REGION 1..7: note = Description of Artificial Sequence: Syntheticpeptide
ETYARPL

SEQ ID NO: 145 - protein, 7 aa, synthetic construct
  REGION 1..7: note = Description of Artificial Sequence: Syntheticpeptide
ETYQQPL

SEQ ID NO: 146 - protein, 12 aa, synthetic construct
  REGION 1..12: note = Description of Artificial Sequence: Syntheticpeptide
EVHSTDRYRSIP

SEQ ID NO: 147 - protein, 12 aa, synthetic construct
  REGION 1..12: note = Description of Artificial Sequence: Syntheticpeptide
FGLQPTGDIARR

SEQ ID NO: 148 - protein, 12 aa, synthetic construct
  REGION 1..12: note = Description of Artificial Sequence: Syntheticpeptide
FSMDDPERVRSP

SEQ ID NO: 149 - protein, 12 aa, synthetic construct
  REGION 1..12: note = Description of Artificial Sequence: Syntheticpeptide
FSPLHTSTYRPS

SEQ ID NO: 150 - protein, 7 aa, synthetic construct
  REGION 1..7: note = Description of Artificial Sequence: Syntheticpeptide
FTLPTIR

SEQ ID NO: 151 - protein, 7 aa, synthetic construct
  REGION 1..7: note = Description of Artificial Sequence: Syntheticpeptide
FVNLLGQ

SEQ ID NO: 152 - protein, 12 aa, synthetic construct
  REGION 1..12: note = Description of Artificial Sequence: Syntheticpeptide
GDFNSGHHTTTR

SEQ ID NO: 153 - protein, 7 aa, synthetic construct
  REGION 1..7: note = Description of Artificial Sequence: Syntheticpeptide
GGGAAAA

SEQ ID NO: 154 - protein, 12 aa, synthetic construct
  REGION 1..12: note = Description of Artificial Sequence: Syntheticpeptide
GIHVPWMPPVAF

SEQ ID NO: 155 - protein, 16 aa, synthetic construct
  REGION 1..16: note = Description of Artificial Sequence: Syntheticpeptide
GIHVPWMPPVAFGGGS

SEQ ID NO: 156 - protein, 12 aa, synthetic construct
  REGION 1..12: note = Description of Artificial Sequence: Syntheticpeptide
GPSNNLPWSNTP

SEQ ID NO: 157 - protein, 12 aa, synthetic construct
  REGION 1..12: note = Description of Artificial Sequence: Syntheticpeptide
GSAGLKYPLYKS

SEQ ID NO: 158 - protein, 7 aa, synthetic construct
  REGION 1..7: note = Description of Artificial Sequence: Syntheticpeptide
GSCPPKK

SEQ ID NO: 159 - protein, 7 aa, synthetic construct
  REGION 1..7: note = Description of Artificial Sequence: Syntheticpeptide
GSLFKAL

SEQ ID NO: 160 - protein, 7 aa, synthetic construct
  REGION 1..7: note = Description of Artificial Sequence: Syntheticpeptide
GTQTPQP

SEQ ID NO: 161 - protein, 7 aa, synthetic construct
  REGION 1..7: note = Description of Artificial Sequence: Syntheticpeptide
GTSRLFS

SEQ ID NO: 162 - protein, 12 aa, synthetic construct
  REGION 1..12: note = Description of Artificial Sequence: Syntheticpeptide
GVHKHFYSRWLG

SEQ ID NO: 163 - protein, 12 aa, synthetic construct
  REGION 1..12: note = Description of Artificial Sequence: Syntheticpeptide
HAPLTRSPAPNL

SEQ ID NO: 164 - protein, 7 aa, synthetic construct
  REGION 1..7: note = Description of Artificial Sequence: Syntheticpeptide
HAPVQPN

SEQ ID NO: 165 - protein, 12 aa, synthetic construct
  REGION 1..12: note = Description of Artificial Sequence: Syntheticpeptide
  VARIANT 8: note = Any amino acid
HGSLTTLXRYEP

SEQ ID NO: 166 - protein, 12 aa, synthetic construct
  REGION 1..12: note = Description of Artificial Sequence: Syntheticpeptide
HHFHLPKLRPPV

SEQ ID NO: 167 - protein, 7 aa, synthetic construct
  REGION 1..7: note = Description of Artificial Sequence: Syntheticpeptide
HHQRSPA

SEQ ID NO: 168 - protein, 12 aa, synthetic construct
  REGION 1..12: note = Description of Artificial Sequence: Syntheticpeptide
HHTWDTRIWQAF

SEQ ID NO: 169 - protein, 7 aa, synthetic construct
  REGION 1..7: note = Description of Artificial Sequence: Syntheticpeptide
HMLAQTF

SEQ ID NO: 170 - protein, 12 aa, synthetic construct
  REGION 1..12: note = Description of Artificial Sequence: Syntheticpeptide
HNVTTRTQRLMP

SEQ ID NO: 171 - protein, 11 aa, synthetic construct
  REGION 1..11: note = Description of Artificial Sequence: Syntheticpeptide
HPTTPIHMPNF

SEQ ID NO: 172 - protein, 12 aa, synthetic construct
  REGION 1..12: note = Description of Artificial Sequence: Syntheticpeptide
HQFISPEPFLIS

SEQ ID NO: 173 - protein, 12 aa, synthetic construct
  VARIANT 5: note = Any amino acid
  REGION 1..12: note = Description of Artificial Sequence: Syntheticpeptide
HQFPXSNLVWKP

SEQ ID NO: 174 - protein, 7 aa, synthetic construct
  REGION 1..7: note = Description of Artificial Sequence: Syntheticpeptide
HQWDHKY

SEQ ID NO: 175 - protein, 13 aa, synthetic construct
  VARIANT 5: note = Any amino acid
  REGION 1..13: note = Description of Artificial Sequence: Syntheticpeptide
  VARIANT 7: note = Any amino acid
  VARIANT 11: note = Any amino acid
HRDPXSXPSAXRP

SEQ ID NO: 176 - protein, 7 aa, synthetic construct
  REGION 1..7: note = Description of Artificial Sequence: Syntheticpeptide
HRLGHMS

SEQ ID NO: 177 - protein, 12 aa, synthetic construct
  REGION 1..12: note = Description of Artificial Sequence: Syntheticpeptide
HSACHASLKHRC

SEQ ID NO: 178 - protein, 12 aa, synthetic construct
  REGION 1..12: note = Description of Artificial Sequence: Syntheticpeptide
HSACKLTTCKDG

SEQ ID NO: 179 - protein, 12 aa, synthetic construct
  REGION 1..12: note = Description of Artificial Sequence: Syntheticpeptide
HSACLSTKTNIC

SEQ ID NO: 180 - protein, 12 aa, synthetic construct
  REGION 1..12: note = Description of Artificial Sequence: Syntheticpeptide
HSMPHMGTYLLT

SEQ ID NO: 181 - protein, 7 aa, synthetic construct
  REGION 1..7: note = Description of Artificial Sequence: Syntheticpeptide
HSTGPTR

SEQ ID NO: 182 - protein, 7 aa, synthetic construct
  REGION 1..7: note = Description of Artificial Sequence: Syntheticpeptide
HTLLSTT

SEQ ID NO: 183 - protein, 7 aa, synthetic construct
  REGION 1..7: note = Description of Artificial Sequence: Syntheticpeptide
HYPTVNF

SEQ ID NO: 184 - protein, 12 aa, synthetic construct
  REGION 1..12: note = Description of Artificial Sequence: Syntheticpeptide
IAHVPETRLAQM

SEQ ID NO: 185 - protein, 12 aa, synthetic construct
  REGION 1..12: note = Description of Artificial Sequence: Syntheticpeptide
IFSMGTALARPL

SEQ ID NO: 186 - protein, 7 aa, synthetic construct
  REGION 1..7: note = Description of Artificial Sequence: Syntheticpeptide
IGYPVLP

SEQ ID NO: 187 - protein, 12 aa, synthetic construct
  REGION 1..12: note = Description of Artificial Sequence: Syntheticpeptide
INFQFLKPSTTR

SEQ ID NO: 188 - protein, 12 aa, synthetic construct
  REGION 1..12: note = Description of Artificial Sequence: Syntheticpeptide
INKHPQQVSTLL

SEQ ID NO: 189 - protein, 7 aa, synthetic construct
  REGION 1..7: note = Description of Artificial Sequence: Syntheticpeptide
IQHQAKT

SEQ ID NO: 190 - protein, 7 aa, synthetic construct
  VARIANT 5: note = Any amino acid
  REGION 1..7: note = Description of Artificial Sequence: Syntheticpeptide
  VARIANT 3: note = Any amino acid
IRXLXIS

SEQ ID NO: 191 - protein, 12 aa, synthetic construct
  REGION 1..12: note = Description of Artificial Sequence: Syntheticpeptide
ISPSHSQAQADL

SEQ ID NO: 192 - protein, 7 aa, synthetic construct
  REGION 1..7: note = Description of Artificial Sequence: Syntheticpeptide
KAFDKHG

SEQ ID NO: 193 - protein, 7 aa, synthetic construct
  REGION 1..7: note = Description of Artificial Sequence: Syntheticpeptide
KATITGM

SEQ ID NO: 194 - protein, 12 aa, synthetic construct
  REGION 1..12: note = Description of Artificial Sequence: Syntheticpeptide
KEIPPIPLLAPS

SEQ ID NO: 195 - protein, 16 aa, synthetic construct
  REGION 1..16: note = Description of Artificial Sequence: Syntheticpeptide
KEIPPIPLLAPSGGGS

SEQ ID NO: 196 - protein, 20 aa, synthetic construct
  REGION 1..20: note = Description of Artificial Sequence: Syntheticpeptide
KIPKACCVPTELSAISMLYL

SEQ ID NO: 197 - protein, 33 aa, synthetic construct
  REGION 1..33: note = Description of Artificial Sequence: Syntheticpolypeptide
KIPKASSVPTELSAIATLYLAAAAEPRRAVAAL

SEQ ID NO: 198 - protein, 20 aa, synthetic construct
  REGION 1..20: note = Description of Artificial Sequence: Syntheticpeptide
KIPKASSVPTELSAISTLYL

SEQ ID NO: 199 - protein, 33 aa, synthetic construct
  REGION 1..33: note = Description of Artificial Sequence: Syntheticpolypeptide
KIPKASSVPTELSAISTLYLAAAAEPRRAVAAL

SEQ ID NO: 200 - protein, 33 aa, synthetic construct
  REGION 1..33: note = Description of Artificial Sequence: Syntheticpolypeptide
KIPKASSVPTELSAISTLYLAAAAEPRRAVAEL

SEQ ID NO: 201 - protein, 33 aa, synthetic construct
  REGION 1..33: note = Description of Artificial Sequence: Syntheticpolypeptide
KIPKASSVPTELSAISTLYLAAAAEPRREVAEL

SEQ ID NO: 202 - protein, 33 aa, synthetic construct
  VARIANT 25: note = Any amino acid
  VARIANT 32: note = Any amino acid
  REGION 1..33: note = Description of Artificial Sequence: Syntheticpolypeptide
  VARIANT 29: note = Any amino acid
KIPKASSVPTELSAISTLYLAAAAXPRRXVAXL

SEQ ID NO: 203 - protein, 29 aa, synthetic construct
  VARIANT 21: note = Any amino acid
  VARIANT 28: note = Any amino acid
  VARIANT 25: note = Any amino acid
  REGION 1..29: note = Description of Artificial Sequence: Syntheticpeptide
KIPKASSVPTELSAISTLYLXPRRXVAXL

SEQ ID NO: 204 - protein, 7 aa, synthetic construct
  REGION 1..7: note = Description of Artificial Sequence: Syntheticpeptide
KLHASLA

SEQ ID NO: 205 - protein, 7 aa, synthetic construct
  REGION 1..7: note = Description of Artificial Sequence: Syntheticpeptide
KLSAWSF

SEQ ID NO: 206 - protein, 15 aa, synthetic construct
  REGION 1..15: note = Description of Artificial Sequence: Syntheticpeptide
KLTWQELYQLKYKGI

SEQ ID NO: 207 - protein, 31 aa, synthetic construct
  REGION 1..31: note = Description of Artificial Sequence: Syntheticpolypeptide
KLTWQELYQLKYKGIGGGAAAAEPRREVAEL

SEQ ID NO: 208 - protein, 7 aa, synthetic construct
  REGION 1..7: note = Description of Artificial Sequence: Syntheticpeptide
KMNHMPN

SEQ ID NO: 209 - protein, 7 aa, synthetic construct
  REGION 1..7: note = Description of Artificial Sequence: Syntheticpeptide
KPMQFVH

SEQ ID NO: 210 - protein, 7 aa, synthetic construct
  REGION 1..7: note = Description of Artificial Sequence: Syntheticpeptide
KTSSWAN

SEQ ID NO: 211 - protein, 7 aa, synthetic construct
  REGION 1..7: note = Description of Artificial Sequence: Syntheticpeptide
LASTTHV

SEQ ID NO: 212 - protein, 12 aa, synthetic construct
  REGION 1..12: note = Description of Artificial Sequence: Syntheticpeptide
LDYPIPQTVLHH

SEQ ID NO: 213 - protein, 12 aa, synthetic construct
  REGION 1..12: note = Description of Artificial Sequence: Syntheticpeptide
LFAAVPSTQFFR

SEQ ID NO: 214 - protein, 12 aa, synthetic construct
  REGION 1..12: note = Description of Artificial Sequence: Syntheticpeptide
LGFDPTSTRFYT

SEQ ID NO: 215 - protein, 7 aa, synthetic construct
  REGION 1..7: note = Description of Artificial Sequence: Syntheticpeptide
LGPGKAF

SEQ ID NO: 216 - protein, 7 aa, synthetic construct
  REGION 1..7: note = Description of Artificial Sequence: Syntheticpeptide
LKPFSGA

SEQ ID NO: 217 - protein, 12 aa, synthetic construct
  REGION 1..12: note = Description of Artificial Sequence: Syntheticpeptide
LLADTTHHRPWP

SEQ ID NO: 218 - protein, 12 aa, synthetic construct
  REGION 1..12: note = Description of Artificial Sequence: Syntheticpeptide
LLADTTHHRPWT

SEQ ID NO: 219 - protein, 16 aa, synthetic construct
  REGION 1..16: note = Description of Artificial Sequence: Syntheticpeptide
LLADTTHHRPWTGGGS

SEQ ID NO: 220 - protein, 7 aa, synthetic construct
  REGION 1..7: note = Description of Artificial Sequence: Syntheticpeptide
LLPLKFK

SEQ ID NO: 221 - protein, 7 aa, synthetic construct
  REGION 1..7: note = Description of Artificial Sequence: Syntheticpeptide
LPFQPPI

SEQ ID NO: 222 - protein, 7 aa, synthetic construct
  REGION 1..7: note = Description of Artificial Sequence: Syntheticpeptide
LPLTPLP

SEQ ID NO: 223 - protein, 12 aa, synthetic construct
  REGION 1..12: note = Description of Artificial Sequence: Syntheticpeptide
LPRDLHATPQQI

SEQ ID NO: 224 - protein, 7 aa, synthetic construct
  REGION 1..7: note = Description of Artificial Sequence: Syntheticpeptide
LPSIHNL

SEQ ID NO: 225 - protein, 12 aa, synthetic construct
  REGION 1..12: note = Description of Artificial Sequence: Syntheticpeptide
LPWAPNLPDSTA

SEQ ID NO: 226 - protein, 12 aa, synthetic construct
  REGION 1..12: note = Description of Artificial Sequence: Syntheticpeptide
LPWTEPSFWRTP

SEQ ID NO: 227 - protein, 16 aa, synthetic construct
  REGION 1..16: note = Description of Artificial Sequence: Syntheticpeptide
LPWTEPSFWRTPGGGS

SEQ ID NO: 228 - protein, 7 aa, synthetic construct
  REGION 1..7: note = Description of Artificial Sequence: Syntheticpeptide
LQKSPSL

SEQ ID NO: 229 - protein, 12 aa, synthetic construct
  REGION 1..12: note = Description of Artificial Sequence: Syntheticpeptide
LQPSQPQRFAPT

SEQ ID NO: 230 - protein, 12 aa, synthetic construct
  REGION 1..12: note = Description of Artificial Sequence: Syntheticpeptide
LRAFPSLPHTVT

SEQ ID NO: 231 - protein, 7 aa, synthetic construct
  REGION 1..7: note = Description of Artificial Sequence: Syntheticpeptide
LSAPMEY

SEQ ID NO: 232 - protein, 7 aa, synthetic construct
  REGION 1..7: note = Description of Artificial Sequence: Syntheticpeptide
LSKNPLL

SEQ ID NO: 233 - protein, 12 aa, synthetic construct
  REGION 1..12: note = Description of Artificial Sequence: Syntheticpeptide
LSLRASAATDFQ

SEQ ID NO: 234 - protein, 12 aa, synthetic construct
  REGION 1..12: note = Description of Artificial Sequence: Syntheticpeptide
LSPPMQLQPTYS

SEQ ID NO: 235 - protein, 12 aa, synthetic construct
  REGION 1..12: note = Description of Artificial Sequence: Syntheticpeptide
LTPTMFNMHGVL

SEQ ID NO: 236 - protein, 7 aa, synthetic construct
  REGION 1..7: note = Description of Artificial Sequence: Syntheticpeptide
LTQTLQY

SEQ ID NO: 237 - protein, 12 aa, synthetic construct
  REGION 1..12: note = Description of Artificial Sequence: Syntheticpeptide
MHNVSDSNDSAI

SEQ ID NO: 238 - protein, 7 aa, synthetic construct
  REGION 1..7: note = Description of Artificial Sequence: Syntheticpeptide
MKVHERS

SEQ ID NO: 239 - protein, 12 aa, synthetic construct
  REGION 1..12: note = Description of Artificial Sequence: Syntheticpeptide
MPQTLVLPRSLL

SEQ ID NO: 240 - protein, 12 aa, synthetic construct
  REGION 1..12: note = Description of Artificial Sequence: Syntheticpeptide
MQFTPAPSPSDH

SEQ ID NO: 241 - protein, 7 aa, synthetic construct
  REGION 1..7: note = Description of Artificial Sequence: Syntheticpeptide
MTSQTLR

SEQ ID NO: 242 - protein, 7 aa, synthetic construct
  REGION 1..7: note = Description of Artificial Sequence: Syntheticpeptide
MYPLPAP

SEQ ID NO: 243 - protein, 7 aa, synthetic construct
  REGION 1..7: note = Description of Artificial Sequence: Syntheticpeptide
NERQMEL

SEQ ID NO: 244 - protein, 7 aa, synthetic construct
  REGION 1..7: note = Description of Artificial Sequence: Syntheticpeptide
NFAMNLR

SEQ ID NO: 245 - protein, 7 aa, synthetic construct
  REGION 1..7: note = Description of Artificial Sequence: Syntheticpeptide
NITQLGS

SEQ ID NO: 246 - protein, 7 aa, synthetic construct
  REGION 1..7: note = Description of Artificial Sequence: Syntheticpeptide
NKPLSTL

SEQ ID NO: 247 - protein, 12 aa, synthetic construct
  REGION 1..12: note = Description of Artificial Sequence: Syntheticpeptide
NNVSQKWQQRLI

SEQ ID NO: 248 - protein, 16 aa, synthetic construct
  REGION 1..16: note = Description of Artificial Sequence: Syntheticpeptide
NNVSQKWQQRLIGGGS

SEQ ID NO: 249 - protein, 16 aa, synthetic construct
  REGION 1..16: note = Description of Artificial Sequence: Syntheticpeptide
NPDHPDIPQDVHGGGK

SEQ ID NO: 250 - protein, 7 aa, synthetic construct
  REGION 1..7: note = Description of Artificial Sequence: Syntheticpeptide
NPMIMNQ

SEQ ID NO: 251 - protein, 7 aa, synthetic construct
  REGION 1..7: note = Description of Artificial Sequence: Syntheticpeptide
NPQMQRS

SEQ ID NO: 252 - protein, 7 aa, synthetic construct
  REGION 1..7: note = Description of Artificial Sequence: Syntheticpeptide
NPRSQAT

SEQ ID NO: 253 - protein, 16 aa, synthetic construct
  REGION 1..16: note = Description of Artificial Sequence: Syntheticpeptide
NPYAPTIPQSVAGGGK

SEQ ID NO: 254 - protein, 12 aa, synthetic construct
  REGION 1..12: note = Description of Artificial Sequence: Syntheticpeptide
NPYHPTIPQSVH

SEQ ID NO: 255 - protein, 16 aa, synthetic construct
  REGION 1..16: note = Description of Artificial Sequence: Syntheticpeptide
NPYHPTIPQSVHGGGK

SEQ ID NO: 256 - protein, 12 aa, synthetic construct
  REGION 1..12: note = Description of Artificial Sequence: Syntheticpeptide
NSMIAHNKTRMH

SEQ ID NO: 257 - protein, 16 aa, synthetic construct
  REGION 1..16: note = Description of Artificial Sequence: Syntheticpeptide
NSMIAHNKTRMHGGGS

SEQ ID NO: 258 - protein, 12 aa, synthetic construct
  REGION 1..12: note = Description of Artificial Sequence: Syntheticpeptide
NSSMLGMLPSSF

SEQ ID NO: 259 - protein, 12 aa, synthetic construct
  REGION 1..12: note = Description of Artificial Sequence: Syntheticpeptide
NTSSSQGTQRLG

SEQ ID NO: 260 - protein, 12 aa, synthetic construct
  REGION 1..12: note = Description of Artificial Sequence: Syntheticpeptide
NTTTDIPSPSQF

SEQ ID NO: 261 - protein, 7 aa, synthetic construct
  REGION 1..7: note = Description of Artificial Sequence: Syntheticpeptide
NYPTLKS

SEQ ID NO: 262 - protein, 12 aa, synthetic construct
  REGION 1..12: note = Description of Artificial Sequence: Syntheticpeptide
NYSHLRVKLPTP

SEQ ID NO: 263 - protein, 16 aa, synthetic construct
  REGION 1..16: note = Description of Artificial Sequence: Syntheticpeptide
NYSHLRVKLPTPGGGS

SEQ ID NO: 264 - protein, 7 aa, synthetic construct
  REGION 1..7: note = Description of Artificial Sequence: Syntheticpeptide
PAKQKAH

SEQ ID NO: 265 - protein, 7 aa, synthetic construct
  REGION 1..7: note = Description of Artificial Sequence: Syntheticpeptide
PDIPLSR

SEQ ID NO: 266 - protein, 12 aa, synthetic construct
  REGION 1..12: note = Description of Artificial Sequence: Syntheticpeptide
PGQWPSSLTLYK

SEQ ID NO: 267 - protein, 7 aa, synthetic construct
  REGION 1..7: note = Description of Artificial Sequence: Syntheticpeptide
PHNPGKL

SEQ ID NO: 268 - protein, 7 aa, synthetic construct
  REGION 1..7: note = Description of Artificial Sequence: Syntheticpeptide
PIDAFFD

SEQ ID NO: 269 - protein, 7 aa, synthetic construct
  REGION 1..7: note = Description of Artificial Sequence: Syntheticpeptide
PLTQPSH

SEQ ID NO: 270 - protein, 7 aa, synthetic construct
  REGION 1..7: note = Description of Artificial Sequence: Syntheticpeptide
PPKDSRG

SEQ ID NO: 271 - protein, 7 aa, synthetic construct
  REGION 1..7: note = Description of Artificial Sequence: Syntheticpeptide
PPNMARA

SEQ ID NO: 272 - protein, 7 aa, synthetic construct
  REGION 1..7: note = Description of Artificial Sequence: Syntheticpeptide
PSMKHWR

SEQ ID NO: 273 - protein, 7 aa, synthetic construct
  REGION 1..7: note = Description of Artificial Sequence: Syntheticpeptide
PTNKPHT

SEQ ID NO: 274 - protein, 7 aa, synthetic construct
  REGION 1..7: note = Description of Artificial Sequence: Syntheticpeptide
PTTMTRW

SEQ ID NO: 275 - protein, 7 aa, synthetic construct
  REGION 1..7: note = Description of Artificial Sequence: Syntheticpeptide
PTTWGHL

SEQ ID NO: 276 - protein, 36 aa, synthetic construct
  VARIANT 20: note = Any amino acid
  VARIANT 29: note = Any amino acid
  VARIANT 23: note = Any amino acid
  VARIANT 17: note = Any amino acid
  VARIANT 14: note = Any amino acid
  REGION 1..36: note = Description of Artificial Sequence: Syntheticpolypeptide
  VARIANT 2: note = Any amino acid
  VARIANT 11: note = Any amino acid
  VARIANT 8: note = Any amino acid
  VARIANT 5: note = Any amino acid
  VARIANT 26: note = Any amino acid
  VARIANT 32: note = Any amino acid
  VARIANT 35: note = Any amino acid
PXGPXGPXGPXGPXGPXAPXGPXGPXGPXGPXGPXG

SEQ ID NO: 277 - protein, 12 aa, synthetic construct
  REGION 1..12: note = Description of Artificial Sequence: Syntheticpeptide
QHNFRGASSSAP

SEQ ID NO: 278 - protein, 12 aa, synthetic construct
  REGION 1..12: note = Description of Artificial Sequence: Syntheticpeptide
QIPQMRILHPYG

SEQ ID NO: 279 - protein, 12 aa, synthetic construct
  REGION 1..12: note = Description of Artificial Sequence: Syntheticpeptide
QIQKPPRTPPSL

SEQ ID NO: 280 - protein, 12 aa, synthetic construct
  REGION 1..12: note = Description of Artificial Sequence: Syntheticpeptide
QLTQTMWKDTTL

SEQ ID NO: 281 - protein, 12 aa, synthetic construct
  REGION 1..12: note = Description of Artificial Sequence: Syntheticpeptide
QNLPPERYSEAT

SEQ ID NO: 282 - protein, 7 aa, synthetic construct
  REGION 1..7: note = Description of Artificial Sequence: Syntheticpeptide
QNPRQIY

SEQ ID NO: 283 - protein, 7 aa, synthetic construct
  REGION 1..7: note = Description of Artificial Sequence: Syntheticpeptide
QNYLLPK

SEQ ID NO: 284 - protein, 7 aa, synthetic construct
  REGION 1..7: note = Description of Artificial Sequence: Syntheticpeptide
QPGLWPS

SEQ ID NO: 285 - protein, 12 aa, synthetic construct
  REGION 1..12: note = Description of Artificial Sequence: Syntheticpeptide
QRSWTLDSALSM

SEQ ID NO: 286 - protein, 16 aa, synthetic construct
  REGION 1..16: note = Description of Artificial Sequence: Syntheticpeptide
QRSWTLDSALSMGGGS

SEQ ID NO: 287 - protein, 12 aa, synthetic construct
  REGION 1..12: note = Description of Artificial Sequence: Syntheticpeptide
QSLSFAGPPAWQ

SEQ ID NO: 288 - protein, 7 aa, synthetic construct
  REGION 1..7: note = Description of Artificial Sequence: Syntheticpeptide
QSSYNPI

SEQ ID NO: 289 - protein, 7 aa, synthetic construct
  REGION 1..7: note = Description of Artificial Sequence: Syntheticpeptide
QTHARHQ

SEQ ID NO: 290 - protein, 7 aa, synthetic construct
  REGION 1..7: note = Description of Artificial Sequence: Syntheticpeptide
QTHSSLW

SEQ ID NO: 291 - protein, 12 aa, synthetic construct
  REGION 1..12: note = Description of Artificial Sequence: Syntheticpeptide
QTTMTPLWPSFS

SEQ ID NO: 292 - protein, 12 aa, synthetic construct
  REGION 1..12: note = Description of Artificial Sequence: Syntheticpeptide
RCMSEVISFNCP

SEQ ID NO: 293 - protein, 7 aa, synthetic construct
  REGION 1..7: note = Description of Artificial Sequence: Syntheticpeptide
RHTLPLH

SEQ ID NO: 294 - protein, 7 aa, synthetic construct
  REGION 1..7: note = Description of Artificial Sequence: Syntheticpeptide
RPHTITN

SEQ ID NO: 295 - protein, 12 aa, synthetic construct
  REGION 1..12: note = Description of Artificial Sequence: Syntheticpeptide
RSPYYNKWSSKF

SEQ ID NO: 296 - protein, 12 aa, synthetic construct
  REGION 1..12: note = Description of Artificial Sequence: Syntheticpeptide
RTPLQPLEDFRP

SEQ ID NO: 297 - protein, 12 aa, synthetic construct
  REGION 1..12: note = Description of Artificial Sequence: Syntheticpeptide
SAGHIHEAHRPL

SEQ ID NO: 298 - protein, 12 aa, synthetic construct
  REGION 1..12: note = Description of Artificial Sequence: Syntheticpeptide
SAISDHRAHRSH

SEQ ID NO: 299 - protein, 7 aa, synthetic construct
  REGION 1..7: note = Description of Artificial Sequence: Syntheticpeptide
SAKGRAD

SEQ ID NO: 300 - protein, 7 aa, synthetic construct
  REGION 1..7: note = Description of Artificial Sequence: Syntheticpeptide
SAKKVFS

SEQ ID NO: 301 - protein, 7 aa, synthetic construct
  REGION 1..7: note = Description of Artificial Sequence: Syntheticpeptide
SASGTPS

SEQ ID NO: 302 - protein, 12 aa, synthetic construct
  REGION 1..12: note = Description of Artificial Sequence: Syntheticpeptide
SEPTYWRPNMSG

SEQ ID NO: 303 - protein, 12 aa, synthetic construct
  REGION 1..12: note = Description of Artificial Sequence: Syntheticpeptide
SFAPDIKYPVPS

SEQ ID NO: 304 - protein, 12 aa, synthetic construct
  REGION 1..12: note = Description of Artificial Sequence: Syntheticpeptide
SFQSMSLMTLVV

SEQ ID NO: 305 - protein, 12 aa, synthetic construct
  REGION 1..12: note = Description of Artificial Sequence: Syntheticpeptide
SFWHHHSPRSPL

SEQ ID NO: 306 - protein, 12 aa, synthetic construct
  REGION 1..12: note = Description of Artificial Sequence: Syntheticpeptide
SGHQLLLNKMPN

SEQ ID NO: 307 - protein, 16 aa, synthetic construct
  REGION 1..16: note = Description of Artificial Sequence: Syntheticpeptide
SGHQLLLNKMPNGGGS

SEQ ID NO: 308 - protein, 12 aa, synthetic construct
  REGION 1..12: note = Description of Artificial Sequence: Syntheticpeptide
SIFAHQTPTHKN

SEQ ID NO: 309 - protein, 12 aa, synthetic construct
  REGION 1..12: note = Description of Artificial Sequence: Syntheticpeptide
SIPKMIPTESLL

SEQ ID NO: 310 - protein, 12 aa, synthetic construct
  REGION 1..12: note = Description of Artificial Sequence: Syntheticpeptide
SIPSHSIHSAKA

SEQ ID NO: 311 - protein, 12 aa, synthetic construct
  REGION 1..12: note = Description of Artificial Sequence: Syntheticpeptide
SIRTSMNPPNLL

SEQ ID NO: 312 - protein, 7 aa, synthetic construct
  REGION 1..7: note = Description of Artificial Sequence: Syntheticpeptide
SKTSSTS

SEQ ID NO: 313 - protein, 7 aa, synthetic construct
  REGION 1..7: note = Description of Artificial Sequence: Syntheticpeptide
SLLTPWL

SEQ ID NO: 314 - protein, 12 aa, synthetic construct
  REGION 1..12: note = Description of Artificial Sequence: Syntheticpeptide
SLPHYIDNPFRQ

SEQ ID NO: 315 - protein, 12 aa, synthetic construct
  REGION 1..12: note = Description of Artificial Sequence: Syntheticpeptide
SLSKANILHLYG

SEQ ID NO: 316 - protein, 12 aa, synthetic construct
  REGION 1..12: note = Description of Artificial Sequence: Syntheticpeptide
SLVTADASFTPS

SEQ ID NO: 317 - protein, 7 aa, synthetic construct
  REGION 1..7: note = Description of Artificial Sequence: Syntheticpeptide
SMAAKSS

SEQ ID NO: 318 - protein, 12 aa, synthetic construct
  REGION 1..12: note = Description of Artificial Sequence: Syntheticpeptide
SMVYGNRLPSAL

SEQ ID NO: 319 - protein, 7 aa, synthetic construct
  REGION 1..7: note = Description of Artificial Sequence: Syntheticpeptide
SMYDTHS

SEQ ID NO: 320 - protein, 7 aa, synthetic construct
  REGION 1..7: note = Description of Artificial Sequence: Syntheticpeptide
SPEMKPR

SEQ ID NO: 321 - protein, 12 aa, synthetic construct
  REGION 1..12: note = Description of Artificial Sequence: Syntheticpeptide
SPNFSWLPLGTT

SEQ ID NO: 322 - protein, 12 aa, synthetic construct
  REGION 1..12: note = Description of Artificial Sequence: Syntheticpeptide
SPNLPWSKLSAY

SEQ ID NO: 323 - protein, 7 aa, synthetic construct
  REGION 1..7: note = Description of Artificial Sequence: Syntheticpeptide
SPNNPRE

SEQ ID NO: 324 - protein, 7 aa, synthetic construct
  REGION 1..7: note = Description of Artificial Sequence: Syntheticpeptide
SPNNTRE

SEQ ID NO: 325 - protein, 12 aa, synthetic construct
  REGION 1..12: note = Description of Artificial Sequence: Syntheticpeptide
SPSLMARSSPYW

SEQ ID NO: 326 - protein, 7 aa, synthetic construct
  REGION 1..7: note = Description of Artificial Sequence: Syntheticpeptide
SQHSTQD

SEQ ID NO: 327 - protein, 12 aa, synthetic construct
  REGION 1..12: note = Description of Artificial Sequence: Syntheticpeptide
SQTLPYSNAPSP

SEQ ID NO: 328 - protein, 7 aa, synthetic construct
  REGION 1..7: note = Description of Artificial Sequence: Syntheticpeptide
SRTGAHH

SEQ ID NO: 329 - protein, 7 aa, synthetic construct
  REGION 1..7: note = Description of Artificial Sequence: Syntheticpeptide
SSHHHRH

SEQ ID NO: 330 - protein, 7 aa, synthetic construct
  REGION 1..7: note = Description of Artificial Sequence: Syntheticpeptide
SSPPRVY

SEQ ID NO: 331 - protein, 7 aa, synthetic construct
  REGION 1..7: note = Description of Artificial Sequence: Syntheticpeptide
SSSMAKM

SEQ ID NO: 332 - protein, 12 aa, synthetic construct
  REGION 1..12: note = Description of Artificial Sequence: Syntheticpeptide
SSTLKTFFGFPD

SEQ ID NO: 333 - protein, 16 aa, synthetic construct
  REGION 1..16: note = Description of Artificial Sequence: Syntheticpeptide
SSTLKTFFGFPDGGGS

SEQ ID NO: 334 - protein, 12 aa, synthetic construct
  REGION 1..12: note = Description of Artificial Sequence: Syntheticpeptide
SSTQAHPFAPQL

SEQ ID NO: 335 - protein, 12 aa, synthetic construct
  REGION 1..12: note = Description of Artificial Sequence: Syntheticpeptide
SSTQVQHTLLQT

SEQ ID NO: 336 - protein, 7 aa, synthetic construct
  REGION 1..7: note = Description of Artificial Sequence: Syntheticpeptide
SSVPGRP

SEQ ID NO: 337 - protein, 7 aa, synthetic construct
  REGION 1..7: note = Description of Artificial Sequence: Syntheticpeptide
SSYEYHA

SEQ ID NO: 338 - protein, 7 aa, synthetic construct
  REGION 1..7: note = Description of Artificial Sequence: Syntheticpeptide
STLASMR

SEQ ID NO: 339 - protein, 12 aa, synthetic construct
  REGION 1..12: note = Description of Artificial Sequence: Syntheticpeptide
STPNSYSLPQAR

SEQ ID NO: 340 - protein, 7 aa, synthetic construct
  REGION 1..7: note = Description of Artificial Sequence: Syntheticpeptide
STQAHPW

SEQ ID NO: 341 - protein, 7 aa, synthetic construct
  REGION 1..7: note = Description of Artificial Sequence: Syntheticpeptide
STSAKHW

SEQ ID NO: 342 - protein, 12 aa, synthetic construct
  REGION 1..12: note = Description of Artificial Sequence: Syntheticpeptide
STVVMQPPPRPA

SEQ ID NO: 343 - protein, 12 aa, synthetic construct
  REGION 1..12: note = Description of Artificial Sequence: Syntheticpeptide
SVFLPTRHSPDL

SEQ ID NO: 344 - protein, 12 aa, synthetic construct
  REGION 1..12: note = Description of Artificial Sequence: Syntheticpeptide
SVQTRPLFHSHF

SEQ ID NO: 345 - protein, 12 aa, synthetic construct
  REGION 1..12: note = Description of Artificial Sequence: Syntheticpeptide
SVSVGMKPSPRP

SEQ ID NO: 346 - protein, 12 aa, synthetic construct
  VARIANT 11: note = Any amino acid
  REGION 1..12: note = Description of Artificial Sequence: Syntheticpeptide
SVSVGMNAESXA

SEQ ID NO: 347 - protein, 12 aa, synthetic construct
  REGION 1..12: note = Description of Artificial Sequence: Syntheticpeptide
SVSVGMNAESYG

SEQ ID NO: 348 - protein, 12 aa, synthetic construct
  VARIANT 11: note = Any amino acid
  REGION 1..12: note = Description of Artificial Sequence: Syntheticpeptide
SVSVGTEAESXA

SEQ ID NO: 349 - protein, 12 aa, synthetic construct
  REGION 1..12: note = Description of Artificial Sequence: Syntheticpeptide
SWPLYSRDSGLG

SEQ ID NO: 350 - protein, 12 aa, synthetic construct
  REGION 1..12: note = Description of Artificial Sequence: Syntheticpeptide
SYIDSMVPSTQT

SEQ ID NO: 351 - protein, 12 aa, synthetic construct
  REGION 1..12: note = Description of Artificial Sequence: Syntheticpeptide
SYKTTDSDTSPL

SEQ ID NO: 352 - protein, 16 aa, synthetic construct
  REGION 1..16: note = Description of Artificial Sequence: Syntheticpeptide
SYSQMDPPRSLPGGGS

SEQ ID NO: 353 - protein, 12 aa, synthetic construct
  REGION 1..12: note = Description of Artificial Sequence: Syntheticpeptide
TAAASNLRAVPP

SEQ ID NO: 354 - protein, 12 aa, synthetic construct
  REGION 1..12: note = Description of Artificial Sequence: Syntheticpeptide
TAPLSHPPRPGA

SEQ ID NO: 355 - protein, 7 aa, synthetic construct
  REGION 1..7: note = Description of Artificial Sequence: Syntheticpeptide
TDHPPKA

SEQ ID NO: 356 - protein, 7 aa, synthetic construct
  REGION 1..7: note = Description of Artificial Sequence: Syntheticpeptide
TGLAKTA

SEQ ID NO: 357 - protein, 12 aa, synthetic construct
  REGION 1..12: note = Description of Artificial Sequence: Syntheticpeptide
TGLLPNSSGAGI

SEQ ID NO: 358 - protein, 12 aa, synthetic construct
  REGION 1..12: note = Description of Artificial Sequence: Syntheticpeptide
TGPPSRQPAPLH

SEQ ID NO: 359 - protein, 7 aa, synthetic construct
  REGION 1..7: note = Description of Artificial Sequence: Syntheticpeptide
TGPTSLS

SEQ ID NO: 360 - protein, 12 aa, synthetic construct
  REGION 1..12: note = Description of Artificial Sequence: Syntheticpeptide
THPVVFEDERLF

SEQ ID NO: 361 - protein, 7 aa, synthetic construct
  REGION 1..7: note = Description of Artificial Sequence: Syntheticpeptide
TIHSKPA

SEQ ID NO: 362 - protein, 7 aa, synthetic construct
  REGION 1..7: note = Description of Artificial Sequence: Syntheticpeptide
TKDWLPS

SEQ ID NO: 363 - protein, 7 aa, synthetic construct
  REGION 1..7: note = Description of Artificial Sequence: Syntheticpeptide
TLAFQTA

SEQ ID NO: 364 - protein, 7 aa, synthetic construct
  REGION 1..7: note = Description of Artificial Sequence: Syntheticpeptide
TLAPTFR

SEQ ID NO: 365 - protein, 12 aa, synthetic construct
  REGION 1..12: note = Description of Artificial Sequence: Syntheticpeptide
TLDKYTRLLSRY

SEQ ID NO: 366 - protein, 7 aa, synthetic construct
  REGION 1..7: note = Description of Artificial Sequence: Syntheticpeptide
TLGLPML

SEQ ID NO: 367 - protein, 7 aa, synthetic construct
  REGION 1..7: note = Description of Artificial Sequence: Syntheticpeptide
TLLRTQV

SEQ ID NO: 368 - protein, 7 aa, synthetic construct
  REGION 1..7: note = Description of Artificial Sequence: Syntheticpeptide
TLMTTPP

SEQ ID NO: 369 - protein, 12 aa, synthetic construct
  REGION 1..12: note = Description of Artificial Sequence: Syntheticpeptide
TLPSPLALLTVH

SEQ ID NO: 370 - protein, 7 aa, synthetic construct
  REGION 1..7: note = Description of Artificial Sequence: Syntheticpeptide
TLQRMGQ

SEQ ID NO: 371 - protein, 12 aa, synthetic construct
  REGION 1..12: note = Description of Artificial Sequence: Syntheticpeptide
TLSNGHRYLELL

SEQ ID NO: 372 - protein, 12 aa, synthetic construct
  REGION 1..12: note = Description of Artificial Sequence: Syntheticpeptide
TMGFTAPRFPHY

SEQ ID NO: 373 - protein, 12 aa, synthetic construct
  REGION 1..12: note = Description of Artificial Sequence: Syntheticpeptide
TMRNPITSLISV

SEQ ID NO: 374 - protein, 16 aa, synthetic construct
  REGION 1..16: note = Description of Artificial Sequence: Syntheticpeptide
TMRNPITSLISVGGGS

SEQ ID NO: 375 - protein, 7 aa, synthetic construct
  REGION 1..7: note = Description of Artificial Sequence: Syntheticpeptide
TMTNMAK

SEQ ID NO: 376 - protein, 12 aa, synthetic construct
  REGION 1..12: note = Description of Artificial Sequence: Syntheticpeptide
TPLSYLKGLVTV

SEQ ID NO: 377 - protein, 12 aa, synthetic construct
  REGION 1..12: note = Description of Artificial Sequence: Syntheticpeptide
TPLTSPSLVRPQ

SEQ ID NO: 378 - protein, 12 aa, synthetic construct
  REGION 1..12: note = Description of Artificial Sequence: Syntheticpeptide
TPSPKLLQVFQA

SEQ ID NO: 379 - protein, 12 aa, synthetic construct
  REGION 1..12: note = Description of Artificial Sequence: Syntheticpeptide
TPSTGLGMSPAV

SEQ ID NO: 380 - protein, 12 aa, synthetic construct
  REGION 1..12: note = Description of Artificial Sequence: Syntheticpeptide
TPVYSLKLGPWP

SEQ ID NO: 381 - protein, 12 aa, synthetic construct
  REGION 1..12: note = Description of Artificial Sequence: Syntheticpeptide
TQTWPQSSSHGL

SEQ ID NO: 382 - protein, 7 aa, synthetic construct
  REGION 1..7: note = Description of Artificial Sequence: Syntheticpeptide
TRFYDSL

SEQ ID NO: 383 - protein, 12 aa, synthetic construct
  REGION 1..12: note = Description of Artificial Sequence: Syntheticpeptide
TRLVPSRYYHHP

SEQ ID NO: 384 - protein, 12 aa, synthetic construct
  REGION 1..12: note = Description of Artificial Sequence: Syntheticpeptide
TSPIPQMRTVPP

SEQ ID NO: 385 - protein, 7 aa, synthetic construct
  REGION 1..7: note = Description of Artificial Sequence: Syntheticpeptide
TTKNFNK

SEQ ID NO: 386 - protein, 7 aa, synthetic construct
  REGION 1..7: note = Description of Artificial Sequence: Syntheticpeptide
TTLSPRT

SEQ ID NO: 387 - protein, 12 aa, synthetic construct
  REGION 1..12: note = Description of Artificial Sequence: Syntheticpeptide
TTNSSMTMQLQR

SEQ ID NO: 388 - protein, 12 aa, synthetic construct
  REGION 1..12: note = Description of Artificial Sequence: Syntheticpeptide
TTTLPVQPTLRN

SEQ ID NO: 389 - protein, 12 aa, synthetic construct
  REGION 1..12: note = Description of Artificial Sequence: Syntheticpeptide
TTTWTTTARWPL

SEQ ID NO: 390 - protein, 7 aa, synthetic construct
  REGION 1..7: note = Description of Artificial Sequence: Syntheticpeptide
TTYNSPP

SEQ ID NO: 391 - protein, 12 aa, synthetic construct
  REGION 1..12: note = Description of Artificial Sequence: Syntheticpeptide
TVAQMPPHWQLT

SEQ ID NO: 392 - protein, 7 aa, synthetic construct
  REGION 1..7: note = Description of Artificial Sequence: Syntheticpeptide
TVLGTFP

SEQ ID NO: 393 - protein, 12 aa, synthetic construct
  REGION 1..12: note = Description of Artificial Sequence: Syntheticpeptide
TWNSNSTQYGNR

SEQ ID NO: 394 - protein, 12 aa, synthetic construct
  REGION 1..12: note = Description of Artificial Sequence: Syntheticpeptide
TWTLPAMHPRPA

SEQ ID NO: 395 - protein, 7 aa, synthetic construct
  REGION 1..7: note = Description of Artificial Sequence: Syntheticpeptide
VHLTHGQ

SEQ ID NO: 396 - protein, 7 aa, synthetic construct
  REGION 1..7: note = Description of Artificial Sequence: Syntheticpeptide
VHPRPSL

SEQ ID NO: 397 - protein, 12 aa, synthetic construct
  REGION 1..12: note = Description of Artificial Sequence: Syntheticpeptide
VHTSLLQKHPLP

SEQ ID NO: 398 - protein, 11 aa, synthetic construct
  REGION 1..11: note = Description of Artificial Sequence: Syntheticpeptide
VLPNIYMTLSA

SEQ ID NO: 399 - protein, 12 aa, synthetic construct
  REGION 1..12: note = Description of Artificial Sequence: Syntheticpeptide
VMDFASPAHVLP

SEQ ID NO: 400 - protein, 12 aa, synthetic construct
  REGION 1..12: note = Description of Artificial Sequence: Syntheticpeptide
VNQEYWFFPRRP

SEQ ID NO: 401 - protein, 15 aa, synthetic construct
  VARIANT 11: note = Any amino acid
  REGION 1..15: note = Description of Artificial Sequence: Syntheticpeptide
  VARIANT 6: note = Any amino acid
  VARIANT 14: note = Any amino acid
VPPISXTFLFXSTXS

SEQ ID NO: 402 - protein, 12 aa, synthetic construct
  REGION 1..12: note = Description of Artificial Sequence: Syntheticpeptide
  VARIANT 11: note = Any amino acid
VPPLHPALSRXN

SEQ ID NO: 403 - protein, 12 aa, synthetic construct
  REGION 1..12: note = Description of Artificial Sequence: Syntheticpeptide
VSPFLSPTPLLF

SEQ ID NO: 404 - protein, 12 aa, synthetic construct
  REGION 1..12: note = Description of Artificial Sequence: Syntheticpeptide
VSRLGTPSMHPS

SEQ ID NO: 405 - protein, 7 aa, synthetic construct
  REGION 1..7: note = Description of Artificial Sequence: Syntheticpeptide
VVKSNGE

SEQ ID NO: 406 - protein, 11 aa, synthetic construct
  REGION 1..11: note = Description of Artificial Sequence: Syntheticpeptide
VYSSPLSQLPR

SEQ ID NO: 407 - protein, 7 aa, synthetic construct
  REGION 1..7: note = Description of Artificial Sequence: Syntheticpeptide
WLPPRTQ

SEQ ID NO: 408 - protein, 12 aa, synthetic construct
  REGION 1..12: note = Description of Artificial Sequence: Syntheticpeptide
WPANKLSTKSMY

SEQ ID NO: 409 - protein, 12 aa, synthetic construct
  REGION 1..12: note = Description of Artificial Sequence: Syntheticpeptide
WPFNHFPWWNVP

SEQ ID NO: 410 - protein, 12 aa, synthetic construct
  REGION 1..12: note = Description of Artificial Sequence: Syntheticpeptide
WPTYLNPSSLKA

SEQ ID NO: 411 - protein, 12 aa, synthetic construct
  REGION 1..12: note = Description of Artificial Sequence: Syntheticpeptide
WSAHIVPYSHKP

SEQ ID NO: 412 - protein, 12 aa, synthetic construct
  REGION 1..12: note = Description of Artificial Sequence: Syntheticpeptide
WWPNSLNWVPRP

SEQ ID NO: 413 - protein, 7 aa, synthetic construct
  REGION 1..7: note = Description of Artificial Sequence: Syntheticpeptide
WYPNHLA

SEQ ID NO: 414 - protein, 7 aa, synthetic construct
  REGION 1..7: note = Description of Artificial Sequence: Syntheticpeptide
  VARIANT 1: note = Any amino acid
  VARIANT 4: note = Any amino acid
XITXGAY

SEQ ID NO: 415 - protein, 9 aa, synthetic construct
  VARIANT 1: note = Any amino acid
  REGION 1..9: note = Description of Artificial Sequence: Syntheticpeptide
XPRRAVAAL

SEQ ID NO: 416 - protein, 9 aa, synthetic construct
  REGION 1..9: note = Description of Artificial Sequence: Syntheticpeptide
  VARIANT 1: note = Any amino acid
  VARIANT 8: note = Any amino acid
XPRRAVAXL

SEQ ID NO: 417 - protein, 9 aa, synthetic construct
  VARIANT 1: note = Any amino acid
  REGION 1..9: note = Description of Artificial Sequence: Syntheticpeptide
  VARIANT 8: note = Any amino acid
  VARIANT 5: note = Any amino acid
XPRRXVAXL

SEQ ID NO: 418 - protein, 7 aa, synthetic construct
  VARIANT 1..2: note = Any amino acid
  VARIANT 6: note = Any amino acid
  REGION 1..7: note = Description of Artificial Sequence: Syntheticpeptide
XXFPLXG

SEQ ID NO: 419 - protein, 12 aa, synthetic construct
  REGION 1..12: note = Description of Artificial Sequence: Syntheticpeptide
YATQHNWRLKHE

SEQ ID NO: 420 - protein, 10 aa, synthetic construct
  REGION 1..10: note = Description of Artificial Sequence: Syntheticpeptide
YCPMRLCTDC

SEQ ID NO: 421 - protein, 12 aa, synthetic construct
  REGION 1..12: note = Description of Artificial Sequence: Syntheticpeptide
YELQMPLTLPLN

SEQ ID NO: 422 - protein, 7 aa, synthetic construct
  REGION 1..7: note = Description of Artificial Sequence: Syntheticpeptide
YEPAAAE

SEQ ID NO: 423 - protein, 12 aa, synthetic construct
  REGION 1..12: note = Description of Artificial Sequence: Syntheticpeptide
YGKGFSPYFHVT

SEQ ID NO: 424 - protein, 12 aa, synthetic construct
  REGION 1..12: note = Description of Artificial Sequence: Syntheticpeptide
YPHYSLPGSSTL

SEQ ID NO: 425 - protein, 12 aa, synthetic construct
  REGION 1..12: note = Description of Artificial Sequence: Syntheticpeptide
YPIMSHTCCHGV

SEQ ID NO: 426 - protein, 7 aa, synthetic construct
  REGION 1..7: note = Description of Artificial Sequence: Syntheticpeptide
YPKALRN

SEQ ID NO: 427 - protein, 12 aa, synthetic construct
  REGION 1..12: note = Description of Artificial Sequence: Syntheticpeptide
YPSLLKMQPQFS

SEQ ID NO: 428 - protein, 12 aa, synthetic construct
  REGION 1..12: note = Description of Artificial Sequence: Syntheticpeptide
YQPRPFVTTSPM

SEQ ID NO: 429 - protein, 12 aa, synthetic construct
  REGION 1..12: note = Description of Artificial Sequence: Syntheticpeptide
YSAPLARSNVVM

SEQ ID NO: 430 - protein, 12 aa, synthetic construct
  REGION 1..12: note = Description of Artificial Sequence: Syntheticpeptide
YTRLSHNPYTLS

SEQ ID NO: 431 - protein, 12 aa, synthetic construct
  REGION 1..12: note = Description of Artificial Sequence: Syntheticpeptide
YTTHVLPFAPSS

SEQ ID NO: 432 - protein, 12 aa, synthetic construct
  REGION 1..12: note = Description of Artificial Sequence: Syntheticpeptide
YTWQTIREQYEM

SEQ ID NO: 433 - protein, 152 aa, synthetic construct
  REGION 1..152: note = Description of Artificial Sequence: Syntheticpolypeptide
ASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 434 - protein, 217 aa, synthetic construct
  REGION 1..217: note = Description of Artificial Sequence: Syntheticpolypeptide
MPIGSLLADTTHHRPWTVIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 435 - protein, 212 aa, synthetic construct
  REGION 1..212: note = Description of Artificial Sequence: Syntheticpolypeptide
LLADTTHHRPWTVIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 436 - protein, 200 aa, synthetic construct
  REGION 1..200: note = Description of Artificial Sequence: Syntheticpolypeptide
VIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 437 - protein, 188 aa, synthetic construct
  REGION 1..188: note = Description of Artificial Sequence: Syntheticpolypeptide
IIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 438 - protein, 176 aa, synthetic construct
  REGION 1..176: note = Description of Artificial Sequence: Syntheticpolypeptide
GLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 439 - protein, 164 aa, synthetic construct
  REGION 1..164: note = Description of Artificial Sequence: Syntheticpolypeptide
ILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 440 - protein, 114 aa, synthetic construct
  REGION 1..114: note = Description of Artificial Sequence: Syntheticpolypeptide
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 441 - protein, 152 aa, synthetic construct; identical to SEQ ID NO: 433
  REGION 1..152: note = Description of Artificial Sequence: Syntheticpolypeptide

SEQ ID NO: 442 - protein, 53 aa, synthetic construct
  REGION 1..53: note = Description of Artificial Sequence: Syntheticpolypeptide
NSDSECPLSHDGYCLHDGVCMYIEALDKYACNCVVGYIGERCQYRDLKWWELR

SEQ ID NO: 443 - protein, 66 aa, synthetic construct
  REGION 1..66: note = Description of Artificial Sequence: Syntheticpolypeptide
EEAEIPREVIERLARSQIHSIRDLQRLLEIDSVGSEDSLDTSLRAHGVHATKHVPEKRPLPIRRKR

SEQ ID NO: 444 - protein, 70 aa, synthetic construct
  REGION 1..70: note = Description of Artificial Sequence: Syntheticpolypeptide
GPETLCGAELVDALQFVCGDRGFYFNKPTGYGSSSRRAPQTGIVDECCFRSCDLRRLEMYCAPLKPAKSA

SEQ ID NO: 445 - protein, 140 aa, synthetic construct
  REGION 1..140: note = Description of Artificial Sequence: Syntheticpolypeptide
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 446 - protein, 146 aa, synthetic construct
  REGION 1..146: note = Description of Artificial Sequence: Syntheticpolypeptide
PALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 447 - protein, 180 aa, synthetic construct
  REGION 1..180: note = Description of Artificial Sequence: Syntheticpolypeptide
EENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 448 - protein, 137 aa, synthetic construct
  REGION 1..137: note = Description of Artificial Sequence: Syntheticpolypeptide
ENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 449 - protein, 50 aa, synthetic construct
  REGION 1..50: note = Description of Artificial Sequence: Syntheticpolypeptide
VVSHFNDCPDSHTQFCFHGTCRFLVQEDKPACVCHSGYVGARCEHADLLA

SEQ ID NO: 450 - protein, 112 aa, synthetic construct
  REGION 1..112: note = Description of Artificial Sequence: Syntheticpolypeptide
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 451 - protein, 112 aa, synthetic construct [sequence omitted]

SEQ ID NO: 452 - protein, 139 aa, synthetic construct
  REGION 1..139: note = Description of Artificial Sequence: Syntheticpolypeptide
AVRPLRRRQPKKSNELPQANRLPGIFDDVHGSHGRQVCRRHELYVSFQDLGWLDWVIAPQGYSAYYCEGECSFPLDSCMNATNHAILQSLVHLMMPDAVPKACCAPTKLSATSVLYYDSSNNVILRKHRNMVVKACGCH

SEQ ID NO: 453 - protein, 139 aa, synthetic construct
  REGION 1..139: note = Description of Artificial Sequence: Syntheticpolypeptide
AVRPLRRRQPKKSNELPQANRLPGIFDDVRGSHGRQVCRRHELYVSFQDLGWLDWVIAPQGYSAYYCEGECSFPLDSCMNATNHAILQSLVHLMKPNAVPKACCAPTKLSATSVLYYDSSNNVILRKHRNMVVKACGCH

SEQ ID NO: 454 - protein, 114 aa, synthetic construct; identical to SEQ ID NO: 440
  REGION 1..114: note = Description of Artificial Sequence: Syntheticpolypeptide

SEQ ID NO: 455 - protein, 110 aa, synthetic construct
  REGION 1..110: note = Description of Artificial Sequence: Syntheticpolypeptide
QWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR

SEQ ID NO: 456 - protein, 116 aa, synthetic construct
  REGION 1..116: note = Description of Artificial Sequence: Syntheticpolypeptide
SPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 457 - protein, 138 aa, synthetic construct
  REGION 1..138: note = Description of Artificial Sequence: Syntheticpolypeptide
AANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 458 - protein, 139 aa, synthetic construct
  REGION 1..139: note = Description of Artificial Sequence: Syntheticpolypeptide
SASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 459 - protein, 139 aa, synthetic construct
  REGION 1..139: note = Description of Artificial Sequence: Syntheticpolypeptide
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 460 - protein, 110 aa, synthetic construct
  REGION 1..110: note = Description of Artificial Sequence: Syntheticpolypeptide
SAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 461 - protein, 108 aa, synthetic construct
  REGION 1..108: note = Description of Artificial Sequence: Syntheticpolypeptide
NAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 462 - protein, 109 aa, synthetic construct
  REGION 1..109: note = Description of Artificial Sequence: Syntheticpolypeptide
NLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 463 - protein, 129 aa, synthetic construct
  REGION 1..129: note = Description of Artificial Sequence: Syntheticpolypeptide
TALAGTRTAQGSGGGAGRGHGRRGRSRCSRKPLHVDFKELGWDDWIIAPLDYEAYHCEGLCDFPLRSHLEPTNHAIIQTLLNSMAPDAAPASCCVPARLSPISILYIDAANNVVYKQYEDMVVEACGCR

SEQ ID NO: 464 - protein, 120 aa, synthetic construct
  REGION 1..120: note = Description of Artificial Sequence: Syntheticpolypeptide
TAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYKQYEDMVVESCGCR

SEQ ID NO: 465 - protein, 125 aa, synthetic construct
  REGION 1..125: note = Description of Artificial Sequence: Syntheticpolypeptide
QADGISAEVTASSSKHSGPENNQCSLHPFQISFRQLGWDHWIIAPPFYTPNYCKGTCLRVLRDGLNSPNHAIIQNLINQLVDQSVPRPSCVPYKYVPISVLMIEANGSILYKEYEGMIAESCTCR

SEQ ID NO: 466 - protein, 447 aa, synthetic construct
  REGION 1..447: note = Description of Artificial Sequence: Syntheticpolypeptide
IPVPQSKPLERHVEKSMNLHLLARSNVSVQDELNASGTIKESGVLVHEGDRGRQENTQDGHKGEGNGSKWAEVGGKSFSTYSTLANEEGNIEGWNGDTGKAETYGHDGIHGKEENITANGIQGQVSIIDNAGATNRSNTNGNTDKNTQNGDVGDAGHNEDVAVVQEDGPQVAGSNNSTDNEDEIIENSCRNEGNTSEITPQINSKRNGTKEAEVTPGTGEDAGLDNSDGSPSGNGADEDEDEGSGDDEDEEAGNGKDSSNNSKGQEGQDHGKEDDHDSSIGQNSDSKEYYDPEGKEDPHNEVDGDKTSKSEENSAGIPEDNGSQRIEDTQKLNHRESKRVENRITKESETHAVGKSQDKGIEIKGPSSGNRNITKEVGKGNEGKEDKGQHGMILGKGNVKTQGEVVNIEGPGQKSEPGNKVGHSNTGSDSNSDGYDSYDFDDKSMQG

SEQ ID NO: 467 - protein, 1286 aa, synthetic construct
  REGION 1..1286: note = Description of Artificial Sequence: Syntheticpolypeptide
IPVPQSKPLERHVEKSMNLHLLARSNVSVQDELNASGTIKESGVLVHEGDRGRQENTQDGHKGEGNGSKWAEVGGKSFSTYSTLANEEGNIEGWNGDTGKAETYGHDGIHGKEENITANGIQGQVSIIDNAGATNRSNTNGNTDKNTQNGDVGDAGHNEDVAVVQEDGPQVAGSNNSTDNEDEIIENSCRNEGNTSEITPQINSKRNGTKEAEVTPGTGEDAGLDNSDGSPSGNGADEDEDEGSGDDEDEEAGNGKDSSNNSKGQEGQDHGKEDDHDSSIGQNSDSKEYYDPEGKEDPHNEVDGDKTSKSEENSAGIPEDNGSQRIEDTQKLNHRESKRVENRITKESETHAVGKSQDKGIEIKGPSSGNRNITKEVGKGNEGKEDKGQHGMILGKGNVKTQGEVVNIEGPGQKSEPGNKVGHSNTGSDSNSDGYDSYDFDDKSMQGDDPNSSDESNGNDDANSESDNNSSSRGDASYNSDESKDNGNGSDSKGAEDDDSDSTSDTNNSDSNGNGNNGNDDNDKSDSGKGKSDSSDSDSSDSSNSSDSSDSSDSDSSDSNSSSDSDSSDSDSSDSSDSDSSDSSNSSDSSDSSDSSDSSDSSDSSDSKSDSSKSESDSSDSDSKSDSSDSNSSDSSDNSDSSDSSNSSNSSDSSDSSDSSDSSSSSDSSNSSDSSDSSDSSNSSESSDSSDSSDSDSSDSSDSSNSNSSDSDSSNSSDSSDSSNSSDSSDSSDSSNSSDSSDSSDSSNSSDSSDSSDSSDSSDSSNSSDSNDSSNSSDSSDSSNSSDSSNSSDSSDSSDSSDSDSSNSSDSSNSSDSSDSSNSSDSSDSSDSSDGSDSDSSNRSDSSNSSDSSDSSDSSNSSDSSDSSDSNESSNSSDSSDSSNSSDSDSSDSSNSSDSSDSSNSSDSSESSNSSDNSNSSDSSNSSDSSDSSDSSNSSDSSNSSDSSNSSDSSDSNSSDSSDSSNSSDSSDSSDSSDSSDSSDSSNSSDSSDSSDSSDSSNSSDSSNSSDSSNSSDSSDSSDSSDSSDSSDSSDSSDSSNSSDSSDSSDSSDSSDSSDSSDSSDSSESSDSSDSSNSSDSSDSSDSSDSSDSSDSSDSSDSSDSSNSSDSSDSSDSSDSSDSSNSSDSSDSSESSDSSDSSDSSDSSDSSDSSDSSDSSDSSNSSDSSDSSDSSDSSDSSDSSDSSDSSDSSDSSDSSDSSDSSDSSDSSDSSDSNESSDSSDSSDSSDSSNSSDSSDSSDSSDSTSDSNDESDSQSKSGNGNNNGSDSDSDSEGSDSNHSTSDD

SEQ ID NO: 468 - protein, 839 aa, synthetic construct
  REGION 1..839: note = Description of Artificial Sequence: Syntheticpolypeptide
DDPNSSDESNGNDDANSESDNNSSSRGDASYNSDESKDNGNGSDSKGAEDDDSDSTSDTNNSDSNGNGNNGNDDNDKSDSGKGKSDSSDSDSSDSSNSSDSSDSSDSDSSDSNSSSDSDSSDSDSSDSSDSDSSDSSNSSDSSDSSDSSDSSDSSDSSDSKSDSSKSESDSSDSDSKSDSSDSNSSDSSDNSDSSDSSNSSNSSDSSDSSDSSDSSSSSDSSNSSDSSDSSDSSNSSESSDSSDSSDSDSSDSSDSSNSNSSDSDSSNSSDSSDSSNSSDSSDSSDSSNSSDSSDSSDSSNSSDSSDSSDSSDSSDSSNSSDSNDSSNSSDSSDSSNSSDSSNSSDSSDSSDSSDSDSSNSSDSSNSSDSSDSSNSSDSSDSSDSSDGSDSDSSNRSDSSNSSDSSDSSDSSNSSDSSDSSDSNESSNSSDSSDSSNSSDSDSSDSSNSSDSSDSSNSSDSSESSNSSDNSNSSDSSNSSDSSDSSDSSNSSDSSNSSDSSNSSDSSDSNSSDSSDSSNSSDSSDSSDSSDSSDSSDSSNSSDSSDSSDSSDSSNSSDSSNSSDSSNSSDSSDSSDSSDSSDSSDSSDSSDSSNSSDSSDSSDSSDSSDSSDSSDSSDSSESSDSSDSSNSSDSSDSSDSSDSSDSSDSSDSSDSSDSSNSSDSSDSSDSSDSSDSSNSSDSSDSSESSDSSDSSDSSDSSDSSDSSDSSDSSDSSNSSDSSDSSDSSDSSDSSDSSDSSDSSDSSDSSDSSDSSDSSDSSDSSDSSDSNESSDSSDSSDSSDSSNSSDSSDSSDSSDSTSDSNDESDSQSKSGNGNNNGSDSDSDSEGSDSNHSTSDD

SEQ ID NO: 469 - protein, 119 aa, synthetic construct
  REGION 1..119: note = Description of Artificial Sequence: Syntheticpolypeptide
DAEPVLGGGPGGACRARRLYVSFREVGWHRWVIAPRGFLANYCQGQCALPVALSGSGGPPALNHAVLRALMHAAAPGAADLPCCVPARLSPISVLFFDNSDNVVLRQYEDMVVDECGCR

SEQ ID NO: 470 - protein, 114 aa, synthetic construct
  REGION 1..114: note = Description of Artificial Sequence: Syntheticpolypeptide
AAIPVPKLSCKNLCHRHQLFINFRDLGWHKWIIAPKGFMANYCHGECPFSLTISLNSSNYAFMQALMHAVDPEIPQAVCIPTKLSPISMLYQDNNDNVILRHYEDMVVDECGCG

SEQ ID NO: 471 - protein, 120 aa, synthetic construct
  REGION 1..120: note = Description of Artificial Sequence: Syntheticpolypeptide
APLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 472 - protein, 109 aa, synthetic construct
  REGION 1..109: note = Description of Artificial Sequence: Syntheticpolypeptide
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 473 - protein, 114 aa, synthetic construct
  REGION 1..114: note = Description of Artificial Sequence: Syntheticpolypeptide
ARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 474 - protein, 206 aa, synthetic construct
  REGION 1..206: note = Description of Artificial Sequence: Syntheticpolypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 475 - protein, 290 aa, synthetic construct
  REGION 1..290: note = Description of Artificial Sequence: Syntheticpolypeptide
FSLMSLLESLDPDWTPDQYDYSYEDYNQEENTSSTLTHAENPDWYYTEDQADPCQPNPCEHGGDCLVHGSTFTCSCLAPFSGNKCQKVQNTCKDNPCGRGQCLITQSPPYYRCVCKHPYTGPSCSQVVPVCRPNPCQNGATCSRHKRRSKFTCACPDQFKGKFCEIGSDDCYVGDGYSYRGKMNRTVNQHACLYWNSHLLLQENYNMFMEDAETHGIGEHNFCRNPDADEKPWCFIKVTNDKVKWEYCDVSACSAQDVAYPEESPTEPSTKLPGFDSCGKTEIAERKIKR

SEQ ID NO: 476 - skipped

SEQ ID NO: 477 - protein, 247 aa, synthetic construct
  REGION 1..247: note = Description of Artificial Sequence: Syntheticpolypeptide
IYGGFKSTAGKHPWQASLQSSLPLTISMPQGHFCGGALIHPCWVLTAAHCTDIKTRHLKVVLGDQDLKKEEFHEQSFRVEKIFKYSHYNERDEIPHNDIALLKLKPVDGHCALESKYVKTVCLPDGSFPSGSECHISGWGVTETGKGSRQLLDAKVKLIANTLCNSRQLYDHMIDDSMICAGNLQKPGQDTCQGDSGGPLTCEKDGTYYVYGIVSWGLECGKRPGVYTQVTKFLNWIKATIKSESGF

SEQ ID NO: 478 - skipped

SEQ ID NO: 479 - protein, 400 aa, synthetic construct
  REGION 1..400: note = Description of Artificial Sequence: Syntheticpolypeptide
AEVKKPAAAAAPGTAEKLSPKAATLAERSAGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFRDKRSALQSINEWAAQTTDGKLPEVTKDVERTDGALLVNAMFFKPHWDEKFHHKMVDNRGFMVTRSYTVGVMMMHRTGLYNYYDDEKEKLQIVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRPKGDKMRDEL

SEQ ID NO: 480 - protein, 163 aa, synthetic construct
  REGION 1..163: note = Description of Artificial Sequence: Syntheticpolypeptide
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 481 - protein, 177 aa, synthetic construct
  REGION 1..177: note = Description of Artificial Sequence: Syntheticpolypeptide
GPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 482 - protein, 243 aa, synthetic construct
  REGION 1..243: note = Description of Artificial Sequence: Syntheticpolypeptide
TNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 483 - protein, 343 aa, synthetic construct
  REGION 1..343: note = Description of Artificial Sequence: Syntheticpolypeptide
ANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 484 - protein, 335 aa, synthetic construct
  REGION 1..335: note = Description of Artificial Sequence: Syntheticpolypeptide
SWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 485 - protein, 333 aa, synthetic construct
  REGION 1..333: note = Description of Artificial Sequence: Syntheticpolypeptide
SWWYIGALGARVICDNIPGLVSRQRQLCQRYPDIMRSVGEGAREWIRECQHQFRHHRWNCTTLDRDHTVFGRVMLRSSREAAFVYAISSAGVVHAITRACSQGELSVCSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDGAVQVMATQDGANFTAARQGYRRATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 486 - protein, 334 aa, synthetic construct
  REGION 1..334: note = Description of Artificial Sequence: Syntheticpolypeptide
GYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYSLFKPPTERDLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 487 - protein, 334 aa, synthetic construct
  REGION 1..334: note = Description of Artificial Sequence: Syntheticpolypeptide
SYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 488 - protein, 329 aa, synthetic construct
  REGION 1..329: note = Description of Artificial Sequence: Syntheticpolypeptide
SNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR

SEQ ID NO: 489 - protein, 319 aa, synthetic construct
  REGION 1..319: note = Description of Artificial Sequence: Syntheticpolypeptide
IIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 490 - protein, 342 aa, synthetic construct
  REGION 1..342: note = Description of Artificial Sequence: Syntheticpolypeptide
QLLTDANSWWSLALNPVQRPEMFIIGAQPVCSQLPGLSPGQRKLCQLYQEHMAYIGEGAKTGIKECQHQFRQRRWNCSTADNASVFGRVMQIGSRETAFTHAVSAAGVVNAISRACREGELSTCGCSRTARPKDLPRDWLWGGCGDNVEYGYRFAKEFVDAREREKNFAKGSEEQGRVLMNLQNNEAGRRAVYKMADVACKCHGVSGSCSLKTCWLQLAEFRKVGDRLKEKYDSAAAMRVTRKGRLELVNSRFTQPTPEDLVYVDPSPDYCLRNESTGSLGTQGRLCNKTSEGMDGCELMCCGRGYNQFKSVQVERCHCKFHWCCFVRCKKCTEIVDQYICK

SEQ ID NO: 491 - protein, 341 aa, synthetic construct
  REGION 1..341: note = Description of Artificial Sequence: Syntheticpolypeptide
LWWAVGSPLVMDPTSICRKARRLAGRQAELCQAEPEVVAELARGARLGVRECQFQFRFRRWNCSSHSKAFGRILQQDIRETAFVFAITAAGASHAVTQACSMGELLQCGCQAPRGRAPPRPSGLPGTPGPPGPAGSPEGSAAWEWGGCGDDVDFGDEKSRLFMDARHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 492 - protein, 318 aa, synthetic construct
  REGION 1..318: note = Description of Artificial Sequence: Syntheticpolypeptide
LGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 493 - protein, 325 aa, synthetic construct
  REGION 1..325: note = Description of Artificial Sequence: Syntheticpolypeptide
ALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 494 - protein, 327 aa, synthetic construct
  REGION 1..327: note = Description of Artificial Sequence: Syntheticpolypeptide
VNNFLITGPKAYLTYTTSVALGAQSGIEECKFQFAWERWNCPENALQLSTHNRLRSATRETSFIHAISSAGVMYIITKNCSMGDFENCGCDGSNNGKTGGHGWIWGGCSDNVEFGERISKLFVDSLEKGKDARALMNLHNNRAGRLAVRATMKRTCKCHGISGSCSIQTCWLQLAEFREMGDYLKAKYDQALKIEMDKRQLRAGNSAEGHWVPAEAFLPSAEAELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGKGSA

SEQ ID NO: 495 - protein, 329 aa, synthetic construct
  REGION 1..329: note = Description of Artificial Sequence: Syntheticpolypeptide
WSVNNFLMTGPKAYLIYSSSVAAGAQSGIEECKYQFAWDRWNCPERALQLSSHGGLRSANRETAFVHAISSAGVMYTLTRNCSLGDFDNCGCDDSRNGQLGGQGWLWGGCSDNVGFGEAISKQFVDALETGQDARAAMNLHNNEAGRKAVKGTMKRTCKCHGVSGSCTTQTCWLQLPEFREVGAHLKEKYHAALKVDLLQGAGNSAAGRGAIADTFRSISTRELVHLEDSPDYCLENKTLGLLGTEGRECLRRGRALGRWERRSCRRLCGDCGLAVEERRAETVSSCNCKFHWCCAVRCEQCRRRVTKYFCSRAERPRGGAAHKPGRKP

SEQ ID NO: 496 - protein, 336 aa, synthetic construct
  REGION 1..336: note = Description of Artificial Sequence: Syntheticpolypeptide
YFGLTGSEPLTILPLTLEPEAAAQAHYKACDRLKLERKQRRMCRRDPGVAETLVEAVSMSALECQFQFRFERWNCTLEGRYRASLLKRGFKETAFLYAISSAGLTHALAKACSAGRMERCTCDEAPDLENREAWQWGGCGDNLKYSSKFVKEFLGRRSSKDLRARVDFHNNLVGVKVIKAGVETTCKCHGVSGSCTVRTCWRQLAPFHEVGKHLKHKYETALKVGSTTNEAAGEAGAISPPRGRASGAGGSDPLPRTPELVHLDDSPSFCLAGRFSPGTAGRRCHREKNCESICCGRGHNTQSRVVTRPCQCQVRWCCYVECRQCTQREEVYTCKG

SEQ ID NO: 497 - protein, 335 aa, synthetic construct
  REGION 1..335: note = Description of Artificial Sequence: Syntheticpolypeptide
SYFGLTGREVLTPFPGLGTAAAPAQGGAHLKQCDLLKLSRRQKQLCRREPGLAETLRDAAHLGLLECQFQFRHERWNCSLEGRMGLLKRGFKETAFLYAVSSAALTHTLARACSAGRMERCTCDDSPGLESRQAWQWGVCGDNLKYSTKFLSNFLGSKRGNKDLRARADAHNTHVGIKAVKSGLRTTCKCHGVSGSCAVRTCWKQLSPFRETGQVLKLRYDSAVKVSSATNEALGRLELWAPARQGSLTKGLAPRSGDLVYMEDSPSFCRPSKYSPGTAGRVCSREASCSSLCCGRGYDTQSRLVAFSCHCQVQWCCYVECQQCVQEELVYTCKH

SEQ ID NO: 498 - protein, 382 aa, synthetic construct
  REGION 1..382: note = Description of Artificial Sequence: Syntheticpolypeptide
MPRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGALLRSRFHRATLIRPHNRNGGQLEPGPAGAPSPAPGAPGPRRRASPADLVYFEKSPDFCEREPRLDSAGTVGRLCNKSSAGSDGCGSMCCGRGHNILRQTRSERCHCRFHWCCFVVCEECRITEWVSVCK

SEQ ID NO: 499 - protein, 361 aa, synthetic construct
  REGION 1..361: note = Description of Artificial Sequence: Syntheticpolypeptide
NEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGRAIFIDTHNRNSGAFQPRLRPRRLSGELVYFEKSPDFCERDPTMGSPGTRGRACNKTSRLLDGCGSLCCGRGHNVLRQTRVERCHCRFHWCCYVLCDECKVTEWVNVCK

SEQ ID NO: 500 - protein, 330 aa, synthetic construct
  REGION 1..330: note = Description of Artificial Sequence: Syntheticpolypeptide
IKWLALSKTPSALALNQTQHCKQLEGLVSAQVQLCRSNLELMHTVVHAAREVMKACRRAFADMRWNCSSIELAPNYLLDLERGTRESAFVYALSAAAISHAIARACTSGDLPGCSCGPVPGEPPGPGNRWGGCADNLSYGLLMGAKFSDAPMKVKKTGSQANKLMRLHNSEVGRQALRASLEMKCKCHGVSGSCSIRTCWKGLQELQDVAADLKTRYLSATKVVHRPMGTRKHLVPKDLDIRPVKDSELVYLQSSPDFCMKNEKVGSHGTQDRQCNKTSNGSDSCDLMCCGRGYNPYTDRVVERCHCKYHWCCYVTCRRCERTVERYVCK

SEQ ID NO: 501 - protein, 336 aa, synthetic construct
  REGION 1..336: note = Description of Artificial Sequence: Syntheticpolypeptide
NWMWLGIASFGVPEKLGCANLPLNSRQKELCKRKPYLLPSIREGARLGIQECGSQFRHERWNCMITAAATTAPMGASPLFGYELSSGTKETAFIYAVMAAGLVHSVTRSCSAGNMTECSCDTTLQNGGSASEGWHWGGCSDDVQYGMWFSRKFLDFPIGNTTGKENKVLLAMNLHNNEAGRQAVAKLMSVDCRCHGVSGSCAVKTCWKTMSSFEKIGHLLKDKYENSIQISDKTKRKMRRREKDQRKIPIHKDDLLYVNKSPNYCVEDKKLGIPGTQGRECNRTSEGADGCNLLCCGRGYNTHVVRHVERCECKFIWCCYVRCRRCESMTDVHTCK

SEQ ID NO: 502 - protein, 685 aa, synthetic construct
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 503 - protein, 498 aa, synthetic construct
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLP

SEQ ID NO: 504 - protein, 191 aa, synthetic construct
SGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYC

(19) United States
(12) Patent Application Publication
(10) Pub. No.: US 2026/0315658 A1
(43) Pub. Date: Oct. 15, 2026

(54) 3D PRINTED CERAMIC COMPOSITIONS AND METHODS OF USE

(71) Applicant: THERADAPTIVE, INC., Frederick, MD (US)

(72) Inventors: Luis ALVAREZ, Lexington, MA (US); Todd HEIL, Mt. Airy, MD (US); David LONG, Ellicott, MD (US)

(21) Appl. No.: 19/105,766

(22) PCT Filed: Aug. 22, 2023
(86) PCT No.: PCT/US2023/072672

§ 371 Date: Feb. 21, 2025

Related U.S. Application Data
(60) Provisional application No. 63/373,278, filed on Aug. 23, 2022.

(51) Int. Cl. A61L 27/46; A61L 27/54; A61L 27/56; A61L 27/58; B29C 64/106; B33Y 70/10; B33Y 80/00

(52) U.S. Cl. CPC A61L 27/46; A61L 27/54; A61L 27/56; A61L 27/58; B29C 64/106; B33Y 70/10; B33Y 80/00; A61L 2300/414; A61L 2430/02; A61L 2430/38

(57) ABSTRACT

The present disclosure provides devices comprising a therapeutic agent bound to a printed three-dimensional structure. Ink formulations for three-dimensional printing are also disclosed. Additionally, provided herein are methods for manufacturing devices and uses thereof, e.g., in treating a condition in a subject in need thereof.

### CROSS-REFERENCE

This application is a U.S. National Phase Application of International Application No. PCT/US2023/072672, filed Aug. 22, 2023, which claims the benefit of U.S. Provisional Application No. 63/373,278 filed on Aug. 23, 2022 which are incorporated herein by reference in their entirety.

### SEQUENCE LISTING

The application contains a Sequence Listing, which is submitted herewith in XML format, and is hereby incorporated by reference in its entirety. The XML copy, created on Mar. 2, 2026, is named 50222-711_831_SL.xml and is 703,762 bytes in size.

### BACKGROUND

Three-dimensional (3D) printing is a manufacturing process of making three dimensional solid objects from a digital file. In the additive process of 3D printing an object is created by laying down successive layers of material until the desired object is created, with up to micrometer accuracy. Paired with computer-aided design (CAD) software, 3D printing enables production of complex functional shapes that can be easily customized compared with traditional manufacturing methods.

Surgically implanting scaffolds and/or other forms of graft materials to promote tissue regeneration is a useful technique if the implant can match the mechanical properties of the native tissue. Various materials can be used as ink in the fabrication of porous 3D-printed structures for implantation, including materials that mimic tissue and enable tissue regrowth. Inks with effective bioactive and mechanical properties are required for regeneration of native tissue, and if used in 3D printing can be customized and adopted for large tissue defect repair.

### SUMMARY

The present disclosure provides ink formulations and methods for 3D printing scaffolds. Further provided are scaffolds that may be generated using such ink formulations and methods. The scaffolds may be coated with therapeutic agents, such as those that promote bone growth, and/or seeded with cells to generate devices for use in tissue replacement and grafting. For some devices, therapeutic agents may be tethered to the scaffold via a targeting moiety that interacts with a scaffold component, such as a ceramic material. Advantages of the materials and methods described herein include providing a 3D-printed, customizable implant, as well as more universal objects, such as strip, block, or cylindrical objects. As the implants are 3D-printed, precise control of the implant geometry is possible. Implants printed with these inks are thus suitable for many different therapies including long bone repair, spinal fusion, maxio-facial structures, etc. The different formulations of the inks produce materials that result in implantable devices with differing properties, including differing porosity and flexibility.

Various scaffolds of the present disclosure are prepared using fused granular fabrication (FGF) 3D printing. The inks for use in such FGF 3D printing methods may be designed with a particular viscosity that minimizes oozing of molten ink out of the 3D Printer nozzle during non-print motions but is low enough viscosity to enable flow through a 100's of microns diameter nozzle when the screw extruder is engaged for print motions. In a non-limiting example, the ink comprises one or more water soluble polymers, wherein the identity and/or molecular weight of the one or more water soluble polymers is increased or decreased to modulate the viscosity of the ink. For instance, the one or more water soluble polymers comprises polyethylene glycol (PEG), where a PEG with a higher molecular weight has a higher melt viscosity. Non-limiting example inks are described in the examples herein.

Various inks and scaffolds of the present disclosure are designed to enhance the accessibility of therapeutic agents and/or targeting moieties to scaffold components, such as ceramic materials like beta-tricalcium phosphate. For instance, ink formulations comprising a sacrificial pore former. Sacrificial pore formers include water soluble polymers such as PEG, as well as particulates described herein. Non-limiting example inks having sacrificial pore formers are described in the examples herein.

Various inks and methods of the present disclosure are designed to improve the manufacturability of 3D printed scaffolds by converting the ink to pellets. Pellets are a common polymer feedstock form that enables easy storage and transport at large scales. Pellets do not require stringent mechanical properties or dimensional tolerances (compared to 3D printing filaments). Pellet feedstock is amenable to large scale, continuous feed via a hopper, in contrast to a finite spool of filament which must be changed periodically.

Various inks and scaffolds of the present disclosure are designed to optimize bioresorption characteristics of the scaffolds. For instance, the copolymers used in certain formulations, e.g., caprolactone/glycolide copolymer (95:5), caprolactone/glycolide copolymer (90:10), poly(D,L-lactide-co-glycolide) copolymer (50:50), have faster resorption rates than other formulations, e.g., polycaprolactone. The resorption rates vary from slowest to fastest as: polycaprolactone, polycaprolactone/polyglycolide copolymer (95:5), polycaprolactone/glycolide copolymer (90:10), polydioxanone/L-lactide copolymer (90:10), poly(D,L-lactide-co-glycolide) copolymer (50:50).

In one aspect, provided herein is an ink formulation comprising about 55% to about 65% by weight beta-tricalcium phosphate (BTCP), about 15% to about 25% by weight caprolactone/glycolide copolymer, about 5% to about 15% polyethylene glycol (PEG) having a molecular weight from about 500 g/mol to about 15,000 g/mol, and about 5% to about 15% PEG having a molecular weight from about 25,000 g/mol to about 50,000 g/mol. In some embodiments, the ink formulation comprises about 60% by weight BTCP, about 20% by weight caprolactone/glycolide copolymer, about 10% by weight PEG having a molecular weight from about 500 g/mol to about 15,000 g/mol, and about 10% PEG having a molecular weight from about 25,000 g/mol to about 50,000 g/mol. In some embodiments, the ink formulation comprises about 60% by weight BTCP, about 20% by weight caprolactone/glycolide copolymer, about 10% by weight PEG having a molecular weight of about 8,000 g/mol, and about 10% PEG having a molecular weight of about 35,000 g/mol. In some embodiments, the caprolactone/glycolide copolymer is caprolactone/glycolide copolymer (95:5). In some embodiments, the caprolactone/glycolide copolymer is caprolactone/glycolide copolymer (90:10).

In some embodiments, provided herein is a method of preparing a three-dimensional structure, the method comprises performing additive manufacturing with the ink formulation. In some embodiments, the ink formulation is in the form of a pellet. In some embodiments, the additive manufacturing comprises fused granular fabrication (FGF).

In some embodiments, provided herein is a structure prepared by additive manufacturing of the ink formulation.

In some embodiments, provided herein is a method of treating a bone defect in a subject in need thereof, the method comprising application of the structure to the defect of the subject. In some embodiments, the defect is present in the spine.

In some embodiments, provided herein is a device comprising a therapeutic agent and the structure. In some embodiments, the therapeutic agent is non-covalently bound to the structure. In some embodiments, the therapeutic agent comprises a growth factor. In some embodiments, the growth factor is selected from Table 1. In some embodiments, the therapeutic agent comprises a bone morphogenetic protein (BMP). In some embodiments, the therapeutic agent comprises a targeting moiety, and the targeting moiety is non-covalently bound to the structure. In some embodiments, the targeting moiety comprises a polypeptide at least about 70%, 75%, 80%, 85%, 90%, 95%, or 100% identical to any one of the sequences of Tables 2-3. In some embodiments, the therapeutic agent comprises a chimeric polypeptide comprising a sequence at least about 70%, 75%, 80%, 85%, 90%, 95%, or 100% identical to any one of SEQ ID NOS: 433-441.

In some embodiments, provided herein is a method of treating a bone defect in a subject in need thereof, the method comprising application of the device to the defect of the subject. In some embodiments, the defect is present in the spine.

In one aspect, provided herein is a three-dimensional structure comprising 70% to about 80% by weight βTCP and about 20% to about 30% by weight polycaprolactone (PCL). In some embodiments, the three-dimensional structure comprises about 75% by weight βTCP and about 25% by weight PCL. In one aspect, provided herein is a three-dimensional structure comprising about 70% to about 80% by weight βTCP and about 20% to about 30% by weight caprolactone/glycolide copolymer. In some embodiments, the three-dimensional structure comprises about 75% by weight βTCP and about 25% by weight caprolactone/glycolide copolymer. In some embodiments, the caprolactone/glycolide copolymer is caprolactone/glycolide copolymer (95:5). In some embodiments, the caprolactone/glycolide copolymer is caprolactone/glycolide copolymer (90:10). In one aspect, provided herein is a three-dimensional structure comprising about 70% to about 80% by weight βTCP and about 20% to about 30% by weight poly(D,L-lactide-co-glycolide) copolymer. In some embodiments, the three-dimensional structure comprises about 75% by weight βTCP and about 25% by weight poly(D,L-lactide-co-glycolide) copolymer. In some embodiments, the caprolactone/glycolide copolymer is poly(D,L-lactide-co-glycolide) copolymer (50:50). In one aspect, provided herein is a three-dimensional structure comprising about 70% to about 80% by weight βTCP and about 20% to about 30% by weight polydioxanone (PDS). In some embodiments, the three-dimensional structure comprises about 75% by weight βTCP and about 25% by weight PDS. In one aspect, provided herein is a three-dimensional structure comprising about 70% to about 80% by weight βTCP and about 20% to about 30% by weight dioxanone/L-lactide copolymer. In some embodiments, the three-dimensional structure comprises about 75% by weight βTCP and about 25% by weight dioxanone/L-lactide copolymer. In some embodiments, the caprolactone/glycolide copolymer is dioxanone/L-lactide copolymer (90:10). In one aspect, provided herein is a three-dimensional structure comprising about 70% to about 80% by weight βTCP and about 20% to about 30% by weight glycolide/L-lactide copolymer. In some embodiments, the three-dimensional structure comprises about 75% by weight βTCP and about 25% by weight glycolide/L-lactide copolymer. In some embodiments, the caprolactone/glycolide copolymer is glycolide/L-lactide copolymer (95:5). In one aspect, provided herein is a three-dimensional structure comprising In one aspect, provided herein is a three-dimensional structure comprising about 70% to about 80% by weight βTCP and about 20% to about 30% by weight poly-1-lactide. In some embodiments, the three-dimensional structure comprises about 75% by weight βTCP and about 25% by weight poly-1-lactide.

In some embodiments of the three-dimensional structures herein, the density is about 1 to about 1.5 g/cm³. In some embodiments of the three-dimensional structures herein, the open porosity is about 25% to about 40%. In some embodiments of the three-dimensional structures herein, the strut diameter is about 300 μm to 800 μm, or about 300 μm, 400 μm, 500 μm, 600 μm, 700 μm, or 800 μm. In some embodiments of the three-dimensional structures herein, the structure comprises a plurality of micropores, wherein the micropores have an average pore size of about 1 micron to about 500 microns or about 1 to about 50 microns.

Further provided are methods of preparing the three-dimensional structures herein, the method comprising additive manufacturing. In some embodiments, the additive manufacturing comprises fused granular fabrication (FGF) or fused filament fabrication (FFF).

Further provided is a device comprising a structure provided herein, and a therapeutic agent. In some embodiments, the therapeutic agent is non-covalently bound to the structure. In some embodiments, the therapeutic agent comprises a growth factor. In some embodiments, the growth factor is selected from Table 1. In some embodiments, the therapeutic agent comprises a bone morphogenetic protein (BMP). In some embodiments, the therapeutic agent comprises a targeting moiety, and the targeting moiety is non-covalently bound to the three-dimensional structure. In some embodiments, the targeting moiety comprises a polypeptide at least about 70%, 75%, 80%, 85%, 90%, 95%, or 100% identical to any one of the sequences of Tables 2-3. In some embodiments, the therapeutic agent comprises a chimeric polypeptide comprising a sequence at least about 70%, 75%, 80%, 85%, 90%, 95%, or 100% identical to any one of SEQ ID NOS: 433-441.

Further provided is a method of treating a condition in a subject in need thereof, the method comprising administering to the subject the structure or device herein. In some embodiments, the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof. In some embodiments, the method comprises spinal fusion. In some embodiments, the spinal fusion comprises posterior lumbar fusion (PLF) and/or interbody fusion. In some embodiments, the method comprises bone repair, dental repair, craniomaxillofacial repair, ankle fusion, kyphoplasty, osteoplasty, scaphoid fracture repair, tenden-osseous repair, costal reconstruction, subchondral bone repair, cartilage repair, or surgical implantation of the three-dimensional structure or device, or a combination thereof.

Further provided is a formulation comprising about 55% to about 65% by weight βTCP, about 15% to about 25% by weight PCL, about 5% to about 15% PEG having a molecular weight from about 500 g/mol to about 15,000 g/mol, and about 5% to about 15% PEG having a molecular weight from about 25,000 g/mol to about 50,000 g/mol. In some embodiments, the formulation comprises about 60% by weight βTCP, about 20% by weight PCL, about 10% by weight PEG having a molecular weight from about 500 g/mol to about 15,000 g/mol, and about 10% PEG having a molecular weight from about 25,000 g/mol to about 50,000 g/mol. In some embodiments, the formulation comprises about 60% by weight βTCP, about 20% by weight PCL, about 10% by weight PEG having a molecular weight of about 8,000 g/mol, and about 10% PEG having a molecular weight of about 35,000 g/mol. In some embodiments, the formulation comprises about 1% to about 10% of a sacrificial pore former. In some embodiments, the sacrificial pore former comprises sucrose.

Further provided is a formulation comprising about 55% to about 65% by weight βTCP, about 15% to about 25% by weight caprolactone/glycolide copolymer, about 5% to about 15% PEG having a molecular weight from about 500 g/mol to about 15,000 g/mol, and about 5% to about 15% PEG having a molecular weight from about 25,000 g/mol to about 50,000 g/mol. In some embodiments, the formulation comprises about 60% by weight βTCP, about 20% by weight caprolactone/glycolide copolymer, about 10% by weight PEG having a molecular weight from about 500 g/mol to about 15,000 g/mol, and about 10% PEG having a molecular weight from about 25,000 g/mol to about 50,000 g/mol. In some embodiments, the formulation comprises about 60% by weight βTCP, about 20% by weight caprolactone/glycolide copolymer, about 10% by weight PEG having a molecular weight of about 8,000 g/mol, and about 10% PEG having a molecular weight of about 35,000 g/mol. In some embodiments, the caprolactone/glycolide copolymer is caprolactone/glycolide copolymer (95:5). In some embodiments, the caprolactone/glycolide copolymer is caprolactone/glycolide copolymer (90:10).

Further provided is a formulation comprising about 55% to about 65% by weight βTCP, about 15% to about 25% by weight poly(D,L-lactide-co-glycolide) copolymer, about 5% to about 15% PEG having a molecular weight from about 500 g/mol to about 15,000 g/mol, and about 5% to about 15% PEG having a molecular weight from about 25,000 g/mol to about 50,000 g/mol. In some embodiments, the formulation comprises about 60% by weight βTCP, about 20% by weight poly(D,L-lactide-co-glycolide) copolymer, about 10% by weight PEG having a molecular weight from about 500 g/mol to about 15,000 g/mol, and about 10% PEG having a molecular weight from about 25,000 g/mol to about 50,000 g/mol. In some embodiments, the formulation comprises about 60% by weight βTCP, about 20% by weight poly(D,L-lactide-co-glycolide) copolymer, about 10% by weight PEG having a molecular weight of about 8,000 g/mol, and about 10% PEG having a molecular weight of about 35,000 g/mol. In some embodiments, the caprolactone/glycolide copolymer is caprolactone/glycolide copolymer (50:50).

Further provided is a formulation comprising about 55% to about 65% by weight βTCP, about 15% to about 25% by weight PDS, about 5% to about 15% PEG having a molecular weight from about 500 g/mol to about 15,000 g/mol, and about 5% to about 15% PEG having a molecular weight from about 25,000 g/mol to about 50,000 g/mol. In some embodiments, the formulation comprises about 60% by weight βTCP, about 20% by weight PDS, about 10% by weight PEG having a molecular weight from about 500 g/mol to about 15,000 g/mol, and about 10% PEG having a molecular weight from about 25,000 g/mol to about 50,000 g/mol. In some embodiments, the formulation comprises about 60% by weight βTCP, about 20% by weight PDS, about 10% by weight PEG having a molecular weight of about 8,000 g/mol, and about 10% PEG having a molecular weight of about 35,000 g/mol.

Further provided is a formulation comprising about 55% to about 65% by weight βTCP, about 15% to about 25% by weight dioxanone/L-lactide copolymer, about 5% to about 15% PEG having a molecular weight from about 500 g/mol to about 15,000 g/mol, and about 5% to about 15% PEG having a molecular weight from about 25,000 g/mol to about 50,000 g/mol. In some embodiments, the formulation comprises about 60% by weight βTCP, about 20% by weight dioxanone/L-lactide copolymer, about 10% by weight PEG having a molecular weight from about 500 g/mol to about 15,000 g/mol, and about 10% PEG having a molecular weight from about 25,000 g/mol to about 50,000 g/mol. In some embodiments, the formulation comprises about 60% by weight βTCP, about 20% by weight dioxanone/L-lactide copolymer, about 10% by weight PEG having a molecular weight of about 8,000 g/mol, and about 10% PEG having a molecular weight of about 35,000 g/mol. In some embodiments, the caprolactone/glycolide copolymer is caprolactone/glycolide copolymer (90:10).

Further provided is a formulation comprising about 55% to about 65% by weight βTCP, about 15% to about 25% by weight glycolide/L-lactide copolymer, about 5% to about 15% PEG having a molecular weight from about 500 g/mol to about 15,000 g/mol, and about 5% to about 15% PEG having a molecular weight from about 25,000 g/mol to about 50,000 g/mol. In some embodiments, the formulation comprises about 60% by weight βTCP, about 20% by weight glycolide/L-lactide copolymer, about 10% by weight PEG having a molecular weight from about 500 g/mol to about 15,000 g/mol, and about 10% PEG having a molecular weight from about 25,000 g/mol to about 50,000 g/mol. In some embodiments, the formulation comprises about 60% by weight βTCP, about 20% by weight glycolide/L-lactide copolymer, about 10% by weight PEG having a molecular weight of about 8,000 g/mol, and about 10% PEG having a molecular weight of about 35,000 g/mol. In some embodiments, the caprolactone/glycolide copolymer is glycolide/L-lactide copolymer (95:5).

Further provided is a formulation comprising about 55% to about 65% by weight βTCP, about 15% to about 25% by weight poly-1-lactide, about 5% to about 15% PEG having a molecular weight from about 500 g/mol to about 15,000 g/mol, and about 5% to about 15% PEG having a molecular weight from about 25,000 g/mol to about 50,000 g/mol. In some embodiments, the formulation comprises about 60% by weight βTCP, about 20% by weight poly-1-lactide, about 10% by weight PEG having a molecular weight from about 500 g/mol to about 15,000 g/mol, and about 10% PEG having a molecular weight from about 25,000 g/mol to about 50,000 g/mol. In some embodiments, the formulation comprises about 60% by weight βTCP, about 20% by weight poly-1-lactide, about 10% by weight PEG having a molecular weight of about 8,000 g/mol, and about 10% PEG having a molecular weight of about 35,000 g/mol.

Further provided is a pellet comprising the formulation. Further provided is a method of preparing a three-dimensional structure, the method comprising using the formation in a three-dimensional printing method.

Further provided is a three-dimensional structure prepared using the ink formulation. In some embodiments, the structure comprises about 70% to about 80% by weight βTCP and about 20% to about 30% by weight polymer selected from PCL, PDS, poly-1-lactide, caprolactone/glycolide copolymer, poly(D,L-lactide-co-glycolide) copolymer, dioxanone/L-lactide copolymer, and glycolide/L-lactide copolymer. Further provided is a method of manufacturing the three-dimensional structure, the method comprising depositing an ink formulation in a three-dimensional form, e.g., additive manufacturing. In some embodiments, the method comprises FFF. In some embodiments, the method comprises FGF. In some embodiments, the ink formulation comprises the ink formulation herein. In some embodiments, the ink formulation is in the form of a pellet. In some embodiments, the ink formulation is in the form of a filament.

Further provided is a method of treating a condition in a subject in need thereof, the method comprising delivering to an organ or tissue of the subject the structure.

Further provided is a method of treating a condition in a subject in need thereof, the method comprising delivering to an organ or tissue of the subject the structure manufactured by a method herein.

In some embodiments, the condition comprises a bone defect, cartilage defect, soft tissue defect, tendon defect, fascia defect, ligament defect, organ defect, osteotendinous tissue defect, dermal defect, osteochondral defect, osteoporosis, avascular necrosis, or congenital skeletal malformation, or a combination thereof. In some embodiments, the method comprises spinal fusion. In some embodiments, the spinal fusion comprises posterior lumbar fusion (PLF) and/or interbody fusion. In some embodiments, the method comprises bone repair, dental repair, craniomaxillofacial repair, ankle fusion, kyphoplasty, osteoplasty, scaphoid fracture repair, tendon-osseous repair, costal reconstruction, subchondral bone repair, cartilage repair, or surgical implantation of the three-dimensional structure or device, or a combination thereof. In some embodiments, the method further comprises treating the subject with a therapeutic agent.

Further provided is a method of delivering a therapeutic agent to a subject in need thereof, the method comprising delivering to an organ or tissue of the subject a device comprising a therapeutic agent and the structure.

In some embodiments, the therapeutic agent comprises a mammalian growth factor or a functional portion thereof. In some embodiments, the therapeutic agent comprises one or more polypeptides selected from Table 1, or a functional portion thereof. In some embodiments, the therapeutic agent comprises a bone morphogenetic protein (BMP). In some embodiments, the therapeutic agent comprises a targeting moiety that non-covalently binds to the structure. In some embodiments, the targeting moiety binds to the printed three-dimensional structure with an affinity of about 1 μM to about 100 μm. In some embodiments, the targeting moiety comprises a polypeptide at least about 70%, 75%, 80%, 85%, 90%, 95%, or 100% identical to any one of the sequences of Tables 5-6. In some embodiments, the targeting moiety comprises about 2, 3, 4, 5, 6, 7, 8, 9, or 10 sequences selected from the sequences of Tables 5-6. In some embodiments, the therapeutic agent comprises or is part of a chimeric polypeptide comprising a sequence at least about 70%, 75%, 80%, 85%, 90%, 95%, or 100% identical to any one of SEQ ID NOS: 433-441.

In some embodiments of a device and/or structure herein, the structure has a density of about 1 g/cm3 to about 1.5 g/cm³. In some embodiments of a device and/or structure herein, the structure has an open porosity of about 25% to about 40%. In some embodiments of a device and/or structure herein the structure has a strut diameter of about 300 μm to about 800 μm.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIGS. 1A-1C are images from scanning electron microscopy (SEM images) of an example 3D printed object made with ink formulation #1 as outlined in Example 2. FIG. 1A and FIG. 1C are SEM images of the surface of the object at increasing magnifications. FIG. 1B is an SEM image of the side of the object.

FIGS. 2A-2C are images from scanning electron microscopy (SEM images) of an example 3D printed object made with ink formulation #2 as outlined in Example 2. FIG. 2A and FIG. 2C are SEM images of the surface of the object at increasing magnifications. FIG. 2B is an SEM image of the side of the object.

FIGS. 3A-3C are images from scanning electron microscopy (SEM images) of an example 3D printed object made with ink formulation #3 as outlined in Example 2. FIG. 3A and FIG. 3C are SEM images of the surface of the object at increasing magnifications. FIG. 3B is an SEM image of the side of the object.

FIGS. 4A-4C are images from scanning electron microscopy (SEM images) of an example 3D printed object made with ink formulation #4 as outlined in Example 2. FIG. 4A and FIG. 4C are SEM images of the surface of the object at increasing magnifications. FIG. 4B is an SEM image of the side of the object.

FIGS. 5A-5C are images from scanning electron microscopy (SEM images) of an example 3D printed object made with ink formulation #5 as outlined in Example 2. FIG. 5A and FIG. 5C are SEM images of the surface of the object at increasing magnifications. FIG. 5B is an SEM image of the side of the object.

FIGS. 6A-6C are images from scanning electron microscopy (SEM images) of an example 3D printed object made with ink formulation #6 as outlined in Example 2. FIG. 6A and FIG. 6C are SEM images of the surface of the object at increasing magnifications. FIG. 6B is an SEM image of the side of the object.

FIGS. 7A-7C are images from scanning electron microscopy (SEM images) of an example 3D printed object made with ink formulation #7 as outlined in Example 2. FIG. 7A and FIG. 7C are SEM images of the surface of the object at increasing magnifications. FIG. 7B is an SEM image of the side of the object.

FIGS. 8A-8C are images from scanning electron microscopy (SEM images) of an example 3D printed object made with ink formulation #8 as outlined in Example 2. FIG. 8A and FIG. 8C are SEM images of the surface of the object at increasing magnifications. FIG. 8B is an SEM image of the side of the object.

FIGS. 9A-9C are images from scanning electron microscopy (SEM images) of an example 3D printed object made with ink formulation #9 as outlined in Example 2. FIG. 9A and FIG. 9C are SEM images of the surface of the object at increasing magnifications. FIG. 9B is an SEM image of the side of the object.

FIGS. 10A-10C are images from scanning electron microscopy (SEM images) of an example 3D printed object made with ink formulation #10 as outlined in Example 2. FIG. 10A and FIG. 10C are SEM images of the surface of the object at increasing magnifications. FIG. 10B is an SEM image of the side of the object.

FIGS. 11A-11C are images from scanning electron microscopy (SEM images) of an example 3D printed object made with ink formulation #11 as outlined in Example 2. FIG. 11A and FIG. 11C are SEM images of the surface of the object at increasing magnifications. FIG. 11B is an SEM image of the side of the object.

FIGS. 12A-12C are images from scanning electron microscopy (SEM images) of an example 3D printed object made with ink formulation #12 as outlined in Example 2. FIG. 12A and FIG. 12C are SEM images of the surface of the object at increasing magnifications. FIG. 12B is an SEM image of the side of the object.

FIG. 13A is a photograph of an example flexible 3-layer membrane made with ink formulation #16 3D printed with a 400 micron nozzle as outlined in Example 2. FIGS. 13B-13D are images from scanning electron microscopy (SEM images) of the flexible 3-layer membrane at increasing magnifications.

FIG. 14A is a photograph of an example gyroid scaffold made with ink formulation #16 3D printed with a 400 micron nozzle as outlined in Example 2. FIGS. 14B-14D are images from scanning electron microscopy (SEM images) of the gyroid scaffold at increasing magnifications.

FIG. 15A is a photograph of an example flexible 3-layer membrane made with ink formulation #18 3D printed with a 400 micron nozzle as outlined in Example 2. FIG. 15B is a photograph of a hollow cylinder made with ink #18 3D printed with a 400 micron nozzle as outline in Example 2.

FIG. 16A is a photograph of an example flexible 3-layer membrane made with ink formulation #19 3D printed with a 400 micron nozzle as outlined in Example 2. FIG. 16B is a photograph of a hollow cylinder made with ink #19 3D printed with a 400 micron nozzle as outline in Example 2.

FIG. 17A are microscope images of L929 mouse fibroblasts following an in vitro cytotoxicity assay that was performed to determine cell response, specifically toxic effects, when exposed to extracts from the 3D printed scaffolds. FIG. 17B shows the results of the cytotoxicity assay as determined using the following cytotoxicity scale defined in ISO 10993-5:2009 standard.

### DETAILED DESCRIPTION

Various formulations and structures are provided herein. The structures may be coated with a tetherable protein (for example, a growth factor) for a desired therapeutic effect, such as promotion of bone growth after implantation of the tethered structure.

### Formulations

In one aspect, provided herein are formulations for fabrication of 3D-printed structures. As a non-limiting example, the formulations include a ceramic material such as calcium phosphate (e.g., tricalcium phosphate, beta tricalcium phosphate, alpha tricalcium phosphate), hydroxyapatite, fluorapatite, bone (e.g., demineralized bone), glasses (bioglasses) such as silicates, vanadates, and related ceramic minerals, or chelated divalent metal ions, or a combination thereof. In some embodiments, the ceramic material comprises beta-tricalcium phosphate (β-TCP). In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent ceramic by weight of the formulation. For instance, the formulation is about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% ceramic by weight. In a non-limiting example, the formulation is about 60% ceramic by weight. In some embodiments, the ceramic is β-TCP. In some embodiments, the β-TCP is introduced into the formulation as a powder. In some embodiments, the formulation comprises one or more additional components. Non-limiting examples of additional components include water, polymer (including copolymer), antifoaming agent, dispersing agent, solvent, particulate or sacrificial pore former, blowing agent, and plasticizer.

In some embodiments, the formulation comprises one or more polymers, e.g., about 1, 2, 3, 4, or 5 polymers. Non-limiting examples of polymers include poly(ethylene oxide), poly(propylene oxide), polyethylene glycol (PEG), and polyester. In some embodiments, the polymer is a water soluble polymer, e.g., PEG. In some embodiments, the formulation comprises a polymer that is about 5-30 percent by weight of the formulation. In some embodiments, the formulation comprises a polymer that is about 10-30 percent by weight of the formulation. In some embodiments, the formulation is about 20-60 percent total polymer by weight. For instance, the total polymer includes two or more polymers in the formulation, where the total percentage of polymers in the formulation is about 20-60 percent of the weight of the formulation. In an example embodiment, a first polymer is present at about 5-15% by weight of the formulation, and a second polymer is present at about 5-15% by weight of the formulation. In some embodiments, the formulation is about 30 to about 50 percent total polymer by weight. As non-limiting examples, the formulation is about 35-45 percent total polymer by weight. In an example, the polymer comprises a poloxamer. Poloxamers are block copolymers of poly(ethylene oxide) (PEO) and poly(propylene oxide) (PPO). A non-limiting example of a poloxamer is poloxamer 407, such as Pluronic® F-127. In some cases, the formulation comprises about 5-20, 5-15, 5-10, 10-20, 10-15, or 15-20 percent poloxamer 407 by weight. As another example, the polymer comprises polyethylene glycol (PEG). In some cases, the formulation comprises about 5-30, 5-25, 5-20, 5-15, 5-10, 10-30, 10-25, 10-20, 10-15, 15-30, 15-25, 15-20, 20-30, 20-25, or 25-30 percent by weight PEG. For instance, the formulation comprises about 5-30, or 5, 6, 7, 8, 9, 10, 11, 12, 13, 14, 15, 16, 17, 18, 19, 20, 21, 22, 23, 24, 25, 26, 27, 28, 29 or 30 percent by weight PEG. In some embodiments, there is a first PEG with a first molecular weight, and a second PEG with a second molecular weight. In some embodiments, PEG can have a molecular weight from 500 g/mol to 35,000 g/mol. In some cases, the molecular weight of PEG is about 500 g/mol, about 1,000 g/mol, about 1,500 g/mol, about 20.00 g/mol, about 2,500 g/mol, about 3,000 g/mol, about 3,500 g/mol, about 4,000 g/mol, about 4,500 g/mol, about 5,000 g/mol, about 5,500 g/mol, about 6,000 g/mol, about 6,500 g/mol, about 7,000 g/mol, about 7,500 g/mol, about 8,000 g/mol, about 8,500 g/mol, about 9,000 g/mol, about 9,500 g/mol, about 10,000 g/mol, about 10,500 g/mol, about 11,000 g/mol, about 11,500 g/mol, about 12,000 g/mol, about 12,500 g/mol, about 13,000 g/mol, about 13,500 g/mol, about 14,000 g/mol, about 14,500 g/mol, about 15,000 g/mol, about 15,500 g/mol, about 16,000 g/mol, about 16,500 g/mol, about 17,000 g/mol, about 17,500 g/mol, about 18,000 g/mol, about 18,500 g/mol, about 19,000 g/mol, about 19,500 g/mol, about 20,000 g/mol, about 20,500 g/mol, about 21,000 g/mol, about 21,500 g/mol, about 22,000 g/mol, about 22,500 g/mol, about 23,000 g/mol, about 23,500 g/mol, about 24,000 g/mol, about 24,500 g/mol, 25,000 g/mol, 25,500 g/mol, 26,000 g/mol, 26,500 g/mol, 27,000 g/mol, 27,500 g/mol, 28,000 g/mol, 28,500 g/mol, 29,000 g/mol, 29,500 g/mol, 30,000 g/mol, 30,500 g/mol, 31,000 g/mol, 31,500 g/mol, 32,000 g/mol, 32,500 g/mol, 33,000 g/mol, 33,500 g/mol, 34,000 g/mol, 34,500 g/mol or about 35,000 g/mol. In a nonlimiting example embodiment, the formulation comprises PEG having a molecular weight of 1,500 g/mol. In a further nonlimiting example embodiment, the formulation comprises PEG having a molecular weight of 8,000 g/mol. In a further nonlimiting example embodiment, the formulation comprises PEG having a molecular weight of 20,000 g/mol. In a further nonlimiting example embodiment, the formulation comprises PEG having a molecular weight of 35,000 g/mol. In a nonlimiting example embodiment, the formulation comprises a first PEG at about 5-15 percent by weight and a second PEG at about 5-15 percent by weight. In a nonlimiting example embodiment, the formulation comprises a first PEG at about 15 percent by weight and a second PEG at about 15 percent by weight. In a nonlimiting example embodiment, the formulation comprises a first PEG at about 5 percent by weight and a second PEG at about 5 percent by weight. In a nonlimiting example embodiment, the formulation comprises a first PEG at about 10 percent by weight and a second PEG at about 10 percent by weight. The first PEG may have a lower molecular weight and a lower melt viscosity than the second PEG. For instance, the first PEG has a molecular weight of about 500-15,000 g/mol and the second PEG has a molecular weight of about 25,000-50,000 g/mol. As another example, the polymer comprises polydioxanone (PDS). In some cases, the formulation comprises about 10-30, 10-25, 10-20, 10-15, 15-30, 15-25, 15-20, 20-30, 20-25, or 25-30 percent by weight PDS. For instance, the formulation comprises about 15-25, or 15, 16, 17, 18, 19, 20, 21, 22, 23, 24, or 25 percent by weight PDS. As another example, the polymer comprises poly-1-lactide. In some cases, the formulation comprises about 10-30, 10-25, 10-20, 10-15, 15-30, 15-25, 15-20, 20-30, 20-25, or 25-30 percent by weight poly-1-lactide. For instance, the formulation comprises about 15-25, or 15, 16, 17, 18, 19, 20, 21, 22, 23, 24, or 25 percent by weight poly-1-lactide. As another example, the polymer comprises a polyester. In some embodiments, the polyester comprises a biodegradable polyester such as polycaprolactone (PCL). In some cases, the formulation comprises about 10-30, 10-25, 10-20, 10-15, 15-30, 15-25, 15-20, 20-30, 20-25, or 25-30 percent by weight PCL. For instance, the formulation comprises about 15-25, or 15, 16, 17, 18, 19, 20, 21, 22, 23, 24, or 25 percent by weight PCL. In some cases, the formulation comprises PCL having a molecular weight of 50,000 g/mol. In some embodiments, the polyester comprises a polyglycolide or poly(glycolic acid) (PGA). In some cases, the formulation comprises about 0.5-20, 0.5-18, 0.5-16, 0.5-14, 0.5-12, 0.5-10, 0.5-8, 0.5-6, 0.5-4, 0.5-2, 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent by weight PGA. For instance, the formulation comprises about 1, 2, 3, 4, 5, 6, 7, 8, 9, 10, 11, 12, 13, 14, or 15 percent by weight PGA. In some cases, the PGA has a molecular weight of about 38,000-54,000. In some embodiments, the polyester comprises a polylactide, such as poly(D,L-lactide). In some cases, the formulation comprises about 0.5-20, 0.5-18, 0.5-16, 0.5-14, 0.5-12, 0.5-10, 0.5-8, 0.5-6, 0.5-4, 0.5-2, 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent by weight polylactide. For instance, the formulation comprises about 1, 2, 3, 4, 5, 6, 7, 8, 9, 10, 11, 12, 13, 14, or 15 percent by weight polylactide.

In some embodiments, the polymer comprises a copolymer. In some embodiments, the copolymer is present in the formulation at about 10-30% by weight. In some cases, the copolymer comprises polyglycolide. In some cases, the copolymer comprises PCL and polyglycolide. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar PCL, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar polyglycolide. In some cases, the copolymer comprises about 90-95 percent by mole PCL and about 5-10 percent by mole polyglycolide. In some cases, the copolymer comprises PDS and polyglycolide. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar PDS, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar polyglycolide. In some cases, the copolymer comprises about 90-95 percent by mole PDS and about 5-10 percent by mole polyglycolide. In some cases, the copolymer comprises PDS-glycolide copolymer. For instance, the formulation comprises about 10-30, 10-25, 10-20, 10-15, 15-30, 15-25, 15-20, 20-30, 20-25, 25-30, or 20 percent by weight PDS-glycolide copolymer. In some cases, the copolymer comprises glycolide/L-lactide. In some cases, the formulation comprises about 10-30, 10-25, 10-20, 10-15, 15-30, 15-25, 15-20, 20-30, 20-25, or 25-30 percent by weight glycolide/L-lactide copolymer. For instance, the formulation comprises about 15-25, or 15, 16, 17, 18, 19, 20, 21, 22, 23, 24, or 25 percent by weight glycolide/L-lactide copolymer. In some cases, the copolymer comprises glycolide and lactide (e.g., L-lactide). For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar glycolide, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar lactide (e.g., L-lactide). In some cases, the copolymer comprises about 85-95 percent by mole glycolide and about 5-15 percent by mole lactide (e.g., L-lactide), e.g., glycolide/L-lactide copolymer (95:5). In some cases, the copolymer comprises caprolactone/glycolide. In some cases, the formulation comprises about 10-30, 10-25, 10-20, 10-15, 15-30, 15-25, 15-20, 20-30, 20-25, or 25-30 percent by weight caprolactone/glycolide copolymer. For instance, the formulation comprises about 15-25, or 15, 16, 17, 18, 19, 20, 21, 22, 23, 24, or 25 percent by weight caprolactone/glycolide copolymer. In some cases, the copolymer comprises caprolactone and glycolide. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar caprolactone, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar glycolide. In some cases, the copolymer comprises about 85-95 percent by mole caprolactone and about 5-15 percent by mole glycolide, e.g., caprolactone/glycolide copolymer (95:5) or caprolactone/glycolide copolymer (90:10). In some cases, the copolymer comprises poly(D,L-lactide-co-glycolide) copolymer. For instance, the formulation comprises about 10-30, 10-25, 10-20, 10-15, 15-30, 15-25, 15-20, 20-30, 20-25, 25-30, or 20 percent by weight poly(D,L-lactide-co-glycolide) copolymer. In some cases, the copolymer comprises lactide (e.g., poly(D,L-lactide)) and polyglycolide. For instance, the copolymer comprises about 35-65, 35-60, 35-55, 35-50, 35-45, 35-40, 40-65, 40-60, 40-55, 40-50, 40-45, 45-65, 45-60, 45-55, 45-50, 50-65, 50-60, 50-55, 55-65, 55-60, 60-65 percent molar lactide (e.g., poly(D,L-lactide)), and about 35-65, 35-60, 35-55, 35-50, 35-45, 35-40, 40-65, 40-60, 40-55, 40-50, 40-45, 45-65, 45-60, 45-55, 45-50, 50-65, 50-60, 50-55, 55-65, 55-60, 60-65 percent molar glycolide, e.g., poly(D,L-lactide-co-glycolide) copolymer (50:50). In some cases, the copolymer comprises lactide (e.g., L-lactide) and PDS. For instance, the copolymer comprises about 35-65, 35-60, 35-55, 35-50, 35-45, 35-40, 40-65, 40-60, 40-55, 40-50, 40-45, 45-65, 45-60, 45-55, 45-50, 50-65, 50-60, 50-55, 55-65, 55-60, 60-65 percent molar PDS, and about 35-65, 35-60, 35-55, 35-50, 35-45, 35-40, 40-65, 40-60, 40-55, 40-50, 40-45, 45-65, 45-60, 45-55, 45-50, 50-65, 50-60, 50-55, 55-65, 55-60, 60-65 percent molar lactide (e.g., L-lactide). In some cases, the copolymer comprises PDS-L-lactide copolymer. For instance, the formulation comprises about 10-30, 10-25, 10-20, 10-15, 15-30, 15-25, 15-20, 20-30, 20-25, 25-30, or 20 percent by weight PDS-L-lactide copolymer. In some cases, the copolymer comprises dioxanone. In some cases, the copolymer comprises dioxanone and lactide (e.g., L-lactide). For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar dioxanone, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar lactide (e.g., L-lactide). In some cases, the copolymer comprises about 85-95 percent by mole dioxanone and about 5-15 percent by mole lactide (e.g., L-lactide), e.g., dioxanone/L-lactide copolymer (90:10).

In some instances, the copolymer has a faster resorption rate than a single polymer. For instance, the copolymers used in example embodiments of ink formulation #2 (polycaprolactone/polyglycolide copolymer (95:5)), #3 ink formulation (polycaprolactone/polyglycolide copolymer (90:10)), and ink formulation #4 (poly(D,L-lactide-co-glycolide) copolymer (50:50)) have faster resorption rates than polycaprolactone. The resorption rates vary from slowest to fastest as: poly-1-lactide, polycaprolactone, polycaprolactone/polyglycolide copolymer (95:5), polycaprolactone/glycolide copolymer (90:10), polydioxanone/L-lactide copolymer (90:10), polydioxanone, glycolide/L-lactide copolymer (95:5), poly(D,L-lactide-co-glycolide) copolymer (50:50).

In some embodiments, the formulation comprises two or more polymers. In some embodiments, the formulation is about 5-30, 5-25, 5-20, 5-15, 5-10, 10-30, 10-25, 10-20, 10-15, 15-30, 15-25, 15-20, 20-30, 20-25, or 25-30 percent by weight of a first polymer, and about 5-30, 5-25, 5-20, 5-15, 5-10, 10-30, 10-25, 10-20, 10-15, 15-30, 15-25, 15-20, 20-30, 20-25, or 25-30 percent by weight of a second polymer. In some embodiments, one polymer is water soluble, and another polymer is not water soluble. For instance, the water soluble polymer is removed from the scaffold after or during manufacture, and the non-water soluble polymer constitutes a structural element of the scaffold. In non-limiting examples, the formulation is about 10-30 percent by weight of a first polymer, and about 10-30 percent by weight of a second polymer, or about 10, 11, 12, 13, 14, 15, 16, 17, 18, 19, 20, 21, 22, 23, 24, 25, 26, 27, 28, 29, or 30 percent by weight of the first polymer, and about 10, 11, 12, 13, 14, 15, 16, 17, 18, 19, 20, 21, 22, 23, 24, 25, 26, 27, 28, 29, or 30 percent by weight of the second polymer. In some cases, the first and/or second polymer comprises PEG. In some cases, the first polymer comprises PCL and the second polymer comprises PEG. In some cases, the first polymer comprises PDS and the second polymer comprises PEG. In some cases, the first polymer comprises poly-1-lactide and the second polymer comprises PEG. In some cases, the first polymer comprises a copolymer and the second polymer comprises PEG. The copolymer may comprise PCL and polyglycolide (e.g., 95 mol % polycaprolactone, 5 mol % polyglycolide; 90 mol % polycaprolactone, 10 mol % polyglycolide). The copolymer may comprise polylactide (e.g., poly(D,L-lactide) and polyglycolide (e.g., 50 mol % poly(D,L-lactide), 50 mol % polyglycolide or poly(D,L-lactide-co-glycolide) copolymer (50:50)). The copolymer may comprise PDS-glycolide copolymer (e.g., 90 mol % PDS, 10 mol % polyglycolide). The copolymer may comprise PDS-L-lactide copolymer (e.g., 90 mol % PDS, 10 mol % L-lactide or dioxanone/L-lactide copolymer (90:10)). The copolymer may comprise glycolide-L-lactide copolymer (e.g., 95 mol % glycolide, 5 mol % L-lactide or glycolide/L-lactide copolymer (95:5)).

In some embodiments, the formulation comprises one or more particulates. The particulate may be a pore former, sometimes referred to as a sacrificial pore former. The particulate may be water soluble. The particulate may comprise a salt and/or sugar. Non-limiting examples of particulates include sodium chloride, calcium chloride, sucrose, trehalose (e.g., α,α trehalose dihydrate), and mannitol (e.g., D-mannitol). Other pore formers include water soluble polymers, such as PEG. In some cases, the particulate comprises sucrose. In some embodiments, the formulation comprises about 1-10, 1-9, 1-8, 1-7, 1-6, 1-5, 2-10, 2-9, 2-8, 2-7, 2-6, 2-5, 3-10, 3-9, 3-8, 3-7, 3-6, 3-5, 4-10, 4-9, 4-8, 4-7, 4-6, 4-5, 5-10, 5-9, 5-8, 5-7, or 5-6 percent by weight particulate. In some cases, the formulation comprises about 1-10%, or about 1%, 2%, 3%, 4%, 5%, 6%, 7%, 8%, 9% or 10% particulate. For instance, about 1-10% sucrose. In some embodiments, the particulate or pore former has an average size of about 1 micron to about 500 microns in diameter. For instance, about 1 micron to about 450 microns, about 1 micron to about 400 microns, about 1 micron to about 350 microns, about 1 micron to about 300 microns, about 1 micron to about 250 microns, about 1 micron to about 200 microns, about 1 micron to about 150 microns, about 50 microns to about 500 microns, about 50 microns to about 450 microns, about 50 microns to about 400 microns, about 50 microns to about 350 microns, about 50 microns to about 300 microns, about 50 microns to about 250 microns, about 50 microns to about 200 microns, about 50 microns to about 150 microns, about 100 microns to about 500 microns, about 100 microns to about 450 microns, about 100 microns to about 400 microns, about 100 microns to about 350 microns, about 100 microns to about 300 microns, about 100 microns to about 250 microns, about 100 microns to about 200 microns, about 100 microns to about 150 microns, about 150 microns to about 500 microns, about 150 microns to about 450 microns, about 150 microns to about 400 microns, about 150 microns to about 350 microns, about 150 microns to about 300 microns, about 150 microns to about 250 microns, or about 150 microns to about 200 microns in diameter. In some cases, the particular or pore former has an average size of about 50 microns to about 250 microns, about 60 microns to about 240 microns, about 70 microns to about 230 microns, about 80 microns to about 220 microns, or about 90 microns to about 210 microns in diameter. In some embodiments, the particulate of pore former has an average size of about 100 microns to about 200 microns, e.g., about 110 microns to about 190 microns, about 120 microns to about 180 microns, about 130 microns to about 170 microns, about 140 microns to about 160 microns, or about 100 microns, about 110 microns, about 120 microns, about 130 microns, about 140 microns, about 150 microns, about 160 microns, about 170 microns, about 180 microns, about 190 microns, or about 200 microns in diameter. In some embodiments, the particulate or pore former has an average size of about 150 microns in diameter. In some embodiments, upon removal of the particulate or pore former, a structure formed from the formulation has micropores that provide additional surface area to the structure for contact with a therapeutic agent as compared to a structure formed with a formulation lacking the particulate or pore former. In some embodiments, the micropores of the structure have an average pore size of about 1 micron to about 500 microns, or about 50 microns to about 250 microns, or about 150 microns in diameter.

In some embodiments, the formulation comprises one or more blowing agents. In some embodiments, the blowing agent comprises about 5-20, 5-18, 5-16, 5-14, 5-12, 5-10, 5-8, 5-6, 6-20, 6-18, 6-16, 6-14, 6-12, 6-10, 6-8, 8-20, 8-18, 8-16, 8-14, 8-12, 8-10, 10-20, 10-18, 10-16, 10-14, 10-12, 5-15, or about 5, 6, 7, 8, 9, 10, 11, 12, 13, 14, or 15 percent by weight blowing agent. In some cases, the blowing agent releases carbon dioxide base during printing to create a foamed structure that can increase porosity of the 3D printed structure. Non-limiting examples of blowing agents include baking powder (e.g., monocalcium phosphate, sodium bicarbonate, corn starch) and azodicarbonamide. In some cases, the blowing agent comprises sodium bicarbonate. In some cases, the formulation comprises about 5-15, or about 5, 6, 7, 8, 9, 10, 11, 12, 13, 14, or 15 percent by weight sodium bicarbonate. In some embodiments, the blowing agent provides for micropores in the structure having an average diameter of about 1 micron to about 500 microns, or about 50 microns to about 250 microns, or about 150 microns.

In some embodiments, a formulation comprises a ceramic material (e.g., β-TCP) and a polymer. Polymers include PEO, PPO, PDS, PEG, polyester, copolymers, or a combination thereof. In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent ceramic material (e.g., β-TCP) by weight, e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% ceramic material (e.g., β-TCP) by weight. In some cases, the formulation comprises about 5-20, 5-15, 5-10, 10-20, 10-15, or 15-20 percent poloxamer 407 by weight. In some cases, the formulation comprises about 10-30, 10-25, 10-20, 10-15, 15-30, 15-25, 15-20, 20-30, 20-25, or 25-30 percent by weight PEG. In some cases, the formulation comprises about 5-15 percent by weight a first PEG and 5-15 percent by weight a second PEG. In some cases, the formulation comprises about 10-30, 10-25, 10-20, 10-15, 15-30, 15-25, 15-20, 20-30, 20-25, or 25-30 percent by weight PCL. In some cases, the formulation comprises about 10-30, 10-25, 10-20, 10-15, 15-30, 15-25, 15-20, 20-30, 20-25, or 25-30 percent by weight PDS. In some cases, the formulation comprises about 10-30, 10-25, 10-20, 10-15, 15-30, 15-25, 15-20, 20-30, 20-25, or 25-30 percent by weight caprolactone/glycolide copolymer (95:5). In some cases, the formulation comprises about 10-30, 10-25, 10-20, 10-15, 15-30, 15-25, 15-20, 20-30, 20-25, or 25-30 percent by weight caprolactone/glycolide copolymer (90:10). In some cases, the formulation comprises about 10-30, 10-25, 10-20, 10-15, 15-30, 15-25, 15-20, 20-30, 20-25, or 25-30 percent by weight poly(D,L-lactide-co-glycolide) copolymer (50:50). In some cases, the formulation comprises about 10-30, 10-25, 10-20, 10-15, 15-30, 15-25, 15-20, 20-30, 20-25, or 25-30 percent by weight dioxanone/L-lactide copolymer (90:10). In some cases, the formulation comprises about 10-30, 10-25, 10-20, 10-15, 15-30, 15-25, 15-20, 20-30, 20-25, or 25-30 percent by weight glycolide/L-lactide copolymer (95:5). In some cases, the formulation comprises about 10-30, 10-25, 10-20, 10-15, 15-30, 15-25, 15-20, 20-30, 20-25, or 25-30 percent by weight poly-1-lactide. In some embodiments, the formulation further comprises an antifoaming agent. In some embodiments, the formulation further comprises a dispersing agent. In some embodiments, the formulation further comprises a solvent. In some embodiments, the formulation further comprises a plasticizer. In some embodiments, the formulation further comprises a particulate or sacrificial pore former. In some cases, the formulation further comprises a blowing agent.

In some embodiments, a formulation comprises a ceramic material (e.g., β-TCP) and a particulate or sacrificial pore former. The particulate may be water soluble. Non-limiting examples of particulates include salts and sugars, e.g., sodium chloride, calcium chloride, sucrose, trehalose (e.g., α,α trehalose dihydrate), and mannitol (e.g., D-mannitol). The pore former may be a water soluble polymer such as PEG. In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent ceramic material (e.g., β-TCP) by weight, e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% ceramic material (e.g., β-TCP) by weight. In some embodiments, the formulation comprises about 1-10, 1-9, 1-8, 1-7, 1-6, 1-5, 2-10, 2-9, 2-8, 2-7, 2-6, 2-5, 3-10, 3-9, 3-8, 3-7, 3-6, 3-5, 4-10, 4-9, 4-8, 4-7, 4-6, 4-5, 5-10, 5-9, 5-8, 5-7, or 5-6 percent by weight particulate. In some embodiments, the particulate comprises sucrose. In some embodiments, the formulation comprises about 10-30% or about 5-15% percent by weight sacrificial pore former, such as a polymer. In some embodiments, the formulation further comprises water. In some embodiments, the formulation further comprises a polymer. In some embodiments, the formulation further comprises an antifoaming agent. In some embodiments, the formulation further comprises a dispersing agent. In some embodiments, the formulation further comprises a solvent. In some embodiments, the formulation further comprises a plasticizer. In some cases, the formulation further comprises a blowing agent.

In some embodiments, a formulation comprises a ceramic material (e.g., β-TCP) and a blowing agent. Non-limiting examples of blowing agents include baking powder (e.g., monocalcium phosphate, sodium bicarbonate, corn starch) and azodicarbonamide. The blowing agent may comprise sodium bicarbonate. In some embodiments, the formulation is about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70 percent ceramic material (e.g., β-TCP) by weight, e.g., about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% ceramic material (e.g., β-TCP) by weight. In some embodiments, the blowing agent comprises about 5-20, 5-18, 5-16, 5-15, 5-14, 5-12, 5-10, 5-8, 5-6, 6-20, 6-18, 6-16, 6-14, 6-12, 6-10, 6-8, 8-20, 8-18, 8-16, 8-14, 8-12, 8-10, 10-20, 10-18, 10-16, 10-14, 10-12, 5-15, or about 5, 6, 7, 8, 9, 10, 11, 12, 13, 14, or 15 percent by weight blowing agent. In some cases, the formulation comprises about 5-15, or about 5, 6, 7, 8, 9, 10, 11, 12, 13, 14, or 15 percent by weight sodium bicarbonate. In some embodiments, the formulation further comprises water. In some embodiments, the formulation further comprises a polymer. In some embodiments, the formulation further comprises an antifoaming agent. In some embodiments, the formulation further comprises a dispersing agent. In some embodiments, the formulation further comprises a solvent. In some embodiments, the formulation further comprises a plasticizer. In some embodiments, the formulation further comprises a particulate or sacrificial pore former.

In another aspect, a formulation comprises a ceramic material and one or more polymers. In some embodiments, the formulation comprises about 30% to about 70% a ceramic material (e.g., β-TCP). For instance, the formulation comprises about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 60-70, 60-65, 65-70, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 percent by weight a ceramic material (e.g., β-TCP). In some embodiments, the formulation comprises a first polymer, e.g., about 10-30, 10-25, 10-20, 10-15, 15-30, 15-25, 15-20, 20-30, 20-25, or 25-30 percent by weight first polymer. The first polymer may be polycaprolactone (PCL). The first polymer may be polydioxanone (PDS). The first polymer may be poly-1-lactide. The first polymer may be a copolymer, e.g., caprolactone/glycolide copolymer, poly(D,L-lactide-co-glycolide) copolymer, dioxanone/L-lactide copolymer, or glycolide/L-lactide copolymer. In some embodiments, the formulation comprises a second polymer, e.g., about 10-30, 10-25, 10-20, 10-15, 15-30, 15-25, 15-20, 20-30, 20-25, or 25-30 percent by weight second polymer. The first and/or second polymer may be water soluble or non-water soluble. In some embodiments, the formulation comprises a first polymer, a second polymer, and/or a third polymer. The third polymer may be water soluble or non-water soluble. The second polymer may be polyethylene glycol (PEG). The second polymer may be PEG with a MW of about 8000 g/mol. The third polymer may be PEG. The third polymer may be PEG with a MW of about 35,000 g/mol. In a non-limiting embodiment, the formulation comprises about 30-70% by weight ceramic, about 10-30% by weight a first polymer, and about 10-30% by weight a second polymer. In a non-limiting embodiment, the formulation comprises about 30-70% by weight ceramic, about 10-30% by weight a first polymer, and about 5-15% by weight a second polymer, and about 5-15% by weight of a third polymer. For example, the formulation may comprise about 30-70% by weight β-TCP, about 10-30% by weight PCL, and about 10-30% by weight PEG. As another example, the formulation may comprise about 30-70% by weight β-TCP, about 10-30% by weight PCL, about 5-15% by weight PEG (8000 MW), and about 5-15% by weight PEG (35000 MW). As another example, the formulation may comprise about 30-70% by weight β-TCP, about 10-30% by weight PDS, and about 10-30% by weight PEG. As another example, the formulation may comprise about 30-70% by weight β-TCP, about 10-30% by weight PDS, about 5-15% by weight PEG (8000 MW), and about 5-15% by weight PEG (35000 MW). As another example, the formulation may comprise about 30-70% by weight β-TCP, about 10-30% by weight poly-1-lactide, and about 10-30% by weight PEG. As another example, the formulation may comprise about 30-70% by weight β-TCP, about 10-30% by weight poly-1-lactide, about 5-15% by weight PEG (8000 MW), and about 5-15% by weight PEG (35000 MW). As another example, the formulation may comprise about 30-70% by weight β-TCP, about 10-30% by weight copolymer, and about 10-30% by weight PEG. As another example, the formulation may comprise about 30-70% by weight β-TCP, about 10-30% by weight copolymer, about 5-15% by weight PEG (8000 MW), and about 5-15% by weight PEG (35000 MW). Non-limiting example co-polymers include caprolactone/glycolide copolymer, poly(D,L-lactide-co-glycolide) copolymer, dioxanone/L-lactide copolymer, or glycolide/L-lactide copolymer.

In some further embodiments, the formulation comprises a particulate and/or pore former. The particulate may be water soluble. In some cases, the particulate comprises sucrose. In some embodiments, the formulation comprises about 1-10, 1-9, 1-8, 1-7, 1-6, 1-5, 2-10, 2-9, 2-8, 2-7, 2-6, 2-5, 3-10, 3-9, 3-8, 3-7, 3-6, 3-5, 4-10, 4-9, 4-8, 4-7, 4-6, 4-5, 5-10, 5-9, 5-8, 5-7, or 5-6 percent by weight particulate. For example, the formulation may comprise about 30-70% by weight β-TCP, about 10-30% by weight PCL, about 10-30% by weight PEG, and about 1-10% by weight particulate. In some embodiments, the formulation may comprise about 30-70% by weight β-TCP, about 10-30% by weight PCL, PDS, poly-1-lactide, caprolactone/glycolide copolymer, poly(D,L-lactide-co-glycolide) copolymer, dioxanone/L-lactide copolymer, or glycolide/L-lactide copolymer, about 5-15% by weight 8000 MW PEG, about 5-15% by weight 35,000 MW PEG, and about 1-10% by weight particulate. In some embodiments, the formulation comprises PEG, and the PEG is a pore former. In some embodiments, the PEG is present at about 10-30% by weight, or about 5-15% by weight 8000 MW PEG and about 5-15% by weight 35,000 MW PEG.

In some further embodiments, the formulation comprises a blowing agent. In some cases, the blowing agent comprises sodium bicarbonate. In some embodiments, the formulation comprises about 5-20, 5-18, 5-16, 5-14, 5-12, 5-10, 5-8, 5-6, 6-20, 6-18, 6-16, 6-14, 6-12, 6-10, 6-8, 8-20, 8-18, 8-16, 8-14, 8-12, 8-10, 10-20, 10-18, 10-16, 10-14, 10-12, 5-15, or about 5, 6, 7, 8, 9, 10, 11, 12, 13, 14, or 15 percent by weight blowing agent. For example, the formulation may comprise about 30-70% by weight β-TCP, about 10-30% by weight PCL, about 10-30% by weight PEG, and about 5-20% by weight blowing agent. For example, the formulation may comprise about 30-70% by weight β-TCP, about 10-30% by weight PCL, PDS, poly-1-lactide, caprolactone/glycolide copolymer, poly(D,L-lactide-co-glycolide) copolymer, dioxanone/L-lactide copolymer, or glycolide/L-lactide copolymer, about 5-15% by weight 8000 MW PEG, about 5-15% by weight 35,000 MW PEG, and about 5-20% by weight blowing agent.

In some further embodiments, the polymer of the formulation is a copolymer, such as a PCL and polyglycolide copolymer. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar PCL, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar polyglycolide. In some cases, the copolymer comprises about 90-95 percent by mole PCL and about 5-10 percent by mole polyglycolide. In an example, the formulation may comprise about 30-70% by weight β-TCP, about 10-30% by weight PCL and polyglycolide copolymer (e.g., 95 mol % polycaprolactone, 5 mol % polyglycolide), and about 10-30% by weight PEG. In an example, the formulation may comprise about 30-70% by weight β-TCP, about 10-30% by weight PCL and polyglycolide copolymer (e.g., 90 mol % polycaprolactone, 10 mol % polyglycolide), and about 10-30% by weight PEG. In an example, the formulation may comprise about 30-70% by weight β-TCP, about 10-30% by weight PCL and polyglycolide copolymer (e.g., 90 mol % polycaprolactone, 10 mol % polyglycolide, which may be referred to as caprolactone/glycolide copolymer (90:10)), and about 5-15% by weight 8000 MW PEG, about 5-15% by weight 35,000 MW PEG. In an example, the formulation may comprise about 30-70% by weight β-TCP, about 10-30% by weight PCL and polyglycolide copolymer (e.g., 95 mol % polycaprolactone, 5 mol % polyglycolide), and about 5-15% by weight 8000 MW PEG, about 5-15% by weight 35,000 MW PEG.

In some further embodiments, the polymer of the formulation is a copolymer, such as a PDS and polyglycolide copolymer. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar PDS, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar polyglycolide. In some cases, the copolymer comprises about 90-95 percent by mole PDS and about 5-10 percent by mole polyglycolide. In an example, the formulation may comprise about 30-70% by weight β-TCP, about 10-30% by weight PDS and polyglycolide copolymer (e.g., 90 mol % PDS, 10 mol % polyglycolide), and about 10-30% by weight PEG. In an example, the formulation may comprise about 30-70% by weight β-TCP, about 10-30% by weight PDS and polyglycolide copolymer (e.g., 90 mol % PDS, 10 mol % polyglycolide), and about 5-15% by weight 8000 MW PEG, about 5-15% by weight 35,000 MW PEG.

In some further embodiments, the polymer of the formulation is a copolymer, such as a poly(D-L-lactide) and glycolide copolymer. For instance, the copolymer comprises about 30-50, 31-49, 32-48, 33-47, 34-46, 35-45, 36-44, 37-43, 38-42, 39-41, 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar poly(D-L-lactide), and about 30-50, 31-49, 32-48, 33-47, 34-46, 35-45, 36-44, 37-43, 38-42, 39-41, 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar glycolide. In some cases, the copolymer comprises about 50 percent by mole poly(D-L-lactide), and about 50 percent by mole glycolide. In an example, the formulation may comprise about 30-70% by weight β-TCP, about 10-30% by weight poly(D-L-lactide) and glycolide copolymer (e.g., 50 mol % poly(D-L-lactide), 50 mol % glycolide), and about 10-30% by weight PEG. In an example, the formulation may comprise about 30-70% by weight β-TCP, about 10-30% by weight poly(D-L-lactide) and glycolide copolymer (e.g., 50 mol % poly(D-L-lactide, 50 mol % polyglycolide), and about 5-15% by weight 8000 MW PEG, about 5-15% by weight 35,000 MW PEG.

In some further embodiments, the polymer of the formulation is a copolymer, such as a PDS and lactide copolymer. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar PDS, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar lactide. In some cases, the copolymer comprises about 90-95 percent by mole PDS and about 5-10 percent by mole lactide. In an example, the formulation may comprise about 30-70% by weight β-TCP, about 10-30% by weight PDS and lactide copolymer (e.g., 90 mol % PDS, 10 mol % lactide), and about 10-30% by weight PEG. In an example, the formulation may comprise about 30-70% by weight β-TCP, about 10-30% by weight PDS and lactide copolymer (e.g., 90 mol % PDS, 10 mol % lactide), and about 5-15% by weight 8000 MW PEG, about 5-15% by weight 35,000 MW PEG.

In some further embodiments, the polymer of the formulation is a copolymer, such as a glycolide and lactide copolymer. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar glycolide, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar lactide. In some cases, the copolymer comprises about 90-95 percent by mole glycolide and about 5-10 percent by mole lactide. In an example, the formulation may comprise about 30-70% by weight β-TCP, about 10-30% by weight glycolide and lactide copolymer (e.g., 95 mol % glycolide, 10 mol % lactide), and about 10-30% by weight PEG. In an example, the formulation may comprise about 30-70% by weight β-TCP, about 10-30% by weight glycolide and lactide copolymer (e.g., 95 mol % glycolide, 5 mol % lactide), and about 5-15% by weight 8000 MW PEG, about 5-15% by weight 35,000 MW PEG.

In another aspect, a formulation comprises a ceramic material and one or more polymers.

In some embodiments, the formulation comprises about 30% to about 70% a ceramic material (e.g., β-TCP). For instance, the formulation comprises about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 60-70, 60-65, 65-70, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 percent by weight a ceramic material (e.g., β-TCP). In some embodiments, the formulation comprises a first polymer, e.g., about 10-30, 10-25, 10-20, 10-15, 15-30, 15-25, 15-20, 20-30, 20-25, or 25-30 percent by weight first polymer. The first polymer may be a copolymer. In some embodiments, the copolymer comprises a lactide (e.g., poly(D,L-lactide)) and polyglycolide. In some embodiments, the copolymer comprises poly(D,L-lactide-co-glycolide) copolymer. In some embodiments, the copolymer comprises about 50 mol % poly(D,L-lactide) and about 50 mol % polyglycolide. In some embodiments, the copolymer comprises caprolactone/glycolide (e.g., 90:10, 95:5) copolymer, poly(D,L-lactide-co-glycolide) (e.g., 50:50) copolymer, dioxanone/L-lactide (e.g., 90:10) copolymer, or glycolide/L-lactide (e.g., 95:5) copolymer. In some embodiments, the formulation comprises a second polymer, e.g., about 10-30, 10-25, 10-20, 10-15, 15-30, 15-25, 15-20, 20-30, 20-25, or 25-30 percent by weight second polymer. The second polymer may be polyethylene glycol (PEG). In a non-limiting embodiment, the formulation comprises about 30-70% by weight ceramic, about 10-30% by weight a first polymer, and about 10-30% by weight a second polymer. For example, the formulation may comprise about 30-70% by weight β-TCP, about 10-30% by weight copolymer, and about 10-30% by weight PEG. In some embodiments, the formulation may comprise about 30-70% by weight β-TCP, about 10-30% by weight copolymer, and about 5-15% by weight 8000 MW PEG, about 5-15% by weight 35,000 MW PEG.

In some further embodiments, a formulation comprises a ceramic material and one or more polymers. In some embodiments, the formulation comprises about 30% to about 70% a ceramic material (e.g., β-TCP). For instance, the formulation comprises about 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 60-70, 60-65, 65-70, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 percent by weight a ceramic material (e.g., β-TCP). In some embodiments, the formulation comprises a first polymer, e.g., about 10-30, 10-25, 10-20, 10-15, 15-30, 15-25, 15-20, 20-30, 20-25, or 25-30 percent by weight first polymer. The first polymer may be a copolymer, such as a dioxanone and lactide (e.g., L-lactide) copolymer. For instance, the copolymer comprises about 80-99, 80-98, 80-97, 80-96, 80-95, 80-94, 80-93, 80-92, 80-91, 80-90, 80-89, 80-88, 80-87, 80-86, 80-85, 85-99, 85-98, 85-97, 85-96, 85-95, 85-94, 85-93, 85-92, 85-91, 85-90, 90-99, 90-98, 90-97, 90-96, 90-95, 90-94, 90-93, 90-92, 90-91, 95-99, 95-98, 95-97, or 95-96 percent molar dioxanone, and about 1-20, 1-18, 1-16, 1-14, 1-12, 1-10, 1-8, 1-6, 1-4, 1-2, 2-20, 2-18, 2-16, 2-14, 2-12, 2-10, 2-8, 2-6, 2-4, 3-20, 3-18, 3-16, 3-14, 3-12, 3-10, 3-8, 3-6, 3-4, 4-20, 4-18, 4-16, 4-14, 4-12, 4-10, 4-8, 4-6, 5-20, 5-18, 5-16, 5-14, 5-12, 5-10, 5-8, 5-6, 8-20, 8-18, 8-16, 8-14, 8-12, or 8-10 percent molar lactide. In some cases, the copolymer comprises about 90-95 percent by mole dioxanone and about 5-10 percent by mole lactide. In some embodiments, the formulation comprises a second polymer, e.g., about 10-30, 10-25, 10-20, 10-15, 15-30, 15-25, 15-20, 20-30, 20-25, or 25-30 percent by weight second polymer. The second polymer may be polyethylene glycol (PEG). In a non-limiting embodiment, the formulation comprises about 30-70% by weight ceramic, about 10-30% by weight a first polymer, and about 10-30% by weight a second polymer. For example, the formulation may comprise about 30-70% by weight β-TCP, about 10-30% by weight copolymer, and about 10-30% by weight PEG In an example, the formulation may comprise about 30-70% by weight β-TCP, about 10-30% by weight PDS and lactide copolymer (e.g., 90 mol % dioxanone, 10 mol % L-lactide), and about 10-30% by weight PEG. In some embodiments, the formulation may comprise about 30-70% by weight β-TCP, about 10-30% by weight PDS and lactide copolymer (e.g., 90 mol % dioxanone, 10 mol % L-lactide), and about 5-15% by weight 8000 MW PEG, about 5-15% by weight 35,000 MW PEG.

In one aspect, the formulation has a low viscosity that may be useful during manufacture for extruding through a small diameter nozzle. The nozzle may have a diameter of about 240 μm to about 500 μm or about 280 to about 450 μm, or about 240 to about 850 μm e.g., about 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, or 850 μm. In a nonlimiting example embodiment, the formulation is melt-mixed in a dual asymmetric centrifugal mixer to create a homogenous liquid ink. A mixture of low and high viscosity PEG can be used to tailor the molten ink viscosity such that oozing of molten ink out of the 3D printer nozzle during non-print motions is minimized while still enabling flow through a 100's of microns diameter nozzle when the screw extruder is engaged for print motions. Higher molecular weight PEG generally is stiffer and stronger than low molecular weight PEG, the incorporation of which also improves the mechanical strength and stiffness of the feedstock material.

In one aspect, the formulation has a higher viscosity that may be useful during manufacture by forming the formulation into a filament. The filament may then be used for fused filament fabrication.

In one aspect, the formulation is in the form of a filament. For instance, as further described in Example 2, ink formulations were prepared into filaments for use in 3D printing structures on a fused filament fabrication (FFF) 3D printer. In some embodiments, the filament formulation has a diameter of about 1 to about 3 mm, or about 1 to about 2.75 mm, about 1 to about 2.5 mm, about 1 to about 2.25 mm, about 1 to about 2 mm, about 1 to about 1.75 mm, about 1 to about 1.5 mm, about 1.25 to about 3 mm, about 1.25 to about 2.75 mm, about 1.25 to about 2.5 mm, about 1.25 to about 2.25 mm, about 1.25 to about 2 mm, about 1.25 to about 1.75 mm, about 1.25 to about 1.5 mm, about 1.5 to about 3 mm, about 1.5 to about 2.75 mm, about 1.5 to about 2.5 mm, about 1.5 to about 2.25 mm, about 1.5 to about 2 mm, about 1.5 to about 1.75 mm, about 1.75 to about 3 mm, about 1.75 to about 2.75 mm, about 1.75 to about 2.5 mm, about 1.75 to about 2.25 mm, about 1.75 to about 2 mm, about 2 to about 3 mm, about 2 to about 2.75 mm, about 2 to about 2.5 mm, or about 2 to about 2.25 mm. As a non-limiting example, the filament formulation has a diameter of about 1.5 mm to about 2 mm, or about 1.5 mm, about 1.75 mm, or about 2 mm.

In one aspect, the formulation is in the form of a pellet. For instance, as further described in Example 2, ink formulations were prepared into pellets for use in 3D structures. In some embodiments, the pellets can be made into filaments. In some embodiments, pellets can be made into powders. In some embodiments, pellets have a length of about 1 to about 6 mm, or about 1 to about 5.5 mm, about 1 to about 5 mm, about 1 to about 4.5 mm, about 1 to about 4 mm, about 1 to about 3.5 mm, about 1 to about 3 mm, about 1 to about 2.5 mm, about 1 to about 2 mm, about 1 to about 1.5 mm, about 1.5 to about 6 mm, about 1.5 to about 5.5 mm, about 1.5 to about 5 mm, about 1.5 to about 4.5 mm, about 1.5 to about 4 mm, about 1.5 to about 3.5 mm, about 1.5 to about 3 mm, about 1.5 to about 2.5 mm, about 2 to about 6 mm, about 2 to about 5.5 mm, about 2 to about 5 mm, about 2 to about 4.5 mm, about 2 to about 4 mm, about 2 to about 3.5 mm, about 2 to about 3 mm, about 2 to about 2.5 mm, about 2.5 to about 6 mm, about 2.5 to about 5.5 mm, about 2.5 to about 5 mm, about 2.5 to about 4.5 mm, about 2.5 to about 4 mm, about 2.5 to about 3.5 mm, about 2.5 to about 3 mm, about 3 to about 6 mm, about 3 to about 5.5 mm, about 3 to about 5 mm, about 3 to about 4.5 mm, about 3 to about 4 mm, about 3 to about 3.5 mm, about 3.5 to about 6 mm, about 3.5 to about 5.5 mm, about 3.5 to about 5 mm, about 3.5 to about 4.5 mm, about 3.5 to about 4 mm, about 4 to about 6 mm, about 4 to about 5.5 mm, about 4 to about 5 mm, about 4 to about 4.5 mm, about 4.5 to about 6 mm, about 4.5 to about 5.5 mm, about 4.5 to about 5 mm, about 5 to about 6 mm, about 5 to about 5.5 mm, or about 5.5 to about 6 mm. As a non-limiting example embodiment, the pellets have a length of about 2.5 mm to about 4.5 mm, or about 2.5 mm, about 3 mm, about 3.5 mm, about 4 mm, or about 4.5 mm. In some embodiments, a pellet encompasses a variety of different shapes including spears, rods, granules, blocks, particles, and particles of any suitable shape.

In one aspect, the formulation is in the form of a powder. In some embodiments, the powder could be produced from a pellet. In a nonlimiting example embodiment, components of the formulation are melt-mixed into homogenous ink, and cryomilled to form powder. In a nonlimiting example embodiment, components of the formulation are dissolved in a solvent-based slurry and spray dried to form a powder. In some embodiments, powders are used in selective laser sintering.

### 3D Printed Structures

In another aspect, provided herein are 3D printed structures. The structures may be prepared using a formulation and/or method of manufacture described herein. As used herein, structures include scaffolds, and vice versa.

In some embodiments a three-dimensional structure has micropores. The micropores may be formed after removal of a particulate or pore former. The micropores may be formed by a use of a blowing agent during formulation. In some embodiments, the micropores provide additional surface area to the structure for contact with a therapeutic agent as compared to a structure lacking micropores. In a non-limiting example, the therapeutic agent comprises a targeting moiety that is non-covalently bound to a ceramic material of the structure.

In some embodiments, the micropores have an average diameter of about 1 micron to about 500 microns. For instance, about 1 micron to about 450 microns, about 1 micron to about 400 microns, about 1 micron to about 350 microns, about 1 micron to about 300 microns, about 1 micron to about 250 microns, about 1 micron to about 200 microns, about 1 micron to about 150 microns, about 50 microns to about 500 microns, about 50 microns to about 450 microns, about 50 microns to about 400 microns, about 50 microns to about 350 microns, about 50 microns to about 300 microns, about 50 microns to about 250 microns, about 50 microns to about 200 microns, about 50 microns to about 150 microns, about 100 microns to about 500 microns, about 100 microns to about 450 microns, about 100 microns to about 400 microns, about 100 microns to about 350 microns, about 100 microns to about 300 microns, about 100 microns to about 250 microns, about 100 microns to about 200 microns, about 100 microns to about 150 microns, about 150 microns to about 500 microns, about 150 microns to about 450 microns, about 150 microns to about 400 microns, about 150 microns to about 350 microns, about 150 microns to about 300 microns, about 150 microns to about 250 microns, or about 150 microns to about 200 microns in diameter. In some cases, the micropores have an average diameter of about 50 microns to about 250 microns, about 60 microns to about 240 microns, about 70 microns to about 230 microns, about 80 microns to about 220 microns, or about 90 microns to about 210 microns. In some embodiments, the micropores an average diameter of about 100 microns to about 200 microns, e.g., about 110 microns to about 190 microns, about 120 microns to about 180 microns, about 130 microns to about 170 microns, about 140 microns to about 160 microns, or about 100 microns, about 110 microns, about 120 microns, about 130 microns, about 140 microns, about 150 microns, about 160 microns, about 170 microns, about 180 microns, about 190 microns, or about 200 microns. In some embodiments, the micropores have an average diameter of about 150 microns.

In some embodiments, the micropores have an average diameter of about 1 micron to about 50 microns. For instance, about 1 micron to about 45 microns, about 1 micron to about 40 microns, about 1 micron to about 35 microns, about 1 micron to about 30 microns, about 1 micron to about 25 microns, about 1 micron to about 20 microns, about 1 micron to about 15 microns, about 1 micron to about 10 microns, about 10 microns to about 50 microns, about 10 microns to about 45 microns, about 10 microns to about 40 microns, about 10 microns to about 35 microns, about 10 microns to about 30 microns, about 10 microns to about 25 microns, about 10 microns to about 20 microns, about 10 microns to about 15 microns, about 20 microns to about 50 microns, about 20 microns to about 45 microns, about 20 microns to about 40 microns, about 20 microns to about 35 microns, about 20 microns to about 30 microns, about 20 microns to about 25 microns, about 30 microns to about 50 microns, about 30 microns to about 45 microns, about 30 microns to about 40 microns, about 30 microns to about 35 microns, about 40 microns to about 50 microns, or about 40 microns to about 45 microns.

In example embodiments, the microporosity of the scaffold results in a hydrophilic scaffold, i.e. liquid readily wicks throughout the scaffold via capillary forces from the interconnected microporosity.

In some embodiments a three-dimensional structure has a density of about 1 g/cm3 to about 3 g/cm³. In some embodiments a three-dimensional structure has a density of about 1 g/cm3 to about 2 g/cm³. (e.g., about 1, 1.1, 1.15, 1.2, 1.25, 1.3, 1.35, 1.4, 1.45, 1.5, 1.5, 1.55, 1.6, 1.65, 1.7, 1.75, 1.8, 1.85, 1.9, 1.95, or 2 g/cm3 or any value therebetween).

In some embodiments a three-dimensional structure has an open porosity of about 15% to about 50%, about 15% to about 45%, about 15% to about 40%, about 20% to about 50%, about 20% to about 45%, about 25% to about 40%, about 25% to about 50%, about 25% to about 45%, or about 25% to about 40%. In some embodiments, the open porosity is about 25% to about 40%, e.g., about 25%, 30%, 35%, or 40%, or any value therebetween).

In some embodiments a three-dimensional structure has a strut diameter of about 300 μm to about 600 μm, about 325 μm to about 600 μm, about 350 μm to about 600 μm, about 375 μm to about 600 μm, about 400 μm to about 600 μm, about 425 μm to about 600 μm, about 450 μm to about 600 μm, about 475 μm to about 600 μm, about 500 μm to about 600 μm, about 525 μm to about 600 μm, about 550 μm to about 600 μm, about 300 μm to about 575 μm, about 325 μm to about 575 μm, about 350 μm to about 575 μm, about 375 μm to about 575 μm, about 400 μm to about 575 μm, about 425 μm to about 575 μm, about 450 μm to about 575 μm, about 475 μm to about 575 μm, about 500 μm to about 575 μm, about 525 μm to about 575 μm, about 550 μm to about 575 μm, about 300 μm to about 550 μm, about 325 μm to about 550 μm, about 350 μm to about 550 μm, about 375 μm to about 550 μm, about 400 μm to about 550 μm, about 425 μm to about 550 μm, about 450 μm to about 550 μm, about 475 μm to about 550 μm, about 500 μm to about 550 μm, about 525 μm to about 550 μm, about 300 μm to about 525 μm, about 325 μm to about 525 μm, about 350 μm to about 525 μm, about 375 μm to about 525 μm, about 400 μm to about 525 μm, about 425 μm to about 525 μm, about 450 μm to about 525 μm, about 475 μm to about 525 μm, about 500 μm to about 525 μm, about 300 μm to about 500 μm, about 325 μm to about 500 μm, about 350 μm to about 500 μm, about 375 μm to about 500 μm, about 400 μm to about 500 μm, about 425 μm to about 500 μm, about 450 μm to about 500 μm, about 475 μm to about 500 μm, about 300 μm to about 475 μm, about 325 μm to about 475 μm, about 350 μm to about 475 μm, about 375 μm to about 475 μm, about 400 μm to about 475 μm, about 425 μm to about 475 μm, about 450 μm to about 475 μm, about 300 μm to about 450 μm, about 325 μm to about 450 μm, about 350 μm to about 450 μm, about 375 μm to about 450 μm, about 400 μm to about 450 μm, about 425 μm to about 450 μm, about 300 μm to about 400 μm, about 325 μm to about 400 μm, about 350 μm to about 400 μm, or about 375 μm to about 400 μm, about 300 to about 850, about 325 to about 850, about 350 to about 850, about 375 to about 850, about 400 to about 850, about 425 to about 850, about 450 to about 850, about 475 to about 850, about 500 to about 850, about 525 to about 850, about 550 to about 850, about 575 to about 850, about 600 to about 850, about 625 to about 850, about 650 to about 850, about 675 to about 850, about 700 to about 850, about 725 to about 850, about 750 to about 850, about 775 to about 850, about 800 to about 850, about 825 to about 850, about 300 to about 825, about 325 to about 825, about 350 to about 825, about 375 to about 825, about 400 to about 825, about 425 to about 825, about 450 to about 825, about 475 to about 825, about 500 to about 825, about 525 to about 825, about 550 to about 825, about 575 to about 825, about 600 to about 825, about 625 to about 825, about 650 to about 825, about 675 to about 825, about 700 to about 825, about 725 to about 825, about 750 to about 825, about 775 to about 825, about 800 to about 825, about 825 to about 825, about 300 to about 800, about 325 to about 800, about 350 to about 800, about 375 to about 800, about 400 to about 800, about 425 to about 800, about 450 to about 800, about 475 to about 800, about 500 to about 800, about 525 to about 800, about 550 to about 800, about 575 to about 800, about 600 to about 800, about 625 to about 800, about 650 to about 800, about 675 to about 800, about 700 to about 800, about 725 to about 800, about 750 to about 800, about 775 to about 800, about 300 to about 775, about 325 to about 775, about 350 to about 775, about 375 to about 775, about 400 to about 775, about 425 to about 775, about 450 to about 775, about 475 to about 775, about 500 to about 775, about 525 to about 775, about 550 to about 775, about 575 to about 775, about 600 to about 775, about 625 to about 775, about 650 to about 775, about 675 to about 775, about 700 to about 775, about 725 to about 775, about 750 to about 775, about 300 to about 750, about 325 to about 750, about 350 to about 750, about 375 to about 750, about 400 to about 750, about 425 to about 750, about 450 to about 750, about 475 to about 750, about 500 to about 750, about 525 to about 750, about 550 to about 750, about 575 to about 750, about 600 to about 750, about 625 to about 750, about 650 to about 750, about 675 to about 750, about 700 to about 750, about 725 to about 750, about 300 to about 725, about 325 to about 725, about 350 to about 725, about 375 to about 725, about 400 to about 725, about 425 to about 725, about 450 to about 725, about 475 to about 725, about 500 to about 725, about 525 to about 725, about 550 to about 725, about 575 to about 725, about 600 to about 725, about 625 to about 725, about 650 to about 725, about 675 to about 725, about 700 to about 725, about 300 to about 700, about 325 to about 700, about 350 to about 700, about 375 to about 700, about 400 to about 700, about 425 to about 700, about 450 to about 700, about 475 to about 700, about 500 to about 700, about 525 to about 700, about 550 to about 700, about 575 to about 700, about 600 to about 700, about 625 to about 700, about 650 to about 700, about 675 to about 700, about 300 to about 675, about 325 to about 675, about 350 to about 675, about 375 to about 675, about 400 to about 675, about 425 to about 675, about 450 to about 675, about 475 to about 675, about 500 to about 675, about 525 to about 675, about 550 to about 675, about 575 to about 675, about 600 to about 675, about 625 to about 675, about 650 to about 675, about 300 to about 650, about 325 to about 650, about 350 to about 650, about 375 to about 650, about 400 to about 650, about 425 to about 650, about 450 to about 650, about 475 to about 650, about 500 to about 650, about 525 to about 650, about 550 to about 650, about 575 to about 650, about 600 to about 650, about 625 to about 650, about 300 to about 625, about 325 to about 625, about 350 to about 625, about 375 to about 625, about 400 to about 625, about 425 to about 625, about 450 to about 625, about 475 to about 625, about 500 to about 625, about 525 to about 625, about 550 to about 625, about 575 to about 625, or about 600 to about 625 μm.

In some embodiments, the structure comprises a ceramic material such as a calcium phosphate. In some embodiments, the structure comprises about 50-100, 50-95, 50-90, 50-85, 50-80, 50-75, 50-70, 50-65, 50-60, 50-55, 55-100, 55-95, 55-90, 55-85, 55-80, 55-75, 55-70, 55-65, 55-60, 60-100, 60-95, 60-90, 60-85, 60-80, 60-75, 60-70, 60-65, 65-100, 65-95, 65-90, 65-85, 65-80, 65-75, 65-70, 70-100, 70-95, 70-90, 70-85, 70-80, 70-75, 75-100, 75-95, 75-90, 75-85, 75-80, 80-100, 80-95, 80-90, 80-85, 85-100, 85-95, 85-90, 90-100, 90-95, 95-100, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent ceramic material. In some cases, the ceramic material is calcium phosphate, such as beta-tricalcium phosphate (β-TCP).

In a non-limiting example, a structure has about 50-90% ceramic material such as β-TCP. In some cases, the structure has about 50, 55, 60, 65, 70, 75, 80, 85, or 90% ceramic material such as β-TCP. In some embodiments, the structure has about 10-50% polymer such as polycaprolactone (PCL) or polydioxanone (PDS), or poly-1-lactide. In some cases, the structure has about 10, 15, 20, 25, 30, 35, 40, 45, or 50% polymer such as PCL or PDS or poly-1-lactide. Example structures include those having: about 85-90% ceramic (e.g., β-TCP) and about 10-15% polymer (e.g., PCL or PDS or poly-1-lactide) by weight, about 80-85% ceramic (e.g., β-TCP) and about 15-20% polymer (e.g., PCL or PDS or poly-1-lactide) by weight, about 75-80% ceramic (e.g., β-TCP) and about 20-25% polymer (e.g., PCL or PDS or poly-1-lactide) by weight, about 70-75% ceramic (e.g., β-TCP) and about 25-30% polymer (e.g., PCL or PDS or poly-1-lactide) by weight, about 65-70% ceramic (e.g., β-TCP) and about 30-35% polymer (e.g., PCL or PDS or poly-1-lactide) by weight, about 60-65% ceramic (e.g., β-TCP) and about 35-40% polymer (e.g., PCL or PDS or poly-1-lactide) by weight, about 55-60% ceramic (e.g., β-TCP) and about 40-45% polymer (e.g., PCL or PDS or poly-1-lactide) by weight, about 50-55% ceramic (e.g., β-TCP) and about 45-50% polymer (e.g., PCL or PDS or poly-1-lactide) by weight, about 90% ceramic (e.g., β-TCP) and about 10% polymer (e.g., PCL or PDS or poly-1-lactide) by weight, about 89% ceramic (e.g., β-TCP) and about 11% polymer (e.g., PCL or PDS or poly-1-lactide) by weight, about 88% ceramic (e.g., β-TCP) and about 12% polymer (e.g., PCL or PDS or poly-1-lactide) by weight, about 87% ceramic (e.g., β-TCP) and about 13% polymer (e.g., PCL or PDS or poly-1-lactide) by weight, about 86% ceramic (e.g., β-TCP) and about 14% polymer (e.g., PCL or PDS or poly-1-lactide) by weight, about 85% ceramic (e.g., β-TCP) and about 15% polymer (e.g., PCL or PDS or poly-1-lactide) by weight, about 84% ceramic (e.g., β-TCP) and about 16% polymer (e.g., PCL or PDS or poly-1-lactide) by weight, about 83% ceramic (e.g., β-TCP) and about 17% polymer (e.g., PCL or PDS or poly-1-lactide) by weight, about 82% ceramic (e.g., β-TCP) and about 18% polymer (e.g., PCL or PDS or poly-1-lactide) by weight, about 81% ceramic (e.g., β-TCP) and about 19% polymer (e.g., PCL or PDS or poly-1-lactide) by weight, about 80% ceramic (e.g., β-TCP) and about 20% polymer (e.g., PCL or PDS or poly-1-lactide) by weight, about 79% ceramic (e.g., β-TCP) and about 21% polymer (e.g., PCL or PDS or poly-1-lactide) by weight, about 78% ceramic (e.g., β-TCP) and about 22% polymer (e.g., PCL or PDS or poly-1-lactide) by weight, about 77% ceramic (e.g., β-TCP) and about 23% polymer (e.g., PCL or PDS or poly-1-lactide) by weight, about 76% ceramic (e.g., β-TCP) and about 24% polymer (e.g., PCL or PDS or poly-1-lactide) by weight, about 75% ceramic (e.g., β-TCP) and about 25% polymer (e.g., PCL or PDS or poly-1-lactide) by weight, about 74% ceramic (e.g., β-TCP) and about 26% polymer (e.g., PCL or PDS or poly-1-lactide) by weight, about 73% ceramic (e.g., β-TCP) and about 27% polymer (e.g., PCL or PDS or poly-1-lactide) by weight, about 72% ceramic (e.g., β-TCP) and about 28% polymer (e.g., PCL or PDS or poly-1-lactide) by weight, about 71% ceramic (e.g., β-TCP) and about 29% polymer (e.g., PCL or PDS or poly-1-lactide) by weight, about 70% ceramic (e.g., β-TCP) and about 30% polymer (e.g., PCL or PDS or poly-1-lactide) by weight, about 69% ceramic (e.g., β-TCP) and about 31% polymer (e.g., PCL or PDS or poly-1-lactide) by weight, about 68% ceramic (e.g., β-TCP) and about 32% polymer (e.g., PCL or PDS or poly-1-lactide) by weight, about 67% ceramic (e.g., β-TCP) and about 33% polymer (e.g., PCL or PDS or poly-1-lactide) by weight, about 66% ceramic (e.g., β-TCP) and about 34% polymer (e.g., PCL or PDS or poly-1-lactide) by weight, about 65% ceramic (e.g., β-TCP) and about 35% polymer (e.g., PCL or PDS or poly-1-lactide) by weight, about 64% ceramic (e.g., β-TCP) and about 36% polymer (e.g., PCL or PDS or poly-1-lactide) by weight, about 63% ceramic (e.g., β-TCP) and about 37% polymer (e.g., PCL or PDS or poly-1-lactide) by weight, about 62% ceramic (e.g., β-TCP) and about 38% polymer (e.g., PCL or PDS or poly-1-lactide) by weight, about 61% ceramic (e.g., β-TCP) and about 39% polymer (e.g., PCL or PDS or poly-1-lactide) by weight, about 60% ceramic (e.g., β-TCP) and about 40% polymer (e.g., PCL or PDS or poly-1-lactide) by weight, about 59% ceramic (e.g., β-TCP) and about 41% polymer (e.g., PCL or PDS or poly-1-lactide) by weight, about 58% ceramic (e.g., β-TCP) and about 42% polymer (e.g., PCL or PDS or poly-1-lactide) by weight, about 57% ceramic (e.g., β-TCP) and about 43% polymer (e.g., PCL or PDS or poly-1-lactide) by weight, about 56% ceramic (e.g., β-TCP) and about 44% polymer (e.g., PCL or PDS or poly-1-lactide) by weight, about 55% ceramic (e.g., β-TCP) and about 45% polymer (e.g., PCL or PDS or poly-1-lactide) by weight, about 54% ceramic (e.g., β-TCP) and about 46% polymer (e.g., PCL or PDS or poly-1-lactide) by weight, about 53% ceramic (e.g., β-TCP) and about 47% polymer (e.g., PCL or PDS or poly-1-lactide) by weight, about 52% ceramic (e.g., β-TCP) and about 48% polymer (e.g., PCL or PDS or poly-1-lactide) by weight, about 51% ceramic (e.g., β-TCP) and about 49% polymer (e.g., PCL or PDS or poly-1-lactide) by weight, and about 50% ceramic (e.g., β-TCP) and about 50% polymer (e.g., PCL or PDS or poly-1-lactide) by weight.

The structure may be manufactured using 3D printing from an ink comprising about 30-70% by weight β-TCP powder, about 10-30% by weight first polymer, and about 10-30% by weight second polymer. In some cases, the structure is manufactured using 3D printing from an ink comprising about 30-70% by weight β-TCP powder, about 10-30% by weight first polymer, and about 5-15% by weight second polymer, and about 5-15% third polymer. In some cases structure may be manufactured using 3D printing from an ink comprising about 30-70% by weight β-TCP powder, about 10-30% by weight first polymer, and about 5-15% by weight second polymer (8000 MW PEG) and about 5-15% by weight third polymer (35,000 MW PEG). In some cases, the first polymer comprises PCL. In some cases, the first polymer comprises PDS. In some cases the first polymer comprises poly-1-lactide. In some cases, the second polymer comprises

PEG. In some cases, the third polymer comprises PEG. In some cases, the ink further comprises about 1-10% by weight particulate (e.g., sucrose). In some cases, the ink further comprises about 5-20% blowing agent (e.g., sodium bicarbonate).

In some embodiments the three-dimensional structure has a density of about 1 g/cm³ to about 2 g/cm³ or about 1 g/cm³ to about 1.5 g/cm³. In some embodiments the three-dimensional structure has an open porosity of about 20% to about 40%, about 25% to about 35%, e.g., about 25%, 26%, 27%, 28%, 29%, 30%, 31%, 32%, 33%, 34%, or 35%. In some embodiments a three-dimensional structure has a strut diameter of about 300 μm to about 900 μm, about 300 μm to about 400 μm, or about 500 μm to about 900 μm.

In some embodiments the three-dimensional structure has a density of about 1 g/cm³ to about 2 g/cm³ or about 1.25 g/cm³ to about 1.75 g/cm³. In some embodiments the three-dimensional structure has an open porosity of about 20% to about 40%, about 25% to about 35%, e.g., about 25%, 26%, 27%, 28%, 29%, 30%, 31%, 32%, 33%, 34%, or 35%. In some embodiments a three-dimensional structure has a strut diameter of about 400 μm to about 500 μm, about 400 μm to about 450 μm, or about 425 μm to about 450 μm.

In some embodiments the three-dimensional structure has a density of about 1 g/cm³ to about 2 g/cm³ or about 1 g/cm³ to about 1.5 g/cm³. In some embodiments the three-dimensional structure has an open porosity of about 30% to about 50%, about 35% to about 45%, e.g., about 35%, 36%, 37%, 38%, 39%, 40%, 41%, 42%, 43%, 44%, or 45%. In some embodiments a three-dimensional structure has a strut diameter of about 325 μm to about 425 μm, about 350 μm to about 400 μm, or about 360 μm to about 390 μm.

In some embodiments the three-dimensional structure has a density of about 1 g/cm³ to about 2 g/cm³ or about 1 g/cm³ to about 1.5 g/cm³. In some embodiments the three-dimensional structure has an open porosity of about 30% to about 50%, about 35% to about 45%, e.g., about 35%, 36%, 37%, 38%, 39%, 40%, 41%, 42%, 43%, 44%, or 45%. In some embodiments a three-dimensional structure has a strut diameter of about 350 μm to about 450 μm, about 350 μm to about 400 μm, or about 380 μm to about 405 μm.

In a non-limiting example, a structure has about 50-90% ceramic material such as β-TCP. In some cases, the structure has about 50, 55, 60, 65, 70, 75, 80, 85, or 90% ceramic material such as β-TCP. In some embodiments, the structure has about 10-50% copolymer such as polycaprolactone/polyglycolide copolymer (PCL/PGA, e.g., 90:10, 95:5), poly(D,L-lactide-co-glycolide) copolymer (PLGA, e.g., 50:50), PDS-glycolide copolymer (PDS/PGA, e.g., 90:10), PDS-L-lactide copolymer (PDS/PLA, e.g., 90:10), or Dioxanone/L-lactide copolymer (e.g., 90:10). In some cases, the structure has about 10, 15, 20, 25, 30, 35, 40, 45, or 50% polymer such as PCL/PGA, PDS/PGA, PDS/PLA, PLGA, Dioxanone/L-lactide, Caprolactone/Glycolide, Glycolide/L-lactide, or Poly(D,L-lactide-co-glycolide). Example structures include those having: about 85-90% ceramic (e.g., β-TCP) and about 10-15% polymer (e.g., PCL/PGA, PDS/PGA, PDS/PLA, PLGA, or Dioxanone/L-lactide) by weight, about 80-85% ceramic (e.g., β-TCP) and about 15-20% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 75-80% ceramic (e.g., β-TCP) and about 20-25% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 70-75% ceramic (e.g., β-TCP) and about 25-30% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 65-70% ceramic (e.g., β-TCP) and about 30-35% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 60-65% ceramic (e.g., β-TCP) and about 35-40% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 55-60% ceramic (e.g., β-TCP) and about 40-45% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 50-55% ceramic (e.g., β-TCP) and about 45-50% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 90% ceramic (e.g., β-TCP) and about 10% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 89% ceramic (e.g., β-TCP) and about 11% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 88% ceramic (e.g., β-TCP) and about 12% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 87% ceramic (e.g., β-TCP) and about 13% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 86% ceramic (e.g., β-TCP) and about 14% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 85% ceramic (e.g., β-TCP) and about 15% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 84% ceramic (e.g., β-TCP) and about 16% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 83% ceramic (e.g., β-TCP) and about 17% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 82% ceramic (e.g., β-TCP) and about 18% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 81% ceramic (e.g., β-TCP) and about 19% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 80% ceramic (e.g., β-TCP) and about 20% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 79% ceramic (e.g., β-TCP) and about 21% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 78% ceramic (e.g., β-TCP) and about 22% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 77% ceramic (e.g., β-TCP) and about 23% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 76% ceramic (e.g., β-TCP) and about 24% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 75% ceramic (e.g., β-TCP) and about 25% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 74% ceramic (e.g., β-TCP) and about 26% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 73% ceramic (e.g., β-TCP) and about 27% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 72% ceramic (e.g., β-TCP) and about 28% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 71% ceramic (e.g., β-TCP) and about 29% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 70% ceramic (e.g., β-TCP) and about 30% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 69% ceramic (e.g., β-TCP) and about 31% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 68% ceramic (e.g., β-TCP) and about 32% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 67% ceramic (e.g., β-TCP) and about 33% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 66% ceramic (e.g., β-TCP) and about 34% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 65% ceramic (e.g., β-TCP) and about 35% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 64% ceramic (e.g., β-TCP) and about 36% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 63% ceramic (e.g., β-TCP) and about 37% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 62% ceramic (e.g., β-TCP) and about 38% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 61% ceramic (e.g., β-TCP) and about 39% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 60% ceramic (e.g., β-TCP) and about 40% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 59% ceramic (e.g., β-TCP) and about 41% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 58% ceramic (e.g., β-TCP) and about 42% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 57% ceramic (e.g., β-TCP) and about 43% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 56% ceramic (e.g., β-TCP) and about 44% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 55% ceramic (e.g., β-TCP) and about 45% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 54% ceramic (e.g., β-TCP) and about 46% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 53% ceramic (e.g., β-TCP) and about 47% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, about 52% ceramic (e.g., β-TCP) and about 48% PDS/PLA, polymer (e.g., PCL/PGA, PDS/PGA, PLGA, Dioxanone/L-lactide, Caprolactone/Glycolide, Glycolide/L-lactide, or Poly(D,L-lactide-co-glycolide)) by weight, about 51% ceramic (e.g., β-TCP) and about 49% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight, and about 50% ceramic (e.g., β-TCP) and about 50% polymer (e.g., PCL/PGA, PDS/PGA, PDS/PLA, PLGA, Dioxanone/L-lactide, Caprolactone/Glycolide, Glycolide/L-lactide, or Poly(D,L-lactide-co-glycolide)) by weight.

In some embodiments, the compositions of ink formulations herein are varied to optimize specific surface area. The surface area may be optimized for combination with a certain therapeutic agent. For example, the structure has a surface area of about 0.2-2 m²/g for combination with a BMP protein (e.g., tBMP-2). In some embodiments, the surface area of a structure herein is about 0.2-2, 0.2-1.8, 0.2-1.6, 0.2-1.4, 0.2-1.2, 0.2-1, 0.2-0.8, 0.2-0.6, 0.2-0.4, 0.4-2, 0.4-1.8, 0.4-1.6, 0.4-1.4, 0.4-1.2, 0.4-1, 0.4-0.8, 0.4-0.6, 0.6-2, 0.6-1.8, 0.6-1.6, 0.6-1.4, 0.6-1.2, 0.6-1, 0.6-0.8, 0.8-2, 0.8-1.8, 0.8-1.6, 0.8-1.4, 0.8-1.2, 0.8-1, 1-2, 1-1.8, 1-1.6, 1-1.4, 1-1.2, 1.2-2, 1.2-1.8, 1.2-1.6, 1.2-1.4, 1.4-2, 1.4-1.8, 1.4-1.6, 1.6-2, 1.6-1.8, or 1.8-2 m²/g. In some embodiments, the surface area is calculated by Brunauer-Emmett-Teller (BET) by gas physisorption.

In some embodiments, the compositions of ink formulations herein are varied to optimize resorption rate of one or more materials of the scaffold. For instance, the polymers are selected based on resorption rate. The resorption rates vary from slowest to fastest as: polycaprolactone, polycaprolactone/polyglycolide copolymer (95:5), polycaprolactone/glycolide copolymer (90:10), polydioxanone/L-lactide copolymer (90:10), poly(D,L-lactide-co-glycolide) copolymer (50:50).

### Methods of Manufacture

In another aspect, provided are methods of manufacturing a structure using 3D printing techniques.

In some embodiments, the method comprises syringe-based melt extrusion bioprinting. Example inks for this method may be low in viscosity for extrusion of the ink through a narrow nozzle. Non-limiting example methods of manufacturing using this method are described in Example 2, for instance, with regard to printing ink formulations #1, #2, #3, #4.

In one aspect, the method is an extrusion based method comprising a 3D printing method that extrudes the material out of a nozzle.

In some embodiments, the extrusion based method encompasses bioprinting (syringe-based pneumatic printing) or Fused Granular Fabrication (FGF) where pellets of feedstock are fed from a hopper into a mini-screw extrusion head which melts and pushes the material out of a fine nozzle. In some embodiments, the inks are formed into small (e.g., 2-5 mm) pellets or granules. In example embodiments, the ink comprises a plurality of pellets or granules having an average diameter of X, wherein at least 90% of the plurality of pellets or granules have an individual diameter of X+/−0.5 mm. For example, the plurality of pellets or granules have an average diameter of 2 mm, where at least 90% of the plurality of pellets or granules have an individual diameter of 1.5-2.5 mm. As another example, the plurality of pellets or granules have an average diameter of 5 mm, where at least 90% of the plurality of pellets or granules have an individual diameter of 4.5-5.5 mm.

In some embodiments, the method comprises fused filament fabrication (FFF). Example inks for this method may be formed into filaments for printing on FFF 3D printers. Non-limiting example methods of manufacturing using this method are described in Example 2, for instance, with regard to printing ink formulations #5 and #6.

In some embodiments, the method comprises pelletized fused deposition modeling. Fused deposition modeling (FDM) is an additive manufacturing process. Three dimensional objects are formed through extrusion and deposition of individual layers of thermoplastic materials. FDM involves the melt extrusion of filament materials through a heated nozzle and deposition as thin solid layers on a platform. A thermoplastic polymer material is fed into a temperature-controlled FDM extrusion head and it is heated to a semi-liquid state. Afterward, the FDM extrusion head extrudes and deposits the material in ultra-thin layers onto a base with precision. The material solidifies, laminating to the preceding layer. In this way, parts are fabricated in layers, where each layer is built by extruding a small bead of material, called a road, in a particular pattern, such that the layer is covered with the adjacent roads. After each layer is completed, extrusion head height is increased and subsequent layers are built to construct the part. Usually, FDM is used to fabricate solid models. In order to fabricate porous structures, raster fill gaps have a positive value which is applied to impart a channel within a build layer. Arranged in a regular manner, the channels are interconnected even in three dimensions. Layer by layer fabrication allows design of a pore morphology which varies across a scaffold structure.

In some embodiments, the method comprises selective laser sintering (SLS). Selective laser sintering (SLS) is a process wherein a dispenser deposits layers of powdered material into a target area. There is a laser control mechanism that typically includes a computer with the article design stored on it. The laser control mechanism modulates and moves a laser beam to selectively irradiate the powder layer within defined boundaries of the design, melting the powder on which the laser beam falls. This is done to selectively sinter sequential powder layers. The method produces a completed article comprised of a plurality of layers sintered together.

In some embodiments, after 3D printing, the resulting subject is soaked in water to dissolve certain components of the ink, e.g., PEG, particulate (e.g., pore forming agent, sucrose), blowing agent (e.g., sodium bicarbonate), or a combination thereof. The structure may then be dried, sterilized, treated with a therapeutic as described elsewhere herein, or a combination thereof.

Any of the 3D-printed structures described herein can be coated with a tetherable protein (for example, tBMP2). Following completion of the structures using any of the methods discussed herein, the structures can be washed in an acidic sodium acetate buffer. This can be one, two, or more washes. The washing can then be followed by a two-hour incubation of the structures in sodium acetate buffer that contains a 1 mg/mL concentration of tBMP2 protein. The tetherable tBMP2 binds to the β-TCP surface of the implantable structures in a monolayer.

In further embodiments, the ink formulations discussed herein can include a light-sensitive resin that is mixed with the ceramic powder for digital light processing (DLP), an additive manufacturing technique that is faster than robocasting or melt extrusion. Components in a photosensitive, ceramic-filled resin for DLP 3D printing of bone implants typically include ceramic powder (e.g., β-TCP, hydroxyapatite, bioglass, typically ≤10 μm particle size), one or more crosslinking acrylates or methacrylates (e.g., polyethylene glycol diacrylate, polycaprolactone methacrylate), a plasticizer to reduce resin viscosity (e.g., water), a dispersant to promote breakdown of powder agglomerates (e.g., Darvan® 821-A), photoinitiator to initiate the photocrosslinking reaction (e.g., Lithium phenyl-2,4,6-trimethylbenzoylphosphinate), and a photoabsorber to retain high x-y resolution (e.g., tartrazine). Once resin formulations are prepared by asymmetric centrifugal mixing of the components, the ink is exposed layer by layer to a DLP image, causing the lighted pixels to selectively solidify when the resin encounters the light. Once the implantable structure has been built up layer by layer, it can be thermally processed to burn out the included polymer and densify the ceramic (e.g., a polyethylene glycol diacrylate-containing resin), or left as-is, resulting in a flexible ceramic/polymer composite implant (e.g., a polycaprolactone methacrylate-containing resin).

### Devices

In another aspect, provided are devices and kits comprising a 3D printed structure (e.g., scaffold) described herein and a therapeutic agent. In some embodiments, a device comprises the therapeutic agent connected to, dispersed within, or otherwise combined with the 3D printed structure. As used herein, a therapeutic agent is inclusive of a plurality of therapeutic agents, such as 2, 3, 4, or 5 therapeutic agents.

### Therapeutic Agents

In some embodiments, the therapeutic agent comprises a mammalian growth factor or a functional portion thereof. Mammalian growth factors can be osteoinductive molecules that are capable of initiating and enhancing the bone repair process. A functional portion of the mammalian growth factor is a region that has a therapeutic effect. For instance, a functional portion of a mammalian growth factor is osteoinductive. As another example, a functional portion of a mammalian growth factor is capable of initiating and/or enhancing bone repair. A functional portion of a mammalian growth factor may have osteogenic activity.

Non-limiting examples of mammalian growth factors are described herein. In some instances, the mammalian growth factor comprises: epidermal growth factor (EGF), platelet derived growth factor (PDGF), insulin like growth factor (IGF-1), fibroblast growth factor (FGF), fibroblast growth factor 2 (FGF2), fibroblast growth factor 18 (FGF18), transforming growth factor alpha (TGF-α), transforming growth factor beta (TGF-β), transforming growth factor beta 1 (TGF-β1), transforming growth factor beta 3 (TGF-β3), osteogenic protein 1 (OP-1), osteogenic protein 2 (OP-2), osteogenic protein 3 (OP-3), bone morphogenetic protein 2 (BMP-2), bone morphogenetic protein 3 (BMP-3), bone morphogenetic protein 4 (BMP-4), bone morphogenetic protein 5 (BMP-5), bone morphogenetic protein 6 (BMP-6), bone morphogenetic protein 7 (BMP-7), bone morphogenetic protein (BMP-9), bone morphogenetic protein 10 (BMP-10), bone morphogenetic protein 11 (BMP-11), bone morphogenetic protein 12 (BMP-12), bone morphogenetic protein 13 (BMP-13), bone morphogenetic protein 15 (BMP-15), dentin phosphoprotein (DPP), vegetal related growth factor (Vgr), growth differentiation factor 1 (GDF-1), growth differentiation factor 3 (GDF-3), growth differentiation factor 5 (GDF-5), growth differentiation factor 6 (GDF-6), growth differentiation factor 7 (GDF-7), growth differentiation factor 8 (GDF8), growth differentiation factor 11 (GDF11), growth differentiation factor 15 (GDF15), vascular endothelial growth factor (VEGF), hyaluronic acid binding protein (HABP), and collagen binding protein (CBP), fibroblast growth factor 18 (FGF-18), keratinocyte growth factor (KGF), tumor necrosis factor alpha (TNFα), tumor necrosis factor (TNF)-related apoptosis inducing ligand (TRAIL), wnt family member 1 (WNT1), wnt family member 2 (WNT2), wnt family member 2B (WNT2B), wnt family member 3 (WNT3), wnt family member 3A (WNT3A), wnt family member 4 (WNT4), wnt family member 5A (WNT5A), wnt family member 5B (WNT5B), wnt family member 6 (WNT6), wnt family member 7A (WNT7A), wnt family member 7B (WNT7B), wnt family member 8A (WNT8A), wnt family member 8B (WNT8B), wnt family member 9A (WNT9A), wnt family member 9B (WNT9B), wnt family member 10A (WNT10A), wnt family member 10B (WNT10B), wnt family member 11 (WNT11), or wnt family member 16 (WNT16), or a mature peptide or functional portion thereof.

In some embodiments, the mammalian growth factor is a human growth factor. Non-limiting examples of human growth factors and mature peptides and/or functional portions thereof are provided in Table 1. In some embodiments, the mammalian growth factor comprises a sequence that is at least 70% identical (e.g., at least 75% identical, at least 80% identical, at least 85% identical, at least 90% identical, at least 95% identical, or at least 99% identical) to any of the sequences in Table 1 or any secreted human growth factor, and has osteogenic activity. In some embodiments, the amino acids in a mammalian growth factor that are conserved between different species are likely important for osteogenic activity and may not be mutated, while amino acids in a mammalian growth factor that are not conserved between different species are not likely important for osteogenic activity and may be mutated.

In some embodiments, the mammalian growth factor comprises BMP-2. In some embodiments, the mammalian growth factor is a mature peptide of BMP-2 (e.g., does not comprise a signal sequence). In some embodiments, the mammalian growth factor comprises a functional portion of BMP-2. In some embodiments, the functional portion of BMP-2 comprises a sequence at least about 70%, 75%, 80%, 85%, 90%, 95%, or 100% identical to: QAKHKQRKRLKSSCKRHPLYVDFSDVGWNDWIVAPPGYHAFYCHGECPFPLADHLNS TNHAIVQTLVNSVNSKIPKACCVPTELSAISMLYLDENEKVVLKNYQDMVVEGCGCR (SEQ ID NO: 454). In some embodiments, the mammalian growth factor comprises a sequence at least about 70%, 75%, 80%, 85%, 90%, 95%, or 100% identical to SEQ ID NO: 454. In some embodiments, the mammalian growth factor comprises a sequence at least about 90% identical to SEQ ID NO: 454. In some embodiments, the mammalian growth factor comprises SEQ ID NO: 454.

In some embodiments, the mammalian growth factor is a non-human mammalian growth factor. The non-human mammalian growth factor may be homologous to a human growth factor, such as one or more of the human growth factors of Table 1. In some embodiments, a non-human mammalian growth factor is homologous to a human growth factor if the non-human mammalian growth factor is at least about 80% identical to the human mammalian growth factor as determined using the NCBI Blast alignment algorithm as of the date of this filing. In some cases, the coverage is at least about 90%. In some embodiments, a non-human mammalian growth factor is homologous to a human growth factor if the non-human mammalian growth factor is at least about 80% positive as compared to the human mammalian growth factor as determined using the NCBI Blast alignment algorithm as of the date of this filing. In some cases, the coverage is at least about 90%. In some embodiments, a non-human mammalian growth factor is homologous to a human growth factor if the non-human mammalian growth factor aligned with the human growth factor using the NCBI Blast as of the date of this filing has an E value of less than about 1E-40, at least about 1E-50, 1E-60, 1E-70, or 1E-10, with a query cover of at least about 90%.

TABLE 1
Therapeutic Growth Factors

| Name | Protein Sequence | SEQ ID NO: |
|---|---|---|
| EGF | NSDSECPLSHDGYCLHDGVCMYIEALDKYACNCVVGYIGERCQYRDLK | 442 |
| | WWELR | |
| PDGF | EEAEIPREVIERLARSQIHSIRDLQRLLEIDSVGSEDSLDTSLRAHGVHATK | 443 |
| | HVPEKRPLPIRRKR | |
| IGF-1 | GPETLCGAELVDALQFVCGDRGFYFNKPTGYGSSSRRAPQTGIVDECCFR | 444 |
| | SCDLRRLEMYCAPLKPAKSA | |
| FGF | FNLPPGNYKKPKLLYCSNGGHFLRILPDGTVDGTRDRSDQHIQLQLSAES | 445 |
| | VGEVYIKSTETGQYLAMDTDGLLYGSQTPNEECLFLERLEENHYNTYISK | |
| | KHAEKNWFVGLKKNGSCKRGPRTHYGQKAILFLPLPVSSD | |
| FGF2 | PALPEDGGSGAFPPGHFKDPKRLYCKNGGFFLRIHPDGRVDGVREKSDPH | 446 |
| | IKLQLQAEERGVVSIKGVCANRYLAMKEDGRLLASKCVTDECFFFERLES | |
| | NNYNTYRSRKYTSWYVALKRTGQYKLGSKTGPGQKAILFLPMSAKS | |
| FGF18 | EENVDFRIHVENQTRARDDVSRKQLRLYQLYSRTSGKHIQVLGRRISARG | 447 |
| | EDGDKYAQLLVETDTFGSQVRIKGKETEFYLCMNRKGKLVGKPDGTSKE | |
| | CVFIEKVLENNYTALMSAKYSGWYVGFTKKGRPRKGPKTRENQQDVHF | |
| | MKRYPKGQPELQKPFKYTTVTKRSRRIRPTHPA | |
| TGF-α | ENSTSPLSADPPVAAAVVSHFNDCPDSHTQFCFHGTCRFLVQEDKPACVC | 448 |
| | HSGYVGARCEHADLLAVVAASQKKQAITALVVVSIVALAVLIITCVLIHC | |
| | CQVRKHCEWCRALICRHEKPSALLKGRTACCHSETVV | |
| TGF-α | VVSHFNDCPDSHTQFCFHGTCRFLVQEDKPACVCHSGYVGARCEHADLL | 449 |
| | A | |
| TGF-ß1 | ALDTNYCFSSTEKNCCVRQLYIDFRKDLGWKWIHEPKGYHANFCLGPCP | 450 |
| | YIWSLDTQYSKVLALYNQHNPGASAAPCCVPQALEPLPIVYYVGRKPKV | |
| | EQLSNMIVRSCKCS | |
| TGF-ß3 | ALDTNYCFRNLEENCCVRPLYIDFRQDLGWKWVHEPKGYYANFCSGPCP | 451 |
| | YLRSADTTHSTVLGLYNTLNPEASASPCCVPQDLEPLTILYYVGRTPKVE | |
| | QLSNMVVKSCKCS | |
| OP-2 (BMP-8) | AVRPLRRRQPKKSNELPQANRLPGIFDDVHGSHGRQVCRRHELYVSFQD | 452 |
| | LGWLDWVIAPQGYSAYYCEGECSFPLDSCMNATNHAILQSLVHLMMPD | |
| | AVPKACCAPTKLSATSVLYYDSSNNVILRKHRNMVVKACGCH | |
| BMP8A | AVRPLRRRQPKKSNELPQANRLPGIFDDVRGSHGRQVCRRHELYVSFQD | 453 |
| | LGWLDWVIAPQGYSAYYCEGECSFPLDSCMNATNHAILQSLVHLMKPN | |
| | AVPKACCAPTKLSATSVLYYDSSNNVILRKHRNMVVKACGCH | |
| BMP-2 | QAKHKQRKRLKSSCKRHPLYVDFSDVGWNDWIVAPPGYHAFYCHGECP | 454 |
| | FPLADHLNSTNHAIVQTLVNSVNSKIPKACCVPTELSAISMLYLDENEKV | |
| | VLKNYQDMVVEGCGCR | |
| BMP-3 | QWIEPRNCARRYLKVDFADIGWSEWIISPKSFDAYYCSGACQFPMPKSLK | 455 |
| | PSNHATIQSIVRAVGVVPGIPEPCCVPEKMSSLSILFFDENKNVVLKVYPN | |
| | MTVESCACR | |
| BMP-4 | SPKHHSQRARKKNKNCRRHSLYVDFSDVGWNDWIVAPPGYQAFYCHGD | 456 |
| | CPFPLADHLNSTNHAIVQTLVNSVNSSIPKACCVPTELSAISMLYLDEYDK | |
| | VVLKNYQEMVVEGCGCR | |
| BMP-5 | AANKRKNQNRNKSSSHQDSSRMSSVGDYNTSEQKQACKKHELYVSFRD | 457 |
| | LGWQDWIIAPEGYAAFYCDGECSFPLNAHMNATNHAIVQTLVHLMFPD | |
| | HVPKPCCAPTKLNAISVLYFDDSSNVILKKYRNMVVRSCGCH | |
| BMP-6/VGR | SASSRRRQQSRNRSTQSQDVARVSSASDYNSSELKTACRKHELYVSFQDL | 458 |
| | GWQDWIIAPKGYAANYCDGECSFPLNAHMNATNHAIVQTLVHLMNPEY | |
| | VPKPCCAPTKLNAISVLYFDDNSNVILKKYRNMVVRACGCH | |
| BMP-7/OP-1 | STGSKQRSQNRSKTPKNQEALRMANVAENSSSDQRQACKKHELYVSFRD | 459 |
| | LGWQDWIIAPEGYAAYYCEGECAFPLNSYMNATNHAIVQTLVHFINPET | |
| | VPKPCCAPTQLNAISVLYFDDSSNVILKKYRNMVVRACGCH | |
| BMP-9 | SAGAGSHCQKTSLRVNFEDIGWDSWIIAPKEYEAYECKGGCFFPLADDVT | 460 |
| | PTKHAIVQTLVHLKFPTKVGKACCVPTKLSPISVLYKDDMGVPTLKYHY | |
| | EGMSVAECGCR | |
| BMP-10 | NAKGNYCKRTPLYIDFKEIGWDSWIIAPPGYEAYECRGVCNYPLAEHLTP | 461 |
| | TKHAIIQALVHLKNSQKASKACCVPTKLEPISILYLDKGVVTYKFKYEGM | |
| | AVSECGCR | |
| BMP- | NLGLDCDEHSSESRCCRYPLTVDFEAFGWDWIIAPKRYKANYCSGQCEY | 462 |
| 11/GDF-11 | MFMQKYPHTHLVQQANPRGSAGPCCTPTKMSPINMLYFNDKQQIIYGKI | |
| | PGMVVDRCGCS | |
| BMP-12 | TALAGTRTAQGSGGGAGRGHGRRGRSRCSRKPLHVDFKELGWDDWIIA | 463 |
| | PLDYEAYHCEGLCDFPLRSHLEPTNHAIIQTLLNSMAPDAAPASCCVPAR | |
| | LSPISILYIDAANNVVYKQYEDMVVEACGCR | |
| BMP- | TAFASRHGKRHGKKSRLRCSKKPLHVNFKELGWDDWIIAPLEYEAYHCE | 464 |
| 13/GDF-6 | GVCDFPLRSHLEPTNHAIIQTLMNSMDPGSTPPSCCVPTKLTPISILYIDAG | |
| | NNVVYKQYEDMVVESCGCR | |
| BMP-15 | QADGISAEVTASSSKHSGPENNQCSLHPFQISFRQLGWDHWIIAPPFYTPN | 465 |
| | YCKGTCLRVLRDGLNSPNHAIIQNLINQLVDQSVPRPSCVPYKYVPISVL | |
| | MIEANGSILYKEYEGMIAESCTCR | |
| DPP isoform | IPVPQSKPLERHVEKSMNLHLLARSNVSVQDELNASGTIKESGVLVHEGD | 466 |
| 1 | RGRQENTQDGHKGEGNGSKWAEVGGKSFSTYSTLANEEGNIEGWNGDT | |
| | GKAETYGHDGIHGKEENITANGIQGQVSIIDNAGATNRSNTNGNTDKNTQ | |
| | NGDVGDAGHNEDVAVVQEDGPQVAGSNNSTDNEDEIIENSCRNEGNTSE | |
| | ITPQINSKRNGTKEAEVTPGTGEDAGLDNSDGSPSGNGADEDEDEGSGDD | |
| | EDEEAGNGKDSSNNSKGQEGQDHGKEDDHDSSIGQNSDSKEYYDPEGKE | |
| | DPHNEVDGDKTSKSEENSAGIPEDNGSQRIEDTQKLNHRESKRVENRITK | |
| | ESETHAVGKSQDKGIEIKGPSSGNRNITKEVGKGNEGKEDKGQHGMILG | |
| | KGNVKTQGEVVNIEGPGQKSEPGNKVGHSNTGSDSNSDGYDSYDFDDKS | |
| | MQG | |
| DPP isoform | IPVPQSKPLERHVEKSMNLHLLARSNVSVQDELNASGTIKESGVLVHEGD | 467 |
| 2 | RGRQENTQDGHKGEGNGSKWAEVGGKSFSTYSTLANEEGNIEGWNGDT | |
| | GKAETYGHDGIHGKEENITANGIQGQVSIIDNAGATNRSNTNGNTDKNTQ | |
| | NGDVGDAGHNEDVAVVQEDGPQVAGSNNSTDNEDEIIENSCRNEGNTSE | |
| | ITPQINSKRNGTKEAEVTPGTGEDAGLDNSDGSPSGNGADEDEDEGSGDD | |
| | EDEEAGNGKDSSNNSKGQEGQDHGKEDDHDSSIGQNSDSKEYYDPEGKE | |
| | DPHNEVDGDKTSKSEENSAGIPEDNGSQRIEDTQKLNHRESKRVENRITK | |
| | ESETHAVGKSQDKGIEIKGPSSGNRNITKEVGKGNEGKEDKGQHGMILG | |
| | KGNVKTQGEVVNIEGPGQKSEPGNKVGHSNTGSDSNSDGYDSYDFDDKS | |
| | MQGDDPNSSDESNGNDDANSESDNNSSSRGDASYNSDESKDNGNGSDS | |
| | KGAEDDDSDSTSDTNNSDSNGNGNNGNDDNDKSDSGKGKSDSSDSDSS | |
| | DSSNSSDSSDSSDSDSSDSNSSSDSDSSDSDSSDSSDSDSSDSSNSSDSSDSS | |
| | DSSDSSDSSDSSDSKSDSSKSESDSSDSDSKSDSSDSNSSDSSDNSDSSDSS | |
| | NSSNSSDSSDSSDSSDSSSSSDSSNSSDSSDSSDSSNSSESSDSSDSSDSDSS | |
| | DSSDSSNSNSSDSDSSNSSDSSDSSNSSDSSDSSDSSNSSDSSDSSDSSNSSD | |
| | SSDSSDSSDSSDSSNSSDSNDSSNSSDSSDSSNSSDSSNSSDSSDSSDSSDSD | |
| | SSNSSDSSNSSDSSDSSNSSDSSDSSDSSDGSDSDSSNRSDSSNSSDSSDSSD | |
| | SSNSSDSSDSSDSNESSNSSDSSDSSNSSDSDSSDSSNSSDSSDSSNSSDSSE | |
| | SSNSSDNSNSSDSSNSSDSSDSSDSSNSSDSSNSSDSSNSSDSSDSNSSDSSD | |
| | SSNSSDSSDSSDSSDSSDSSDSSNSSDSSDSSDSSDSSNSSDSSNSSDSSNSS | |
| | DSSDSSDSSDSSDSSDSSDSSDSSNSSDSSDSSDSSDSSDSSDSSDSSDSSES | |
| | SDSSDSSNSSDSSDSSDSSDSSDSSDSSDSSDSSDSSNSSDSSDSSDSSDSSD | |
| | SSNSSDSSDSSESSDSSDSSDSSDSSDSSDSSDSSDSSDSSNSSDSSDSSDSS | |
| | DSSDSSDSSDSSDSSDSSDSSDSSDSSDSSDSSDSSDSSDSNESSDSSDSSDS | |
| | SDSSNSSDSSDSSDSSDSTSDSNDESDSQSKSGNGNNNGSDSDSDSEGSDS | |
| | NHSTSDD | |
| DPP isoform | DDPNSSDESNGNDDANSESDNNSSSRGDASYNSDESKDNGNGSDSKGAE | 468 |
| 3 | DDDSDSTSDTNNSDSNGNGNNGNDDNDKSDSGKGKSDSSDSDSSDSSNS | |
| | SDSSDSSDSDSSDSNSSSDSDSSDSDSSDSSDSDSSDSSNSSDSSDSSDSSDS | |
| | SDSSDSSDSKSDSSKSESDSSDSDSKSDSSDSNSSDSSDNSDSSDSSNSSNS | |
| | SDSSDSSDSSDSSSSSDSSNSSDSSDSSDSSNSSESSDSSDSSDSDSSDSSDSS | |
| | NSNSSDSDSSNSSDSSDSSNSSDSSDSSDSSNSSDSSDSSDSSNSSDSSDSSD | |
| | SSDSSDSSNSSDSNDSSNSSDSSDSSNSSDSSNSSDSSDSSDSSDSDSSNSSD | |
| | SSNSSDSSDSSNSSDSSDSSDSSDGSDSDSSNRSDSSNSSDSSDSSDSSNSSD | |
| | SSDSSDSNESSNSSDSSDSSNSSDSDSSDSSNSSDSSDSSNSSDSSESSNSSD | |
| | NSNSSDSSNSSDSSDSSDSSNSSDSSNSSDSSNSSDSSDSNSSDSSDSSNSSD | |
| | SSDSSDSSDSSDSSDSSNSSDSSDSSDSSDSSNSSDSSNSSDSSNSSDSSDSS | |
| | DSSDSSDSSDSSDSSDSSNSSDSSDSSDSSDSSDSSDSSDSSDSSESSDSSDS | |
| | SNSSDSSDSSDSSDSSDSSDSSDSSDSSDSSNSSDSSDSSDSSDSSDSSNSSD | |
| | SSDSSESSDSSDSSDSSDSSDSSDSSDSSDSSDSSNSSDSSDSSDSSDSSDSS | |
| | DSSDSSDSSDSSDSSDSSDSSDSSDSSDSSDSSDSNESSDSSDSSDSSDSSNS | |
| | SDSSDSSDSSDSTSDSNDESDSQSKSGNGNNNGSDSDSDSEGSDSNHSTSD | |
| | D | |
| GDF-1 | DAEPVLGGGPGGACRARRLYVSFREVGWHRWVIAPRGFLANYCQGQCA | 469 |
| | LPVALSGSGGPPALNHAVLRALMHAAAPGAADLPCCVPARLSPISVLFFD | |
| | NSDNVVLRQYEDMVVDECGCR | |
| GDF-3 | AAIPVPKLSCKNLCHRHQLFINFRDLGWHKWIIAPKGFMANYCHGECPFS | 470 |
| | LTISLNSSNYAFMQALMHAVDPEIPQAVCIPTKLSPISMLYQDNNDNVILR | |
| | HYEDMVVDECGCG | |
| GDF-5 | APLATRQGKRPSKNLKARCSRKALHVNFKDMGWDDWIIAPLEYEAFHC | 471 |
| | EGLCEFPLRSHLEPTNHAVIQTLMNSMDPESTPPTCCVPTRLSPISILFIDSA | |
| | NNVVYKQYEDMVVESCGCR | |
| GDF8 | DFGLDCDEHSTESRCCRYPLTVDFEAFGWDWIIAPKRYKANYCSGECEF | 472 |
| | VFLQKYPHTHLVHQANPRGSAGPCCTPTKMSPINMLYFNGKEQIIYGKIP | |
| | AMVVDRCGCS | |
| GDF15 | ARARNGDHCPLGPGRCCRLHTVRASLEDLGWADWVLSPREVQVTMCIG | 473 |
| | ACPSQFRAANMHAQIKTSLHRLKPDTVPAPCCVPASYNPMVLIQKTDTG | |
| | VSLQTYDDLLAKDCHCI | |
| VEGF | APMAEGGGQNHHEVVKFMDVYQRSYCHPIETLVDIFQEYPDEIEYIFKPS | 474 |
| | CVPLMRCGGCCNDEGLECVPTEESNITMQIMRIKPHQGQHIGEMSFLQHN | |
| | KCECRPKKDRARQEKKSVRGKGKGQKRKRKKSRYKSWSVYVGARCCL | |
| | MPWSLPGPHPCGPCSERRKHLFVQDPQTCKCSCKNTDSRCKARQLELNE | |
| | RTCRCDKPRR | |
| HABP | FSLMSLLESLDPDWTPDQYDYSYEDYNQEENTSSTLTHAENPDWYYTED | 475 |
| Isoform 1 | QADPCQPNPCEHGGDCLVHGSTFTCSCLAPFSGNKCQKVQNTCKDNPCG | |
| | RGQCLITQSPPYYRCVCKHPYTGPSCSQVVPVCRPNPCQNGATCSRHKRR | |
| | SKFTCACPDQFKGKFCEIGSDDCYVGDGYSYRGKMNRTVNQHACLYWN | |
| | SHLLLQENYNMFMEDAETHGIGEHNFCRNPDADEKPWCFIKVTNDKVK | |
| | WEYCDVSACSAQDVAYPEESPTEPSTKLPGFDSCGKTEIAERKIKR | |
| HABP | IYGGFKSTAGKHPWQASLQSSLPLTISMPQGHFCGGALIHPCWVLTAAHC | 477 |
| Isoform 2 | TDIKTRHLKVVLGDQDLKKEEFHEQSFRVEKIFKYSHYNERDEIPHNDIAL | |
| | LKLKPVDGHCALESKYVKTVCLPDGSFPSGSECHISGWGVTETGKGSRQ | |
| | LLDAKVKLIANTLCNSRQLYDHMIDDSMICAGNLQKPGQDTCQGDSGGP | |
| | LTCEKDGTYYVYGIVSWGLECGKRPGVYTQVTKFLNWIKATIKSESGF | |
| CBP | AEVKKPAAAAAPGTAEKLSPKAATLAERSAGLAFSLYQAMAKDQAVEN | 479 |
| | ILVSPVVVASSLGLVSLGGKATTASQAKAVLSAEQLRDEEVHAGLGELLR | |
| | SLSNSTARNVTWKLGSRLYGPSSVSFADDFVRSSKQHYNCEHSKINFRDK | |
| | RSALQSINEWAAQTTDGKLPEVTKDVERTDGALLVNAMFFKPHWDEKF | |
| | HHKMVDNRGFMVTRSYTVGVMMMHRTGLYNYYDDEKEKLQIVEMPL | |
| | AHKLSSLIILMPHHVEPLERLEKLLTKEQLKIWMGKMQKKAVAISLPKGV | |
| | VEVTHDLQKHLAGLGLTEAIDKNKADLSRMSGKKDLYLASVFHATAFEL | |
| | DTDGNPFDQDIYGREELRSPKLFYADHPFIFLVRDTQSGSLLFIGRLVRPK | |
| | GDKMRDEL | |
| KGF | CNDMTPEQMATNVNCSSPERHTRSYDYMEGGDIRVRRLFCRTQWYLRID | 480 |
| | KRGKVKGTQEMKNNYNIMEIRTVAVGIVAIKGVESEFYLAMNKEGKLY | |
| | AKKECNEDCNFKELILENHYNTYASAKWTHNGGEMFVALNQKGIPVRG | |
| | KKTKKEQKTAHFLPMAIT | |
| TNFα | GPQREEFPRDLSLISPLAQAVRSSSRTPSDKPVAHVVANPQAEGQLQWLN | 481 |
| | RRANALLANGVELRDNQLVVPSEGLYLIYSQVLFKGQGCPSTHVLLTHTI | |
| | SRIAVSYQTKVNLLSAIKSPCQRETPEGAEAKPWYEPIYLGGVFQLEKGD | |
| | RLSAEINRPDYLDFAESGQVYFGIIAL | |
| TRAIL | TNELKQMQDKYSKSGIACFLKEDDSYWDPNDEESMNSPCWQVKWQLR | 482 |
| | QLVRKMILRTSEETISTVQEKQQNISPLVRERGPQRVAAHITGTRGRSNTL | |
| | SSPNSKNEKALGRKINSWESSRSGHSFLSNLHLRNGELVIHEKGFYYIYSQ | |
| | TYFRFQEEIKENTKNDKQMVQYIYKYTSYPDPILLMKSARNSCWSKDAE | |
| | YGLYSIYQGGIFELKENDRIFVSVTNEHLIDMDHEASFFGAFLVG | |
| WNT1 | ANSSGRWWGIVNVASSTNLLTDSKSLQLVLEPSLQLLSRKQRRLIRQNPG | 483 |
| | ILHSVSGGLQSAVRECKWQFRNRRWNCPTAPGPHLFGKIVNRGCRETAFI | |
| | FAITSAGVTHSVARSCSEGSIESCTCDYRRRGPGGPDWHWGGCSDNIDFG | |
| | RLFGREFVDSGEKGRDLRFLMNLHNNEAGRTTVFSEMRQECKCHGMSG | |
| | SCTVRTCWMRLPTLRAVGDVLRDRFDGASRVLYGNRGSNRASRAELLR | |
| | LEPEDPAHKPPSPHDLVYFEKSPNFCTYSGRLGTAGTAGRACNSSSPALD | |
| | GCELLCCGRGHRTRTQRVTERCNCTFHWCCHVSCRNCTHTRVLHECL | |
| WNT2 | SWWYMRATGGSSRVMCDNVPGLVSSQRQLCHRHPDVMRAISQGVAEW | 484 |
| | TAECQHQFRQHRWNCNTLDRDHSLFGRVLLRSSRESAFVYAISSAGVVF | |
| | AITRACSQGEVKSCSCDPKKMGSAKDSKGIFDWGGCSDNIDYGIKFARAF | |
| | VDAKERKGKDARALMNLHNNRAGRKAVKRFLKQECKCHGVSGSCTLR | |
| | TCWLAMADFRKTGDYLWRKYNGAIQVVMNQDGTGFTVANERFKKPTK | |
| | NDLVYFENSPDYCIRDREAGSLGTAGRVCNLTSRGMDSCEVMCCGRGY | |
| | DTSHVTRMTKCGCKFHWCCAVRCQDCLEALDVHTCKAPKNADWTTAT | |
| WNT2B | SWWYIGALGARVICDNIPGLVSRQRQLCQRYPDIMRSVGEGAREWIREC | 485 |
| | QHQFRHHRWNCTTLDRDHTVFGRVMLRSSREAAFVYAISSAGVVHAITR | |
| | ACSQGELSVCSCDPYTRGRHHDQRGDFDWGGCSDNIHYGVRFAKAFVD | |
| | AKEKRLKDARALMNLHNNRCGRTAVRRFLKLECKCHGVSGSCTLRTCW | |
| | RALSDFRRTGDYLRRRYDGAVQVMATQDGANFTAARQGYRRATRTDL | |
| | VYFDNSPDYCVLDKAAGSLGTAGRVCSKTSKGTDGCEIMCCGRGYDTT | |
| | RVTRVTQCECKFHWCCAVRCKECRNTVDVHTCKAPKKAEWLDQT | |
| WNT3 | GYPIWWSLALGQQYTSLGSQPLLCGSIPGLVPKQLRFCRNYIEIMPSVAEG | 486 |
| | VKLGIQECQHQFRGRRWNCTTIDDSLAIFGPVLDKATRESAFVHAIASAG | |
| | VAFAVTRSCAEGTSTICGCDSHHKGPPGEGWKWGGCSEDADFGVLVSRE | |
| | FADARENRPDARSAMNKHNNEAGRTTILDHMHLKCKCHGLSGSCEVKT | |
| | CWWAQPDFRAIGDFLKDKYDSASEMVVEKHRESRGWVETLRAKYSLFK | |
| | PPTERDLVYYENSPNFCEPNPETGSFGTRDRTCNVTSHGIDGCDLLCCGR | |
| | GHNTRTEKRKEKCHCIFHWCCYVSCQECIRIYDVHTCK | |
| WNT3A | SYPIWWSLAVGPQYSSLGSQPILCASIPGLVPKQLRFCRNYVEIMPSVAEG | 487 |
| | IKIGIQECQHQFRGRRWNCTTVHDSLAIFGPVLDKATRESAFVHAIASAG | |
| | VAFAVTRSCAEGTAAICGCSSRHQGSPGKGWKWGGCSEDIEFGGMVSRE | |
| | FADARENRPDARSAMNRHNNEAGRQAIASHMHLKCKCHGLSGSCEVKT | |
| | CWWSQPDFRAIGDFLKDKYDSASEMVVEKHRESRGWVETLRPRYTYFK | |
| | VPTERDLVYYEASPNFCEPNPETGSFGTRDRTCNVSSHGIDGCDLLCCGR | |
| | GHNARAERRREKCRCVFHWCCYVSCQECTRVYDVHTCK | |
| WNT4 | SNWLYLAKLSSVGSISEEETCEKLKGLIQRQVQMCKRNLEVMDSVRRGA | 488 |
| | QLAIEECQYQFRNRRWNCSTLDSLPVFGKVVTQGTREAAFVYAISSAGV | |
| | AFAVTRACSSGELEKCGCDRTVHGVSPQGFQWSGCSDNIAYGVAFSQSF | |
| | VDVRERSKGASSSRALMNLHNNEAGRKAILTHMRVECKCHGVSGSCEV | |
| | KTCWRAVPPFRQVGHALKEKFDGATEVEPRRVGSSRALVPRNAQFKPHT | |
| | DEDLVYLEPSPDFCEQDMRSGVLGTRGRTCNKTSKAIDGCELLCCGRGF | |
| | HTAQVELAERCSCKFHWCCFVKCRQCQRLVELHTCR | |
| WNT5A | IIGAQPLCSQLAGLSQGQKKLCHLYQDHMQYIGEGAKTGIKECQYQFRH | 489 |
| | RRWNCSTVDNTSVFGRVMQIGSRETAFTYAVSAAGVVNAMSRACREGE | |
| | LSTCGCSRAARPKDLPRDWLWGGCGDNIDYGYRFAKEFVDARERERIHA | |
| | KGSYESARILMNLHNNEAGRRTVYNLADVACKCHGVSGSCSLKTCWLQ | |
| | LADFRKVGDALKEKYDSAAAMRLNSRGKLVQVNSRFNSPTTQDLVYIDP | |
| | SPDYCVRNESTGSLGTQGRLCNKTSEGMDGCELMCCGRGYDQFKTVQT | |
| | ERCHCKFHWCCYVKCKKCTEIVDQFVCK | |
| WNT5B | QLLTDANSWWSLALNPVQRPEMFIIGAQPVCSQLPGLSPGQRKLCQLYQ | 490 |
| | EHMAYIGEGAKTGIKECQHQFRQRRWNCSTADNASVFGRVMQIGSRETA | |
| | FTHAVSAAGVVNAISRACREGELSTCGCSRTARPKDLPRDWLWGGCGD | |
| | NVEYGYRFAKEFVDAREREKNFAKGSEEQGRVLMNLQNNEAGRRAVY | |
| | KMADVACKCHGVSGSCSLKTCWLQLAEFRKVGDRLKEKYDSAAAMRV | |
| | TRKGRLELVNSRFTQPTPEDLVYVDPSPDYCLRNESTGSLGTQGRLCNKT | |
| | SEGMDGCELMCCGRGYNQFKSVQVERCHCKFHWCCFVRCKKCTEIVDQ | |
| | YICK | |
| WNT6 | LWWAVGSPLVMDPTSICRKARRLAGRQAELCQAEPEVVAELARGARLG | 491 |
| | VRECQFQFRFRRWNCSSHSKAFGRILQQDIRETAFVFAITAAGASHAVTQ | |
| | ACSMGELLQCGCQAPRGRAPPRPSGLPGTPGPPGPAGSPEGSAAWEWGG | |
| | CGDDVDFGDEKSRLFMDARHKRGRGDIRALVQLHNNEAGRLAVRSHTR | |
| | TECKCHGLSGSCALRTCWQKLPPFREVGARLLERFHGASRVMGTNDGK | |
| | ALLPAVRTLKPPGRADLLYAADSPDFCAPNRRTGSPGTRGRACNSSAPDL | |
| | SGCDLLCCGRGHRQESVQLEENCLCRFHWCCVVQCHRCRVRKELSLCL | |
| WNT7A | LGASIICNKIPGLAPRQRAICQSRPDAIIVIGEGSQMGLDECQFQFRNGRW | 492 |
| | NCSALGERTVFGKELKVGSREAAFTYAIIAAGVAHAITAACTQGNLSDCG | |
| | CDKEKQGQYHRDEGWKWGGCSADIRYGIGFAKVFVDAREIKQNARTLM | |
| | NLHNNEAGRKILEENMKLECKCHGVSGSCTTKTCWTTLPQFRELGYVLK | |
| | DKYNEAVHVEPVRASRNKRPTFLKIKKPLSYRKPMDTDLVYIEKSPNYCE | |
| | EDPVTGSVGTQGRACNKTAPQASGCDLMCCGRGYNTHQYARVWQCNC | |
| | KFHWCCYVKCNTCSERTEMYTCK | |
| WNT7B | ALSSVVALGANIICNKIPGLAPRQRAICQSRPDAIIVIGEGAQMGINECQYQ | 493 |
| | FRFGRWNCSALGEKTVFGQELRVGSREAAFTYAITAAGVAHAVTAACSQ | |
| | GNLSNCGCDREKQGYYNQAEGWKWGGCSADVRYGIDFSRRFVDAREIK | |
| | KNARRLMNLHNNEAGRKVLEDRMQLECKCHGVSGSCTTKTCWTTLPKF | |
| | REVGHLLKEKYNAAVQVEVVRASRLRQPTFLRIKQLRSYQKPMETDLVY | |
| | IEKSPNYCEEDAATGSVGTQGRLCNRTSPGADGCDTMCCGRGYNTHQYT | |
| | KVWQCNCKFHWCCFVKCNTCSERTEVFTCK | |
| WNT8A | VNNFLITGPKAYLTYTTSVALGAQSGIEECKFQFAWERWNCPENALQLST | 494 |
| | HNRLRSATRETSFIHAISSAGVMYIITKNCSMGDFENCGCDGSNNGKTGG | |
| | HGWIWGGCSDNVEFGERISKLFVDSLEKGKDARALMNLHNNRAGRLAV | |
| | RATMKRTCKCHGISGSCSIQTCWLQLAEFREMGDYLKAKYDQALKIEMD | |
| | KRQLRAGNSAEGHWVPAEAFLPSAEAELIFLEESPDYCTCNSSLGIYGTE | |
| | GRECLQNSHNTSRWERRSCGRLCTECGLQVEERKTEVISSCNCKFQWCC | |
| | TVKCDQCRHVVSKYYCARSPGSAQSLGKGSA | |
| WNT8B | WSVNNFLMTGPKAYLIYSSSVAAGAQSGIEECKYQFAWDRWNCPERAL | 495 |
| | QLSSHGGLRSANRETAFVHAISSAGVMYTLTRNCSLGDFDNCGCDDSRN | |
| | GQLGGQGWLWGGCSDNVGFGEAISKQFVDALETGQDARAAMNLHNNE | |
| | AGRKAVKGTMKRTCKCHGVSGSCTTQTCWLQLPEFREVGAHLKEKYHA | |
| | ALKVDLLQGAGNSAAGRGAIADTFRSISTRELVHLEDSPDYCLENKTLGL | |
| | LGTEGRECLRRGRALGRWERRSCRRLCGDCGLAVEERRAETVSSCNCKF | |
| | HWCCAVRCEQCRRRVTKYFCSRAERPRGGAAHKPGRKP | |
| WNT9A | YFGLTGSEPLTILPLTLEPEAAAQAHYKACDRLKLERKQRRMCRRDPGV | 496 |
| | AETLVEAVSMSALECQFQFRFERWNCTLEGRYRASLLKRGFKETAFLYAI | |
| | SSAGLTHALAKACSAGRMERCTCDEAPDLENREAWQWGGCGDNLKYSS | |
| | KFVKEFLGRRSSKDLRARVDFHNNLVGVKVIKAGVETTCKCHGVSGSCT | |
| | VRTCWRQLAPFHEVGKHLKHKYETALKVGSTTNEAAGEAGAISPPRGRA | |
| | SGAGGSDPLPRTPELVHLDDSPSFCLAGRFSPGTAGRRCHREKNCESICCG | |
| | RGHNTQSRVVTRPCQCQVRWCCYVECRQCTQREEVYTCKG | |
| WNT9B | SYFGLTGREVLTPFPGLGTAAAPAQGGAHLKQCDLLKLSRRQKQLCRRE | 497 |
| | PGLAETLRDAAHLGLLECQFQFRHERWNCSLEGRMGLLKRGFKETAFLY | |
| | AVSSAALTHTLARACSAGRMERCTCDDSPGLESRQAWQWGVCGDNLK | |
| | YSTKFLSNFLGSKRGNKDLRARADAHNTHVGIKAVKSGLRTTCKCHGVS | |
| | GSCAVRTCWKQLSPFRETGQVLKLRYDSAVKVSSATNEALGRLELWAP | |
| | ARQGSLTKGLAPRSGDLVYMEDSPSFCRPSKYSPGTAGRVCSREASCSSL | |
| | CCGRGYDTQSRLVAFSCHCQVQWCCYVECQQCVQEELVYTCKH | |
| WNT10A | MPRSAPNDILDLRLPPEPVLNANTVCLTLPGLSRRQMEVCVRHPDVAASA | 498 |
| | IQGIQIAIHECQHQFRDQRWNCSSLETRNKIPYESPIFSRGFRESAFAYAIA | |
| | AAGVVHAVSNACALGKLKACGCDASRRGDEEAFRRKLHRLQLDALQRG | |
| | KGLSHGVPEHPALPTASPGLQDSWEWGGCSPDMGFGERFSKDFLDSREP | |
| | HRDIHARMRLHNNRVGRQAVMENMRRKCKCHGTSGSCQLKTCWQVTP | |
| | EFRTVGALLRSRFHRATLIRPHNRNGGQLEPGPAGAPSPAPGAPGPRRRA | |
| | SPADLVYFEKSPDFCEREPRLDSAGTVGRLCNKSSAGSDGCGSMCCGRG | |
| | HNILRQTRSERCHCRFHWCCFVVCEECRITEWVSVCK | |
| WNT10B | NEILGLKLPGEPPLTANTVCLTLSGLSKRQLGLCLRNPDVTASALQGLHIA | 499 |
| | VHECQHQLRDQRWNCSALEGGGRLPHHSAILKRGFRESAFSFSMLAAGV | |
| | MHAVATACSLGKLVSCGCGWKGSGEQDRLRAKLLQLQALSRGKSFPHS | |
| | LPSPGPGSSPSPGPQDTWEWGGCNHDMDFGEKFSRDFLDSREAPRDIQAR | |
| | MRIHNNRVGRQVVTENLKRKCKCHGTSGSCQFKTCWRAAPEFRAVGAA | |
| | LRERLGRAIFIDTHNRNSGAFQPRLRPRRLSGELVYFEKSPDFCERDPTMG | |
| | SPGTRGRACNKTSRLLDGCGSLCCGRGHNVLRQTRVERCHCRFHWCCY | |
| | VLCDECKVTEWVNVCK | |
| WNT11 | IKWLALSKTPSALALNQTQHCKQLEGLVSAQVQLCRSNLELMHTVVHA | 500 |
| | AREVMKACRRAFADMRWNCSSIELAPNYLLDLERGTRESAFVYALSAA | |
| | AISHAIARACTSGDLPGCSCGPVPGEPPGPGNRWGGCADNLSYGLLMGA | |
| | KFSDAPMKVKKTGSQANKLMRLHNSEVGRQALRASLEMKCKCHGVSGS | |
| | CSIRTCWKGLQELQDVAADLKTRYLSATKVVHRPMGTRKHLVPKDLDIR | |
| | PVKDSELVYLQSSPDFCMKNEKVGSHGTQDRQCNKTSNGSDSCDLMCC | |
| | GRGYNPYTDRVVERCHCKYHWCCYVTCRRCERTVERYVCK | |
| WNT16 | NWMWLGIASFGVPEKLGCANLPLNSRQKELCKRKPYLLPSIREGARLGIQ | 501 |
| | ECGSQFRHERWNCMITAAATTAPMGASPLFGYELSSGTKETAFIYAVMA | |
| | AGLVHSVTRSCSAGNMTECSCDTTLQNGGSASEGWHWGGCSDDVQYG | |
| | MWFSRKFLDFPIGNTTGKENKVLLAMNLHNNEAGRQAVAKLMSVDCRC | |
| | HGVSGSCAVKTCWKTMSSFEKIGHLLKDKYENSIQISDKTKRKMRRREK | |
| | DQRKIPIHKDDLLYVNKSPNYCVEDKKLGIPGTQGRECNRTSEGADGCNL | |
| | LCCGRGYNTHVVRHVERCECKFIWCCYVRCRRCESMTDVHTCK | |
| DLL4 | MAAASRSASGWALLLLVALWQQRAAGSGVFQLQLQEFINERGVLASGR | 502 |
| | PCEPGCRTFFRVCLKHFQAVVSPGPCTFGTVSTPVLGTNSFAVRDDSSGG | |
| | GRNPLQLPFNFTWPGTFSLIIEAWHAPGDDLRPEALPPDALISKIAIQGSLA | |
| | VGQNWLLDEQTSTLTRLRYSYRVICSDNYYGDNCSRLCKKRNDHFGHY | |
| | VCQPDGNLSCLPGWTGEYCQQPICLSGCHEQNGYCSKPAECLCRPGWQG | |
| | RLCNECIPHNGCRHGTCSTPWQCTCDEGWGGLFCDQDLNYCTHHSPCK | |
| | NGATCSNSGQRSYTCTCRPGYTGVDCELELSECDSNPCRNGGSCKDQED | |
| | GYHCLCPPGYYGLHCEHSTLSCADSPCFNGGSCRERNQGANYACECPPN | |
| | FTGSNCEKKVDRCTSNPCANGGQCLNRGPSRMCRCRPGFTGTYCELHVS | |
| | DCARNPCAHGGTCHDLENGLMCTCPAGFSGRRCEVRTSIDACASSPCFN | |
| | RATCYTDLSTDTFVCNCPYGFVGSRCEFPVGLPPSFPWVAVSLGVGLAVL | |
| | LVLLGMVAVAVRQLRLRRPDDGSREAMNNLSDFQKDNLIPAAQLKNTN | |
| | QKKELEVDCGLDKSNCGKQQNHTLDYNLAPGPLGRGTMPGKFPHSDKS | |
| | LGEKAPLRLHSEKPECRISAICSPRDSMYQSVCLISEERNECVIATEV | |
| DLL4 | SGVFQLQLQEFINERGVLASGRPCEPGCRTFFRVCLKHFQAVVSPGPCTF | 503 |
| | GTVSTPVLGTNSFAVRDDSSGGGRNPLQLPFNFTWPGTFSLIIEAWHAPG | |
| | DDLRPEALPPDALISKIAIQGSLAVGQNWLLDEQTSTLTRLRYSYRVICSD | |
| | NYYGDNCSRLCKKRNDHFGHYVCQPDGNLSCLPGWTGEYCQQPICLSG | |
| | CHEQNGYCSKPAECLCRPGWQGRLCNECIPHNGCRHGTCSTPWQCTCDE | |
| | GWGGLFCDQDLNYCTHHSPCKNGATCSNSGQRSYTCTCRPGYTGVDCE | |
| | LELSECDSNPCRNGGSCKDQEDGYHCLCPPGYYGLHCEHSTLSCADSPCF | |
| | NGGSCRERNQGANYACECPPNFTGSNCEKKVDRCTSNPCANGGQCLNR | |
| | GPSRMCRCRPGFTGTYCELHVSDCARNPCAHGGTCHDLENGLMCTCPA | |
| | GFSGRRCEVRTSIDACASSPCFNRATCYTDLSTDTFVCNCPYGFVGSRCEF | |
| | PVGLP | |
| DLL4 | SGVFQLQLQEFINERGVLASGRPCEPGCRTFFRVCLKHFQAVVSPGPCTF | 504 |
| | GTVSTPVLGTNSFAVRDDSSGGGRNPLQLPFNFTWPGTFSLIIEAWHAPG | |
| | DDLRPEALPPDALISKIAIQGSLAVGQNWLLDEQTSTLTRLRYSYRVICSD | |
| | NYYGDNCSRLCKKRNDHFGHYVCQPDGNLSCLPGWTGEYC | |

### Targeting Moieties

In some embodiments, the device or kit comprises a targeting moiety that tethers the therapeutic agent to the structure. In some embodiments, the targeting moiety is connected to the therapeutic agent, and the moiety non-covalently binds to the structure. As a non-limiting example, the targeting moiety is covalently connected to the therapeutic agent via a peptide bond. For instance, targeting moiety comprises a targeting peptide, and the targeting peptide is linked to the therapeutic agent via a peptide bond.

In some embodiments, the targeting moiety has an affinity for the structure, or a component of the structure, e.g., to a ceramic material of the structure such as calcium phosphate. In some embodiments, the dissociation constant (KD) for binding between the targeting moiety and the structure or component thereof is: (i) at least about 1 fM, at least about 10 fM, at least about 100 fM, or at least about 1 pM; and (ii) less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 5 μM, less than about 1 μM, or less than about 100 μM. For example, the targeting moiety may bind to beta-tricalcium phosphate with an affinity of about 100 fM to about 100 μM, about 1 pM to about 100 μM, about 10 μM to about 100 μM, about 100 pM to about 100 μM, or about 1 μM to about 100 μM.

In some embodiments, the targeting moiety comprises one or more targeting peptides that each bind to the structure. In some embodiments, the targeting peptide binds to the ceramic material of the structure. For example, the targeting peptide binds to calcium phosphate (e.g., tricalcium phosphate, beta tricalcium phosphate, alpha tricalcium phosphate), hydroxyapatite, fluorapatite, bone (e.g., demineralized bone), glasses (bioglasses) such as silicates, vanadates, and related ceramic minerals, or chelated divalent metal ions, or a combination thereof. In some embodiments, the targeting peptide comprises two or more targeting peptides. In some embodiments, two or more targeting peptides is no more than about 50, 45, 40, 35, 30, 25, 20, 15, or 10 targeting peptides. In some embodiments, two or more targeting peptides is about 2, 3, 4, 5, 6, 7, 8, 9, 10, 11, 12, 13, 14, 15, 20, 25, or 30 targeting peptides. In some embodiments, two or more targeting peptides is about 2 to about 10 targeting peptides. In some embodiments, two or more targeting peptides is about 5 targeting peptides.

In some embodiments, the targeting peptide comprises a sequence at least about 70%, 75%, 80%, 85%, 90%, 95%, or 100% identical to SEQ ID NO: 1. In some embodiments, the targeting peptide comprises a sequence at least about 70%, 75%, 80%, 85%, 90%, 95%, or 100% identical to SEQ ID NO: 2. In some embodiments, the targeting peptide comprises a sequence at least about 70%, 75%, 80%, 85%, 90%, 95%, or 100% identical to SEQ ID NO: 3. In some embodiments, the targeting peptide comprises a sequence at least about 70%, 75%, 80%, 85%, 90%, 95%, or 100% identical to SEQ ID NO: 4. In some embodiments, the targeting peptide comprises a sequence at least about 70%, 75%, 80%, 85%, 90%, 95%, or 100% identical to SEQ ID NO: 5. In some embodiments, the targeting peptide comprises a sequence at least about 70%, 75%, 80%, 85%, 90%, 95%, or 100% identical to SEQ ID NO: 6. In some embodiments, the targeting peptide comprises a sequence at least about 70%, 75%, 80%, 85%, 90%, 95%, or 100% identical to SEQ ID NO: 7. In some embodiments, the targeting peptide comprises a sequence at least about 70%, 75%, 80%, 85%, 90%, 95%, or 100% identical to SEQ ID NO: 8. In some embodiments, the targeting peptide comprises a sequence at least about 70%, 75%, 80%, 85%, 90%, 95%, or 100% identical to SEQ ID NO: 9. In some embodiments, the targeting peptide comprises a sequence at least about 70%, 75%, 80%, 85%, 90%, 95%, or 100% identical to SEQ ID NO: 10. In some embodiments, the targeting peptide comprises a sequence at least about 70%, 75%, 80%, 85%, 90%, 95%, or 100% identical to SEQ ID NO: 11. In some embodiments, the targeting peptide comprises a sequence at least about 70%, 75%, 80%, 85%, 90%, 95%, or 100% identical to SEQ ID NO: 12. In some embodiments, the targeting peptide comprises a sequence at least about 70%, 75%, 80%, 85%, 90%, 95%, or 100% identical to SEQ ID NO: 13. In some embodiments, the targeting peptide comprises a sequence at least about 70%, 75%, 80%, 85%, 90%, 95%, or 100% identical to SEQ ID NO: 14. In some embodiments, the targeting peptide comprises a sequence at least about 70%, 75%, 80%, 85%, 90%, 95%, or 100% identical to SEQ ID NO: 15. In some embodiments, the targeting peptide comprises a sequence at least about 70%, 75%, 80%, 85%, 90%, 95%, or 100% identical to SEQ ID NO: 16. In some embodiments, the targeting peptide comprises a sequence at least about 70%, 75%, 80%, 85%, 90%, 95%, or 100% identical to SEQ ID NO: 17. In some embodiments, the targeting peptide comprises a sequence at least about 70%, 75%, 80%, 85%, 90%, 95%, or 100% identical to SEQ ID NO: 18. In some embodiments, the targeting peptide comprises a sequence at least about 70%, 75%, 80%, 85%, 90%, 95%, or 100% identical to SEQ ID NO: 19. In some embodiments, the targeting peptide comprises a sequence at least about 70%, 75%, 80%, 85%, 90%, 95%, or 100% identical to SEQ ID NO: 20. In some embodiments, the targeting peptide comprises a sequence at least about 70%, 75%, 80%, 85%, 90%, 95%, or 100% identical to SEQ ID NO: 21. In some embodiments, the targeting peptide comprises a sequence at least about 70%, 75%, 80%, 85%, 90%, 95%, or 100% identical to SEQ ID NO: 22. In some embodiments, the targeting peptide comprises a sequence at least about 70%, 75%, 80%, 85%, 90%, 95%, or 100% identical to SEQ ID NO: 23. In some embodiments, the targeting peptide comprises a sequence at least about 70%, 75%, 80%, 85%, 90%, 95%, or 100% identical to SEQ ID NO: 24. In some embodiments, the targeting peptide comprises a sequence at least about 70%, 75%, 80%, 85%, 90%, 95%, or 100% identical to SEQ ID NO: 25. In some embodiments, the targeting peptide comprises a sequence at least about 70%, 75%, 80%, 85%, 90%, 95%, or 100% identical to SEQ ID NO: 26. In some embodiments, the targeting peptide comprises a sequence at least about 70%, 75%, 80%, 85%, 90%, 95%, or 100% identical to SEQ ID NO: 27. In some embodiments, the targeting peptide comprises a sequence at least about 70%, 75%, 80%, 85%, 90%, 95%, or 100% identical to SEQ ID NO: 28. In some embodiments, the targeting peptide comprises a sequence at least about 70%, 75%, 80%, 85%, 90%, 95%, or 100% identical to SEQ ID NO: 29. In some embodiments, the targeting peptide comprises a sequence at least about 70%, 75%, 80%, 85%, 90%, 95%, or 100% identical to SEQ ID NO: 30. In some embodiments, the targeting peptide comprises a sequence at least about 70%, 75%, 80%, 85%, 90%, 95%, or 100% identical to SEQ ID NO: 31. In some embodiments, the targeting peptide comprises a sequence at least about 70%, 75%, 80%, 85%, 90%, 95%, or 100% identical to SEQ ID NO: 32. In some embodiments, the targeting peptide comprises a sequence at least about 70%, 75%, 80%, 85%, 90%, 95%, or 100% identical to SEQ ID NO: 33. In some embodiments, the targeting peptide comprises a sequence at least about 70%, 75%, 80%, 85%, 90%, 95%, or 100% identical to SEQ ID NO: 34. In some embodiments, the targeting peptide comprises a sequence at least about 70%, 75%, 80%, 85%, 90%, 95%, or 100% identical to SEQ ID NO: 35. In some embodiments, the targeting peptide comprises a sequence at least about 70%, 75%, 80%, 85%, 90%, 95%, or 100% identical to SEQ ID NO: 36. In some embodiments, the targeting peptide comprises a sequence at least about 70%, 75%, 80%, 85%, 90%, 95%, or 100% identical to SEQ ID NO: 37. In some embodiments, the targeting peptide comprises a sequence at least about 70%, 75%, 80%, 85%, 90%, 95%, or 100% identical to SEQ ID NO: 38. In some embodiments, the targeting peptide comprises a sequence at least about 70%, 75%, 80%, 85%, 90%, 95%, or 100% identical to SEQ ID NO: 39. In some embodiments, the targeting peptide comprises a sequence at least about 70%, 75%, 80%, 85%, 90%, 95%, or 100% identical to SEQ ID NO: 40. In some embodiments, the targeting peptide comprises a sequence at least about 70%, 75%, 80%, 85%, 90%, 95%, or 100% identical to SEQ ID NO: 41. In some embodiments, the targeting peptide comprises a sequence at least about 70%, 75%, 80%, 85%, 90%, 95%, or 100% identical to SEQ ID NO: 42. In some embodiments, the targeting peptide comprises a sequence at least about 70%, 75%, 80%, 85%, 90%, 95%, or 100% identical to SEQ ID NO: 43. In some embodiments, the targeting peptide comprises a sequence at least about 70%, 75%, 80%, 85%, 90%, 95%, or 100% identical to SEQ ID NO: 44. In some embodiments, the targeting peptide comprises a sequence at least about 70%, 75%, 80%, 85%, 90%, 95%, or 100% identical to SEQ ID NO: 45. In some embodiments, the targeting peptide comprises a sequence at least about 70%, 75%, 80%, 85%, 90%, 95%, or 100% identical to SEQ ID NO: 46. In some embodiments, the targeting peptide comprises a sequence at least about 70%, 75%, 80%, 85%, 90%, 95%, or 100% identical to SEQ ID NO: 47. In some embodiments, the targeting peptide comprises a sequence at least about 70%, 75%, 80%, 85%, 90%, 95%, or 100% identical to SEQ ID NO: 48. In some embodiments, the targeting peptide comprises a sequence at least about 70%, 75%, 80%, 85%, 90%, 95%, or 100% identical to SEQ ID NO: 49. In some embodiments, the targeting peptide comprises a sequence at least about 70%, 75%, 80%, 85%, 90%, 95%, or 100% identical to SEQ ID NO: 50. In some embodiments, the targeting peptide comprises a sequence at least about 70%, 75%, 80%, 85%, 90%, 95%, or 100% identical to SEQ ID NO: 51. In some embodiments, the targeting peptide comprises a sequence at least about 70%, 75%, 80%, 85%, 90%, 95%, or 100% identical to SEQ ID NO: 52. In some embodiments, the targeting peptide comprises a sequence at least about 70%, 75%, 80%, 85%, 90%, 95%, or 100% identical to SEQ ID NO: 53. In some embodiments, the targeting peptide comprises a sequence at least about 70%, 75%, 80%, 85%, 90%, 95%, or 100% identical to SEQ ID NO: 54. In some embodiments, the targeting peptide comprises a sequence at least about 70%, 75%, 80%, 85%, 90%, 95%, or 100% identical to SEQ ID NO: 55. In some embodiments, the targeting peptide comprises a sequence at least about 70%, 75%, 80%, 85%, 90%, 95%, or 100% identical to SEQ ID NO: 56. In some embodiments, the targeting peptide comprises a sequence at least about 70%, 75%, 80%, 85%, 90%, 95%, or 100% identical to SEQ ID NO: 57. In some embodiments, the targeting peptide comprises a sequence at least about 70%, 75%, 80%, 85%, 90%, 95%, or 100% identical to SEQ ID NO: 58. In some embodiments, the targeting peptide comprises a sequence at least about 70%, 75%, 80%, 85%, 90%, 95%, or 100% identical to SEQ ID NO: 59. In some embodiments, the targeting peptide comprises a sequence at least about 70%, 75%, 80%, 85%, 90%, 95%, or 100% identical to SEQ ID NO: 60. In some embodiments, the targeting peptide comprises a sequence at least about 70%, 75%, 80%, 85%, 90%, 95%, or 100% identical to SEQ ID NO: 61.

TABLE 2
Targeting Peptides

| | SEQ |
|---|---|
| Sequence | ID NO: |
| LLADTTHHRPWT | 1 |
| VIGESTHHRPWS | 2 |
| LIADSTHHSPWT | 3 |
| ILAESTHHKPWT | 4 |
| ILAETTHHRPWS | 5 |
| IIGESSHHKPFT | 6 |
| GLGDTTHHRPWG | 7 |
| VLGDTTHHKPWT | 8 |
| IVADSTHHRPWT | 9 |
| STADTSHHRPS | 10 |
| TSGGESTHHRPS | 11 |
| TSGGESSHHKPS | 12 |
| TGSGDSSHHRPS | 13 |
| GSSGESTHHKPST | 14 |
| VGADSTHHRPVT | 15 |
| GAADTTHHRPVT | 16 |
| AGADTTHHRPVT | 17 |
| GGADTTHHRPAT | 18 |
| GGADTTHHRPGT | 19 |
| LLADTTHHRPWTVIGESTHHRPWS | 20 |
| LLADTTHHRPWTVIGESTHHRPWSIIGESSHHKPFT | 21 |
| LLADTTHHRPWTVIGESTHHRPWSIIGESSHHKPFTGLGDTTHHRPWGILAESTHHKPWT | 22 |
| LLADTTHHRPWTILAESTHHKPWT | 23 |
| LLADTTHHRPWTILAESTHHKPWTLLADTTHHRPWTILAESTHHKPWTLLADTTHHRPWT | 24 |
| LLADTTHHRPWTGLGDTTHHRPWG | 25 |
| LLADTTHHRPWTGLGDTTHHRPWGLLADTTHHRPWT | 26 |
| LLADTTHHRPWTGLGDTTHHRPWGLLADTTHHRPWTGLGDTTHHRPWGLLADTTHHRPWT | 27 |
| LLADTTHHRPWTGLGDTTHHRPWGLLADTTHHRPWTGLGDTTHHRPWGLLADTTHHRPWTGLGDTTHHRPWGLLADTTHHRPWT | 28 |
| STADTSHHRPSTSGGESTHHRPSTSGGESSHHKPSTGSGDSSHHRPSGSSGESTHHKPST | 29 |
| VGADSTHHRPVTGAADTTHHRPVTAGADTTHHRPVTGGADTTHHRPATGGADTTHHRPGT | 30 |
| STADTSHHRPSLLADTTHHRPWTTSGGESTHHRPSVGADSTHHRPVTTSGGESSHHKPSG | 31 |
| AADTTHHRPVTTGSGDSSHHRPSGSSGESTHHKPSTGGADTTHHRPATAAADTTHHRPWT | 32 |
| AAADTTHHRPWTAAADTTHHRPWTAAADTTHHRPWTAAADTTHHRPWTAAADTTHHRPWT | 33 |
| LLADAAHHRPWTLLADAAHHRPWTLLADAAHHRPWTLLADAAHHRPWTLLADAAHHRPWT | 34 |
| LLADTTAARPWTLLADTTAARPWTLLADTTAARPWTLLADTTAARPWTLLADTTAARPWT | 35 |
| LLADTTHHRPWTLLADTTHHRPWT | 36 |
| LLADTTHHRPWTLLADTTHHRPWTLLADTTHHRPWT | 37 |
| LLADTTHHRPWTLLADTTHHRPWTLLADTTHHRPWTLLADTTHHRPWTLLADTTHHRPWT | 38 |
| STSGSTVIGESTHHRPWSLIADSTHHSPWTILAESTHHKPWTILAETTHHRPWSIIGESSHHKPFTGLGDTTHHRPWGVLGDTTHHKP | 39 |
| WTIVADSTHHRPWTGQVLPTTTPSSPSTTSGS | |
| LLADTTHHRPWTVIGESTHHRPWSIIGESSHHKPFTGLGDTTHHRPWG | 40 |
| VIGESTHHRPWSIIGESSHHKPFTGLGDTTHHRPWGILAESTHHKPWT | 41 |
| (X1)(X2), wherein X1 comprises SEQ ID NO: 1 and X2 comprises one or more of SEQ ID NOS: | 42 |
| 1-41. | |
| (X1)(X2), wherein X1 comprises SEQ ID NO: 2 and X2 comprises one or more of SEQ ID NOS: | 43 |
| 1-41. | |
| (X1)(X2), wherein X1 comprises SEQ ID NO: 4 and X2 comprises one or more of SEQ ID NOS: | 44 |
| 1-41. | |
| (X1)(X2), wherein X1 comprises SEQ ID NO: 6 and X2 comprises one or more of SEQ ID NOS: | 45 |
| 1-41. | |
| (X1)(X2), wherein X1 comprises SEQ ID NO: 7 and X2 comprises one or more of SEQ ID NOS: | 46 |
| 1-41. | |
| (X1)(X2), wherein X1 comprises SEQ ID NO: 1 and X2 comprises one or more of SEQ ID NOS: | 47 |
| 2, 4, 6, or 7. | |
| (X1)(X2), wherein X1 comprises SEQ ID NO: 2 and X2 comprises one or more of SEQ ID NOS: | 48 |
| 1, 4, 6, or 7. | |
| (X1)(X2), wherein X1 comprises SEQ ID NO: 4 and X2 comprises one or more of SEQ ID NOS: | 49 |
| 1, 2, 6, or 7. | |
| (X1)(X2), wherein X1 comprises SEQ ID NO: 6 and X2 comprises one or more of SEQ ID NOS: | 50 |
| 1, 4, 2, or 7. | |
| (X1)(X2), wherein X1 comprises SEQ ID NO: 7 and X2 comprises one or more of SEQ ID NOS: | 51 |
| 1, 4, 6, or 2. | |
| (X1)(X2), wherein X1 comprises SEQ ID NO: 1 and X2 comprises two or more of SEQ ID NOS: | 52 |
| 2, 4, 6, or 7. | |
| (X1)(X2), wherein X1 comprises SEQ ID NO: 2 and X2 comprises two or more of SEQ ID NOS: | 53 |
| 1, 4, 6, or 7. | |
| (X1)(X2), wherein X1 comprises SEQ ID NO: 4 and X2 comprises two or more of SEQ ID NOS: | 54 |
| 1, 2, 6, or 7. | |
| (X1)(X2), wherein X1 comprises SEQ ID NO: 6 and X2 comprises two or more of SEQ ID NOS: | 55 |
| 1, 4, 2, or 7. | |
| (X1)(X2), wherein X1 comprises SEQ ID NO: 7 and X2 comprises two or more of SEQ ID NOS: | 56 |
| 1, 4, 6, or 2. | |
| (X1)(X2), wherein X1 comprises SEQ ID NO: 1 and X2 comprises three or more of SEQ ID NOS: | 57 |
| 2, 4, 6, or 7. | |
| (X1)(X2), wherein X1 comprises SEQ ID NO: 2 and X2 comprises three or more of SEQ ID NOS: | 58 |
| 1, 4, 6, or 7. | |
| (X1)(X2), wherein X1 comprises SEQ ID NO: 4 and X2 comprises three or more of SEQ ID NOS: | 59 |
| 1, 2, 6, or 7. | |
| (X1)(X2), wherein X1 comprises SEQ ID NO: 6 and X2 comprises three or more of SEQ ID NOS: | 60 |
| 1, 4, 2, or 7. | |
| (X1)(X2), wherein X1 comprises SEQ ID NO: 7 and X2 comprises three or more of SEQ ID NOS: | 61 |
| 1, 4, 6, or 2. | |

In some embodiments, a targeting peptide comprises one or more sequences of Table 2. In some embodiments, the targeting peptide comprises a sequence at least about 70%, 75%, 80%, 85%, 90%, 95%, or 100% identical to a sequence of Table 2.

TABLE 3
Additional Targeting Peptides

| Sequence | SEQ ID NO |
|---|---|
| ACAPLMFSQC | 62 |
| ACHASLKHRC | 63 |
| ACLSTKTNIC | 64 |
| ACTTPSKHQC | 65 |
| AHFSPNLLLGG | 66 |
| AHSLKSITNHGL | 67 |
| AKQTVPV | 68 |
| AKTLMPSPFPRT | 69 |
| AMSQTMTAAIEK | 70 |
| ANPPLSL | 71 |
| ANPYHRH | 72 |
| APLSLSL | 73 |
| APYHPTIPASVHGGGK | 74 |
| ASAVGSLSIRWX | 75 |
| ASGPTNV | 76 |
| ASHNPKL | 77 |
| ASWVDSRQPSAA | 78 |
| ATFSPPL | 79 |
| ATWSHHLSSAGL | 80 |
| ATWSHHLSSAGLGGGS | 81 |
| CAHLSPHKC | 82 |
| CDIPWRNEC | 83 |
| CDPLRQHSC | 84 |
| CDSLGHWLC | 85 |
| CDYTTRHSC | 86 |
| CHGTLNPEC | 87 |
| CHHNLSWEC | 88 |
| CHIWTLASC | 89 |
| CHNTFSPRC | 90 |
| CIPLHASLC | 91 |
| CITTTSLSC | 92 |
| CKLTTCKDC | 93 |
| CKNHTTFWC | 94 |
| CLKLLSRSC | 95 |
| CLLKAHPSC | 96 |
| CLNQLKQAC | 97 |
| CLSTKTNIC | 98 |
| CMNFPSPHC | 99 |
| CNYPTLKSC | 100 |
| CPQLTVGQHRT | 101 |
| CPQSPTYTC | 102 |
| CPSSAIHTC | 103 |
| CPTSTARIC | 104 |
| CQASSFPSC | 105 |
| CQPYFWYRC | 106 |
| CQTLTPSIC | 107 |
| CSKLGHLWC | 108 |
| CSKTPERIX | 109 |
| CSNNNRMTC | 110 |
| CSPILSLSC | 111 |
| CSPTNFTRC | 112 |
| CSRPAMNVC | 113 |
| CSTKAYPNC | 114 |
| CSTSSCGSC | 115 |
| CSYWGHRDC | 116 |
| CTAHDANAC | 117 |
| CTANSEKTC | 118 |
| CTHPKASMC | 119 |
| CTKTINGKC | 120 |
| CTNMQSPLC | 121 |
| CTPFTKLPC | 122 |
| CTPTTDSIC | 123 |
| CTQQNGHPC | 124 |
| CTTPSKHQC | 125 |
| CTYNVAKPC | 126 |
| DKLHRLA | 127 |
| DLNYFTLSSKRE | 128 |
| DLPPTLHTTGSP | 129 |
| DMRQQRS | 130 |
| DQYWGLR | 131 |
| DSSNPIFWRPSS | 132 |
| DSSNPIFWRPSSGGGS | 133 |
| EFLGVPASLVNP | 134 |
| EPNHTRF | 135 |
| EPRRAVAAL | 136 |
| EPRRAVAEL | 137 |
| EPRREVAEL | 138 |
| EPRREVCEL | 139 |
| ESDLTHALHWLG | 140 |
| ESLKSIS | 141 |
| ETRTQLL | 142 |
| ETVCASS | 143 |
| ETYARPL | 144 |
| ETYQQPL | 145 |
| EVHSTDRYRSIP | 146 |
| FGLQPTGDIARR | 147 |
| FSMDDPERVRSP | 148 |
| FSPLHTSTYRPS | 149 |
| FTLPTIR | 150 |
| FVNLLGQ | 151 |
| GDFNSGHHTTTR | 152 |
| GGGAAAA | 153 |
| GIHVPWMPPVAF | 154 |
| GIHVPWMPPVAFGGGS | 155 |
| GPSNNLPWSNTP | 156 |
| GSAGLKYPLYKS | 157 |
| GSCPPKK | 158 |
| GSLFKAL | 159 |
| GTQTPQP | 160 |
| GTSRLFS | 161 |
| GVHKHFYSRWLG | 162 |
| HAPLTRSPAPNL | 163 |
| HAPVQPN | 164 |
| HGSLTTLXRYEP | 165 |
| HHFHLPKLRPPV | 166 |
| HHQRSPA | 167 |
| HHTWDTRIWQAF | 168 |
| HMLAQTF | 169 |
| HNVTTRTQRLMP | 170 |
| HPTTPIHMPNF | 171 |
| HQFISPEPFLIS | 172 |
| HQFPXSNLVWKP | 173 |
| HQWDHKY | 174 |
| HRDPXSXPSAXRP | 175 |
| HRLGHMS | 176 |
| HSACHASLKHRC | 177 |
| HSACKLTTCKDG | 178 |
| HSACLSTKTNIC | 179 |
| HSMPHMGTYLLT | 180 |
| HSTGPTR | 181 |
| HTLLSTT | 182 |
| HYPTVNF | 183 |
| IAHVPETRLAQM | 184 |
| IFSMGTALARPL | 185 |
| IGYPVLP | 186 |
| INFQFLKPSTTR | 187 |
| INKHPQQVSTLL | 188 |
| IQHQAKT | 189 |
| IRXLXIS | 190 |
| ISPSHSQAQADL | 191 |
| KAFDKHG | 192 |
| KATITGM | 193 |
| KEIPPIPLLAPS | 194 |
| KEIPPIPLLAPSGGGS | 195 |
| KIPKACCVPTELSAISMLYL | 196 |
| KIPKASSVPTELSAIATLYLAAAAEPRRAVAAL | 197 |
| KIPKASSVPTELSAISTLYL | 198 |
| KIPKASSVPTELSAISTLYLAAAAEPRRAVAAL | 199 |
| KIPKASSVPTELSAISTLYLAAAAEPRRAVAEL | 200 |
| KIPKASSVPTELSAISTLYLAAAAEPRREVAEL | 201 |
| KIPKASSVPTELSAISTLYLAAAAXPRRXVAXL | 202 |
| KIPKASSVPTELSAISTLYLXPRRXVAXL | 203 |
| KLHASLA | 204 |
| KLSAWSF | 205 |
| KLTWQELYQLKYKGI | 206 |
| KLTWQELYQLKYKGIGGGAAAAEPRREVAEL | 207 |
| KMNHMPN | 208 |
| KPMQFVH | 209 |
| KTSSWAN | 210 |
| LASTTHV | 211 |
| LDYPIPQTVLHH | 212 |
| LFAAVPSTQFFR | 213 |
| LGFDPTSTRFYT | 214 |
| LGPGKAF | 215 |
| LKPFSGA | 216 |
| LLADTTHHRPWP | 217 |
| LLADTTHHRPWT | 218 |
| LLADTTHHRPWTGGGS | 219 |
| LLPLKFK | 220 |
| LPFQPPI | 221 |
| LPLTPLP | 222 |
| LPRDLHATPQQI | 223 |
| LPSIHNL | 224 |
| LPWAPNLPDSTA | 225 |
| LPWTEPSFWRTP | 226 |
| LPWTEPSFWRTPGGGS | 227 |
| LQKSPSL | 228 |
| LQPSQPQRFAPT | 229 |
| LRAFPSLPHTVT | 230 |
| LSAPMEY | 231 |
| LSKNPLL | 232 |
| LSLRASAATDFQ | 233 |
| LSPPMQLQPTYS | 234 |
| LTPTMFNMHGVL | 235 |
| LTQTLQY | 236 |
| MHNVSDSNDSAI | 237 |
| MKVHERS | 238 |
| MPQTLVLPRSLL | 239 |
| MQFTPAPSPSDH | 240 |
| MTSQTLR | 241 |
| MYPLPAP | 242 |
| NERQMEL | 243 |
| NFAMNLR | 244 |
| NITQLGS | 245 |
| NKPLSTL | 246 |
| NNVSQKWQQRLI | 247 |
| NNVSQKWQQRLIGGGS | 248 |
| NPDHPDIPQDVHGGGK | 249 |
| NPMIMNQ | 250 |
| NPQMQRS | 251 |
| NPRSQAT | 252 |
| NPYAPTIPQSVAGGGK | 253 |
| NPYHPTIPQSVH | 254 |
| NPYHPTIPQSVHGGGK | 255 |
| NSMIAHNKTRMH | 256 |
| NSMIAHNKTRMHGGGS | 257 |
| NSSMLGMLPSSF | 258 |
| NTSSSQGTQRLG | 259 |
| NTTTDIPSPSQF | 260 |
| NYPTLKS | 261 |
| NYSHLRVKLPTP | 262 |
| NYSHLRVKLPTPGGGS | 263 |
| PAKQKAH | 264 |
| PDIPLSR | 265 |
| PGQWPSSLTLYK | 266 |
| PHNPGKL | 267 |
| PIDAFFD | 268 |
| PLTQPSH | 269 |
| PPKDSRG | 270 |
| PPNMARA | 271 |
| PSMKHWR | 272 |
| PTNKPHT | 273 |
| PTTMTRW | 274 |
| PTTWGHL | 275 |
| PXGPXGPXGPXGPXGPXAPXGPXGPXGPXGPXGPXG | 276 |
| QHNFRGASSSAP | 277 |
| QIPQMRILHPYG | 278 |
| QIQKPPRTPPSL | 279 |
| QLTQTMWKDTTL | 280 |
| QNLPPERYSEAT | 281 |
| QNPRQIY | 282 |
| QNYLLPK | 283 |
| QPGLWPS | 284 |
| QRSWTLDSALSM | 285 |
| QRSWTLDSALSMGGGS | 286 |
| QSLSFAGPPAWQ | 287 |
| QSSYNPI | 288 |
| QTHARHQ | 289 |
| QTHSSLW | 290 |
| QTTMTPLWPSFS | 291 |
| RCMSEVISFNCP | 292 |
| RHTLPLH | 293 |
| RPHTITN | 294 |
| RSPYYNKWSSKF | 295 |
| RTPLQPLEDFRP | 296 |
| SAGHIHEAHRPL | 297 |
| SAISDHRAHRSH | 298 |
| SAKGRAD | 299 |
| SAKKVFS | 300 |
| SASGTPS | 301 |
| SEPTYWRPNMSG | 302 |
| SFAPDIKYPVPS | 303 |
| SFQSMSLMTLVV | 304 |
| SFWHHHSPRSPL | 305 |
| SGHQLLLNKMPN | 306 |
| SGHQLLLNKMPNGGGS | 307 |
| SIFAHQTPTHKN | 308 |
| SIPKMIPTESLL | 309 |
| SIPSHSIHSAKA | 310 |
| SIRTSMNPPNLL | 311 |
| SKTSSTS | 312 |
| SLLTPWL | 313 |
| SLPHYIDNPFRQ | 314 |
| SLSKANILHLYG | 315 |
| SLVTADASFTPS | 316 |
| SMAAKSS | 317 |
| SMVYGNRLPSAL | 318 |
| SMYDTHS | 319 |
| SPEMKPR | 320 |
| SPNFSWLPLGTT | 321 |
| SPNLPWSKLSAY | 322 |
| SPNNPRE | 323 |
| SPNNTRE | 324 |
| SPSLMARSSPYW | 325 |
| SQHSTQD | 326 |
| SQTLPYSNAPSP | 327 |
| SRTGAHH | 328 |
| SSHHHRH | 329 |
| SSPPRVY | 330 |
| SSSMAKM | 331 |
| SSTLKTFFGFPD | 332 |
| SSTLKTFFGFPDGGGS | 333 |
| SSTQAHPFAPQL | 334 |
| SSTQVQHTLLQT | 335 |
| SSVPGRP | 336 |
| SSYEYHA | 337 |
| STLASMR | 338 |
| STPNSYSLPQAR | 339 |
| STQAHPW | 340 |
| STSAKHW | 341 |
| STVVMQPPPRPA | 342 |
| SVFLPTRHSPDL | 343 |
| SVQTRPLFHSHF | 344 |
| SVSVGMKPSPRP | 345 |
| SVSVGMNAESXA | 346 |
| SVSVGMNAESYG | 347 |
| SVSVGTEAESXA | 348 |
| SWPLYSRDSGLG | 349 |
| SYIDSMVPSTQT | 350 |
| SYKTTDSDTSPL | 351 |
| SYSQMDPPRSLPGGGS | 352 |
| TAAASNLRAVPP | 353 |
| TAPLSHPPRPGA | 354 |
| TDHPPKA | 355 |
| TGLAKTA | 356 |
| TGLLPNSSGAGI | 357 |
| TGPPSRQPAPLH | 358 |
| TGPTSLS | 359 |
| THPVVFEDERLF | 360 |
| TIHSKPA | 361 |
| TKDWLPS | 362 |
| TLAFQTA | 363 |
| TLAPTFR | 364 |
| TLDKYTRLLSRY | 365 |
| TLGLPML | 366 |
| TLLRTQV | 367 |
| TLMTTPP | 368 |
| TLPSPLALLTVH | 369 |
| TLQRMGQ | 370 |
| TLSNGHRYLELL | 371 |
| TMGFTAPRFPHY | 372 |
| TMRNPITSLISV | 373 |
| TMRNPITSLISVGGGS | 374 |
| TMTNMAK | 375 |
| TPLSYLKGLVTV | 376 |
| TPLTSPSLVRPQ | 377 |
| TPSPKLLQVFQA | 378 |
| TPSTGLGMSPAV | 379 |
| TPVYSLKLGPWP | 380 |
| TQTWPQSSSHGL | 381 |
| TRFYDSL | 382 |
| TRLVPSRYYHHP | 383 |
| TSPIPQMRTVPP | 384 |
| TTKNFNK | 385 |
| TTLSPRT | 386 |
| TTNSSMTMQLQR | 387 |
| TTTLPVQPTLRN | 388 |
| TTTWTTTARWPL | 389 |
| TTYNSPP | 390 |
| TVAQMPPHWQLT | 391 |
| TVLGTFP | 392 |
| TWNSNSTQYGNR | 393 |
| TWTLPAMHPRPA | 394 |
| VHLTHGQ | 395 |
| VHPRPSL | 396 |
| VHTSLLQKHPLP | 397 |
| VLPNIYMTLSA | 398 |
| VMDFASPAHVLP | 399 |
| VNQEYWFFPRRP | 400 |
| VPPISXTFLFXSTXS | 401 |
| VPPLHPALSRXN | 402 |
| VSPFLSPTPLLF | 403 |
| VSRLGTPSMHPS | 404 |
| VVKSNGE | 405 |
| VYSSPLSQLPR | 406 |
| WLPPRTQ | 407 |
| WPANKLSTKSMY | 408 |
| WPFNHFPWWNVP | 409 |
| WPTYLNPSSLKA | 410 |
| WSAHIVPYSHKP | 411 |
| WWPNSLNWVPRP | 412 |
| WYPNHLA | 413 |
| XITXGAY | 414 |
| XPRRAVAAL | 415 |
| XPRRAVAXL | 416 |
| XPRRXVAXL | 417 |
| XXFPLXG | 418 |
| YATQHNWRLKHE | 419 |
| YCPMRLCTDC | 420 |
| YELQMPLTLPLN | 421 |
| YEPAAAE | 422 |
| YGKGFSPYFHVT | 423 |
| YPHYSLPGSSTL | 424 |
| YPIMSHTCCHGV | 425 |
| YPKALRN | 426 |
| YPSLLKMQPQFS | 427 |
| YQPRPFVTTSPM | 428 |
| YSAPLARSNVVM | 429 |
| YTRLSHNPYTLS | 430 |
| YTTHVLPFAPSS | 431 |
| YTWQTIREQYEM | 432 |

In some embodiments, a targeting peptide comprises one or more sequences of Table 3. In some embodiments, the targeting peptide comprises a sequence at least about 70%, 75%, 80%, 85%, 90%, 95%, or 100% identical to a sequence of Table 3.

Additional targeting peptides useful in the present disclosure include any one of SEQ ID NO: 1 to SEQ ID NO: 558 of U.S. Pat. No. 7,572,766. In some embodiments, the targeting peptide comprises a sequence at least about 70%, 75%, 80%, 85%, 90%, 95%, or 100% identical to any one of SEQ ID NO: 1 to SEQ ID NO: 558 of U.S. Pat. No. 7,572,766.

In some embodiments, the device or kit comprises a chimeric polypeptide comprising the targeting peptide and a targeting moiety. In some cases, the chimeric polypeptide comprises a sequence at least about 70%, 75%, 80%, 85%, 90%, 95%, or 100% identical to SEQ ID NO: 433 (ASGAGGSEGGGSEGGTSGATGAGTSTSGGGASTGGGTGQAKHKQRKRLKSSCKRHPL YVDFSDVGWNDWIV APPGYHAFYCHGECPFPLADHLNSTNHAIVQTLVNSVNSKIPKA CCVPTELSAISMLYLDENEKVVLKNYQDMVVEGCGCR). In some cases, the chimeric polypeptide comprises a sequence at least about 70%, 75%, 80%, 85%, 90%, 95%, or 100% identical to SEQ ID NO: 434 (MPIGSLLADTTHHRPWTVIGESTHHRPWSIIGESSHHKPFTGLGDTTHHRPWGILAESTH HKPWTASGAGGSEGGGSEGGTSGATGAGTSTSGGGASTGGGTGQAKHKQRKRLKSSC KRHPLYVDFSDVGWNDWIVAPPGYHAFYCHGECPFPLADHLNSTNHAIVQTLVNSVNS KIPKACCVPTELSAISMLYLDENEKVVLKNYQDMVVEGCGCR). In some cases, the chimeric polypeptide comprises a sequence at least about 70%, 75%, 80%, 85%, 90%, 95%, or 100% identical to SEQ ID NO: 435 (LLADTTHHRPWTVIGESTHHRPWSIIGESSHHKPFTGLGDTTHHRPWGILAESTHHKPW TASGAGGSEGGGSEGGTSGATGAGTSTSGGGASTGGGTGQAKHKQRKRLKSSCKRHPL YVDFSDVGWNDWIV APPGYHAFYCHGECPFPLADHLNSTNHAIVQTLVNSVNSKIPKA CCVPTELSAISMLYLDENEKVVLKNYQDMVVEGCGCR). In some cases, the chimeric polypeptide comprises a sequence at least about 70%, 75%, 80%, 85%, 90%, 95%, or 100% identical to SEQ ID NO: 436 (VIGESTHHRPWSIIGESSHHKPFTGLGDTTHHRPWGILAESTHHKPWTASGAGGSEGGG SEGGTSGATGAGTSTSGGGASTGGGTGQAKHKQRKRLKSSCKRHPLYVDFSDVGWND WIV APPGYHAFYCHGECPFPLADHLNSTNHAIVQTLVNSVNSKIPKACCVPTELSAISML YLDENEKVVLKNYQDMVVEGCGCR). In some cases, the chimeric polypeptide comprises a sequence at least about 70%, 75%, 80%, 85%, 90%, 95%, or 100% identical to SEQ ID NO: 437 (IIGESSHHKPFTGLGDTTHHRPWGILAESTHHKPWTASGAGGSEGGGSEGGTSGATGA GTSTSGGGASTGGGTGQAKHKQRKRLKSSCKRHPLYVDFSDVGWNDWIVAPPGYHAF YCHGECPFPLADHLNSTNHAIVQTLVNSVNSKIPKACCVPTELSAISMLYLDENEKVVL KNYQDMVVEGCGCR). In some cases, the chimeric polypeptide comprises a sequence at least about 70%, 75%, 80%, 85%, 90%, 95%, or 100% identical to SEQ ID NO: 438 (GLGDTTHHRPWGILAESTHHKPWTASGAGGSEGGGSEGGTSGATGAGTSTSGGGAST GGGTGQAKHKQRKRLKSSCKRHPLYVDFSDVGWNDWIVAPPGYHAFYCHGECPFPLA DHLNSTNHAIVQTLVNSVNSKIPKACCVPTELSAISMLYLDENEKVVLKNYQDMVVEG CGCR). In some cases, the chimeric polypeptide comprises a sequence at least about 70%, 75%, 80%, 85%, 90%, 95%, or 100% identical to SEQ ID NO: 439 (ILAESTHHKPWTASGAGGSEGGGSEGGTSGATGAGTSTSGGGASTGGGTGQAKHKQR KRLKSSCKRHPLYVDFSDVGWNDWIVAPPGYHAFYCHGECPFPLADHLNSTNHAIVQT LVNSVNSKIPKACCVPTELSAISMLYLDENEKVVLKNYQDMVVEGCGCR). In some cases, the chimeric polypeptide comprises a sequence at least about 70%, 75%, 80%, 85%, 90%, 95%, or 100% identical to SEQ ID NO: 440 ((X) QAKHKQRKRLKSSCKRHPLYVDFSDVGWNDWIVAPPGYHAFYCHGECPFPLADHL NSTNHAIVQTLVNSVNSKIPKACCVPTELSAISMLYLDENEKVVLKNYQDMVVEGCGC R), wherein X comprises a targeting peptide and optionally a linker. For example, the targeting peptide comprises one or more of SEQ ID NOS: 1-41. In some cases, the chimeric polypeptide comprises a sequence at least about 70%, 75%, 80%, 85%, 90%, 95%, or 100% identical to SEQ ID NO: 441 ((X) ASGAGGSEGGGSEGGTSGATGAGTSTSGGGASTGGGTGQAKHKQRKRLKSSCKRH PLYVDFSDVGWNDWIVAPPGYHAFYCHGECPFPLADHLNSTNHAIVQTLVNSVNSKIP KACCVPTELSAISMLYLDENEKVVLKNYQDMVVEGCGCR), wherein X comprises a targeting peptide and optionally a linker. For example, the targeting peptide comprises one or more of SEQ ID NOS: 1-41.

In some embodiments, a therapeutic agent is not connected to a structure using a targeting moiety. For example, the therapeutic agent may interact with the structure via non-covalent bonds. The therapeutic agent may be connected to a structure by hydrogen bonding, ionic bonding, hydrophobic interactions, or van der Waals forces. The therapeutic agent may also be connected to a structure using covalent bonds. Examples of methods for connecting using covalent bonds includes chemical linkers and spacers that are used for modifying active groups within proteins such as amines, thiols and carbohydrates.

In some embodiments, provided is a device comprising a structure that is seeded with cells. Non-limiting examples of cells include osteocytes and other bone cells, chondrocytes, and meniscal cells. In some instances, the cells can be added to the completed implantable structures.

### Device Manufacture

Further provided herein are methods of manufacturing a device comprising a structure (e.g., scaffold) and a therapeutic agent. Some methods comprise: (a) providing a first solution of a therapeutic agent (e.g., a chimeric polypeptide comprising the therapeutic agent and a targeting moiety), (b) providing a 3D structure, and (c) combining (a) and (b). In some embodiments, the method further comprises (d) washing the 3D structure of step (c) with a second solution, such as phosphate buffered saline (PBS). In some embodiments, the method further comprises drying the 3D structure of step (c) or step (d).

In some embodiments, the mass of the therapeutic agent (e.g., a therapeutic agent alone or a therapeutic agent connected to a targeting moiety) per cubic centimeter of the structure in a device is between about 0.05 and 50 (mg/cc), e.g., about 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, or 50 mg/cc or any number therebetween. For example, the therapeutic agent is about 1, 1.1, 1.2, 1.3, 1.4, 1.5, 1.6, 1.7, 1.8, 1.9, or 2 mg per cubic centimeter device. One method of measuring the amount of therapeutic peptide bound to the structure includes: (1) measuring the mass of therapeutic peptide input in the first solution, (2) measuring the mass of the therapeutic agent remaining in the first solution after combination with and removal from the structure, (3) measuring the mass of the therapeutic agent in the second solution if a wash step is included, (4) summing (2) and (3); and subtracting the sum of (4) from (1).

### Methods of Treatment

In another aspect, provided are methods of treating a subject with a structure herein. In some methods, the subject is treated with a device comprising a therapeutic peptide and the structure. In some instances, the subject has a bone fracture or a bone defect. In some instances, the subject requires a vertebral fusion of the spine. In some instances, the subject has a cartilage tear or cartilage defect. In some instances, the subject has cartilage loss.

In some embodiments, the subject is suffering from a defect in bone, cartilage, soft tissue, tendon, fascia, ligament, organ, osteotendinous tissue, dermal, or osteochondral, or a combination of one or more of the aforementioned defects. In some embodiments, a defect is a lack of bone, cartilage, soft tissue, tendon, fascia, ligament, organ, osteotendinous tissue, dermal, or osteochondral, or a combination of one or more of the aforementioned defects. In some embodiments, a defect in the subject arises from trauma. In some embodiments, a defect in the subject arises due to a congenital condition. In some embodiments, a defect in the subject arises due to an acquired condition. In some embodiments, a defect refers to the absence, loss, and/or break in a tissue and/or organ of the body. In some embodiments, a “bone defect” refers to the absence or loss (e.g., partial loss) of bone at an anatomical location in a subject where it would otherwise be present in a control healthy subject. A bone defect may be the result of an infection (e.g., osteomyelitis), a tumor, a trauma, or an adverse event of a treatment. A bone defect may also affect the muscles, soft tissue, tendons, or joints surrounding the bone defect and cause injury. In some embodiments, a bone defect includes damage to a soft tissue. In some embodiments, a “cartilage defect” refers to the absence or loss (e.g., partial loss) of cartilage at an anatomical location in a subject where it would otherwise be present in a control healthy subject. A cartilage defect may be the result of disease, osteochondritis, osteonecrosis, or trauma. For example, a cartilage defect may affect the knee joint.

Non-limiting examples of conditions suitable for treatment with a structure or device described herein include osteoarthritis, disc degeneration, congenital defect, spinal stenosis, spondylolisthesis, spondylosis, bone fracture, scoliosis, kyphosis, spinal fusion (PLF, and interbody fusions), trauma repair of bone, dental repair, craniomaxillofacial repair, ankle fusion, kyphoplasty, balloon osteoplasty, scaphoid facture repair, tendon-osseous repair, osteoporosis, avascular necrosis, congenital skeletal malformations, costal reconstruction, subchondral bone repair, cartilage repair (e.g., at low doses), or trauma, or a combination thereof. BMP2 is also involved in hair follicle development, therefore the methods may comprise treatment to hair follicles. The trauma may be to the bone, cartilage, soft tissue, tendon, fascia, ligament, organ, osteotendinous tissue, or dermal tissue, or osteochondral tissue. In some embodiments, the method is to treat an osteochondral injury.

The methods of treatment may comprise spinal fusion. In some embodiments, spinal fusion is a surgical technique to join two or more vertebrae. In some embodiments, the spinal fusion comprises PLF. In some embodiments, the spinal fusion comprises interbody fusions.

Provided herein are methods of promoting bone or cartilage formation in a subject in need thereof that include administering to the subject a therapeutically effective amount of any of the structures or devices described herein. Some embodiments of these methods can further include first selecting a subject in need of bone or cartilage formation. In some embodiments, the structure or device is administered to the subject proximal to the desired site of bone or cartilage formation in the subject.

Also provided herein are methods of replacing and/or repairing bone or cartilage in a subject in need thereof that include administering to the subject a therapeutically effective amount of any of the structure or devices described herein. Some embodiments of these methods can further include first selecting a subject in need of bone replacement, bone repair, cartilage replacement, or cartilage repair. In some embodiments, the structure or device is administered to the subject proximal to the desired site of bone or cartilage replacement or repair in the subject.

Also provided herein are methods of treating a bone fracture or bone loss in a subject in need thereof, the method comprising administering to the subject a therapeutically effective amount of any of the structure or devices described herein. Some embodiments of these methods can further include first selecting a subject having a bone fracture or bone loss. In some embodiments, the structure or device is administered to the subject proximal to the bone fracture or the site of bone loss in the subject.

Also provided herein are methods of repairing soft tissue in a subject in need thereof that include administering to the subject a therapeutically effective amount of any of the structure or devices described herein. Some embodiments of these methods can further include first selecting a subject having a bone fracture or bone loss. In some embodiments, the composition is administered to the subject proximal to the bone fracture or the site of bone loss in the subject.

Also provided herein are methods of localized delivery of a therapeutic to a subject in need thereof that include administering to the subject a therapeutically effective amount of any of the structure or devices described herein. Some embodiments of these methods can further include first selecting a subject having a bone fracture or bone loss. In some embodiments, the structure or device is administered to the subject proximal to the bone fracture or the site of bone loss in the subject.

Methods of determining the efficacy of treatment of a bone fracture or bone loss in a subject are known in the art and include, e.g., imaging techniques (e.g., magnetic resonance imaging, X-ray, or computed tomography).

Methods of detecting bone or cartilage formation, or replacement or repair of bone or cartilage in a subject are also known in the art and include, e.g., imaging techniques (e.g., magnetic resonance imaging, X-ray, or computed tomography).

Suitable animal models for treatment of a bone fraction or bone loss, bone or cartilage formation, or bone or cartilage replacement or repair are known in the art. Non-limiting examples of such animal models are described in the Examples and in, e.g., Drosse et al., Tissue Engineering Part C 14 (1): 79-88, 2008; Histing et al., Bone 49:591-599, 2011; and Poser et al., Hindawi Publishing Corporation, BioMed Research International; Article ID 348635, 2014.

As used herein, a method of treatment comprises administering to the subject a structure or device herein. In some embodiments, administration comprises implanting a polypeptide or composition herein.

In some embodiments, a polypeptide and/or composition herein comprising BMP-2 is administered to the subject. In some embodiments, the BMP2 comprises a sequence at least about 70%, 75%, 80%, 85%, 90%, 95%, or 100% identical to SEQ ID NO: 454. In some embodiments, the BMP-2 is administered to induce formation of bone in the subject. In some embodiments, the BMP-2 is administered to induce formation of cartilage. In some embodiments, the BMP-2 is administered in a spinal fusion.

The terminology used herein is for the purpose of describing particular cases only and is not intended to be limiting. The singular forms “a”, “an” and “the” are intended to include the plural forms as well, unless the context clearly indicates otherwise. To the extent that the terms “including”, “includes”, “having”, “has”, “with”, or variants thereof are used in either the detailed description and/or the claims, such terms are intended to be inclusive in a manner similar to the term “comprising.”

In some embodiments, the term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value.

The term “subject” as used herein refers to any mammal. A subject therefore refers to, for example, mice, rats, dogs, cats, horses, cows, pigs, guinea pigs, rats, humans, monkeys, and the like. When the subject is a human, the subject may be referred to herein as a patient. In some embodiments, the subject or “subject in need of treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon), a human, or a rodent (e.g., a mouse, a guinea pig, a hamster, or a rat). In some embodiments, the subject or “subject in need of treatment” may be a non-human mammal, especially mammals that are conventionally used as models for demonstrating therapeutic efficacy in humans (e.g., murine, lapine, porcine, canine, or primate animals) may be employed.

In some embodiments, the term “therapeutically effective amount” refers to an amount of a polypeptide or composition effective to “treat” a disease, condition or disorder in a subject. In some cases, therapeutically effective amount of the polypeptide or composition reduces the severity of symptoms of the disease, condition or disorder. In some instances, the disease, condition or disorder comprises a defect in an organ or tissue.

In some embodiments, “affinity” refers to the strength of the sum total of non-covalent interactions between a β-TCP binding sequence (or a chimeric polypeptide or polypeptide comprising a β-TCP binding sequence) and its binding partner (e.g., β-TCP). Affinity can be measured by common methods known in the art, including those described herein. Affinity can be determined, for example, using surface plasmon resonance (SPR) technology (e.g., BIACORE®) or biolayer interferometry (e.g., FORTEBIOR).

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Further Embodiments

1. A three-dimensional structure comprising about 70% to about 80% by weight βTCP and about 20% to about 30% by weight polycaprolactone (PCL).
2. The three-dimensional structure of embodiment 1, comprising about 75% by weight βTCP and about 25% by weight PCL.
3. A three-dimensional structure comprising about 70% to about 80% by weight βTCP and about 20% to about 30% by weight caprolactone/glycolide copolymer.
4. The three-dimensional structure of embodiment 3, comprising about 75% by weight βTCP and about 25% by weight caprolactone/glycolide copolymer.
5. The three-dimensional structure of embodiment 3 or embodiment 4, wherein the caprolactone/glycolide copolymer is caprolactone/glycolide copolymer (95:5).
6. The three-dimensional structure of embodiment 3 or embodiment 4, wherein the caprolactone/glycolide copolymer is caprolactone/glycolide copolymer (90:10).
7. A three-dimensional structure comprising about 70% to about 80% by weight βTCP and about 20% to about 30% by weight poly(D,L-lactide-co-glycolide) copolymer.
8. The three-dimensional structure of embodiment 7, comprising about 75% by weight βTCP and about 25% by weight poly(D,L-lactide-co-glycolide) copolymer.
9. The three-dimensional structure of embodiment 7 or embodiment 8, wherein the caprolactone/glycolide copolymer is poly(D,L-lactide-co-glycolide) copolymer (50:50).
10. A three-dimensional structure comprising about 70% to about 80% by weight βTCP and about 20% to about 30% by weight polydioxanone (PDS).
11. The three-dimensional structure of embodiment 10, comprising about 75% by weight βTCP and about 25% by weight PDS.
12. A three-dimensional structure comprising about 70% to about 80% by weight βTCP and about 20% to about 30% by weight dioxanone/L-lactide copolymer.
13. The three-dimensional structure of embodiment 12, comprising about 75% by weight βTCP and about 25% by weight dioxanone/L-lactide copolymer.
14. The three-dimensional structure of embodiment 12 or embodiment 13, wherein the caprolactone/glycolide copolymer is dioxanone/L-lactide copolymer (90:10).
15. A three-dimensional structure comprising about 70% to about 80% by weight βTCP and about 20% to about 30% by weight glycolide/L-lactide copolymer.
16. The three-dimensional structure of embodiment 15, comprising about 75% by weight βTCP and about 25% by weight glycolide/L-lactide copolymer.
17. The three-dimensional structure of embodiment 15 or embodiment 16, wherein the caprolactone/glycolide copolymer is glycolide/L-lactide copolymer (95:5).
18. A three-dimensional structure comprising about 70% to about 80% by weight βTCP and about 20% to about 30% by weight poly-1-lactide.
19. The three-dimensional structure of embodiment 18, comprising about 75% by weight βTCP and about 25% by weight poly-1-lactide.
20. The three-dimensional structure of any one of embodiments 1-19, wherein the density is about 1 to about 1.5 g/cm³.
21. The three-dimensional structure of any one of embodiments 1-20, wherein the open porosity is about 25% to about 40%.
22. The three-dimensional structure of embodiment any one of embodiments 1-21, wherein the strut diameter is about 300 μm to 800 μm, or about 300 μm, 400 μm, 500 μm, 600 μm, 700 μm, or 800 μm.
23. The three-dimensional structure of any one of embodiments 1-20, comprising a plurality of micropores, wherein the micropores have an average pore size of about 1 micron to about 500 microns or about 1 to about 50 microns.
24. A method of preparing the three-dimensional structure of any one of embodiments 1-22, wherein the method comprises additive manufacturing.
25. The method of embodiment 24, wherein the additive manufacturing comprises fused granular fabrication (FGF) or fused filament fabrication (FFF).
26. An ink formulation comprising about 55% to about 65% by weight βTCP, about 15% to about 25% by weight PCL, about 5% to about 15% PEG having a molecular weight from about 500 g/mol to about 15,000 g/mol, and about 5% to about 15% PEG having a molecular weight from about 25,000 g/mol to about 50,000 g/mol.
27. The ink formulation of embodiment 26, comprising about 60% by weight βTCP, about 20% by weight PCL, about 10% by weight PEG having a molecular weight from about 500 g/mol to about 15,000 g/mol, and about 10% PEG having a molecular weight from about 25,000 g/mol to about 50,000 g/mol.
28. The ink formulation of embodiment 27, comprising about 60% by weight βTCP, about 20% by weight PCL, about 10% by weight PEG having a molecular weight of about 8,000 g/mol, and about 10% PEG having a molecular weight of about 35,000 g/mol.
29. The ink formulation of any one of embodiments 26-28, comprising about 1% to about 10% of a sacrificial pore former.
30. The ink formulation of embodiment 29, wherein the sacrificial pore former comprises sucrose.
31. An ink formulation comprising about 55% to about 65% by weight βTCP, about 15% to about 25% by weight caprolactone/glycolide copolymer, about 5% to about 15% PEG having a molecular weight from about 500 g/mol to about 15,000 g/mol, and about 5% to about 15% PEG having a molecular weight from about 25,000 g/mol to about 50,000 g/mol.
32. The ink formulation of embodiment 31, comprising about 60% by weight βTCP, about 20% by weight caprolactone/glycolide copolymer, about 10% by weight PEG having a molecular weight from about 500 g/mol to about 15,000 g/mol, and about 10% PEG having a molecular weight from about 25,000 g/mol to about 50,000 g/mol.
33. The ink formulation of embodiment 32, comprising about 60% by weight βTCP, about 20% by weight caprolactone/glycolide copolymer, about 10% by weight PEG having a molecular weight of about 8,000 g/mol, and about 10% PEG having a molecular weight of about 35,000 g/mol.
34. The ink formulation of any one of embodiments 31-33, wherein the caprolactone/glycolide copolymer is caprolactone/glycolide copolymer (95:5).
35. The ink formulation of any one of embodiments 31-33, wherein the caprolactone/glycolide copolymer is caprolactone/glycolide copolymer (90:10).
36. An ink formulation comprising about 55% to about 65% by weight βTCP, about 15% to about 25% by weight poly(D,L-lactide-co-glycolide) copolymer, about 5% to about 15% PEG having a molecular weight from about 500 g/mol to about 15,000 g/mol, and about 5% to about 15% PEG having a molecular weight from about 25,000 g/mol to about 50,000 g/mol.
37. The ink formulation of embodiment 36, comprising about 60% by weight βTCP, about 20% by weight poly(D,L-lactide-co-glycolide) copolymer, about 10% by weight PEG having a molecular weight from about 500 g/mol to about 15,000 g/mol, and about 10% PEG having a molecular weight from about 25,000 g/mol to about 50,000 g/mol.
38. The ink formulation of embodiment 37, comprising about 60% by weight βTCP, about 20% by weight poly(D,L-lactide-co-glycolide) copolymer, about 10% by weight PEG having a molecular weight of about 8,000 g/mol, and about 10% PEG having a molecular weight of about 35,000 g/mol.
39. The ink formulation of any one of embodiments 36-38, wherein the caprolactone/glycolide copolymer is caprolactone/glycolide copolymer (50:50).
40. An ink formulation comprising about 55% to about 65% by weight βTCP, about 15% to about 25% by weight PDS, about 5% to about 15% PEG having a molecular weight from about 500 g/mol to about 15,000 g/mol, and about 5% to about 15% PEG having a molecular weight from about 25,000 g/mol to about 50,000 g/mol.
41. The ink formulation of embodiment 40, comprising about 60% by weight βTCP, about 20% by weight PDS, about 10% by weight PEG having a molecular weight from about 500 g/mol to about 15,000 g/mol, and about 10% PEG having a molecular weight from about 25,000 g/mol to about 50,000 g/mol.
42. The ink formulation of embodiment 41, comprising about 60% by weight βTCP, about 20% by weight PDS, about 10% by weight PEG having a molecular weight of about 8,000 g/mol, and about 10% PEG having a molecular weight of about 35,000 g/mol.
43. An ink formulation comprising about 55% to about 65% by weight βTCP, about 15% to about 25% by weight dioxanone/L-lactide copolymer, about 5% to about 15% PEG having a molecular weight from about 500 g/mol to about 15,000 g/mol, and about 5% to about 15% PEG having a molecular weight from about 25,000 g/mol to about 50,000 g/mol.
44. The ink formulation of embodiment 43, comprising about 60% by weight βTCP, about 20% by weight dioxanone/L-lactide copolymer, about 10% by weight PEG having a molecular weight from about 500 g/mol to about 15,000 g/mol, and about 10% PEG having a molecular weight from about 25,000 g/mol to about 50,000 g/mol.
45. The ink formulation of embodiment 44, comprising about 60% by weight βTCP, about 20% by weight dioxanone/L-lactide copolymer, about 10% by weight PEG having a molecular weight of about 8,000 g/mol, and about 10% PEG having a molecular weight of about 35,000 g/mol.
46. The ink formulation of any one of embodiments 43-45, wherein the caprolactone/glycolide copolymer is caprolactone/glycolide copolymer (90:10).
47. An ink formulation comprising about 55% to about 65% by weight βTCP, about 15% to about 25% by weight glycolide/L-lactide copolymer, about 5% to about 15% PEG having a molecular weight from about 500 g/mol to about 15,000 g/mol, and about 5% to about 15% PEG having a molecular weight from about 25,000 g/mol to about 50,000 g/mol.
48. The ink formulation of embodiment 47, comprising about 60% by weight βTCP, about 20% by weight glycolide/L-lactide copolymer, about 10% by weight PEG having a molecular weight from about 500 g/mol to about 15,000 g/mol, and about 10% PEG having a molecular weight from about 25,000 g/mol to about 50,000 g/mol.
49. The ink formulation of embodiment 48, comprising about 60% by weight βTCP, about 20% by weight glycolide/L-lactide copolymer, about 10% by weight PEG having a molecular weight of about 8,000 g/mol, and about 10% PEG having a molecular weight of about 35,000 g/mol.
50. The ink formulation of any one of embodiments 47-49, wherein the caprolactone/glycolide copolymer is glycolide/L-lactide copolymer (95:5).
51. An ink formulation comprising about 55% to about 65% by weight βTCP, about 15% to about 25% by weight poly-1-lactide, about 5% to about 15% PEG having a molecular weight from about 500 g/mol to about 15,000 g/mol, and about 5% to about 15% PEG having a molecular weight from about 25,000 g/mol to about 50,000 g/mol.
52. The ink formulation of embodiment 51, comprising about 60% by weight βTCP, about 20% by weight poly-1-lactide, about 10% by weight PEG having a molecular weight from about 500 g/mol to about 15,000 g/mol, and about 10% PEG having a molecular weight from about 25,000 g/mol to about 50,000 g/mol.
53. The ink formulation of embodiment 52, comprising about 60% by weight βTCP, about 20% by weight poly-1-lactide, about 10% by weight PEG having a molecular weight of about 8,000 g/mol, and about 10% PEG having a molecular weight of about 35,000 g/mol.
54. A method of preparing a three-dimensional structure, the method comprises performing additive manufacturing with the ink formulation of any one of embodiments 26-53.
55. The method of embodiment 54, wherein the ink formulation is in the form of a pellet. 56. The method of embodiment 55, wherein the additive manufacturing comprises fused granular fabrication (FGF).
57. The method of embodiment 54, wherein the ink formulation is in the form of a filament.
58. The method of embodiment 57, wherein the additive manufacturing comprises fused filament fabrication (FFF).
59. A device comprising a therapeutic agent and the structure of any one of embodiments 1-25 or embodiments 54-58.
60. The device of embodiment 59, wherein the therapeutic agent is non-covalently bound to the structure.
61. The device of embodiment 59 or embodiment 60, wherein the therapeutic agent comprises a growth factor.
62. The device of embodiment 61, wherein the growth factor is selected from Table 1.
63. The device of any one of embodiments 59-62, wherein the therapeutic agent comprises a bone morphogenetic protein (BMP).
64. The device of any one of embodiments 59-63, wherein the therapeutic agent comprises a targeting moiety, and the targeting moiety is non-covalently bound to the structure.
65. The device of embodiment 64, wherein the targeting moiety comprises a polypeptide at least about 70%, 75%, 80%, 85%, 90%, 95%, or 100% identical to any one of the sequences of Tables 2-3.
66. The device of any one of embodiments 59-65, wherein the therapeutic agent comprises a chimeric polypeptide comprising a sequence at least about 70%, 75%, 80%, 85%, 90%, 95%, or 100% identical to any one of SEQ ID NOS: 433-441.
67. A method of treating a bone defect in a subject in need thereof, the method comprising application of the structure of any one of embodiments 1-25 or embodiments 54-58 to the defect of the subject.
68. A method of treating a bone defect in a subject in need thereof, the method comprising application of the device of any one of embodiments 59-66 to the defect of the subject.
69. The method of embodiment 67 or embodiment 68, wherein the defect is present in the spine.

Each of the embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

A number of embodiments of the disclosure have been described. Nevertheless, it will be understood that various modifications may be made without departing from the spirit and scope of the disclosure. Additionally, while specific formulations for the inks are described, variations of the specific quantities of each ink ingredient are possible. Accordingly, other embodiments are within the scope of the following claims.

### EXAMPLES

### Example 1: Ink Formulations and 3D Printed Scaffolds

### Ink Formulation and Scaffold #1:

This 3D printing ink material is a flexible, polymer-ceramic composite material containing β-TCP that may be tethered to a therapeutic agent, such as tBMP2. This formulation contains two sacrificial pore formers (water soluble polyethylene glycol and water soluble sucrose) to expose more β-TCP surface area for tBMP2 binding. This ink is a low viscosity formulation that was extruded through a 400 μm diameter nozzle on an Allevi 3 pneumatic bioprinter. After 3D printing was complete, the resulting scaffold was soaked in water to dissolve the sacrificial polyethylene glycol and sucrose pore formers. The resulting scaffold #1 contains 75% by weight β-TCP powder, and 25% by weight polycaprolactone.

This ink is also converted into a filament form and utilized to prepare a 3D printed scaffold using a fused filament fabrication (FFF or FDM) 3D printer. After 3D printing is complete, the resulting scaffold is soaked in water to dissolve the sacrificial polyethylene glycol and sucrose pore formers. The resulting scaffold contains 75% by weight β-TCP powder, and 25% by weight polycaprolactone.

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 4
Example ink formulation #1

| | Wt % | Component | |
|---|---|---|---|
| Component | in ink | characteristics | Purpose |
| β-TCP Powder | 57 | Spray dried powder, 10-38 | Spherical β-TCP Powder for |
| | | micron particle size | good extrudability during 3D |
| | | | printing, binding sites for |
| | | | targeting moiety |
| Polycaprolactone | 19 | 50,000 MW, fine powder, | Flexible polymer binder for |
| | | Tmelt = 60 C | β-TCP powder; not water soluble |
| Polyethylene | 19 | 1500 MW, flake, Tmelt = | Water soluble polymer with low |
| Glycol | | 60 C | melt viscosity for good extrusion |
| Sucrose | 5 | Fine powder | Water soluble particulate, |
| | | | sacrificial pore former |

### Ink Formulation #2

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This formulation employs a 95 mol % caprolactone 5 mol % glycolide copolymer for faster bioresorption characteristics compared to polycaprolactone. This ink is a low viscosity formulation that was extruded through a 320 μm diameter nozzle on an Allevi 3 pneumatic bioprinter. After 3D printing is complete, the resulting scaffold was soaked in water to dissolve the sacrificial polyethylene glycol pore former. The resulting scaffold #2 contains 75% by weight β-TCP powder, and 25% by weight caprolactone/glycolide copolymer (95:5).

This ink is also converted into a filament form and utilized to prepare a 3D printed scaffold using a fused filament fabrication (FFF or FDM) 3D printer. After 3D printing is complete, the resulting scaffold is soaked in water to dissolve the sacrificial polyethylene glycol pore former. The resulting scaffold contains 75% by weight β-TCP powder, and 25% by weight caprolactone/glycolide copolymer (95:5).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 5
Example ink formulation #2

| | Wt % | Component | |
|---|---|---|---|
| Component | in ink | characteristics | Purpose |
| β-TCP Powder | 60 | Spray dried powder, 10-38 | Spherical β-TCP Powder for |
| | | micron particle size | good extrudability during 3D |
| | | | printing, binding sites for |
| | | | therapeutic |
| Caprolactone/ | 20 | 95 mol % polycaprolactone, | Flexible polymer binder for |
| Glycolide | | 5 mol % polyglycolide, | β-TCP powder; not water |
| copolymer | | pellets, Tmelt = 52-62 C | soluble; faster bioresorption |
| | | | compared to polycaprolactone |
| Polyethylene | 20 | 1500 MW, flake, Tmelt = | Water soluble polymer with |
| Glycol | | 60 C | low melt viscosity for good |
| | | | extrusion |

### Ink Formulation #3

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This formulation employs a 90 mol % caprolactone 10 mol % glycolide copolymer for fast bioresorption characteristics compared to polycaprolactone. This ink is a low viscosity formulation that was extruded through a 320 μm diameter nozzle on an Allevi 3 pneumatic bioprinter. After 3D printing was complete, the resulting scaffold was soaked in water to dissolve the sacrificial polyethylene glycol pore former. The resulting scaffold #3 contains 75% by weight β-TCP powder, and 25% by weight caprolactone/glycolide copolymer (90:10).

This ink is also converted into a filament form and utilized to prepare a 3D printed scaffold using a fused filament fabrication (FFF or FDM) 3D printer. After 3D printing is complete, the resulting scaffold is soaked in water to dissolve the sacrificial polyethylene glycol pore former. The resulting scaffold contains 75% by weight β-TCP powder, and 25% by weight caprolactone/glycolide copolymer (90:10).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 6
Example ink formulation #3

| | Wt % | Component | |
|---|---|---|---|
| Component | in ink | characteristics | Purpose |
| β-TCP Powder | 60 | Spray dried powder, 10-38 | Spherical β-TCP Powder for |
| | | micron particle size | good extrudability during 3D |
| | | | printing, binding sites for |
| | | | therapeutic |
| Caprolactone/ | 20 | 90 mol % polycaprolactone, | Flexible polymer binder for |
| Glycolide | | 10 mol % polyglycolide, | β-TCP powder; not water |
| copolymer | | chips | soluble; fast bioresorption |
| | | | compared to polycaprolactone |
| Polyethylene | 20 | 1500 MW, flake, Tmelt = | Water soluble polymer with |
| Glycol | | 60 C | low melt viscosity for good |
| | | | extrusion |

### Ink Formulation #4

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This formulation employs a 50 mol %: 50 mol % poly(D,L-lactide-co-glycolide) copolymer for fast bioresorption characteristics compared to polycaprolactone. This ink is a low viscosity formulation that was extruded through a 400 μm diameter nozzle on an Allevi 3 pneumatic bioprinter. After 3D printing was complete, the resulting scaffold was soaked in water to dissolve the sacrificial polyethylene glycol pore former. The resulting scaffold #4 contains 75% by weight β-TCP powder, and 25% by weight poly(D,L-lactide-co-glycolide) copolymer (50:50).

This ink is also converted into a filament form and utilized to prepare a 3D printed scaffold using a fused filament fabrication (FFF or FDM) 3D printer. After 3D printing is complete, the resulting scaffold is soaked in water to dissolve the sacrificial polyethylene glycol pore former. The resulting scaffold contains 75% by weight β-TCP powder, and 25% by weight poly(D,L-lactide-co-glycolide) copolymer (50:50).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 7
Example ink formulation #4

| | | Component | |
|---|---|---|---|
| Component | Wt % in ink | characteristics | Purpose |
| β-TCP Powder | 60 | Spray dried powder, 10-38 | Spherical β-TCP Powder for |
| | | micron particle size | good extrudability during 3D |
| | | | printing, binding sites for |
| | | | therapeutic |
| poly(D,L-lactide-co- | 20 | 50 mol % poly(D,L-lactide), | Flexible polymer binder for |
| glycolide) copolymer | | 50 mol % polyglycolide, | β-TCP powder; not water |
| | | chunks, 38,000-54,000 | soluble; fast bioresorption |
| | | MW, Tg = 46-50 C | compared to |
| | | | polycaprolactone |
| Polyethylene Glycol | 20 | 1500 MW, flake, Tmelt = | Water soluble polymer with |
| | | 60 C | low melt viscosity for good |
| | | | extrusion |

### Ink Formulation #5

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This ink is a moderate viscosity formulation that was formed into a 1.75 mm filament for 3D printing on a RepRap style FFF 3D printers (e.g. Prusa i3 MK3S 3D printer) with a 400 μm diameter nozzle. The higher molecular weight polyethylene glycol (8000 MW for FFF 3D printing vs. 1500 MW for syringe-based bioprinting) results in a higher viscosity material which aids in extrusion of 1.75 mm diameter filaments. After 3D printing was complete, the resulting scaffold was soaked in water to dissolve the sacrificial polyethylene glycol pore former. The resulting scaffold #5 contains 75% by weight β-TCP powder, and 25% by weight polycaprolactone.

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 8
Example ink formulation #5

| | | Component | |
|---|---|---|---|
| Component | Wt % in ink | characteristics | Purpose |
| β-TCP Powder | 60 | Spray dried powder, 10-38 | Spherical β-TCP Powder for |
| | | micron particle size | good extrudability during 3D |
| | | | printing, binding sites for |
| | | | therapeutic |
| Polycaprolactone | 20 | 50,000 MW, fine powder, | Flexible polymer binder for |
| | | Tmelt = 60 C | β-TCP powder; not water |
| | | | soluble |
| Polyethylene Glycol | 20 | 8,000 MW, flake, Tmelt = | Water soluble polymer with |
| | | 60 C | low melt viscosity for good |
| | | | extrusion |

### Ink Formulation #6

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This ink also contains a blowing agent (sodium bicarbonate) which thermally decomposes and releases CO2 gas during 3D printing to create a foamed structure, thus increasing the porosity of the 3D printed scaffold. This ink is a moderate viscosity formulation that was formed into a 1.75 mm filament for 3D printing on a RepRap style FFF 3D printers (e.g. Prusa i3 MK3S 3D printer) with a 400 μm diameter nozzle. The higher molecular weight polyethylene glycol (8000 MW for FFF 3D printing vs. 1500 MW for syringe-based bioprinting) results in a higher viscosity material which aids in extrusion of 1.75 mm diameter filaments. After 3D printing was complete, the resulting scaffold was soaked in water to dissolve the sacrificial polyethylene glycol pore former and sodium carbonate by-product from the sodium bicarbonate thermal decomposition. The resulting scaffold #6 contains 75% by weight β-TCP powder, and 25% by weight polycaprolactone.

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 9
Example ink formulation #6

| | | Component | |
|---|---|---|---|
| Component | Wt % in ink | characteristics | Purpose |
| β-TCP Powder | 54 | Spray dried powder, 10-38 | Spherical β-TCP Powder for |
| | | micron particle size | good extrudability during 3D |
| | | | printing, binding sites for |
| | | | therapeutic |
| Polycaprolactone | 18 | 50,000 MW, fine powder, | Flexible polymer binder for |
| | | Tmelt = 60 C | β-TCP powder; not water |
| | | | soluble |
| Polyethylene Glycol | 18 | 8,000 MW, flake, Tmelt = | Water soluble polymer with |
| | | 60 C | low melt viscosity for good |
| | | | extrusion |
| Sodium Bicarbonate | 10 | Fine powder, thermal | Blowing agent |
| | | decomposition temperature | |
| | | 80 C | |

### Ink Formulation #7

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This formulation employs a 90 mol %: 10 mol % poly(dioxanone-co-L-lactide) copolymer for faster bioresorption characteristics compared to polycaprolactone. This ink is a low viscosity formulation that can be extruded through a 400 μm diameter nozzle on an Allevi 3 pneumatic bioprinter. After 3D printing is complete, the resulting scaffold is soaked in water to dissolve the sacrificial polyethylene glycol pore former. The resulting scaffold contains 75% by weight β-TCP powder, and 25% by weight dioxanone/L-lactide copolymer (90:10).

This ink is also converted into a filament form and utilized to prepare a 3D printed scaffold using a fused filament fabrication (FFF or FDM) 3D printer. After 3D printing is complete, the resulting scaffold is soaked in water to dissolve the sacrificial polyethylene glycol pore former. The resulting scaffold contains 75% by weight β-TCP powder, and 25% by weight dioxanone/L-lactide copolymer (90:10).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 10
Example ink formulation #7

| | | Component | |
|---|---|---|---|
| Component | Wt % in ink | characteristics | Purpose |
| β-TCP Powder | 60 | Spray dried powder, 10-38 | Spherical β-TCP Powder for |
| | | micron particle size | good extrudability during 3D |
| | | | printing, binding sites for |
| | | | therapeutic |
| Dioxanone/L-lactide | 20 | 90 mol % polydioxanone, | Flexible polymer binder for |
| copolymer | | 10 mol % poly-L-lactide, | β-TCP powder; not water |
| | | white chips, Tmelt = ~110 | soluble; faster bioresorption |
| | | C | compared to |
| | | | polycaprolactone |
| Polyethylene Glycol | 20 | 1500 MW, flake, Tmelt = | Water soluble polymer with |
| | | 60 C | low melt viscosity for good |
| | | | extrusion |

### Ink Formulation #8

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This ink is a moderate viscosity formulation that can be formed into a 1.75 mm filament for 3D printing on a RepRap style FFF 3D printers (e.g. Prusa i3 MK3S 3D printer) with a 400 μm diameter nozzle. The higher molecular weight blend of polyethylene glycol (8000 MW and 20000 MW for FFF 3D printing vs. 1500 MW for syringe-based bioprinting) results in a higher viscosity material which aids in extrusion of 1.75 mm diameter filaments. After 3D printing is complete, the resulting scaffold is soaked in water to dissolve the sacrificial polyethylene glycol pore former. The resulting scaffold contains 75% by weight β-TCP powder, and 25% by weight polycaprolactone.

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 11
Example ink formulation #8

| | | Component | |
|---|---|---|---|
| Component | Wt % in ink | characteristics | Purpose |
| β-TCP Powder | 60 | Spray dried powder, 10-38 | Spherical β-TCP Powder for |
| | | micron particle size | good extrudability during 3D |
| | | | printing, binding sites for |
| | | | therapeutic |
| Polycaprolactone | 20 | 50,000 MW, fine powder, | Flexible polymer binder for |
| | | Tmelt = 60 C | β-TCP powder; not water |
| | | | soluble |
| Polyethylene Glycol | 10 | 8,000 MW, flake, Tmelt = | Water soluble polymer with |
| | | 60 C | low melt viscosity |
| Polyethylene Glycol | 10 | 20,000 MW, flake, | Water soluble polymer with |
| | | Tmelt = 60 C | moderate melt viscosity |

### Ink Formulation #9

This 3D printing ink material is a flexible, polymer-ceramic composite material containing β-TCP that may be tethered to a therapeutic agent, such as tBMP2. This formulation contains two sacrificial pore formers (water soluble polyethylene glycol and water soluble glucose) to expose more β-TCP surface area for agent binding. This ink is a moderate viscosity formulation that can be formed into a 1.75 mm filament for 3D printing on a RepRap style FFF 3D printers (e.g. Prusa i3 MK3S 3D printer) with a 400 μm diameter nozzle. The higher molecular weight blend of polyethylene glycol (8000 MW and 20000 MW for FFF 3D printing vs. 1500 MW for syringe-based bioprinting) results in a higher viscosity material, which aids in extrusion of 1.75 mm diameter filaments. After 3D printing is complete, the resulting scaffold is soaked in water to dissolve the sacrificial polyethylene glycol and sucrose pore formers. The resulting scaffold contains 75% by weight β-TCP powder, and 25% by weight polycaprolactone.

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 12
Example ink formulation #9

| | | Component | |
|---|---|---|---|
| Component | Wt % in ink | characteristics | Purpose |
| β-TCP Powder | 60 | Spray dried powder, 10-38 | Spherical β-TCP Powder for |
| | | micron particle size | good extrudability during 3D |
| | | | printing, binding sites for |
| | | | therapeutic |
| Polycaprolactone | 20 | 50,000 MW, fine powder, | Flexible polymer binder for |
| | | Tmelt = 60 C | β-TCP powder; not water |
| | | | soluble |
| Polyethylene Glycol | 10 | 8,000 MW, flake, Tmelt = | Water soluble polymer with |
| | | 60 C | low melt viscosity |
| Polyethylene Glycol | 10 | 20,000 MW, flake, | Water soluble polymer with |
| | | Tmelt = 60 C | moderate melt viscosity |
| Sucrose | 5 | Fine powder | Water soluble particulate, |
| | | | sacrificial pore former |

### Ink Formulation #10

This 3D printing ink material is a flexible, polymer-ceramic composite material containing β-TCP that may be tether to a therapeutic agent, such a tBMP2. This formulation contains a sacrificial pore former (water soluble polyethylene glycol) to expose more β-TCP surface area for agent binding. This formulation employs a 95 mol % caprolactone 5 mol % glycolide copolymer for faster bioresorption characteristics compared to polycaprolactone. This ink is a moderate viscosity formulation that can be formed into a 1.75 mm filament for 3D printing on a RepRap style FFF 3D printers (e.g. Prusa i3 MK3S 3D printer) with a 400 μm diameter nozzle. The higher molecular weight blend of polyethylene glycol (8000 MW and 20000 MW for FFF 3D printing vs. 1500 MW for syringe-based bioprinting) results in a higher viscosity material which aids in extrusion of 1.75 mm diameter filaments. After 3D printing is complete, the resulting scaffold is soaked in water to dissolve the sacrificial polyethylene glycol pore former. The resulting scaffold contains 75% by weight β-TCP powder, and 25% by weight caprolactone/glycolide copolymer (95:5).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 13
Example ink formulation #10

| | | Component | |
|---|---|---|---|
| Component | Wt % in ink | characteristics | Purpose |
| β-TCP Powder | 60 | Spray dried powder, 10-38 | Spherical β-TCP Powder for |
| | | micron particle size | good extrudability during 3D |
| | | | printing, binding sites for |
| | | | therapeutic |
| Caprolactone/Glycolide | 20 | 95 mol % polycaprolactone, | Flexible polymer binder for |
| copolymer (95:5) | | 5 mol % polyglycolide, | β-TCP powder; not water |
| | | pellets, Tmelt = 52-62 C | soluble; faster bioresorption |
| | | | compared to |
| | | | polycaprolactone |
| Polyethylene Glycol | 10 | 8,000 MW, flake, Tmelt = | Water soluble polymer with |
| | | 60 C | low melt viscosity |
| Polyethylene Glycol | 10 | 20,000 MW, flake, | Water soluble polymer with |
| | | Tmelt = 60 C | moderate melt viscosity |

### Ink Formulation #11

This 3D printing ink material is a flexible, polymer-ceramic composite material containing β-TCP that may be tether to a therapeutic agent, such as tBMP2. This formulation contains a sacrificial pore former (water soluble polyethylene glycol) to expose more β-TCP surface area for agent binding. This formulation employs a 90 mol % caprolactone 10 mol % glycolide copolymer for fast bioresorption characteristics compared to polycaprolactone. This ink is a moderate viscosity formulation that can be formed into a 1.75 mm filament for 3D printing on a RepRap style FFF 3D printers (e.g. Prusa i3 MK3S 3D printer) with a 400 μm diameter nozzle. The higher molecular weight blend of polyethylene glycol (8000 MW and 20000 MW for FFF 3D printing vs. 1500 MW for syringe-based bioprinting) results in a higher viscosity material which aids in extrusion of 1.75 mm diameter filaments. After 3D printing is complete, the resulting scaffold is soaked in water to dissolve the sacrificial polyethylene glycol pore former. The resulting scaffold contains 75% by weight β-TCP powder, and 25% by weight caprolactone/glycolide copolymer (90:10).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 14
Example ink formulation #11

| | | Component | |
|---|---|---|---|
| Component | Wt % in ink | characteristics | Purpose |
| β-TCP Powder | 60 | Spray dried powder, 10-38 | Spherical β-TCP Powder for |
| | | micron particle size | good extrudability during 3D |
| | | | printing, binding sites for |
| | | | therapeutic |
| Caprolactone/Glycolide | 20 | 90 mol % polycaprolactone, | Flexible polymer binder for |
| copolymer (90:10) | | 10 mol % polyglycolide, | β-TCP powder; not water |
| | | chips | soluble; fast bioresorption |
| | | | compared to |
| | | | polycaprolactone |
| Polyethylene Glycol | 10 | 8,000 MW, flake, Tmelt = | Water soluble polymer with |
| | | 60 C | low melt viscosity |
| Polyethylene Glycol | 10 | 20,000 MW, flake, | Water soluble polymer with |
| | | Tmelt = 60 C | moderate melt viscosity |

### Ink Formulation #12

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. This formulation employs a 50 mol %: 50 mol % poly(D,L-lactide-co-glycolide) copolymer for fast bioresorption characteristics compared to polycaprolactone. This ink is a moderate viscosity formulation that can be formed into a 1.75 mm filament for 3D printing on a RepRap style FFF 3D printers (e.g. Prusa i3 MK3S 3D printer) with a 400 μm diameter nozzle. The higher molecular weight blend of polyethylene glycol (8000 MW and 20000 MW for FFF 3D printing vs. 1500 MW for syringe-based bioprinting) results in a higher viscosity material which aids in extrusion of 1.75 mm diameter filaments. After 3D printing is complete, the resulting scaffold is soaked in water to dissolve the sacrificial polyethylene glycol pore former. The resulting scaffold contains 75% by weight β-TCP powder, and 25% by weight poly(D,L-lactide-co-glycolide) copolymer (50:50).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 15
Example ink formulation #12

| | | Component | |
|---|---|---|---|
| Component | Wt % in ink | characteristics | Purpose |
| β-TCP Powder | 60 | Spray dried powder, 10-38 | Spherical β-TCP Powder for |
| | | micron particle size | good extrudability during 3D |
| | | | printing, binding sites for |
| | | | therapeutic. |
| poly(D,L-lactide-co- | 20 | 50 mol % poly(D,L-lactide), | Flexible polymer binder for |
| glycolide) copolymer | | 50 mol % polyglycolide, | β-TCP powder; not water |
| | | chunks, 38,000-54,000 | soluble; fast bioresorption |
| | | MW, Tg = 46-50 C | compared to |
| | | | polycaprolactone |
| Polyethylene Glycol | 10 | 8,000 MW, flake, Tmelt = | Water soluble polymer with |
| | | 60 C | low melt viscosity |
| Polyethylene Glycol | 10 | 20,000 MW, flake, | Water soluble polymer with |
| | | Tmelt = 60 C | moderate melt viscosity |

### Ink Formulation #13

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. 3D printing is performed using the formulation in a syringe ink and filament form. After 3D printing is complete, the resulting scaffold is soaked in water to dissolve the sacrificial polyethylene glycol pore former. The resulting scaffold contains β-TCP and PDS.

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 16
Example ink formulation #13

| Component | Wt % in ink |
|---|---|
| β-TCP Powder | 50-80 |
| Polydioxanone (PDS) | 10-30 |
| Polyethylene Glycol | 10-30 |

### Ink Formulation #14

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. 3D printing is performed using the formulation in a syringe ink and filament form. After 3D printing is complete, the resulting scaffold is soaked in water to dissolve the sacrificial polyethylene glycol pore former. The resulting scaffold contains β-TCP and PDS-glycolide copolymer (90:10).

This ink is also converted into pellets (e.g., a filament is formed into small pellets) and fed from a hopper into a mini-screw extrusion head which melts and pushes the material out of a fine nozzle

This ink is also cryomilled into a fine powder. A laser heats the powder into the desired configuration using the SLS method.

TABLE 17
Example ink formulation #14

| Component | Wt % in ink |
|---|---|
| β-TCP Powder | 50-80 |
| PDS-Glycolide Copolymer (90:10) | 10-30 |
| Polyethylene Glycol | 10-30 |

### Ink Formulation #15

This 3D printing ink material is a flexible, polymer-ceramic composite material containing β-TCP that may be tethered to a therapeutic agent, such as tBMP2. This formulation contains a sacrificial pore former (water soluble polyethylene glycol) to expose more β-TCP surface area for agent binding. 3D printing is performed using the formulation in a syringe ink and filament form. After 3D printing is complete, the resulting scaffold is soaked in water to dissolve the sacrificial polyethylene glycol pore former. The resulting scaffold contains β-TCP and -L-Lactide Copolymer (90:10).

This ink is also converted into pellets (e.g., a filament is formed into small pellets) and fed from a hopper into a mini-screw extrusion head which melts and pushes the material out of a fine nozzle.

This ink is also cryomilled into a fine powder. A laser heats the powder into the desired configuration using the SLS method.

TABLE 18
Example ink formulation #15

| Component | Wt % in ink |
|---|---|
| β-TCP Powder | 50-80 |
| PDS-L-Lactide Copolymer (90:10) | 10-30 |
| Polyethylene Glycol | 10-30 |

### Ink Formulation #16

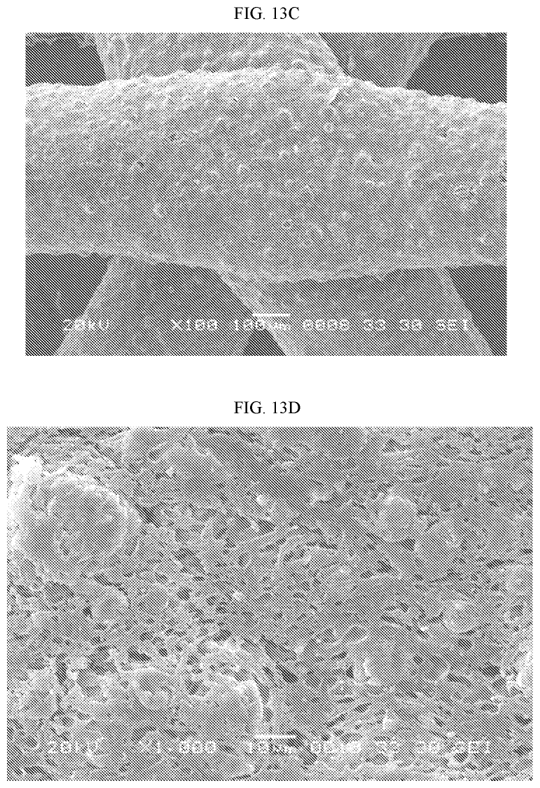

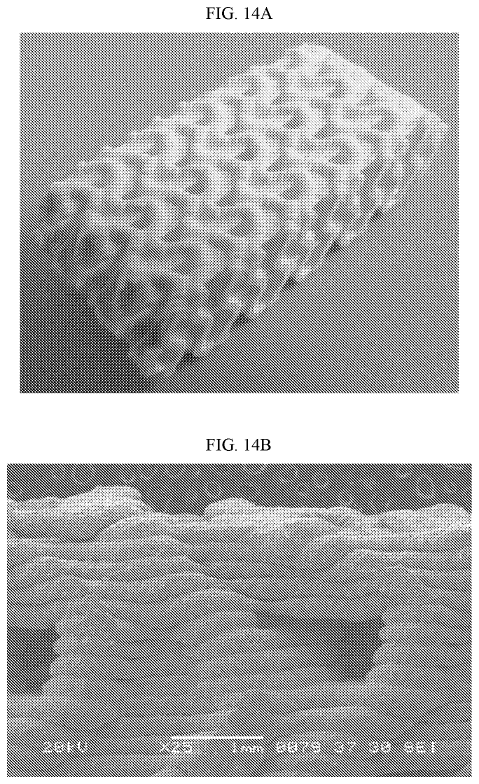

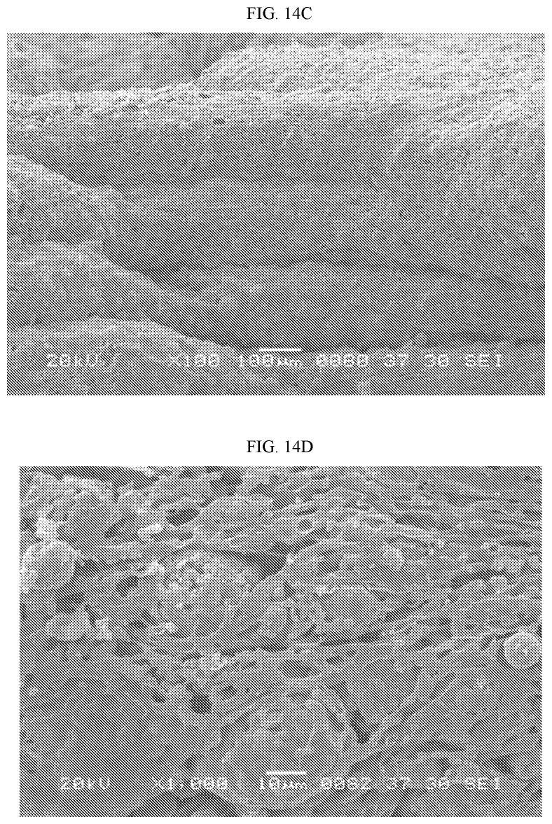

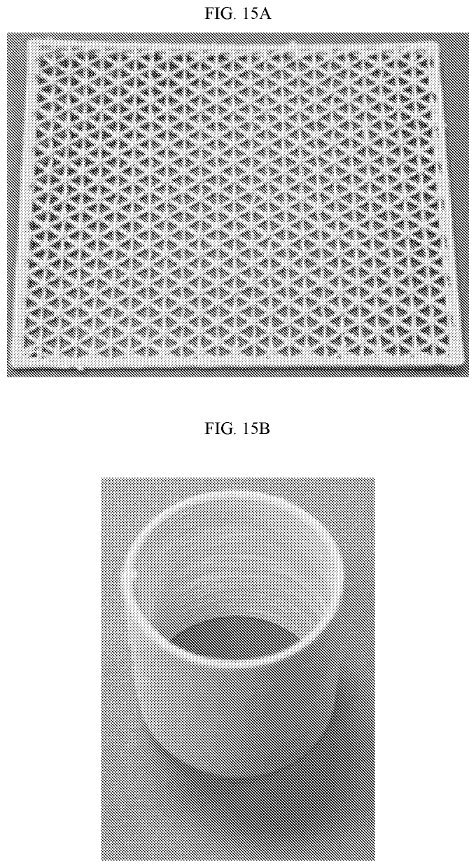

This 3D printing ink material is a flexible, polymer-ceramic composite material containing β-TCP that may be tethered to a therapeutic agent, such as tBMP2. This formulation contains sacrificial pore formers (water soluble polyethylene glycol components) to expose more β-TCP surface area for agent binding. This ink is a moderate viscosity formulation that can be formed into ~3-4 mm diameter pellets to be used as feedstock in a fused granular fabrication (FGF) 3D printer (e.g. Piocreat G5) with a 300-1,000 μm diameter nozzle. After 3D printing is complete, the resulting scaffold is soaked in water to dissolve the sacrificial polyethylene glycol pore former. The higher molecular weight blend of polyethylene glycol (8000 MW and 35000 MW for FGF 3D printing vs. 1500 MW for syringe-based bioprinting) results in a higher viscosity material. After 3D printing is complete, the resulting scaffold is soaked in water to dissolve the sacrificial polyethylene glycol pore formers. The resulting scaffold contains 75% by weight β-TCP powder, and 25% by weight polycaprolactone. Two example scaffolds were prepared using this ink via FGF 3D printing. Images of the scaffolds are shown in FIGS. 13A-13D, and FIGS. 14A-14D.

This ink is prepared in pellet or filament form for FGF or FFF printing, respectively. This ink is cryomilled into a fine powder, e.g., for SLS printing.

TABLE 19
Example ink formulation #16 - new pellet formulation

| | | Component | |
|---|---|---|---|
| Component | Wt % in ink | characteristics | Purpose |
| β-TCP Powder | 60 | Spray dried powder, 10-38 | Spherical β-TCP Powder for |
| | | micron particle size | good extrudability during 3D |
| | | | printing, binding sites for |
| | | | therapeutic |
| Polycaprolactone | 20 | 50,000 MW, fine powder, | Flexible polymer binder for |
| | | Tmelt = 60 C | β-TCP powder; not water |
| | | | soluble |
| Polyethylene Glycol | 10 | 8,000 MW, flake, Tmelt = | Water soluble polymer with |
| | | 60 C | low melt viscosity |
| Polyethylene Glycol | 10 | 35,000 MW, flake, | Water soluble polymer with |
| | | Tmelt = 60 C | moderate melt viscosity |

### Ink Formulation #17

This 3D printing ink material is a flexible, polymer-ceramic composite material containing β-TCP that may be tethered to a therapeutic agent, such as tBMP2. This formulation contains sacrificial pore formers (water soluble polyethylene glycol components) to expose more β-TCP surface area for agent binding. This ink is a moderate viscosity formulation that can be formed into ~3-4 mm diameter pellets to be used as feedstock in a fused granular fabrication (FGF) 3D printer (e.g. Piocreat G5) with a 300-1,000 μm diameter nozzle. After 3D printing is complete, the resulting scaffold is soaked in water to dissolve the sacrificial polyethylene glycol pore former. The higher molecular weight blend of polyethylene glycol (8000 MW and 35000 MW for FGF 3D printing vs. 1500 MW for syringe-based bioprinting) results in a higher viscosity material. After 3D printing is complete, the resulting scaffold is soaked in water to dissolve the sacrificial PEG and sucrose. The resulting scaffold contains 50-88% by weight β-TCP powder, and 13-50% by weight polycaprolactone.

This ink is prepared in pellet or filament form for FGF or FFF printing, respectively. This ink is cryomilled into a fine powder, e.g., for SLS printing.

TABLE 20
Example ink formulation #17

| Component | Wt % in ink |
|---|---|
| β-TCP Powder | 30-70 |
| Polycaprolactone | 10-30 |
| Polyethylene Glycol (8000 MW) | 5-15 |
| Polyethylene Glycol (35000 MW) | 5-15 |
| Sucrose | 1-10 |

### Ink Formulation #18

This 3D printing ink material is a flexible, polymer-ceramic composite material containing β-TCP that may be tethered to a therapeutic agent, such as tBMP2. This formulation contains sacrificial pore formers (water soluble polyethylene glycol components) to expose more β-TCP surface area for agent binding. This ink is a moderate viscosity formulation that can be formed into ~2-4 mm diameter pellets to be used as feedstock in a fused granular fabrication (FGF) 3D printer (e.g. Piocreat G5) with a 300-1,000 μm diameter nozzle. After 3D printing is complete, the resulting scaffold is soaked in water to dissolve the sacrificial polyethylene glycol pore former. The higher molecular weight blend of polyethylene glycol (8000 MW and 35000 MW for FGF 3D printing vs. 1500 MW for syringe-based bioprinting) results in a higher viscosity material. After 3D printing is complete, the resulting scaffold is soaked in water to dissolve the sacrificial PEG. The resulting scaffold contains 55-88% by weight β-TCP powder, and 13-50% by weight Caprolactone/Glycolide copolymer (95:5).

This ink is prepared in pellet or filament form for FGF or FFF printing, respectively. This ink is cryomilled into a fine powder, e.g., for SLS printing.

TABLE 21
Specific Ink Formulation #18

| | Wt % in | Component | |
|---|---|---|---|
| Component | ink | characteristics | Purpose |
| β-TCP Powder | 60 | Spray dried powder, 10- | Spherical β-TCP Powder |
| | | 38 micron particle size | for good extrudability |
| | | | during 3D printing, |
| | | | binding sites for |
| | | | therapeutic protein |
| Caprolactone/Glycolide | 20 | 95 mol % | Flexible polymer binder |
| copolymer (95:5) | | polycaprolactone, | for β-TCP powder; not |
| | | 5 mol % polyglycolide, | water soluble |
| | | pellets, Tmelt = 52-62 C | |
| Polyethylene Glycol | 10 | 8,000 MW, flake, Tmelt = | Water soluble polymer |
| | | 60 C | with low melt viscosity |
| Polyethylene Glycol | 10 | 35,000 MW, flake, | Water soluble polymer |
| | | Tmelt = 60 C | with moderate melt |
| | | | viscosity |

TABLE 22
Example ink formulation #18

| Component | Wt % in ink |
|---|---|
| β-TCP Powder | 30-70 |
| Caprolactone/Glycolide copolymer (95:5) | 10-30 |
| Polyethylene Glycol (8000 MW) | 5-15 |
| Polyethylene Glycol (35000 MW) | 5-15 |

TABLE 23
Composition of prepared scaffold material
after post-processing (soaking and drying)

| Component | Wt % |
|---|---|
| β-TCP Powder | 75 |
| Caprolactone/Glycolide copolymer (95:5) | 25 |

TABLE 24
General composition ranges for prepared
scaffold material after post-processing

| Component | Wt % |
|---|---|
| β-TCP Powder | 50-88 |
| Caprolactone/Glycolide copolymer (95:5) | 13-50 |

### Ink Formulation #19

This 3D printing ink material is a flexible, polymer-ceramic composite material containing β-TCP that may be tethered to a therapeutic agent, such as tBMP2. This formulation contains sacrificial pore formers (water soluble polyethylene glycol components) to expose more β-TCP surface area for agent binding. This ink is a moderate viscosity formulation that can be formed into ~2-4 mm diameter pellets to be used as feedstock in a fused granular fabrication (FGF) 3D printer (e.g. Piocreat G5) with a 300-1,000 μm diameter nozzle. After 3D printing is complete, the resulting scaffold is soaked in water to dissolve the sacrificial polyethylene glycol pore former. The higher molecular weight blend of polyethylene glycol (8000 MW and 35000 MW for FGF 3D printing vs. 1500 MW for syringe-based bioprinting) results in a higher viscosity material. After 3D printing is complete, the resulting scaffold is soaked in water to dissolve the sacrificial PEG. The resulting scaffold contains 55-88% by weight β-TCP powder, and 13-50% by weight Caprolactone/Glycolide copolymer (90:10).

This ink is prepared in pellet or filament form for FGF or FFF printing, respectively. This ink is cryomilled into a fine powder, e.g., for SLS printing.

TABLE 25
Specific Ink Formulation #19

| | Wt % | Component | |
|---|---|---|---|
| Component | in ink | characteristics | Purpose |
| β-TCP Powder | 60 | Spray dried powder, 10- | Spherical β-TCP Powder |
| | | 38 micron particle size | for good extrudability |
| | | | during 3D printing, |
| | | | binding sites for |
| | | | therapeutic protein |
| Caprolactone/ | 20 | 90 mol % polycaprolactone, | Flexible polymer binder |
| Glycolide copolymer | | 10 mol % polyglycolide, | for β-TCP powder; not |
| (90:10) | | chips | water soluble |
| Polyethylene Glycol | 10 | 8,000 MW, flake, | Water soluble polymer |
| | | Tmelt = 60 C | with low melt viscosity |
| Polyethylene Glycol | 10 | 35,000 MW, flake, | Water soluble polymer |
| | | Tmelt = 60 C | with moderate melt |
| | | | viscosity |

TABLE 26
Example ink formulation #19

| Component | Wt % in ink |
|---|---|
| β-TCP Powder | 30-70 |
| Caprolactone/Glycolide copolymer (90:10) | 10-30 |
| Polyethylene Glycol (8000 MW) | 5-15 |
| Polyethylene Glycol (35000 MW) | 5-15 |

TABLE 27
Composition of prepared scaffold material
after post-processing (soaking and drying)

| Component | Wt % |
|---|---|
| β-TCP Powder | 75 |
| Caprolactone/Glycolide copolymer (90:10) | 25 |

TABLE 28
General composition ranges for prepared
scaffold material after post-processing

| Component | Wt % |
|---|---|
| β-TCP Powder | 50-88 |
| Caprolactone/Glycolide copolymer (90:10) | 13-50 |

### Ink Formulation #20

This 3D printing ink material is a flexible, polymer-ceramic composite material containing β-TCP that may be tethered to a therapeutic agent, such as tBMP2. This formulation contains sacrificial pore formers (water soluble polyethylene glycol components) to expose more β-TCP surface area for agent binding. This ink is a moderate viscosity formulation that can be formed into ~3-4 mm diameter pellets to be used as feedstock in a fused granular fabrication (FGF) 3D printer (e.g. Piocreat G5) with a 300-1,000 μm diameter nozzle. After 3D printing is complete, the resulting scaffold is soaked in water to dissolve the sacrificial polyethylene glycol pore former. The higher molecular weight blend of polyethylene glycol (8000 MW and 35000 MW for FGF 3D printing vs. 1500 MW for syringe-based bioprinting) results in a higher viscosity material. After 3D printing is complete, the resulting scaffold is soaked in water to dissolve the sacrificial PEG. The resulting scaffold contains 55-88% by weight β-TCP powder, and 13-50% by weight Poly(D,L-lactide-co-glycolide) copolymer (50:50).

This ink is prepared in pellet or filament form for FGF or FFF printing, respectively. This ink is cryomilled into a fine powder, e.g., for SLS printing.

TABLE 29
Example ink formulation #20

| Component | Wt % in ink |
|---|---|
| β-TCP Powder | 30-70 |
| Poly(D,L-lactide-co-glycolide) | 10-30 |
| copolymer (50:50) | |
| Polyethylene Glycol (8000 MW) | 5-15 |
| Polyethylene Glycol (35000 MW) | 5-15 |

### Ink Formulation #21

This 3D printing ink material is a flexible, polymer-ceramic composite material containing β-TCP that may be tethered to a therapeutic agent, such as tBMP2. This formulation contains sacrificial pore formers (water soluble polyethylene glycol components) to expose more β-TCP surface area for agent binding. This ink is a moderate viscosity formulation that can be formed into ~3-4 mm diameter pellets to be used as feedstock in a fused granular fabrication (FGF) 3D printer (e.g. Piocreat G5) with a 300-1,000 μm diameter nozzle. After 3D printing is complete, the resulting scaffold is soaked in water to dissolve the sacrificial polyethylene glycol pore former. The higher molecular weight blend of polyethylene glycol (8000 MW and 35000 MW for FGF 3D printing vs. 1500 MW for syringe-based bioprinting) results in a higher viscosity material. After 3D printing is complete, the resulting scaffold is soaked in water to dissolve the sacrificial PEG. The resulting scaffold contains 55-88% by weight β-TCP powder, and 13-50% by weight PDS.

This ink is prepared in pellet or filament form for FGF or FFF printing, respectively. This ink is cryomilled into a fine powder, e.g., for SLS printing.

TABLE 30
Example ink formulation #21

| Component | Wt % in ink |
|---|---|
| β-TCP Powder | 30-70 |
| Polydioxanone (PDS) | 10-30 |
| Polyethylene Glycol (8000 MW) | 5-15 |
| Polyethylene Glycol (35000 MW) | 5-15 |

### Ink Formulation #22

This 3D printing ink material is a flexible, polymer-ceramic composite material containing β-TCP that may be tethered to a therapeutic agent, such as tBMP2. This formulation contains sacrificial pore formers (water soluble polyethylene glycol components) to expose more β-TCP surface area for agent binding. This ink is a moderate viscosity formulation that can be formed into ~3-4 mm diameter pellets to be used as feedstock in a fused granular fabrication (FGF) 3D printer (e.g. Piocreat G5) with a 300-1,000 μm diameter nozzle. After 3D printing is complete, the resulting scaffold is soaked in water to dissolve the sacrificial polyethylene glycol pore former. The higher molecular weight blend of polyethylene glycol (8000 MW and 35000 MW for FGF 3D printing vs. 1500 MW for syringe-based bioprinting) results in a higher viscosity material. After 3D printing is complete, the resulting scaffold is soaked in water to dissolve the sacrificial PEG. The resulting scaffold contains 55-88% by weight β-TCP powder, and 13-50% by weight Dioxanone/L-lactide copolymer (90:10).

This ink is prepared in pellet or filament form for FGF or FFF printing, respectively. This ink is cryomilled into a fine powder, e.g., for SLS printing.

TABLE 31
Example ink formulation #22

| Component | Wt % in ink |
|---|---|
| β-TCP Powder | 30-70 |
| Dioxanone/L-lactide copolymer (90:10) | 10-30 |
| Polyethylene Glycol (8000 MW) | 5-15 |
| Polyethylene Glycol (35000 MW) | 5-15 |

### Ink Formulation #23

This 3D printing ink material is a flexible, polymer-ceramic composite material containing β-TCP that may be tethered to a therapeutic agent, such as tBMP2. This formulation contains sacrificial pore formers (water soluble polyethylene glycol components) to expose more β-TCP surface area for agent binding. This ink is a moderate viscosity formulation that can be formed into ~3-4 mm diameter pellets to be used as feedstock in a fused granular fabrication (FGF) 3D printer (e.g. Piocreat G5) with a 300-1,000 μm diameter nozzle. After 3D printing is complete, the resulting scaffold is soaked in water to dissolve the sacrificial polyethylene glycol pore former. The higher molecular weight blend of polyethylene glycol (8000 MW and 35000 MW for FGF 3D printing vs. 1500 MW for syringe-based bioprinting) results in a higher viscosity material. After 3D printing is complete, the resulting scaffold is soaked in water to dissolve the sacrificial PEG. The resulting scaffold contains 55-88% by weight β-TCP powder, and 13-50% by weight Glycolide/L-lactide copolymer (95:5).

This ink is prepared in pellet or filament form for FGF or FFF printing, respectively. This ink is cryomilled into a fine powder, e.g., for SLS printing.

TABLE 32
Example ink formulation #23

| Component | Wt % in ink |
|---|---|
| β-TCP Powder | 30-70 |
| Glycolide/L-lactide copolymer (95:5) | 10-30 |
| Polyethylene Glycol (8000 MW) | 5-15 |
| Polyethylene Glycol (35000 MW) | 5-15 |

### Ink Formulation #24

This 3D printing ink material is a flexible, polymer-ceramic composite material containing β-TCP that may be tethered to a therapeutic agent, such as tBMP2. This formulation contains sacrificial pore formers (water soluble polyethylene glycol components) to expose more β-TCP surface area for agent binding. This ink is a moderate viscosity formulation that can be formed into ~3-4 mm diameter pellets to be used as feedstock in a fused granular fabrication (FGF) 3D printer (e.g. Piocreat G5) with a 300-1,000 μm diameter nozzle. After 3D printing is complete, the resulting scaffold is soaked in water to dissolve the sacrificial polyethylene glycol pore former. The higher molecular weight blend of polyethylene glycol (8000 MW and 35000 MW for FGF 3D printing vs. 1500 MW for syringe-based bioprinting) results in a higher viscosity material. After 3D printing is complete, the resulting scaffold is soaked in water to dissolve the sacrificial PEG. The resulting scaffold contains 55-88% by weight β-TCP powder, and 13-50% by weight Poly-1-lactide.

This ink is prepared in pellet or filament form for FGF or FFF printing, respectively. This ink is cryomilled into a fine powder, e.g., for SLS printing.

TABLE 33
Example ink formulation #24

| Component | Wt % in ink |
|---|---|
| β-TCP Powder | 30-70 |
| Poly-1-lactide | 10-30 |
| Polyethylene Glycol (8000 MW) | 5-15 |
| Polyethylene Glycol (35000 MW) | 5-15 |

### Example 2: Ink Preparation and Scaffold Manufacture by 3D Printing

### Ink Formulation and Scaffold #1

Method: To make 5.3 cc batch of ink, 5.6 g of β-TCP powder, 1.87 g of polycaprolactone powder, 1.87 g of polyethylene glycol flake and 0.49 g sucrose were added to a glass mixing container. The glass jar was placed in a dual asymmetric centrifugal mixer (FlackTek Speedmixer) and mixed at low intensity (300 rpm) for 2 min to homogenize the powder blend before high rpm mixing. The mixer was mixed for 5 min at high intensity (3500 rpm). During mixing, the internal friction causes the polycaprolactone and polyethylene glycol to melt, changing the ink to a viscous molten liquid. This liquid phase mixing facilitates intimate dispersion of the β-TCP powder and sucrose powder into the molten polymer blend. The blended ink was allowed to cool for 10-15 min, then mixed for 5 more minutes at 3500 rpm. The mixing/cooling process was repeated for a total of four 5 min mixes at 3500 rpm. After the fourth mix at 3500 rpm, the ink charge was poured out on a glass plate and two spatulas were used to form into a roughly 1 cm diameter×6 cm long cylinder. While ink was still semi-molten, it was cut into several ~1-2 cm long pieces with straight razor.

3D Printing: solid polymer/β-TCP pieces were transferred to a 5 cc stainless steel syringe (for use in Allevi 3 Bioprinter). Extruder CORE printing head was heated to 135° C. and allowed to dwell for approximately 30 min to ensure melting of the ink. The ink was printed with 400 micron I.D. conical metallic Luer lock tip using 70 psi pressure and 7 mm/s nozzle velocity. The scaffold was 3D printed on painter's tape applied to a smooth glass or polymer surface, such as a glass microscope slide, larger glass plate, or 96 well plate lid.

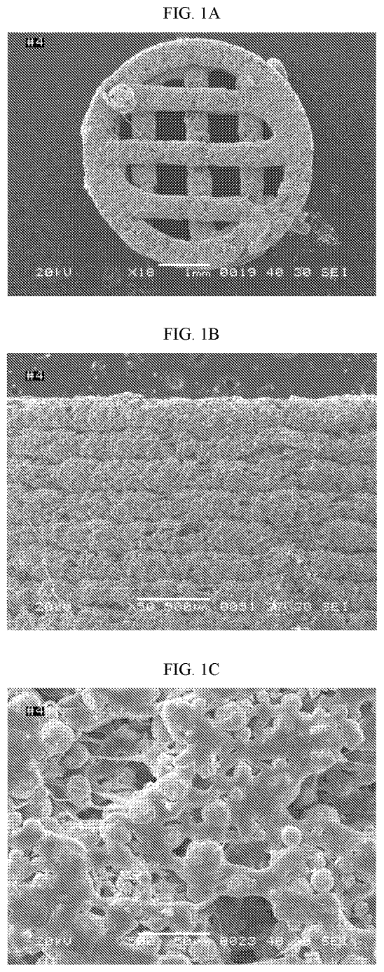

Post Processing: 3D printed structures were soaked overnight in distilled water to dissolve the polyethylene glycol and sucrose from the printed material, thus creating a porous and flexible β-TCP/polycaprolactone composite. Scaffolds were dried for at least twelve hours to ensure residual water has evaporated from the porous scaffold before binding with a therapeutic agent such as tBMP2 protein. Scaffolds were sterilized by soaking for 2-4 hours in a 70% ethanol solution and allowed to dry in biosafety cabinet for approximately 12 hours. Images of the scaffold are shown in FIGS. 1A-1C.

3D printing is also performed using a FFF 3D printer. The ink #1 is extruded into filaments and the filaments are loaded in a FFF 3D printer to generate a 3D printed scaffold. The scaffold is processed using the post processing method outlined above.

### Ink Formulation and Scaffold #2

Method: To make a 5 cc batch of ink, 5.6 g of β-TCP powder, 1.87 g of 95:5 caprolactone/glycolide copolymer pellets, and 1.87 g of polyethylene glycol flake were added to a glass mixing container. The glass jar was placed in dual asymmetric centrifugal mixer (FlackTek Speedmixer) and mixed at low intensity (300 rpm) for 2 min to homogenize the powder blend before high rpm mixing. The mixture was mixed for 5 min at high intensity (3500 rpm). During mixing, the internal friction causes the 95:5 caprolactone/glycolide copolymer and polyethylene glycol to melt, changing the ink to a viscous molten liquid. This liquid phase mixing facilitates intimate dispersion of the β-TCP powder into the molten polymer blend. The blended ink was allowed to cool for 10-15 min, and then mixed for 5 more minutes at 3500 rpm. The mixing/cooling process was repeated for a total of four 5 min mixes at 3500 rpm. After the fourth mix at 3500 rpm, the ink charge was poured out on a glass plate and two spatulas were used to form into a roughly 1 cm diameter×6 cm long cylinder. While ink was still semi-molten, it was cut into several ~1-2 cm long pieces with straight razor.

3D Printing: The solid polymer/β-TCP pieces were transferred to a 5 cc stainless steel syringe (for use in Allevi 3 Bioprinter). Extruder CORE printing head was heated to 130° C. and allowed to dwell for approximately 30 min to ensure melting of the ink. Ink was printed with 320 micron I.D. conical metallic Luer lock tip using 80 psi pressure and 6 mm/s nozzle velocity. Scaffolds were 3D printed on painter's tape applied to a smooth glass or polymer surface, such as a glass microscope slide, larger glass plate, or 96 well plate lid.

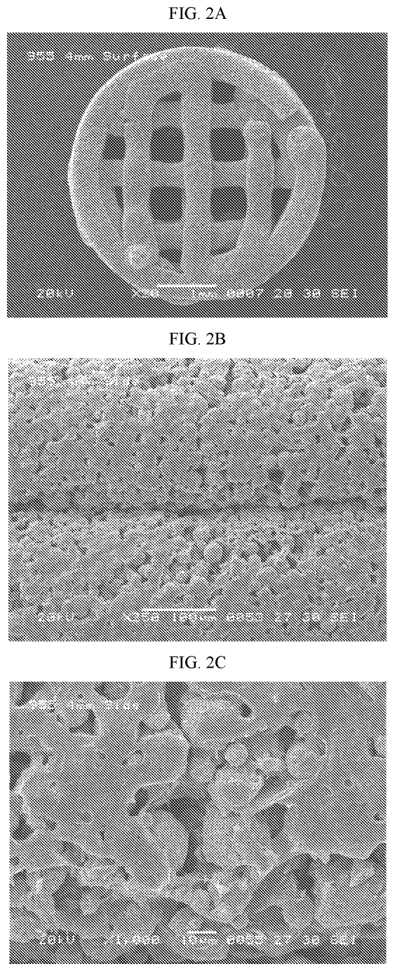

Post Processing: 3D printed structures were soaked overnight in distilled water to dissolve the polyethylene glycol from the printed material, thus creating a porous and flexible β-TCP/95:5 caprolactone/glycolide copolymer composite. The scaffolds were dried for at least twelve hours to ensure residual water evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 2A-2C.

3D printing is also performed using a FFF 3D printer. The ink #2 is extruded into filaments and the filaments are loaded in a FFF 3D printer to generate a 3D printed scaffold. The scaffold is processed using the post processing method outlined above.

### Ink Formulation and Scaffold #3

Method: To make a 5 cc batch of ink, 5.6 g of β-TCP powder, 1.87 g of 90:10 caprolactone/glycolide copolymer chips, and 1.87 g of polyethylene glycol flake were added to a glass mixing container. The mixture was placed in a glass jar in dual asymmetric centrifugal mixer (FlackTek Speedmixer) and mixed at low intensity (300 rpm) for 2 min to homogenize the powder blend before high rpm mixing. It was then mixed for 5 min at high intensity (3500 rpm). During mixing, the internal friction causes the 90:10 caprolactone/glycolide copolymer and polyethylene glycol to melt, changing the ink to a viscous molten liquid. This liquid phase mixing facilitates intimate dispersion of the β-TCP powder into the molten polymer blend. The blended ink was allowed to cool for 10-15 min and then mixed for 5 more minutes at 3500 rpm. The mixing/cooling process was repeated for a total of four 5 min mixes at 3500 rpm. After the fourth mix at 3500 rpm, the ink charge was poured out on a glass plate and two spatulas were used to form into a roughly 1 cm diameter×6 cm long cylinder. While the ink was still semi-molten, it was cut into several ~1-2 cm long pieces with straight razor.

3D Printing: the solid polymer/β-TCP pieces were transferred to a 5 cc stainless steel syringe (for use in Allevi 3 Bioprinter). Extruder CORE printing head was heated to 130° C. and allowed to dwell for approximately 30 min to ensure melting of the ink. Ink was printed with 320 micron I.D. conical metallic Luer lock tip using 45 psi pressure and 7 mm/s nozzle velocity. Scaffolds were 3D printed on painter's tape applied to a smooth glass or polymer surface, such as a glass microscope slide, larger glass plate, or 96 well plate lid.

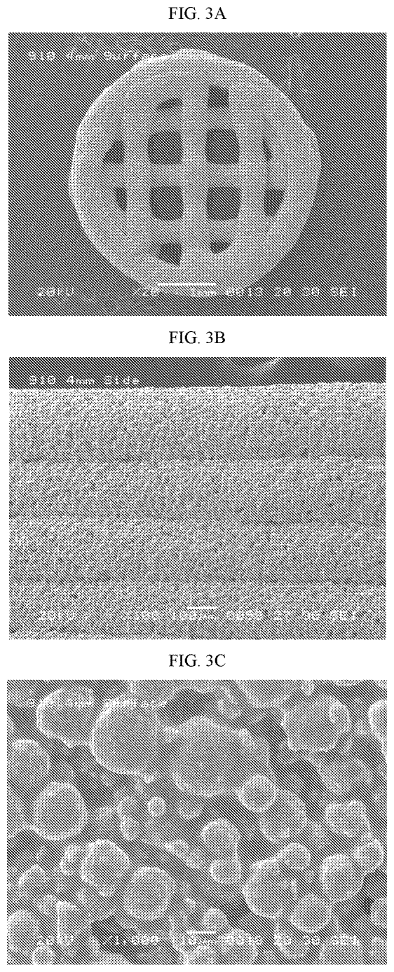

Post Processing: 3D printed structures were soaked overnight in distilled water to dissolve the polyethylene glycol from the printed material, thus creating a porous and flexible β-TCP/90:10 caprolactone/glycolide copolymer composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 3A-3C.

3D printing is also performed using a FFF 3D printer. The ink #3 is extruded into filaments and the filaments are loaded in a FFF 3D printer to generate a 3D printed scaffold. The scaffold is processed using the post processing method outlined above.

### Ink Formulation and Scaffold #4

Method: To make a 2.5 cc batch of ink, 2.8 g of β-TCP powder, 0.94 g of 50:50 poly(D,L-lactide-co-glycolide) copolymer chunks, and 0.94 g of polyethylene glycol flake were added to a glass mixing container. The glass jar was placed in dual asymmetric centrifugal mixer (FlackTek Speedmixer) and mixed at low intensity (300 rpm) for 2 min to homogenize the powder blend before high rpm mixing. It was mixed for 5 min at high intensity (3500 rpm). During mixing, the internal friction causes the 50:50 poly(D,L-lactide-co-glycolide) copolymer and polyethylene glycol to flow, changing the ink to a viscous molten liquid. This liquid phase mixing facilitates intimate dispersion of the β-TCP powder into the molten polymer blend. The blended ink was allowed to cool for 10-15 min and then mixed for 5 more minutes at 3500 rpm. The mixing/cooling process was repeated for a total of four 5 min mixes at 3500 rpm. After the fourth mix at 3500 rpm, the ink charge was poured out on a glass plate and two spatulas were used to form into a roughly 1 cm diameter×3 cm long cylinder. While ink was still semi-molten, it was cut into several ~1-2 cm long pieces with straight razor.

3D Printing: The solid polymer/β-TCP pieces were transferred to a 5 cc stainless steel syringe (for use in Allevi 3 Bioprinter). Extruder CORE printing head was heated to 85° C. and allowed to dwell for approximately 30 min to ensure melting of the ink. Ink was printed with a 400 micron I.D. conical metallic Luer lock tip using 60 psi pressure and 7 mm/s nozzle velocity. Scaffolds were 3D printed on painter's tape applied to a smooth glass or polymer surface, such as a glass microscope slide, larger glass plate, or 96 well plate lid.

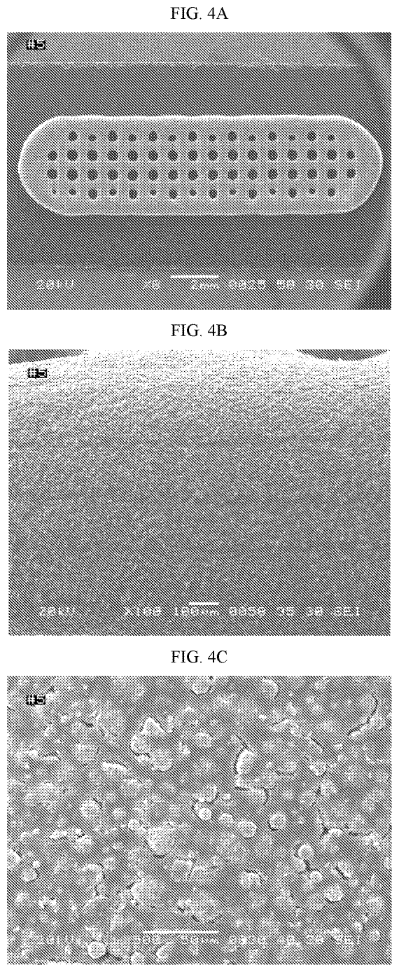

Post Processing: 3D printed structures are soaked overnight in distilled water to dissolve the polyethylene glycol from the printed material, thus creating a porous and flexible β-TCP/50:50 poly(D,L-lactide-co-glycolide) composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 4A-4C.

3D printing is also performed using a FFF 3D printer. The ink #4 is extruded into filaments and the filaments are loaded in a FFF 3D printer to generate a 3D printed scaffold. The scaffold is processed using the post processing method outlined above.

### Ink Formulation and Scaffold #5

Method: To make a 5 cc batch of ink, 5.6 g of β-TCP powder, 1.87 g of polycaprolactone powder, and 1.87 g of polyethylene glycol flake were added to a glass mixing container. The glass jar was placed in dual asymmetric centrifugal mixer (FlackTek Speedmixer) and mix at low intensity (300 rpm) for 2 min to homogenize the powder blend before high rpm mixing. It was mixed for 5 min at high intensity (3500 rpm). During mixing, the internal friction causes the polycaprolactone and polyethylene glycol to melt, changing the ink to a viscous molten liquid. This liquid phase mixing facilitates intimate dispersion of the β-TCP powder into the molten polymer blend. The molten ink was poured onto a glass plate and flatten with a spatula to approximately 3 mm thick layer for cooling. After cooling, shears were used to cut into approximately 3-4 mm pellets. This was repeated for two additional 5 cc batches to create a total 15 cc of pellets for filament extrusion.

Filament Fabrication: 15 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3S 3D printer. This filament material was printed using a 400 micron brass nozzle, 105° C. extruder temperature, and 15 mm/s print speed.

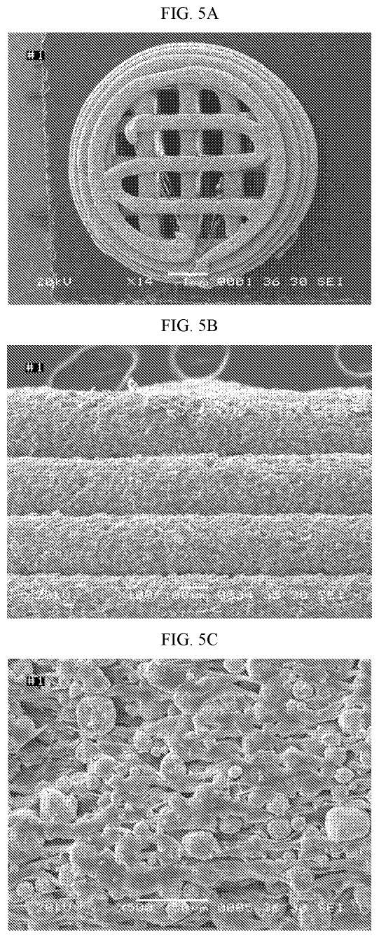

Post Processing: 3D printed structures were soaked overnight in distilled water to dissolve the polyethylene glycol from the printed material, thus creating a porous and flexible β-TCP/polycaprolactone composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 5A-5C.

### Ink Formulation and Scaffold #6

Method: To make a 5.5 cc batch of ink, 5.6 g of β-TCP powder, 1.87 g of polycaprolactone powder, 1.87 g of polyethylene glycol flake, and 1.04 g of sodium bicarbonate were added to a glass mixing container. The glass jar was placed in dual asymmetric centrifugal mixer (FlackTek Speedmixer) and mixed at low intensity (300 rpm) for 2 min to homogenize the powder blend before high rpm mixing. It was mixed for 2 min at high intensity (3500 rpm). During mixing, the internal friction causes the polycaprolactone and polyethylene glycol to melt, changing the ink to a viscous molten liquid. This liquid phase mixing facilitates intimate dispersion of the β-TCP powder and sodium bicarbonate powder into the molten polymer blend. The molten ink was poured onto a glass plate and flattened with a spatula to approximately 3 mm thick layer for cooling. After cooling, shears were used to cut into approximately 3-4 mm pellets. The was repeated for two additional 5.5 cc batches to create a total 16.5 cc of pellets for filament extrusion.

Filament Fabrication: 16.5 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3S 3D printer. This filament material was printed using a 400 micron brass nozzle, 155° C. extruder temperature, and 15 mm/s print speed.

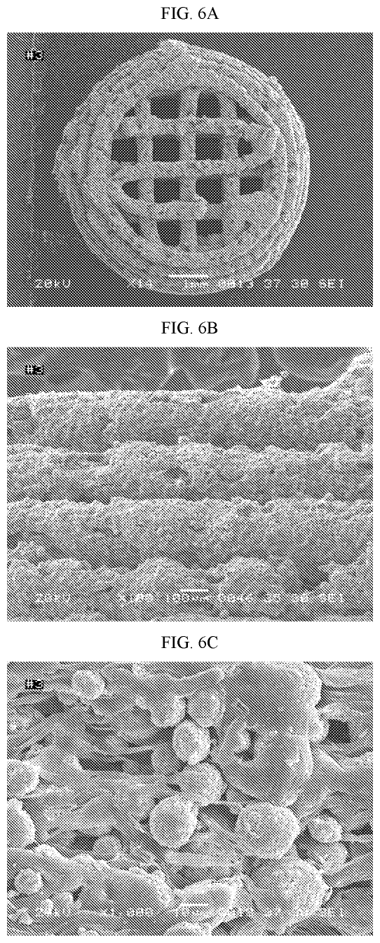

Post Processing: 3D printed structures were soaked overnight in distilled water to dissolve the polyethylene glycol and sodium carbonate (by-product of the sodium bicarbonate thermal decomposition) from the foamy printed material, thus creating a porous and flexible β-TCP/polycaprolactone composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 6A-6C.

### Ink Formulation and Scaffold #7

Method: To make a 10 cc batch of ink, 11.2 g of β-TCP powder, 3.74 g of Dioxanone/L-lactide (90:10) copolymer chips, and 3.74 g of polyethylene glycol flake were added to a glass mixing container. The glass jar was placed in a dual asymmetric centrifugal mixer (FlackTek Speedmixer) and mixed at low intensity (300 rpm) for 2 min to homogenize the powder blend before high rpm mixing. The glass jar was transferred to a hot plate and heated until it reached 185° C. (measured with IR thermometer). Next, the glass jar was immediately transferred back to the dual asymmetric centrifugal mixer and mixed for 5 min at high intensity (3500 rpm). Liquid phase mixing facilitates intimate dispersion of the β-TCP powder into the molten polymer blend. Next, the glass jar was transferred back to the hotplate and the temperature was increased to 185° C. Upon reaching the temperature, the glass jar was immediately transferred back to the dual asymmetric centrifugal mixer and mixed for 5 more minutes at 3500 rpm. The mixing/heating process was repeated for a total of four 5 min mixes at 3500 rpm. After the fourth mix at 3500 rpm, the ink charge was poured out on a glass plate and two spatulas were used to form into a roughly 1 cm diameter×6 cm long cylinder. While ink was still semi-molten, it was cut into several ~1-2 cm long pieces with straight razor.

3D Printing: solid polymer/β-TCP pieces were transferred to a 5 cc stainless steel syringe (for use in Allevi 3 Bioprinter). Extruder CORE printing head was heated to 110° C. and allowed to dwell for approximately 30 min to ensure melting of the ink. The ink was printed with 400 micron I.D. conical metallic Luer lock tip using 15 psi pressure and 10 mm/s nozzle velocity. The scaffold was 3D printed on painter's tape applied to a smooth glass or polymer surface, such as a glass microscope slide, larger glass plate, or 96 well plate lid.

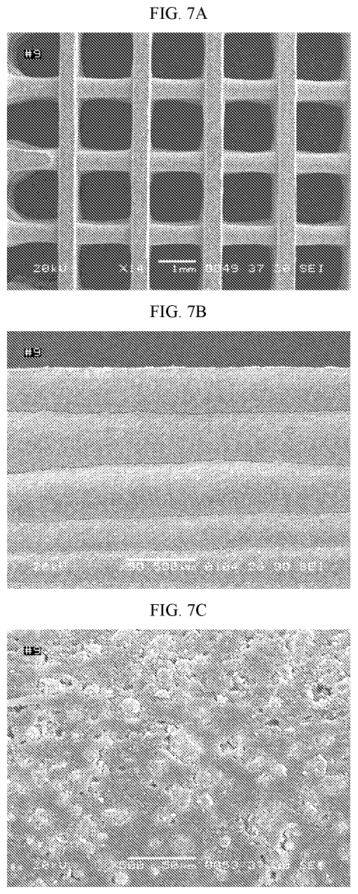

Post Processing: 3D printed structures were soaked overnight in distilled water to dissolve the polyethylene glycol from the printed material. The process created a porous and flexible β-TCP/90:10 dioxanone-L-lactide copolymer composite. Scaffolds were dried for at least twelve hours to ensure residual water has evaporated from the porous scaffold before binding with a therapeutic agent such as tBMP2 protein. Scaffolds were sterilized by soaking for 2-4 hours in a 70% ethanol solution and allowed to dry in biosafety cabinet for approximately 12 hours. Images of the scaffold are shown in FIGS. 7A-7C.

3D printing is also performed using a FFF 3D printer. The ink #7 is extruded into filaments and the filaments are loaded in a FFF 3D printer to generate a 3D printed scaffold. The scaffold is processed using the post processing method outlined above.

### Ink Formulation and Scaffold #8

Method: To make a 16 cc batch of ink, 18 g of β-TCP powder, 6 g of polycaprolactone powder, 3 g of polyethylene glycol (8,000 MW) flake and 3 g of polyethylene glycol (20,000 MW) flake were added to a teflon mixing container. The teflon container was placed in dual asymmetric centrifugal mixer (FlackTek Speedmixer) and mixed at low intensity (300 rpm) for 2 min to homogenize the powder blend before high rpm mixing. Next, the teflon container was mixed for 2.5 min at high intensity (3500 rpm). During mixing, the internal friction causes the polycaprolactone and polyethylene glycol to melt, changing the ink to a viscous molten liquid. This liquid phase mixing facilitates intimate dispersion of the β-TCP powder into the molten polymer blend. The molten ink was poured onto a glass plate and flatten with a spatula to approximately 3 mm thick layer for cooling. It was cooled for 10-15 min. The mixture was returned to the teflon container and mixed for 2.5 more minutes at a high intensity (3500 rpm). The mixing/cooling process was repeated for a total of four 2.5 min mixes at 3500 rpm. After the ink was cooled after the fourth and final mix, shears were used to cut it into approximately 3-4 mm pellets.

Filament Fabrication: 16 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3S 3D printer. This filament material was printed using a 400 micron brass nozzle, 105° C. extruder temperature, and 15 mm/s print speed.

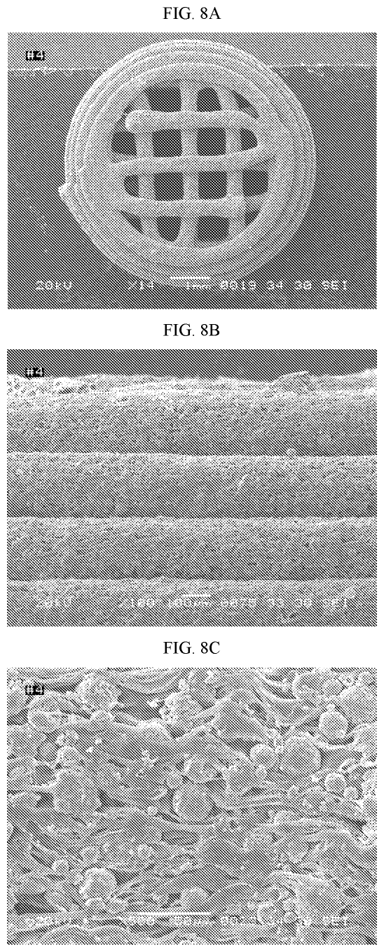

Post Processing: 3D printed structures were soaked overnight in distilled water to dissolve the polyethylene glycol from the printed material, thus creating a porous and flexible β-TCP/polycaprolactone composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 8A-8C.

### Ink Formulation and Scaffold #9

Method: To make a 16.3 cc batch of ink, 17.1 g of β-TCP powder, 5.7 g of polycaprolactone powder, 2.85 g of polyethylene glycol (8,000 MW) flake, 2.85 g of polyethylene glycol (20,000 MW) flake, and 1.5 g of sucrose were added to a teflon mixing container. The teflon container was placed in dual asymmetric centrifugal mixer (FlackTek Speedmixer) and mixed at low intensity (300 rpm) for 2 min to homogenize the powder blend before high rpm mixing. Next, the teflon container was mixed for 2 min at high intensity (3500 rpm). During mixing, the internal friction causes the polycaprolactone and polyethylene glycol to melt, changing the ink to a viscous molten liquid. This liquid phase mixing facilitates intimate dispersion of the β-TCP powder into the molten polymer blend. The molten ink was poured onto a glass plate and flatten with a spatula to approximately 3 mm thick layer for cooling. It was cooled for 10-15 min. The mixture was returned to the teflon container and mixed for 2 more minutes at a high intensity (3500 rpm). The mixing/cooling process was repeated for a total of four 2 min mixes at 3500 rpm. After the ink was cooled after the fourth and final mix, shears were used to cut it into approximately 3-4 mm pellets.

Filament Fabrication: 16.3 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3S 3D printer. This filament material was printed using a 400 micron brass nozzle, 140° C. extruder temperature, and 10 mm/s print speed.

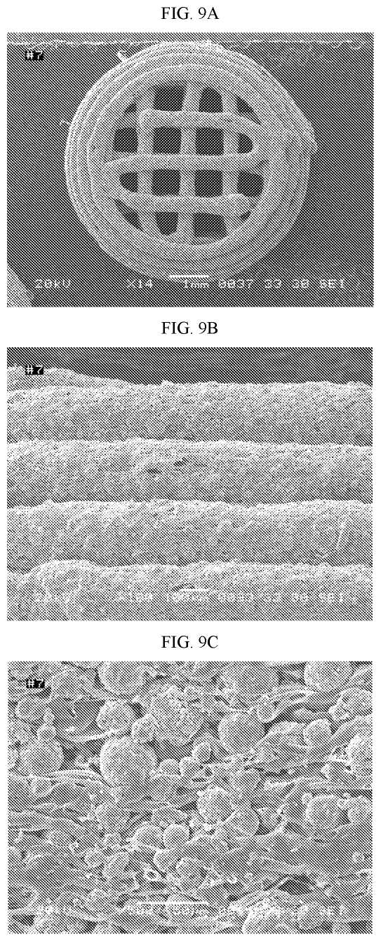

Post Processing: 3D printed structures were soaked overnight in distilled water to dissolve the polyethylene glycol and sucrose from the printed material, thus creating a porous and flexible β-TCP/polycaprolactone composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 9A-9C.

### Ink Formulation and Scaffold #10

Method: To make a 16 cc batch of ink, 18 g of β-TCP powder, 6 g of caprolactone/glycolide copolymer (95:5) pellets, 3 g of polyethylene glycol (8,000 MW) flake and 3 g of polyethylene glycol (20,000 MW) flake were added to a teflon mixing container. The teflon container was placed in dual asymmetric centrifugal mixer (FlackTek Speedmixer) and mixed at low intensity (300 rpm) for 2 min to homogenize the powder blend before high rpm mixing. Next, the teflon container was mixed for 2.5 min at high intensity (3500 rpm). During mixing, the internal friction causes the caprolactone/glycolide copolymer (95:5) and polyethylene glycol to melt, changing the ink to a viscous molten liquid. This liquid phase mixing facilitates intimate dispersion of the β-TCP powder into the molten polymer blend. The molten ink was poured onto a glass plate and flatten with a spatula to approximately 3 mm thick layer for cooling. It was cooled for 10-15 min. The mixture was returned to the teflon container and mixed for 4 more minutes at a high intensity (3500 rpm). The mixing/cooling process was repeated for a total of one 2.5 minute mix and three 4 min mixes at 3500 rpm. After the ink was cooled after the fourth and final mix, shears were used to cut it into approximately 3-4 mm pellets.

Filament Fabrication: 16 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3S 3D printer. This filament material was printed using a 400 micron brass nozzle, 150° C. extruder temperature, and 10 mm/s print speed.

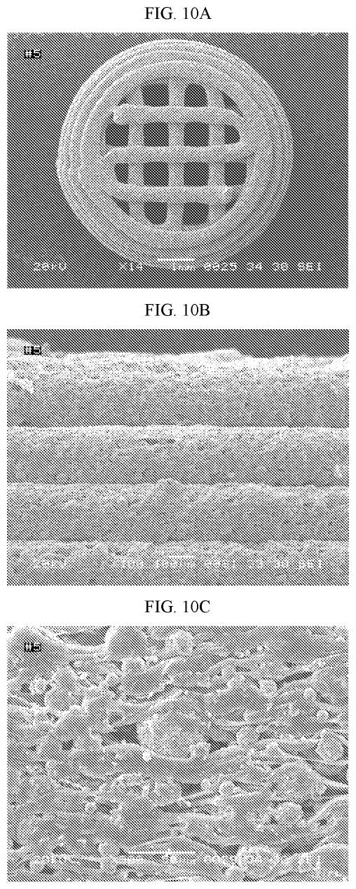

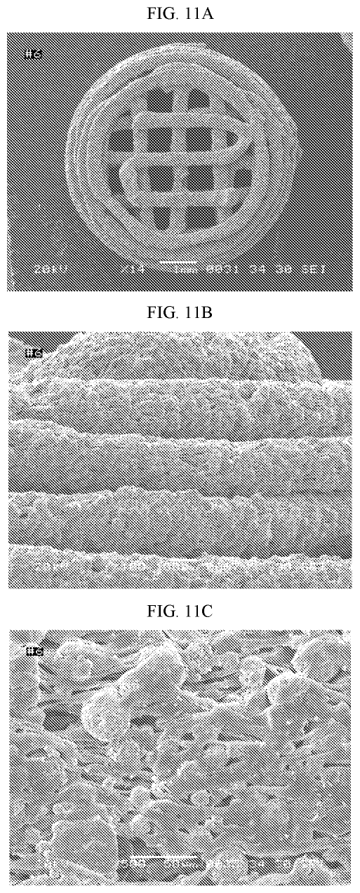

Post Processing: 3D printed structures were soaked overnight in distilled water to dissolve the polyethylene glycol from the printed material, thus creating a porous and flexible β-TCP/caprolactone/glycolide copolymer (95:5) composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 10A-10C.

### Ink Formulation and Scaffold #11

Method: To make a 16 cc batch of ink, 18 g of β-TCP powder, 6 g of caprolactone/glycolide copolymer (90:10) chips, 3 g of polyethylene glycol (8,000 MW) flake and 3 g of polyethylene glycol (20,000 MW) flake were added to a teflon mixing container. The teflon container was placed in dual asymmetric centrifugal mixer (FlackTek Speedmixer) and mixed at low intensity (300 rpm) for 2 min to homogenize the powder blend before high rpm mixing. Next, the teflon container was mixed for 2 min at high intensity (3500 rpm). During mixing, the internal friction causes the caprolactone/glycolide copolymer (90:10) and polyethylene glycol to melt, changing the ink to a viscous molten liquid. This liquid phase mixing facilitates intimate dispersion of the β-TCP powder into the molten polymer blend. The molten ink was poured onto a glass plate and flatten with a spatula to approximately 3 mm thick layer for cooling. It was cooled for 10-15 min. The mixture was returned to the teflon container and mixed for 5 more minutes at a high intensity (3500 rpm). The mixing/cooling process was repeated for a total of one 2 minute mix and three 5 min mixes at 3500 rpm. After the ink was cooled after the fourth and final mix, shears were used to cut it into approximately 3-4 mm pellets.

Filament Fabrication: 16 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3S 3D printer. This filament material was printed using a 400 micron brass nozzle, 150° C. extruder temperature, print bed temperature of 40° C., and 10 mm/s print speed.

Post Processing: 3D printed structures were soaked overnight in distilled water to dissolve the polyethylene glycol from the printed material, thus creating a porous and flexible β-TCP/caprolactone/glycolide copolymer (90:10) composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 11A-11C.

### Ink Formulation and Scaffold #12

Method: To make a 8 cc batch of ink, 9 g of β-TCP powder, 3 g of Poly(D,L-lactide-co-glycolide) copolymer (50:50) chunks, 1.5 g of polyethylene glycol (8,000 MW) flake, and 1.5 g of polyethylene glycol (20,000 MW) flake were added to a teflon mixing container. The teflon container was placed in dual asymmetric centrifugal mixer (FlackTek Speedmixer) and mixed at low intensity (300 rpm) for 2 min to homogenize the powder blend before high rpm mixing. Next, the teflon container was mixed for 2 min at high intensity (3500 rpm). During mixing, the internal friction causes the Poly(D,L-lactide-co-glycolide) copolymer (50:50) and polyethylene glycol to melt, changing the ink to a viscous molten liquid. This liquid phase mixing facilitates intimate dispersion of the β-TCP powder into the molten polymer blend. The molten ink was poured onto a glass plate and flatten with a spatula to approximately 3 mm thick layer for cooling. It was cooled for 10-15 min. The mixture was returned to the teflon container and mixed for 2.5 more minutes at a high intensity (3500 rpm). The mixing/cooling process was repeated for a total of one 2 minute mix and two 2.5 min mixes at 3500 rpm. After the ink was cooled after the third and final mix, shears were used to cut it into approximately 3-4 mm pellets.

Filament Fabrication: 8 cc of 3-4 mm pellets were loaded into the hopper of a filament extruder (Filabot EX2 Filament Extruder). A 3× length extended melt filter nozzle (with filter screens removed) with 1.75 mm diameter hole size was used. A cooling fan was placed near the extrusion nozzle to speed up solidification of the extruded filament. The 1.75 mm diameter filament was extruded at extrusion temperature to 62° C. and speed at ½ on the analog dial (approximately 1 cm/second extrusion speed).

3D Printing: The 1.75 mm filament was loaded in a Prusa i3 MK3S 3D printer. This filament material was printed using a 400 micron brass nozzle, 140° C. extruder temperature, and 10 mm/s print speed.

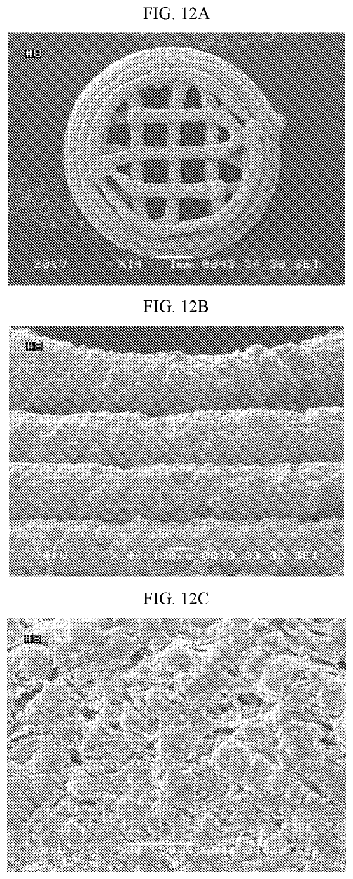

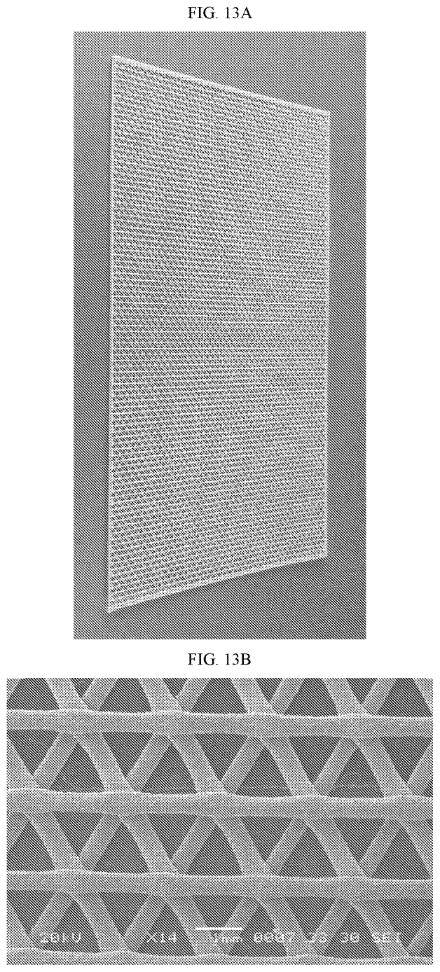

Post Processing: 3D printed structures were soaked overnight in distilled water to dissolve the polyethylene glycol from the printed material, thus creating a porous and flexible β-TCP/Poly(D,L-lactide-co-glycolide) copolymer (50:50) composite. Scaffolds were dried for at least twelve hours to ensure residual water has evaporated from the porous scaffold before binding with a therapeutic agent like tBMP2. Scaffolds were sterilized by soaking for 2-4 hours in a 70% ethanol solution and allowed to dry in biosafety cabinet for approximately 12 hours. Images of the scaffold are shown in FIGS. 12A-12C.

### Ink Formulation and Scaffold #13

Ink #13 is printed using a syringe-based melt extrusion printing method, for example, using methods as described for printing inks #1-#4. Ink #13 is printed using fused filament fabrication 3D printing, for example, using methods as described for printing ink #5 and ink #6.

### Ink Formulation and Scaffold #14

Ink #14 is printed using a syringe-based melt extrusion printing method, for example, using methods as described for printing inks #1-#4. Ink #14 is printed using fused filament fabrication 3D printing, for example, using methods as described for printing ink #5 and ink #6.

### Ink Formulation and Scaffold #15

Ink #9 is printed using a syringe-based melt extrusion printing method, for example, using methods as described for printing inks #1-#4. Ink #15 is printed using fused filament fabrication 3D printing, for example, using methods as described for printing ink #5 and ink #6.

### Ink Formulation and Scaffold #16

Method: To make a 16 cc batch of ink, 18 g of β-TCP powder, 6 g of polycaprolactone powder, 3 g of polyethylene glycol (8,000 MW) flake and 3 g of polyethylene glycol (35,000 MW) flake were added to a teflon mixing container. The teflon container was placed into a dual asymmetric centrifugal mixer (FlackTek Speedmixer) and mixed at low intensity (300 rpm) for 2 min to homogenize the powder blend before high rpm mixing. The powder blend was then mixed for 2.5 min at high intensity (3500 rpm). During mixing, the internal friction causes the polycaprolactone and polyethylene glycol to melt, changing the ink to a viscous molten liquid. This liquid phase mixing facilitates intimate dispersion of the β-TCP powder into the molten polymer blend. The molten ink was poured onto a glass plate and flattened with a spatula to approximately 3 mm thick layer for cooling. The ink was allowed to cool for 10-15 min. After this cooling step, the ink was mixed again for 2.5 min at 3500 rpm. This mixing and cooling process (10-15 min cooling followed by 2.5 min mixing at 3500 rpm) was repeated four times. After the ink was cooled following the fourth and final mix, shears were used to cut the ink into about 4 mm to about 6 mm feedstock squares for pellet formation.

Pellet Fabrication: A 850 μm diameter nozzle was attached to the metal barrel of a heated, pneumatic extrusion device (e.g. an Allevi 3 bioprinter with a 5 mL stainless steel syringe barrel). 3-4 cc of 4-6 mm feedstock squares were loaded into the barrel. The barrel was heated to about 80 to about 120° C. temperature, for example 100° C. After 15 min, additional feedstock squares were added to the barrel and pushed down with a spatula into the molten material until the barrel was full. After 10 additional minutes, the ink was checked to ensure full melting. The pneumatic pressure of the pneumatic extrusion device was then set to about 50 to about 100 psi, for example 55 psi. A platform was installed below the extrusion tip as a substrate on which to extrude pellets. In one example, the platform can be smooth silicone sheet material. A manually generated Marlin G-code was used to extrude up to 120 pellets in a rectangular array, resulting in pellets having about 3 mm to about 4 mm diameter and being roughly equiaxed.

3D Printing: The pellets were then loaded into the hopper of a Piocreat G5 FGF 3D printer. The material was printed using a nozzle of about 300 μm to about 1000 μm at about 105 to about 145° C. nozzle temperature using an about 5 mm/s to about 30 mm/s printing speed.

Post Processing: The 3D printed structures were soaked for at least 16 hours in distilled water to dissolve the polyethylene glycol from the printed material, thus creating a porous and flexible β-TCP/polycaprolactone composite. The scaffolds were dried for at least twelve hours to ensure residual water has evaporated from the porous scaffold before binding with TBMP2 protein. Scaffolds were sterilized by soaking for 2-4 hours in a 70% ethanol solution and allowed to dry in a biosafety cabinet for about 12 hours. Images of example scaffolds are shown in FIGS. 13A-13D and 14A-14D. An example scaffold is a gyroid scaffold, which has an interconnected, isotropic porous structure with no sharp corners or straight edges. These properties enable sufficient fluid transport within the scaffold structure and are amenable to cell attachment.

### Ink Formulation and Scaffold #17-#24

Inks #17 and #20-24 are printed into scaffolds using the pellet-based method as described for ink formulation and scaffold #16.

### Ink Formulation and Scaffold #18

Method: To make a 16 cc batch of ink, 18 g of β-TCP powder, 6 g of Caprolactone/Glycolide copolymer (95:5) pellets, 3 g of polyethylene glycol (8,000 MW) flake and 3 g of polyethylene glycol (35,000 MW) flake was added to a teflon mixing container. The teflon container was placed in dual asymmetric centrifugal mixer (FlackTek Speedmixer) and mixed at low intensity (300 rpm) for 2 min to homogenize the powder blend before high rpm mixing. The composition was mixed for 1.5 min at high intensity (3500 rpm). During mixing, the internal friction caused the caprolactone/glycolide copolymer and polyethylene glycol to melt, changing the ink to a viscous molten liquid. This liquid phase mixing facilitated intimate dispersion of the β-TCP powder into the molten polymer blend. The molten ink was poured onto a glass plate and flattened with a spatula to an approximately 3 mm thick layer for cooling. The ink was cooled for 10-15 min, and mixed for 1.5 more minutes at 3500 rpm. This mixing/cooling process was repeated for a total of four 1.5-minute mixes at 3500 rpm. After the ink was cooled following the fourth and final mix, shears were used to cut the ink into approximately 4-6 mm squares for pelletizing feedstock.

Pellet Fabrication: An 850 μm diameter nozzle was attached to the metal barrel of a heated, pneumatic extrusion device (e.g. an Allevi 3 bioprinter with a 5 mL stainless steel syringe barrel) and 3-4 cc of 4-6 mm ink feedstock squares were loaded into the barrel. The barrel was heated to 80-120° C. temperature, for example 100° C., and left for 15 minutes. After 15 minutes more feedstock was added to the barrel and pushed down with a spatula into molten material until the barrel was full and left for 10 minutes. After 10 minutes the barrel was checked to ensure the ink was fully melted. The pneumatic pressure of the pneumatic extrusion device was then set to about 50-100 psi, for example 55 psi. A platform was installed below the extrusion tip as a substrate on which to extrude pellets. In one example, the platform can be smooth silicone sheet material. A manually generated Marlin G-code was used to extrude up to 120 pellets in a rectangular array, resulting in pellets having about 2 mm to about 3 mm diameter and being roughly equiaxed.

3D Printing: The pellets were then loaded into the hopper of a Piocreat G5 FGF 3D printer. The material was printed using a nozzle of about 300 μm to about 1000 μm at about 105 to about 145° C. nozzle temperature using an about 5 mm/s to about 30 mm/s printing speed.

Post Processing: The 3D printed structures were soaked for at least 16 hours in distilled water to dissolve the polyethylene glycol from the printed material, thus creating a porous and flexible β-TCP and caprolactone/glycolide copolymer composite. The scaffolds were dried for at least twelve hours to ensure residual water has evaporated from the porous scaffold before binding with TBMP2 protein. Scaffolds were sterilized by soaking for 2-4 hours in a 70% ethanol solution and allowed to dry in a biosafety cabinet for about 12 hours. Images of example scaffolds are shown in FIGS. 15A-15B. After soaking and drying, the scaffold contained 75 Wt % β-TCP powder and 25 Wt % Caprolactone/Glycolide copolymer (95:5).

### Ink Formulation and Scaffold #19

Method: To make a 16 cc batch of ink, 18 g of β-TCP powder, 6 g of Caprolactone/Glycolide copolymer (90:10) chips, 3 g of polyethylene glycol (8,000 MW) flake and 3 g of polyethylene glycol (35,000 MW) flake was added to a teflon mixing container. The teflon container was placed in dual asymmetric centrifugal mixer (FlackTek Speedmixer) and mixed at low intensity (300 rpm) for 2 min to homogenize the powder blend before high rpm mixing. The composition was mixed for 1.5 min at high intensity (3500 rpm). During mixing, the internal friction caused the caprolactone/glycolide copolymer and polyethylene glycol to melt, changing the ink to a viscous molten liquid. This liquid phase mixing facilitated intimate dispersion of the β-TCP powder into the molten polymer blend. The molten ink was poured onto a glass plate and flattened with a spatula to an approximately 3 mm thick layer for cooling. The ink was cooled for 10-15 min, and mixed for 1.5 more minutes at 3500 rpm. This mixing/cooling process was repeated for a total of four 1.5-minute mixes at 3500 rpm. After the ink was cooled following the fourth and final mix, shears were used to cut the ink into approximately 4-6 mm squares for pelletizing feedstock.

Pellet Fabrication: An 850 μm diameter nozzle was attached to the metal barrel of a heated, pneumatic extrusion device (e.g. an Allevi 3 bioprinter with a 5 mL stainless steel syringe barrel) and 3-4 cc of 4-6 mm ink feedstock squares were loaded into the barrel. The barrel was heated to 80-120° C. temperature, for example 100° C., and left for 15 minutes. After 15 minutes more feedstock was added to the barrel and pushed down with a spatula into molten material until the barrel was full and left for 10 minutes. After 10 minutes the barrel was checked to ensure the ink was fully melted. The pneumatic pressure of the pneumatic extrusion device was then set to about 50-100 psi, for example 55 psi. A platform was installed below the extrusion tip as a substrate on which to extrude pellets. In one example, the platform can be smooth silicone sheet material. A manually generated Marlin G-code was used to extrude up to 120 pellets in a rectangular array, resulting in pellets having about 2 mm to about 3 mm diameter and being roughly equiaxed.

3D Printing: The pellets were then loaded into the hopper of a Piocreat G5 FGF 3D printer. The material was printed using a nozzle of about 300 μm to about 1000 μm at about 105 to about 145° C. nozzle temperature using an about 5 mm/s to about 30 mm/s printing speed.

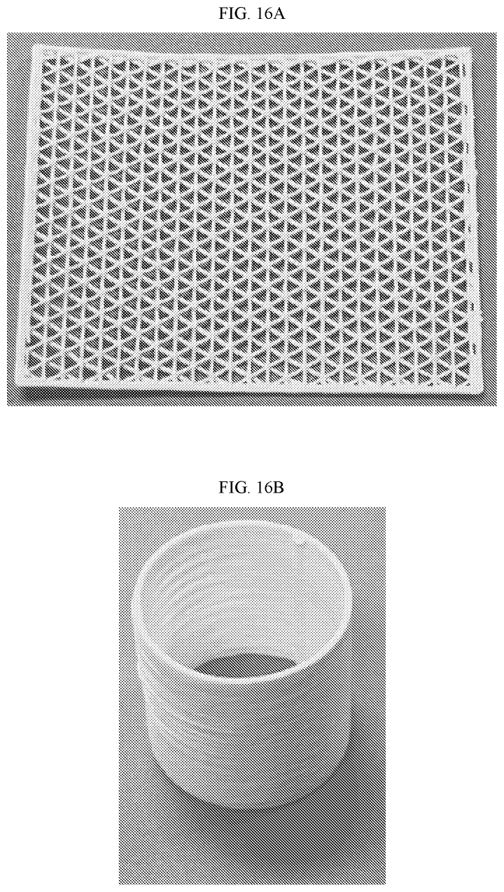

Post Processing: The 3D printed structures were soaked for at least 16 hours in distilled water to dissolve the polyethylene glycol from the printed material, thus creating a porous and flexible β-TCP and caprolactone/glycolide copolymer composite. The scaffolds were dried for at least twelve hours to ensure residual water has evaporated from the porous scaffold before binding with TBMP2 protein. Scaffolds were sterilized by soaking for 2-4 hours in a 70% ethanol solution and allowed to dry in a biosafety cabinet for about 12 hours. Images of example scaffolds are shown in FIGS. 16A-16B. After soaking and drying, the scaffold contained 75 Wt % β-TCP powder and 25 Wt % Caprolactone/Glycolide copolymer (90:10).

### Structure Properties

Physical properties of example scaffolds made using inks #1-#12 and 18-19 were determined and are outlined in Table 34.

Physical properties of example scaffolds made using inks #16, 18, and 19 were determined and outline in Table 35.

TABLE 34
Properties of Examples Scaffolds

| | Density | Open | Strut |
|---|---|---|---|
| Ink | (g/cm³) | Porosity (%) | Diameter (μm) |
| Ink #1 | 1.45 | 30 | 434 |
| Ink #2 | 1.56 | 23 | 397 |
| Ink #3 | 1.70 | 25 | 569 |
| Ink #4 | 1.49 | 32 | 464 |
| Ink #5 | 1.23 | 39 | 378 |
| Ink #6 | 1.22 | 39 | 392 |
| Ink #7 | 1.50 | 33 | 499 |
| Ink #8 | 1.22 | 37 | 359 |
| Ink #9 | 1.23 | 37 | 351 |
| Ink #10 | 1.28 | 37 | 374 |
| Ink #11 | 1.45 | 29 | 335 |
| Ink #12 | 1.30 | 37 | 364 |
| Ink # 18 | 1.51 | 28 | 406 |
| Ink # 19 | 1.54 | 29 | 460 |

TABLE 35
Properties of Example Scaffolds

| | | | Open | Strut |
|---|---|---|---|---|
| | | Density | Porosity | Diameter |
| Ink | Scaffold | (g/cm³) | (%) | (μm) |
| Ink #16 | Flexible 3-layer membrane 3D | 1.24 | 32 | 320 |
| | printed with a 300 μm nozzle | | | |
| Ink #16 | Flexible 3-layer membrane 3D | 1.31 | 35 | 394 |
| | printed with a 400 μm nozzle | | | |
| Ink #16 | Flexible 3-layer membrane 3D | 1.38 | 30 | 814 |
| | printed with a 800 μm nozzle | | | |
| Ink # 18 | Flexible 3-layer membrane 3D | 1.51 | 28 | 406 |
| | printed with a 400 μm nozzle | | | |
| Ink # 19 | Flexible 3-layer membrane 3D | 1.54 | 29 | 460 |
| | printed with a 400 μm nozzle | | | |

The structures of this example are tested using Brunauer-Emmett-Teller (BET) surface area analysis by gas physisorption.

A compression test is also performed on the structures.

### Example 3: Therapeutic Agent

A chimeric polypeptide comprising the BMP therapeutic peptide connected to five beta-tricalcium phosphate binding peptides was expressed and purified using standard expression and purification methods. The chimeric polypeptide is referred to as tBMP-2 and has the following sequence:

(SEQ ID NO: 434)
MPIGSLLADTTHHRPWTVIGESTHHRPWSIIGESSHHKPFTGLGDTTH
HRPWGILAESTHHKPWTASGAGGSEGGGSEGGTSGATGAGTSTSGGGA
STGGGTGQAKHKQRKRLKSSCKRHPLYVDFSDVGWNDWIVAPPGYHAF
YCHGECPFPLADHLNSTNHAIVQTLVNSVNSKIPKACCVPTELSAISM
LYLDENEKVVLKNYQDMVVEGCGCR.

### Example 4: Device Manufacture

The 3D printed structure “Flexible 3-layer membrane 3D printed with a 400 μm nozzle” of Example 2 was combined with the tBMP-2 therapeutic agent of Example 3 to create a device. The scaffold was combined with tBMP-2 in a binding solution and unbound tBMP-2 was washed off the scaffold. The resulting device comprised the scaffold with bound tBMP-2. The device was lyophilized. Similar devices are prepared using 3D printed structures and growth factors herein.

### Example 5: Animal Models

One or more devices of Example 4 are tested in an animal model to demonstrate bone regeneration through μCT imaging and histological analysis. Indications can include lumbar spinal fusion (a 3D printed insert for spinal fusion cage), posterolateral (PLF) spine fusion (3D printed scaffold that spans transverse processes), tibial segmental defects (a 3D printed scaffold based on patient CT data), and/or alveolar ridge augmentation (a 3D printed thin barrier membrane).

The first study is a lapine posterolateral fusion model. The objective of this study is to evaluate the in vivo performance of test devices at varying concentrations of growth factor and masses of scaffold. The test groups are evaluated for spine fusion rate, new bone formation, and residual graft using radiographic plain films, microCT, biomechanical and histological endpoints at 8 weeks following implantation.

A dorsal midline skin incision, approximately 15 centimeters long, is made in each rabbit from L1 to the sacrum, and then the fascia and muscle are incised over the L5-L6 transverse processes (TPs). The TPs are then decorticated with a high-speed burr. The test devices are placed over the transverse processes and fascia and skin are closed and stapled. To ensure the animals are comfortable, analgesics are administered according to IACUC approval. Animals are fed ad libitum and allowed to move about their cages without restriction. No postoperative immobilization devices are used. During the following weeks, the animals are observed closely and given additional pain medication based on their mobility, diet, disposition, and general activity that would signify increased pain. The rabbits are radiographed postoperatively and at 8 weeks. MicroCT morphometry analysis is performed using a region of interest (ROI) placed across the fusion site and areas of bone are calculated. Fusion sites of each animal are processed for histology at 8 weeks.

The second study is a sheep interbody fusion model to evaluate test devices in the interbody space of sheep lumbar spine. Sheep undergo interbody fusion through a lateral approach. Implanted motion segments are stabilized with pedicle screw and rod fixation. PEEK interbody spacers are placed in the interbody space following discectomy and endplate prep. Spacers are filled with test device. Sheep are euthanized at 6 months post-op after review of in-life MDCTs at 4, 8, and 12 weeks post operation. After euthanasia, μCT, manual palpation of implanted motion segments, calcified (plastic embedded) standard histology (H&E and Trichrome), IHC (growth factor), and histomorphometry is performed.

### Example 6: Cytotoxicity Assay

An in vitro cytotoxicity assay was performed to determine cell response, specifically toxic effects, when exposed to extracts from the 3D printed scaffolds. The outcome of this in vitro study provides insight on the toxicity of the biomaterial components of the scaffolds (shown in Table 36) when implanted in the body. All cytotoxicity tests were performed using guidance from the International Organization for Standardization (ISO) 10993-5:2009 standard.

TABLE 36
Scaffold Fomulations for Cytotoxicity Assay

| Sample name and description | Material |
|---|---|
| 95:5 (3D printed 3-layer | Ink Formulation #2 (β-TCP and |
| membrane scaffold) | Caprolactone/Glycolide copolymer |
| | (95:5) composite) |
| 90:10 (3D printed 3-layer | Ink Formulation #3 (β-TCP and |
| membrane scaffold) | Caprolactone/Glycolide copolymer |
| | (90:10) composite) |
| OT (3D printed 3-layer | β-TCP and Polycaprolactone |
| membrane scaffold) | composite |
| HDPE (negative control) | High Density Polyethylene |
| ZDEC (positive control) | 0.1% Zincdiethyldithiocarbamate |

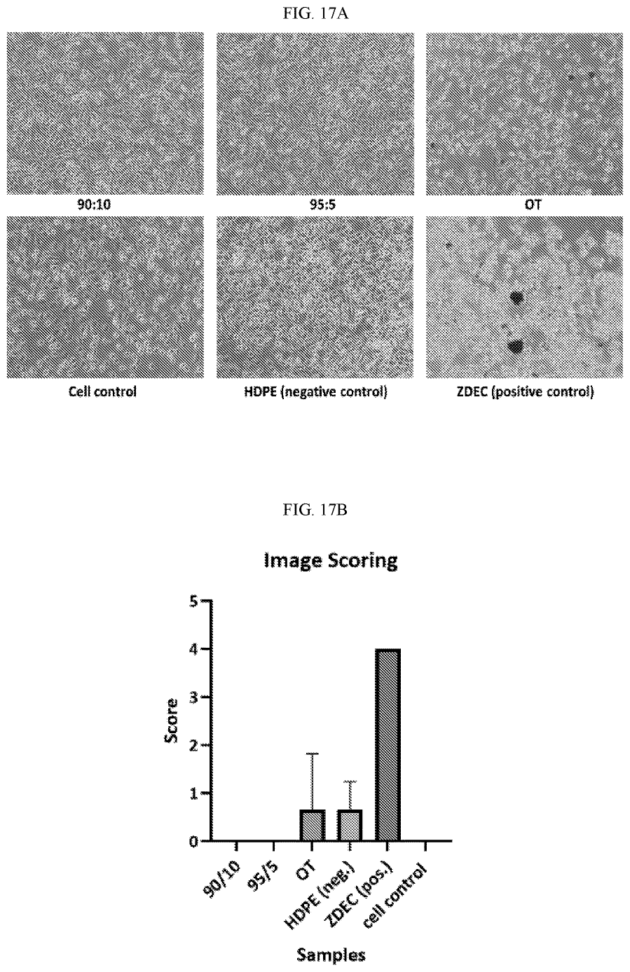

Briefly, L929 mouse fibroblast cells were seeded in 24-well plates at seeding density of 1×10⁵ cells/per well and placed in a humidified incubator at 37° C. and 5% CO₂ overnight. Sterile scaffold extracts (scaffolds shown in table 36) were prepared by first submerging scaffolds in media for 24 hours then adding these extracts to the 24-well plates seeded with the L929 mouse fibroblast cell line. After 24 hours, visual examination of cells exposed to the extracts was used to determine if there was a cytotoxic cell response after cells were exposed to scaffolds. Cytotoxicity was considered present if cells were detached, lysed, or had changes in morphology. Microscopy images of cells exposed to the scaffolds of table 36 are shown in FIG. 17A. The cells were examined and cytotoxicity scores were calculated according to the following cytotoxicity scale defined in ISO 10993-5:2009 standard: Scale 0=Non cytotoxic, Scale 1=slightly cytotoxic, Scale 2=mildly cytotoxic, Scale 3=moderately cytotoxic, Scale 4=severely cytotoxic.

High Density Polyethylene (HDPE) (negative control) scored an average of less than 1 which is not cytotoxic to slightly cytotoxic. 0.1% Zincdiethyldithiocarbamate (ZDEC) (positive control) scored an average of 4 which is severely cytotoxic, and had widespread cell detachment. 95:5 and 90:10 scored 0 indicating that they are non cytotoxic. The OT sample scored less than 1, indicating that it is non cytotoxic to slightly cytotoxic. There was minimal to no cell detachment in all of the 3D printed scaffold samples and they all were similar in appearance to the cell-only control. Image scores are shown in FIG. 17B.

1. An ink formulation comprising about 55% to about 65% by weight beta-tricalcium phosphate (βTCP), about 15% to about 25% by weight caprolactone/glycolide copolymer, about 5% to about 15% polyethylene glycol (PEG) having a molecular weight from about 500 g/mol to about 15,000 g/mol, and about 5% to about 15% PEG having a molecular weight from about 25,000 g/mol to about 50,000 g/mol.

2. The ink formulation of claim 1, comprising about 60% by weight βTCP, about 20% by weight caprolactone/glycolide copolymer, about 10% by weight PEG having a molecular weight from about 500 g/mol to about 15,000 g/mol, and about 10% PEG having a molecular weight from about 25,000 g/mol to about 50,000 g/mol.

3. The ink formulation of claim 2, comprising about 60% by weight βTCP, about 20% by weight caprolactone/glycolide copolymer, about 10% by weight PEG having a molecular weight of about 8,000 g/mol, and about 10% PEG having a molecular weight of about 35,000 g/mol.

4. The ink formulation of claim 1, wherein the caprolactone/glycolide copolymer is caprolactone/glycolide copolymer (95:5).

5. The ink formulation of claim 1, wherein the caprolactone/glycolide copolymer is caprolactone/glycolide copolymer (90:10).

6. A method of preparing a three-dimensional structure, the method comprises performing additive manufacturing with the ink formulation of claim 1.

7. The method of claim 6, wherein the ink formulation is in the form of a pellet.

8. The method of claim 6, wherein the additive manufacturing comprises fused granular fabrication (FGF).

9. A structure prepared by additive manufacturing of the ink formulation of claim 1.

10. A device comprising a therapeutic agent and the structure of claim 9.

11. The device of claim 10, wherein the therapeutic agent is non-covalently bound to the structure.

12. The device of claim 10, wherein the therapeutic agent comprises a growth factor.

13. The device of claim 12, wherein the growth factor is selected from Table 1.

14. The device of claim 10, wherein the therapeutic agent comprises a bone morphogenetic protein (BMP).

15. The device of claim 10, wherein the therapeutic agent comprises a targeting moiety, and the targeting moiety is non-covalently bound to the structure.

16. The device of claim 15, wherein the targeting moiety comprises a polypeptide 80%, 85%, 90%, 95%, or 100% identical to any one of the sequences of Tables 2-3.

17. The device of claim 10, wherein the therapeutic agent comprises a chimeric polypeptide comprising a sequence at least about 80%, 85%, 90%, 95%, or 100% identical to any one of SEQ ID NOS: 433-441.

18. A method of treating a bone defect in a subject in need thereof, the method comprising application of the structure of claim 9 to the defect of the subject.

19. A method of treating a bone defect in a subject in need thereof, the method comprising application of the device of claim 1 to the defect of the subject.

20. The method of claim 19, wherein the defect is present in the spine.
